(19) United States
(12) Patent Application Publication
(10) Pub. No.: US 2024/0300926 A1
(43) Pub. Date: Sep. 12, 2024

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR); Donghee KIM, Daefeon (KR); Joongsuk OH, Daejeon (KR); Da Jung LEE, Daejeon (KR)

(21) Appl. No.: 18/569,407

(22) PCT Filed: May 11, 2022
(86) PCT No.: PCT/KR2022/006739

§ 371 Date: Dec. 12, 2023

(30) Foreign Application Priority Data
Jul. 19, 2021 (KR) 10-2021-0094248

(51) Int. Cl. C07D 405/04; C07D 405/10; C07D 409/14; C09K 11/06; H10K 50/11; H10K 85/60

(52) U.S. Cl. CPC C07D 405/04; C07D 405/10; C07D 409/14; C09K 11/06; H10K 50/11; H10K 85/622; H10K 85/654; H10K 85/6574; C09K 2211/1066

(57) ABSTRACT

A compound of Chemical Formula 1 and an organic light emitting device including the same:

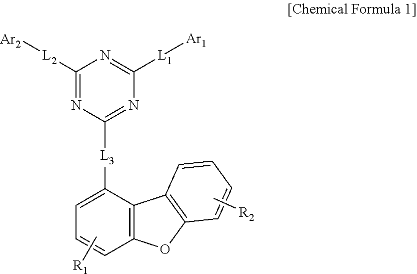

wherein: Ar₁ is unsubstituted benzophenanthrenyl, chrysenyl, or fluoranthenyl; Ar₂ is a substituted or unsubstituted C₆₋₆₀ aryl, or a substituted or unsubstituted C₆₋₆₀ heteroaryl containing any one or more of N, O and S; L₁, L₂, and L₃ each independently is a direct bond or a substituted or unsubstituted C₆₋₆₀ arylene; and R₁ and R₂ are each independently hydrogen, deuterium, a substituted or unsubstituted C₁₋₁₂ alkyl, or a substituted or unsubstituted C₆₋₁₄ aryl. The compound of Chemical Formula 1 can be used as a material for an organic material layer of an organic light emitting device for hole injection, hole transport, hole injection and transport, electron blocking, light emission, electron transport, or electron injection, and can improve the efficiency, achieve low driving voltage and/or improve lifetime characteristics of the device.

### CROSS-REFERENCE TO RELATED APPLICATION(S)

This application is a National Stage Application of International Application No. PCT/KR2022/006739 filed on May 11, 2022, which claims priority to and the benefit of Korean Patent Application No. 10-2021-0094248 filed on Jul. 19, 2021 with the Korean Intellectual Property Office, the content of which is incorporated herein by reference in its entirety.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

For the organic materials used in the organic light emitting devices as described above, the development of new materials is continuously required.

Meanwhile, recently, in order to reduce process costs, an organic light emitting device using a solution process, particularly an inkjet process, has been developed instead of a conventional deposition process. In the initial stage of development, attempts have been made to develop organic light emitting devices by coating all organic light emitting device layers by a solution process, but current technology has limitations. Therefore, only HIL, HTL, and EML are processed by a solution process, and a hybrid process utilizing traditional deposition processes is being studied as a subsequent process.

Therefore, the present disclosure provides a novel material for an organic light emitting device that can be used for an organic light emitting device and at the same time, can be used for a solution process.

### PRIOR ART LITERATURE

### Patent Literature

(Patent Literature 0001) Korean Unexamined Patent Publication No. 10-2000-0051826

### DETAILED DESCRIPTION OF THE INVENTION

### Technical Problem

It is an object of the present disclosure to provide a novel compound and an organic light emitting device comprising the same.

### Technical Solution

According to an aspect of the present disclosure, provided is a compound of Chemical Formula 1:

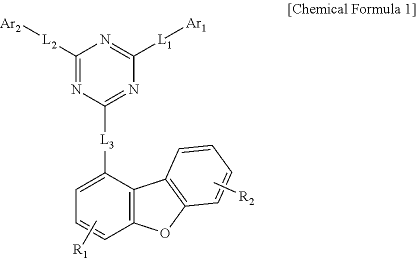

wherein, in Chemical Formula 1:
Ar₁ is unsubstituted benzophenanthrenyl, chrysenyl, or fluoranthenyl;
Ar₂ is a substituted or unsubstituted C₆₋₆₀ aryl, or a substituted or unsubstituted C₆₋₆₀ heteroaryl containing any one or more selected from the group consisting of N, O and S;
L₁ is a direct bond or a substituted or unsubstituted C₆₋₆₀ arylene;
L₂ is a direct bond; or a substituted or unsubstituted C₆₋₆₀ arylene;
L₃ is a direct bond or a substituted or unsubstituted C₆₋₆₀ arylene; and
R₁ and R₂ are each independently hydrogen, deuterium, a substituted or unsubstituted C₁₋₁₂ alkyl, or a substituted or unsubstituted C₆₋₁₄ aryl.

According to another aspect of the present disclosure, provided is an organic light emitting device comprising: a first electrode; a second electrode that is opposite to the first electrode; and an organic material layer that is between the first electrode and the second electrode, wherein the organic material layer includes the compound of Chemical Formula 1. Specifically, the organic material layer comprising the compound can be an electronic light emitting layer.

### Advantageous Effects

The above-mentioned compound of Chemical Formula 1 can be used as a material for an organic material layer of an organic light emitting device, and can improve the efficiency, achieve low driving voltage and/or improve lifetime characteristics in the organic light emitting device. In particular, the compound of Chemical Formula 1 described above can be used as a material for hole injection, hole transport, hole injection and transport, electron blocking, light emission, electron transport, or electron injection.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a light emitting layer 7, an electron injection and transport layer 8, and a cathode 4.

### DETAILED DESCRIPTION OF THE EMBODIMENTS

Hereinafter, embodiments of the present disclosure will be described in more detail to facilitate understanding of the invention.

### Definition of Terms

As used herein, the notation

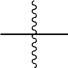

and [symbol] mean a bond linked to another substituent group.

As used herein, the term “substituted or unsubstituted” means being unsubstituted or substituted with one or more substituents selected from the group consisting of deuterium, a halogen group, a cyano group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenyl group can be an aryl group, or it can also be interpreted as a substituent in which two phenyl groups are linked.

In the present disclosure, the carbon number of a carbonyl group is not particularly limited, but is preferably 1 to 40. Specifically, the carbonyl group can be a substituent group having the following structural formulas, but is not limited thereto:

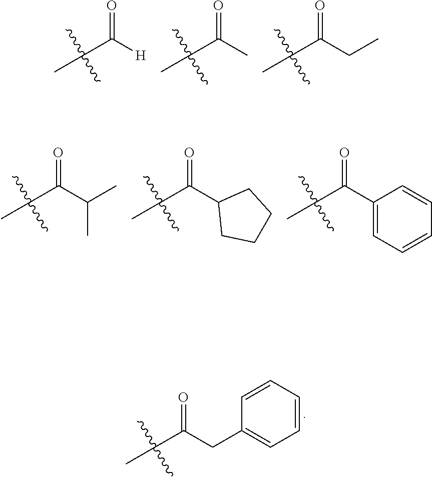

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a substituent group having the following structural formulas, but is not limited thereto:

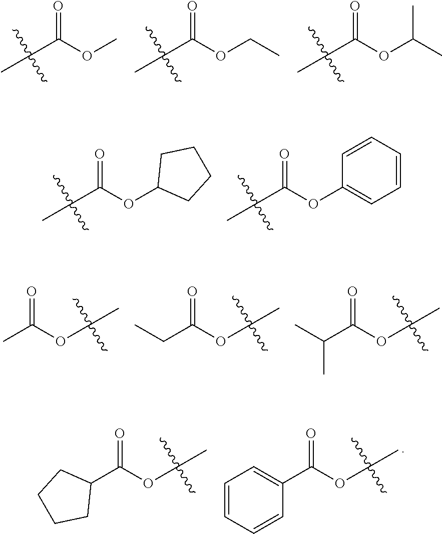

In the present disclosure, the carbon number of an imide group is not particularly limited, but is preferably 1 to 25. Specifically, the imide group can be a substituent group having the following structural formulas, but is not limited thereto:

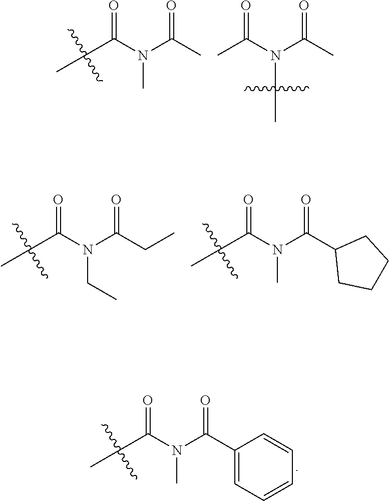

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group can be substituted, and two substituents can be connected to each other to form a spiro structure. In the case where the fluorenyl group is substituted,

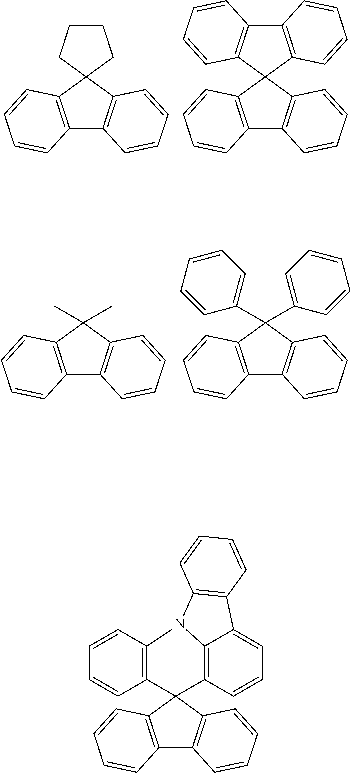

and the like can be formed. However, the structure is not limited thereto

In the present disclosure, a heteroaryl is a heteroaryl containing at least one of O, N, Si and S as a heteroatom, and the carbon number thereof is not particularly limited, but is preferably 2 to 60. Examples of the heteroaryl include xanthene, thioxanthene, a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, the arylamine group and the arylsily group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine can be applied to the above-mentioned description of the heteroaryl. In the present disclosure, the alkenyl group in the aralkenyl group is the same as the above-mentioned examples of the alkenyl group. In the present disclosure, the above-mentioned description of the aryl group can be applied except that the arylene is a divalent group. In the present disclosure, the above-mentioned description of the heteroaryl can be applied except that the heteroarylene is a divalent group. In the present disclosure, the above-mentioned description of the aryl group or cycloalkyl group can be applied except that the hydrocarbon ring is not a monovalent group but formed by combining two substituent groups. In the present disclosure, the above-mentioned description of the heteroaryl can be applied, except that the heterocyclic group is not a monovalent group but formed by combining two substituent groups.

### Compound

The present disclosure provides the compound of Chemical Formula 1.

In Chemical Formula 1, Ar₁ is unsubstituted benzophenanthrenyl, chrysenyl, or fluoranthenyl. Preferably, it can be 3,4-benzophenanthrenyl, chrysenyl or fluoranthenyl.

The Ar₂ is a substituted or unsubstituted C₆₋₆₀ aryl, or a substituted or unsubstituted C₆₋₆₀ heteroaryl containing any one or more heteroatoms selected from the group consisting of N, O and S. Preferably, it can be substituted or unsubstituted phenyl, biphenyl, naphthyl, dibenzofuranyl, or dibenzothiophenyl.
L₁ is a direct bond or a substituted or unsubstituted C₆₋₆₀ arylene. Preferably, L₁ is a direct bond or phenylene.
L₂ is a direct bond or substituted or unsubstituted C₆₋₆₀ arylene. Preferably, L₂ is a direct bond; or phenylene.
L₃ is a direct bond or a substituted or unsubstituted C₆₋₆₀ arylene. Preferably, L₃ is a direct bond phenylene; or naphthylene.
R₁ and R₂ are each independently hydrogen, deuterium, a substituted or unsubstituted C₁₋₁₂ alkyl, or a substituted or unsubstituted C₆₋₁₄ aryl. Preferably, R₁ and R₂ can be each independently hydrogen, deuterium, phenyl, biphenyl or naphthyl.

Representative examples of the compound of Chemical Formula 1 are as follows:

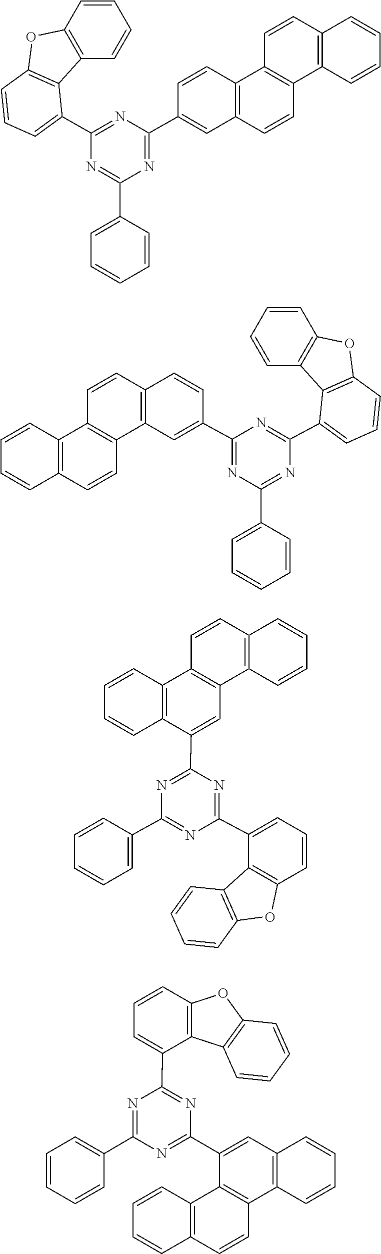

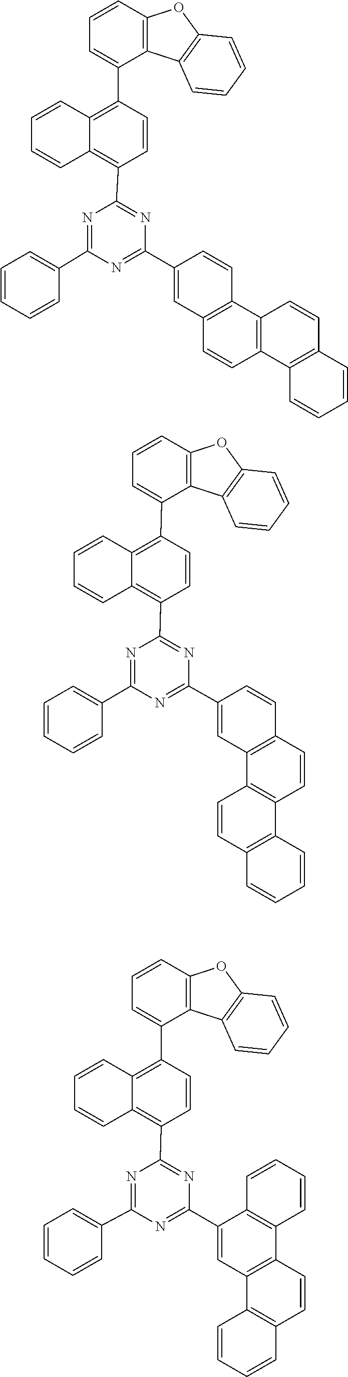 [SMILES: c1ccc(-c2nc(-c3ccc4c(ccc5c6ccccc6ccc45)c3)nc(-c3ccc(-c4cccc5oc6ccccc6c45)c4ccccc34)n2)cc1 ; c1ccc(-c2nc(-c3ccc4ccc5c6ccccc6ccc5c4c3)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3cc4c5ccccc5ccc4c4ccccc34)n2)cc1]

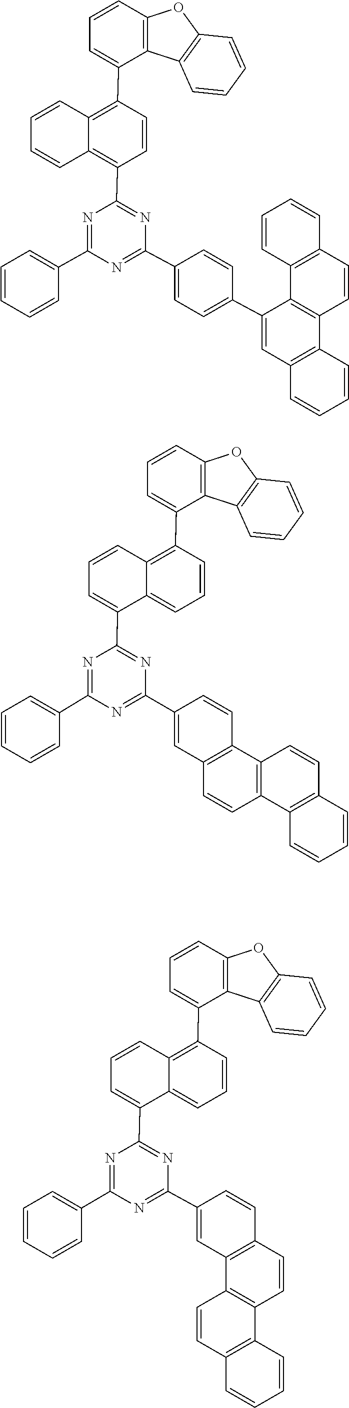 [SMILES: c1ccc(-c2nc(-c3ccc(-c4cc5ccccc5c5ccc6ccccc6c45)cc3)nc(-c3ccc(-c4cccc5oc6ccccc6c45)c4ccccc34)n2)cc1 ; c1ccc(-c2nc(-c3ccc4c(ccc5c6ccccc6ccc45)c3)nc(-c3cccc4c(-c5cccc6oc7ccccc7c56)cccc34)n2)cc1 ; c1ccc(-c2nc(-c3ccc4ccc5c6ccccc6ccc5c4c3)nc(-c3cccc4c(-c5cccc6oc7ccccc7c56)cccc34)n2)cc1]

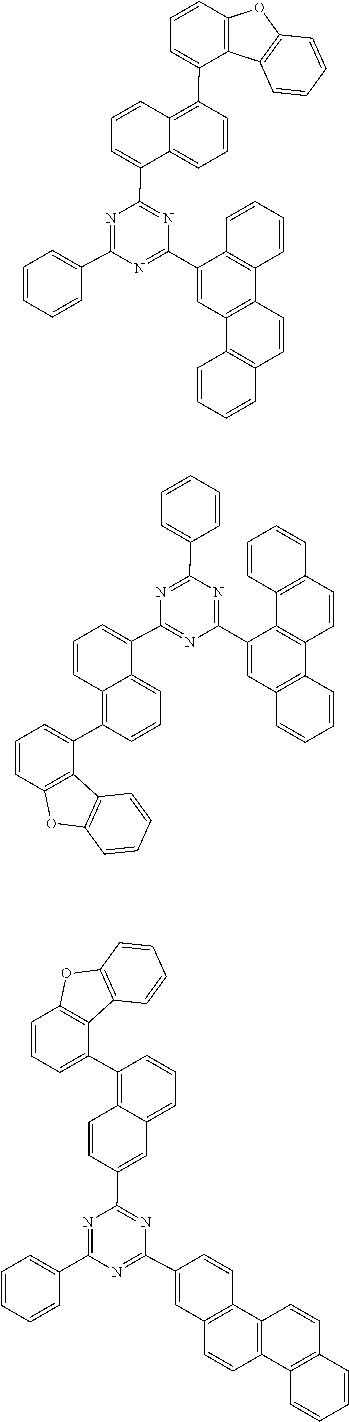 [SMILES: c1ccc(-c2nc(-c3cccc4c(-c5cccc6oc7ccccc7c56)cccc34)nc(-c3cc4c5ccccc5ccc4c4ccccc34)n2)cc1 ; c1ccc(-c2nc(-c3cccc4c(-c5cccc6oc7ccccc7c56)cccc34)nc(-c3cc4ccccc4c4ccc5ccccc5c34)n2)cc1 ; c1ccc(-c2nc(-c3ccc4c(-c5cccc6oc7ccccc7c56)cccc4c3)nc(-c3ccc4c(ccc5c6ccccc6ccc45)c3)n2)cc1]

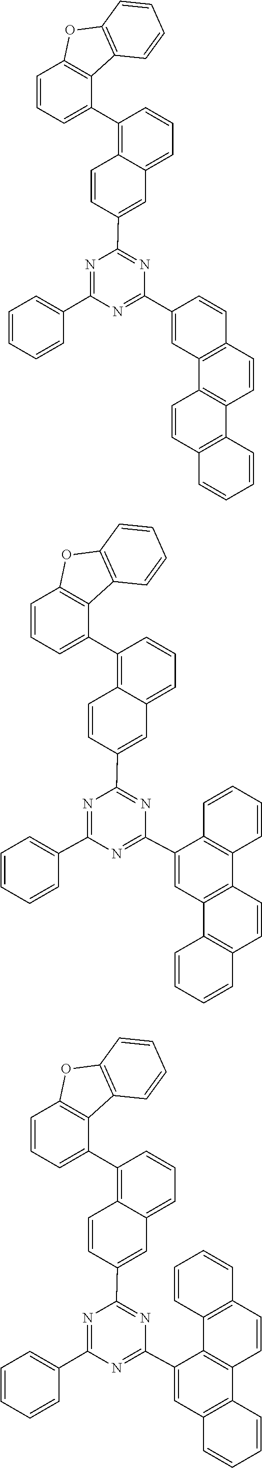 [SMILES: c1ccc(-c2nc(-c3ccc4c(-c5cccc6oc7ccccc7c56)cccc4c3)nc(-c3ccc4ccc5c6ccccc6ccc5c4c3)n2)cc1 ; c1ccc(-c2nc(-c3ccc4c(-c5cccc6oc7ccccc7c56)cccc4c3)nc(-c3cc4c5ccccc5ccc4c4ccccc34)n2)cc1 ; c1ccc(-c2nc(-c3ccc4c(-c5cccc6oc7ccccc7c56)cccc4c3)nc(-c3cc4ccccc4c4ccc5ccccc5c34)n2)cc1]

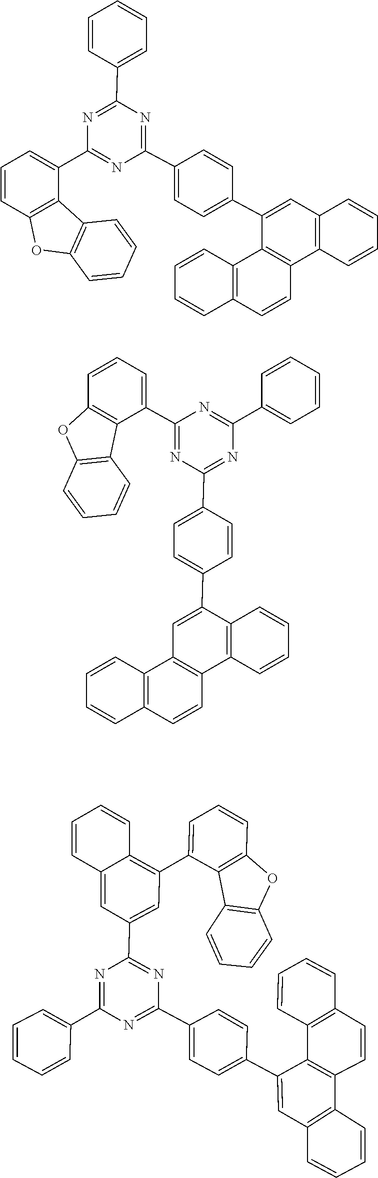 [SMILES: c1ccc(-c2nc(-c3ccc(-c4cc5ccccc5c5ccc6ccccc6c45)cc3)nc(-c3cccc4oc5ccccc5c34)n2)cc1 ; c1ccc(-c2nc(-c3ccc(-c4cc5c6ccccc6ccc5c5ccccc45)cc3)nc(-c3cccc4oc5ccccc5c34)n2)cc1 ; c1ccc(-c2nc(-c3ccc(-c4cc5ccccc5c5ccc6ccccc6c45)cc3)nc(-c3cc(-c4cccc5oc6ccccc6c45)c4ccccc4c3)n2)cc1]

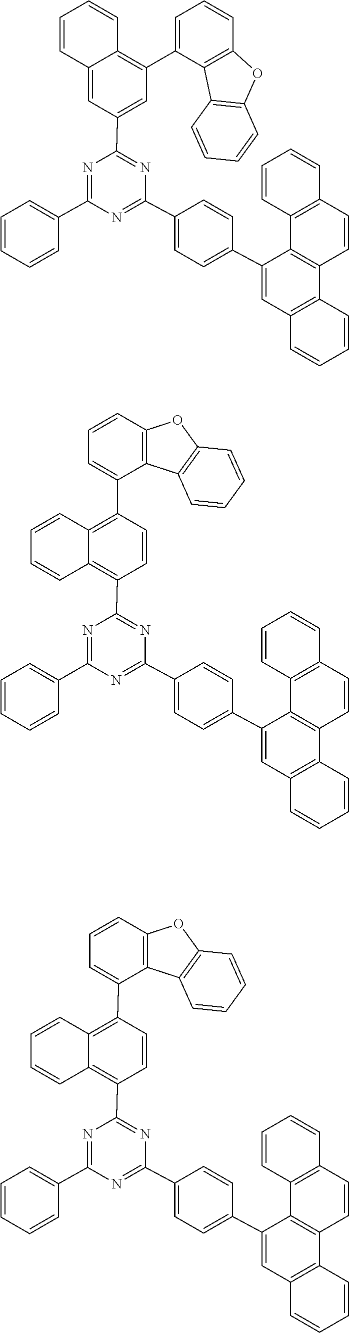 [SMILES: c1ccc(-c2nc(-c3ccc(-c4cc5ccccc5c5ccc6ccccc6c45)cc3)nc(-c3cc(-c4cccc5oc6ccccc6c45)c4ccccc4c3)n2)cc1 ; c1ccc(-c2nc(-c3ccc(-c4cc5ccccc5c5ccc6ccccc6c45)cc3)nc(-c3ccc(-c4cccc5oc6ccccc6c45)c4ccccc34)n2)cc1 ; c1ccc(-c2nc(-c3ccc(-c4cc5ccccc5c5ccc6ccccc6c45)cc3)nc(-c3ccc(-c4cccc5oc6ccccc6c45)c4ccccc34)n2)cc1]

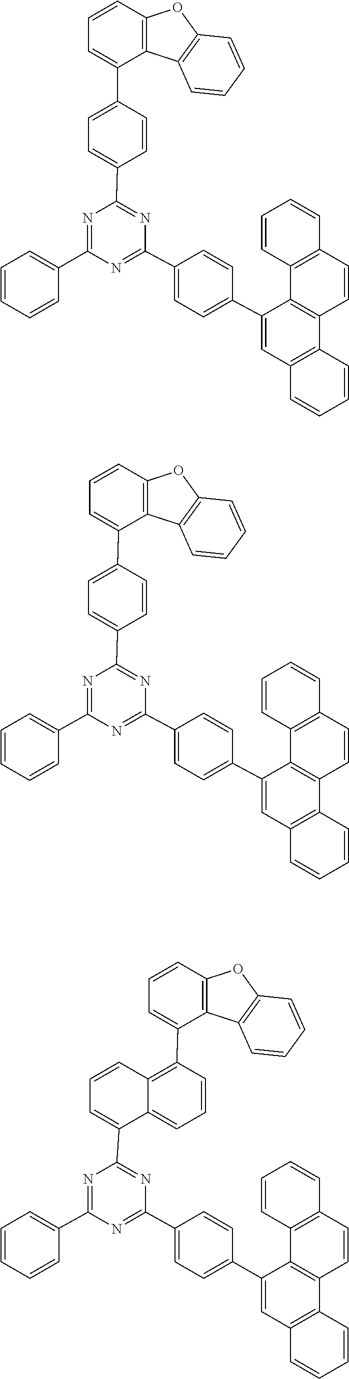 [SMILES: c1ccc(-c2nc(-c3ccc(-c4cccc5oc6ccccc6c45)cc3)nc(-c3ccc(-c4cc5ccccc5c5ccc6ccccc6c45)cc3)n2)cc1 ; c1ccc(-c2nc(-c3ccc(-c4cccc5oc6ccccc6c45)cc3)nc(-c3ccc(-c4cc5ccccc5c5ccc6ccccc6c45)cc3)n2)cc1 ; c1ccc(-c2nc(-c3ccc(-c4cc5ccccc5c5ccc6ccccc6c45)cc3)nc(-c3cccc4c(-c5cccc6oc7ccccc7c56)cccc34)n2)cc1]

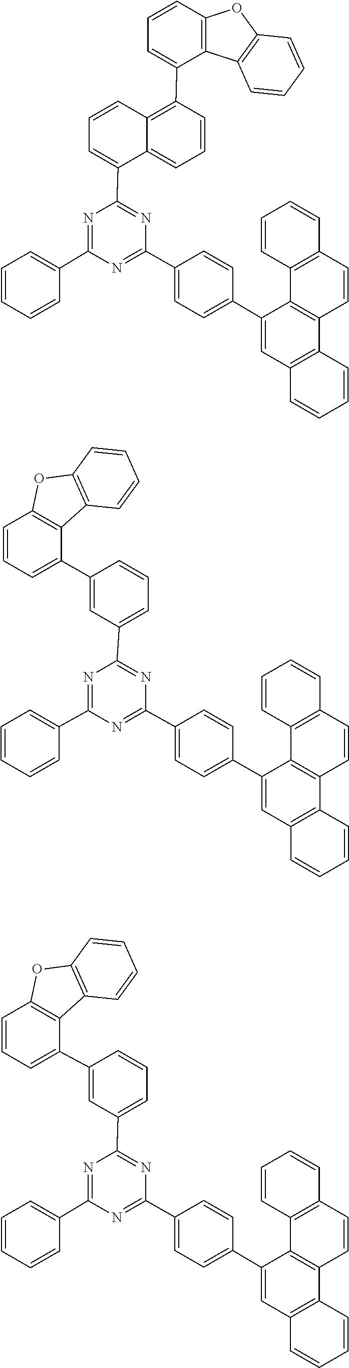 [SMILES: c1ccc(-c2nc(-c3ccc(-c4cc5ccccc5c5ccc6ccccc6c45)cc3)nc(-c3cccc4c(-c5cccc6oc7ccccc7c56)cccc34)n2)cc1 ; c1ccc(-c2nc(-c3ccc(-c4cc5ccccc5c5ccc6ccccc6c45)cc3)nc(-c3cccc(-c4cccc5oc6ccccc6c45)c3)n2)cc1 ; c1ccc(-c2nc(-c3ccc(-c4cc5ccccc5c5ccc6ccccc6c45)cc3)nc(-c3cccc(-c4cccc5oc6ccccc6c45)c3)n2)cc1]

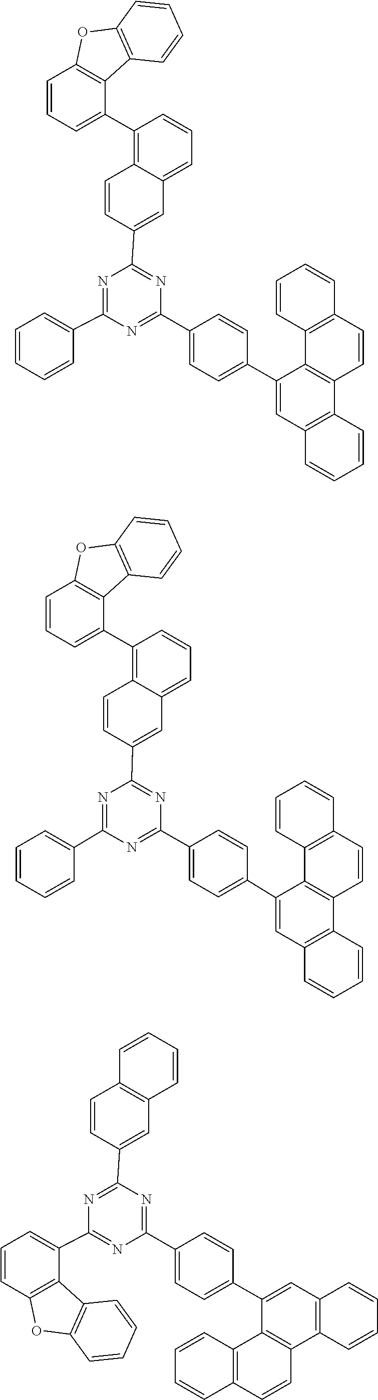 [SMILES: c1ccc(-c2nc(-c3ccc(-c4cc5ccccc5c5ccc6ccccc6c45)cc3)nc(-c3ccc4c(-c5cccc6oc7ccccc7c56)cccc4c3)n2)cc1 ; c1ccc(-c2nc(-c3ccc(-c4cc5ccccc5c5ccc6ccccc6c45)cc3)nc(-c3ccc4c(-c5cccc6oc7ccccc7c56)cccc4c3)n2)cc1 ; c1ccc2cc(-c3nc(-c4ccc(-c5cc6ccccc6c6ccc7ccccc7c56)cc4)nc(-c4cccc5oc6ccccc6c45)n3)ccc2c1]

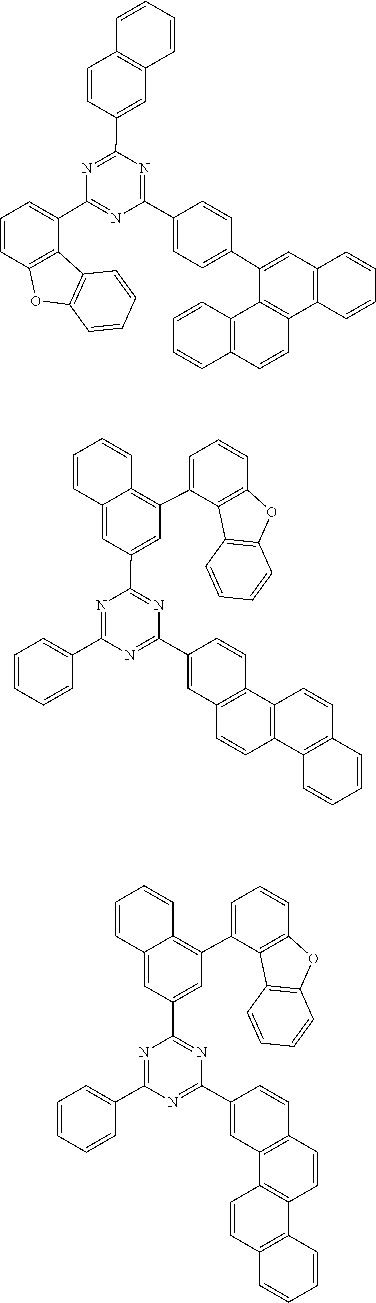 [SMILES: c1ccc2cc(-c3nc(-c4ccc(-c5cc6ccccc6c6ccc7ccccc7c56)cc4)nc(-c4cccc5oc6ccccc6c45)n3)ccc2c1 ; c1ccc(-c2nc(-c3cc(-c4cccc5oc6ccccc6c45)c4ccccc4c3)nc(-c3ccc4c(ccc5c6ccccc6ccc45)c3)n2)cc1 ; c1ccc(-c2nc(-c3cc(-c4cccc5oc6ccccc6c45)c4ccccc4c3)nc(-c3ccc4ccc5c6ccccc6ccc5c4c3)n2)cc1]

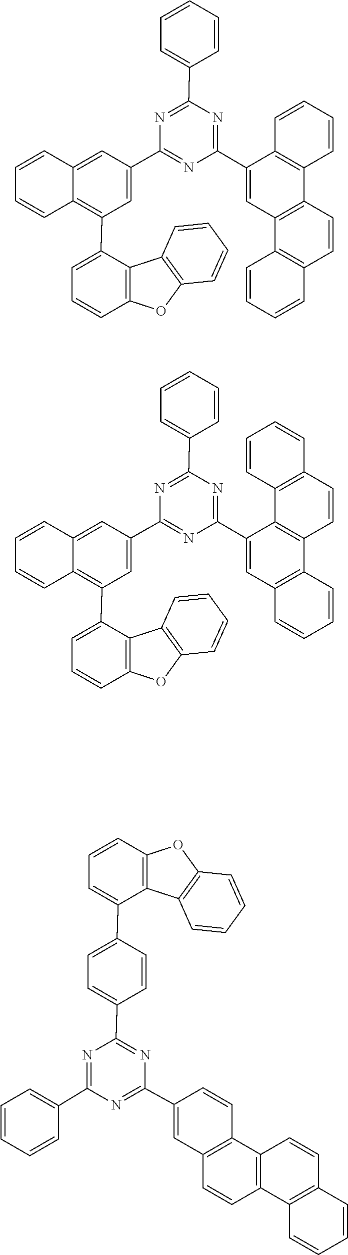 [SMILES: c1ccc(-c2nc(-c3cc(-c4cccc5oc6ccccc6c45)c4ccccc4c3)nc(-c3cc4c5ccccc5ccc4c4ccccc34)n2)cc1 ; c1ccc(-c2nc(-c3cc(-c4cccc5oc6ccccc6c45)c4ccccc4c3)nc(-c3cc4ccccc4c4ccc5ccccc5c34)n2)cc1 ; c1ccc(-c2nc(-c3ccc(-c4cccc5oc6ccccc6c45)cc3)nc(-c3ccc4c(ccc5c6ccccc6ccc45)c3)n2)cc1]

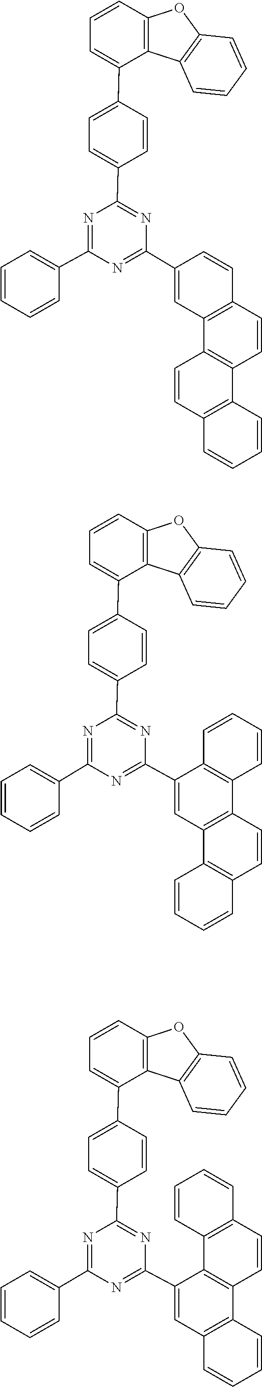 [SMILES: c1ccc(-c2nc(-c3ccc(-c4cccc5oc6ccccc6c45)cc3)nc(-c3ccc4ccc5c6ccccc6ccc5c4c3)n2)cc1 ; c1ccc(-c2nc(-c3ccc(-c4cccc5oc6ccccc6c45)cc3)nc(-c3cc4c5ccccc5ccc4c4ccccc34)n2)cc1 ; c1ccc(-c2nc(-c3ccc(-c4cccc5oc6ccccc6c45)cc3)nc(-c3cc4ccccc4c4ccc5ccccc5c34)n2)cc1]

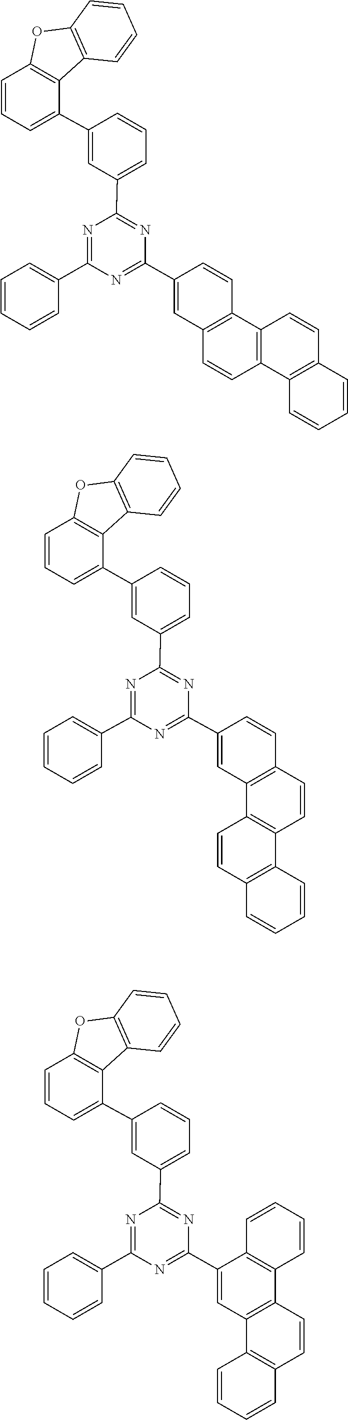 [SMILES: c1ccc(-c2nc(-c3cccc(-c4cccc5oc6ccccc6c45)c3)nc(-c3ccc4c(ccc5c6ccccc6ccc45)c3)n2)cc1 ; c1ccc(-c2nc(-c3cccc(-c4cccc5oc6ccccc6c45)c3)nc(-c3ccc4ccc5c6ccccc6ccc5c4c3)n2)cc1 ; c1ccc(-c2nc(-c3cccc(-c4cccc5oc6ccccc6c45)c3)nc(-c3cc4c5ccccc5ccc4c4ccccc34)n2)cc1]

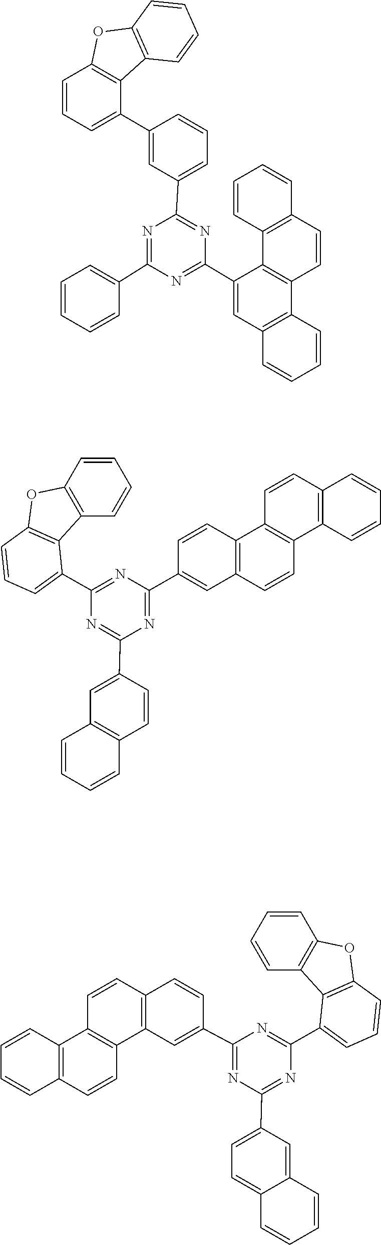 [SMILES: c1ccc(-c2nc(-c3cccc(-c4cccc5oc6ccccc6c45)c3)nc(-c3cc4ccccc4c4ccc5ccccc5c34)n2)cc1 ; c1ccc2cc(-c3nc(-c4ccc5c(ccc6c7ccccc7ccc56)c4)nc(-c4cccc5oc6ccccc6c45)n3)ccc2c1 ; c1ccc2cc(-c3nc(-c4ccc5ccc6c7ccccc7ccc6c5c4)nc(-c4cccc5oc6ccccc6c45)n3)ccc2c1]

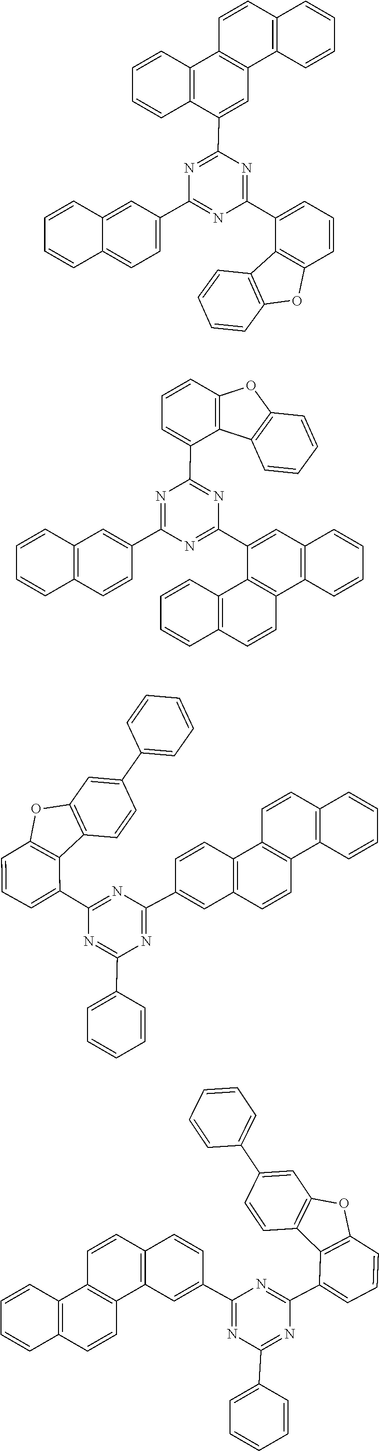 [SMILES: c1ccc2cc(-c3nc(-c4cc5c6ccccc6ccc5c5ccccc45)nc(-c4cccc5oc6ccccc6c45)n3)ccc2c1 ; c1ccc2cc(-c3nc(-c4cccc5oc6ccccc6c45)nc(-c4cc5ccccc5c5ccc6ccccc6c45)n3)ccc2c1 ; c1ccc(-c2ccc3c(c2)oc2cccc(-c4nc(-c5ccccc5)nc(-c5ccc6c(ccc7c8ccccc8ccc67)c5)n4)c23)cc1 ; c1ccc(-c2ccc3c(c2)oc2cccc(-c4nc(-c5ccccc5)nc(-c5ccc6ccc7c8ccccc8ccc7c6c5)n4)c23)cc1]

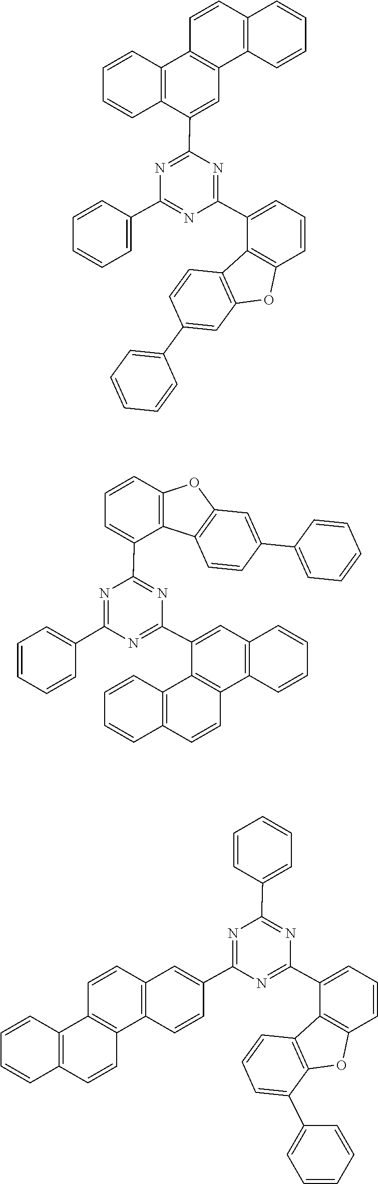 [SMILES: c1ccc(-c2ccc3c(c2)oc2cccc(-c4nc(-c5ccccc5)nc(-c5cc6c7ccccc7ccc6c6ccccc56)n4)c23)cc1 ; c1ccc(-c2ccc3c(c2)oc2cccc(-c4nc(-c5ccccc5)nc(-c5cc6ccccc6c6ccc7ccccc7c56)n4)c23)cc1 ; c1ccc(-c2nc(-c3ccc4c(ccc5c6ccccc6ccc45)c3)nc(-c3cccc4oc5c(-c6ccccc6)cccc5c34)n2)cc1]

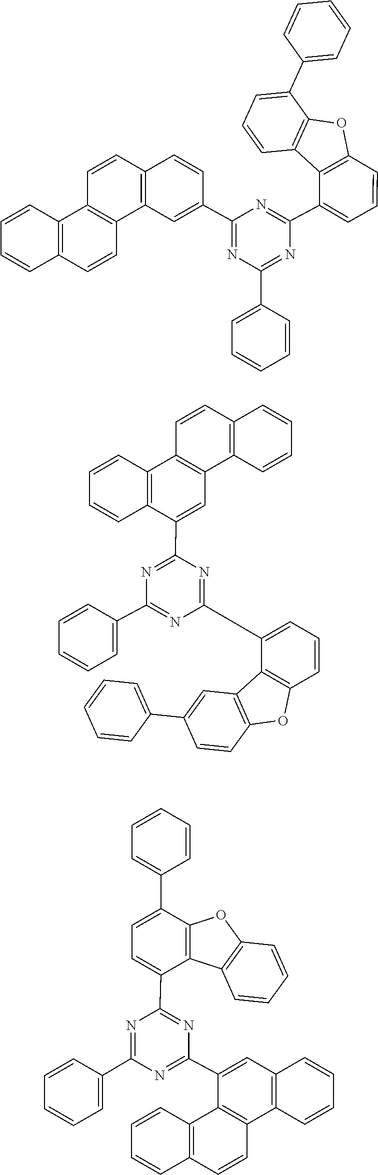

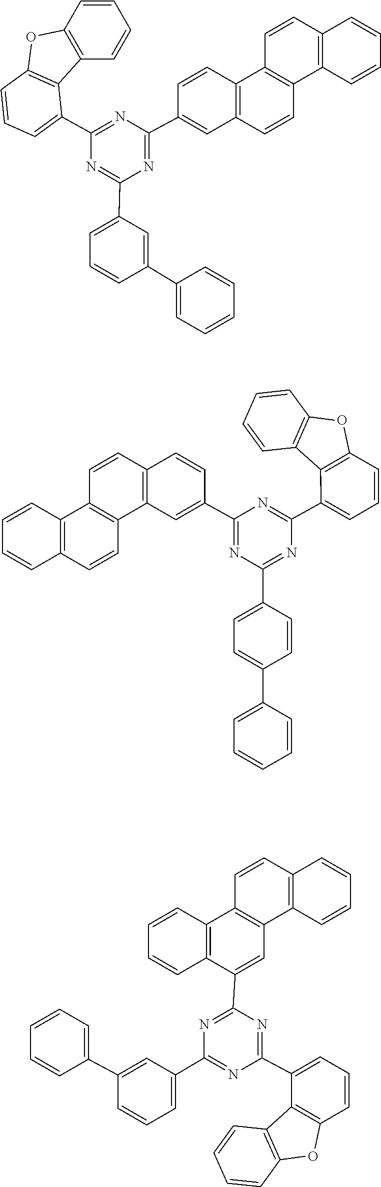 [SMILES: c1ccc(-c2cccc(-c3nc(-c4ccc5c(ccc6c7ccccc7ccc56)c4)nc(-c4cccc5oc6ccccc6c45)n3)c2)cc1 ; c1ccc(-c2ccc(-c3nc(-c4ccc5ccc6c7ccccc7ccc6c5c4)nc(-c4cccc5oc6ccccc6c45)n3)cc2)cc1 ; c1ccc(-c2cccc(-c3nc(-c4cc5c6ccccc6ccc5c5ccccc45)nc(-c4cccc5oc6ccccc6c45)n3)c2)cc1]

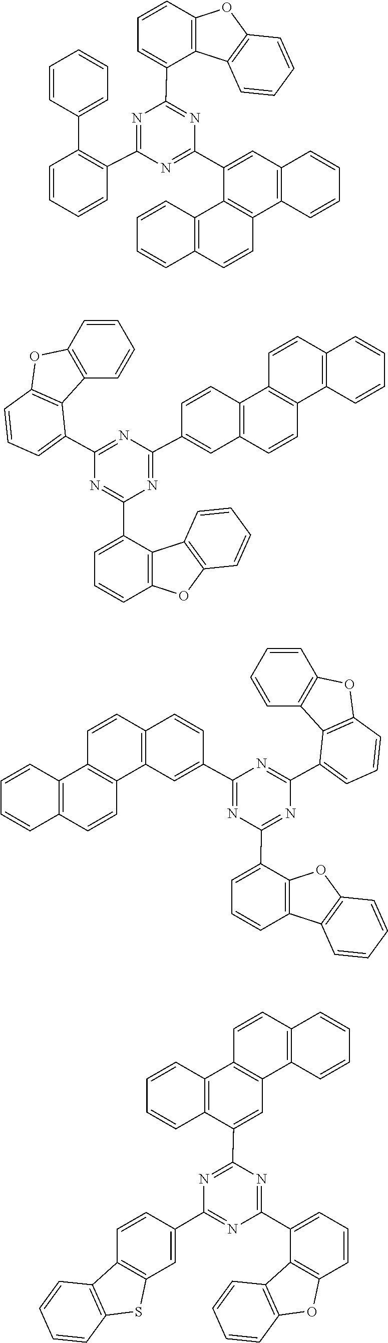 [SMILES: c1ccc(-c2ccccc2-c2nc(-c3cccc4oc5ccccc5c34)nc(-c3cc4ccccc4c4ccc5ccccc5c34)n2)cc1 ; c1ccc2c(c1)ccc1c3ccc(-c4nc(-c5cccc6oc7ccccc7c56)nc(-c5cccc6oc7ccccc7c56)n4)cc3ccc21 ; c1ccc2c(c1)ccc1c3cc(-c4nc(-c5cccc6c5oc5ccccc56)nc(-c5cccc6oc7ccccc7c56)n4)ccc3ccc21 ; c1ccc2c(c1)ccc1c3ccccc3c(-c3nc(-c4ccc5c(c4)sc4ccccc45)nc(-c4cccc5oc6ccccc6c45)n3)cc21]

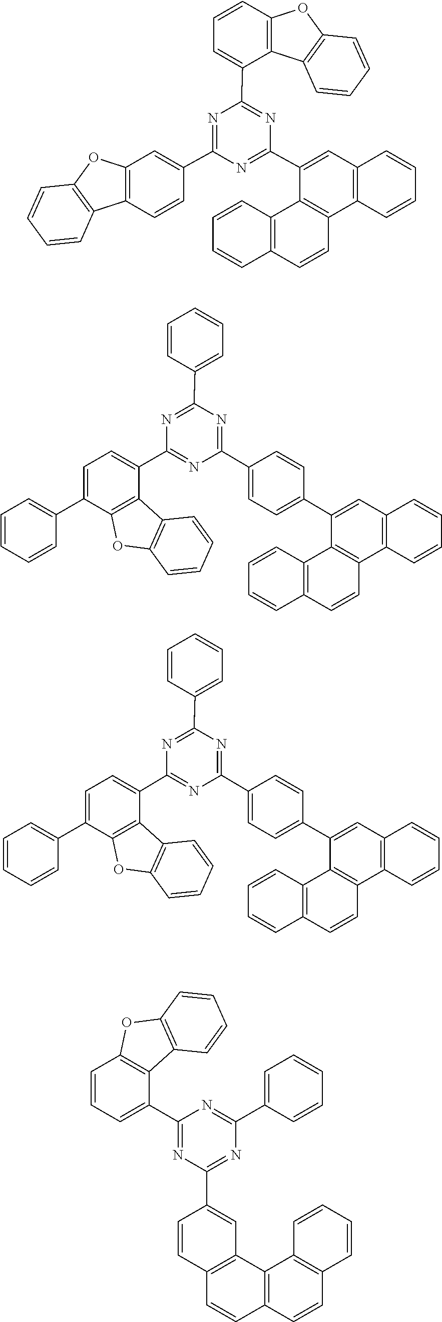 [SMILES: c1ccc2c(c1)cc(-c1nc(-c3ccc4c(c3)oc3ccccc34)nc(-c3cccc4oc5ccccc5c34)n1)c1c3ccccc3ccc21 ; c1ccc(-c2nc(-c3ccc(-c4cc5ccccc5c5ccc6ccccc6c45)cc3)nc(-c3ccc(-c4ccccc4)c4oc5ccccc5c34)n2)cc1 ; c1ccc(-c2nc(-c3ccc(-c4cc5ccccc5c5ccc6ccccc6c45)cc3)nc(-c3ccc(-c4ccccc4)c4oc5ccccc5c34)n2)cc1 ; c1ccc(-c2nc(-c3ccc4ccc5ccc6ccccc6c5c4c3)nc(-c3cccc4oc5ccccc5c34)n2)cc1]

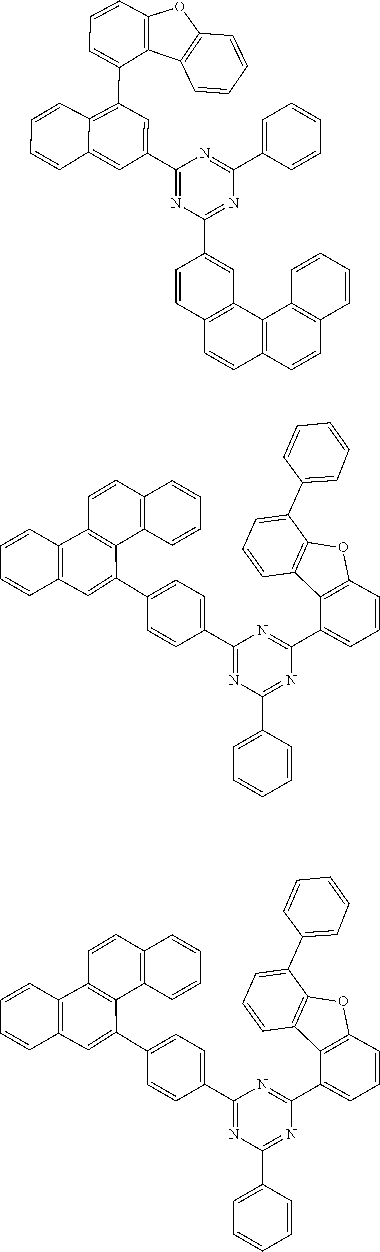 [SMILES: c1ccc(-c2nc(-c3cc(-c4cccc5oc6ccccc6c45)c4ccccc4c3)nc(-c3ccc4ccc5ccc6ccccc6c5c4c3)n2)cc1 ; c1ccc(-c2nc(-c3ccc(-c4cc5ccccc5c5ccc6ccccc6c45)cc3)nc(-c3cccc4oc5c(-c6ccccc6)cccc5c34)n2)cc1 ; c1ccc(-c2nc(-c3ccc(-c4cc5ccccc5c5ccc6ccccc6c45)cc3)nc(-c3cccc4oc5c(-c6ccccc6)cccc5c34)n2)cc1]

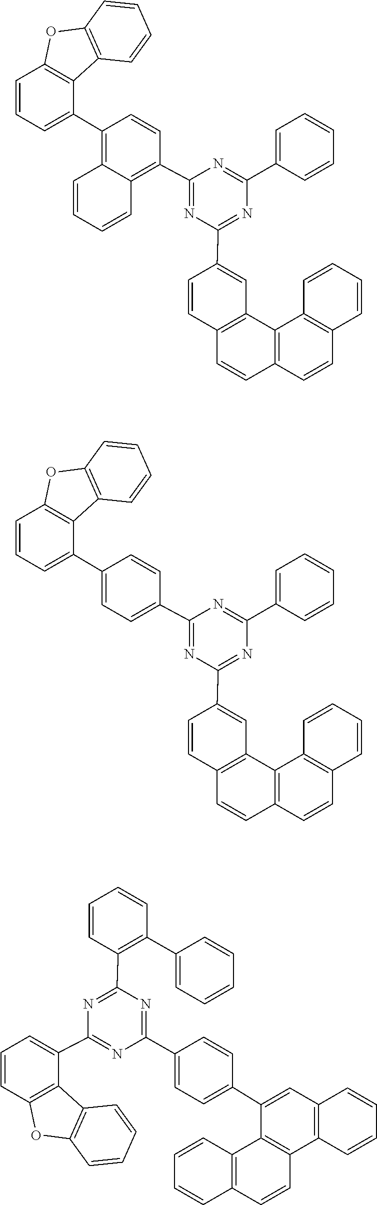 [SMILES: c1ccc(-c2nc(-c3ccc4ccc5ccc6ccccc6c5c4c3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccc5ccc6ccccc6c5c4c3)n2)cc1 ; c1ccc(-c2ccccc2-c2nc(-c3ccc(-c4cc5ccccc5c5ccc6ccccc6c45)cc3)nc(-c3cccc4oc5ccccc5c34)n2)cc1]

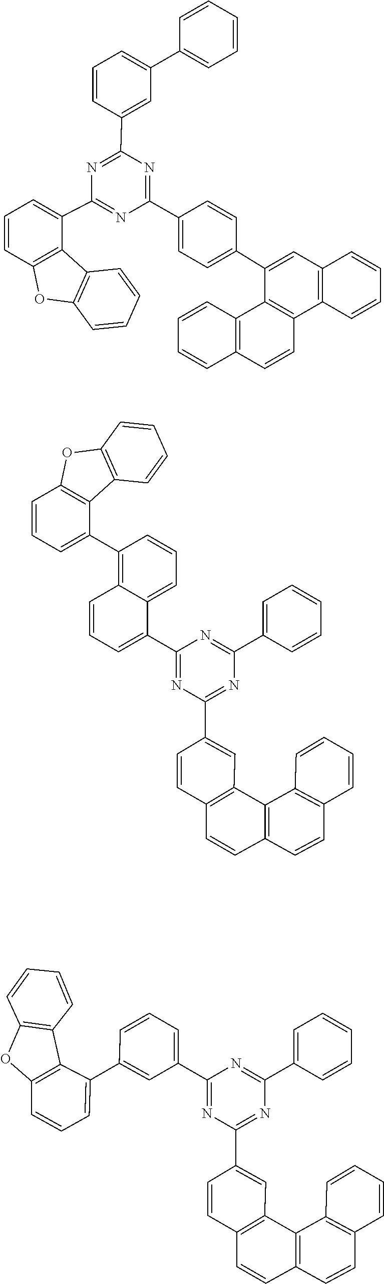 [SMILES: c1ccc(-c2cccc(-c3nc(-c4ccc(-c5cc6ccccc6c6ccc7ccccc7c56)cc4)nc(-c4cccc5oc6ccccc6c45)n3)c2)cc1 ; c1ccc(-c2nc(-c3ccc4ccc5ccc6ccccc6c5c4c3)nc(-c3cccc4c(-c5cccc6oc7ccccc7c56)cccc34)n2)cc1 ; c1ccc(-c2nc(-c3cccc(-c4cccc5oc6ccccc6c45)c3)nc(-c3ccc4ccc5ccc6ccccc6c5c4c3)n2)cc1]

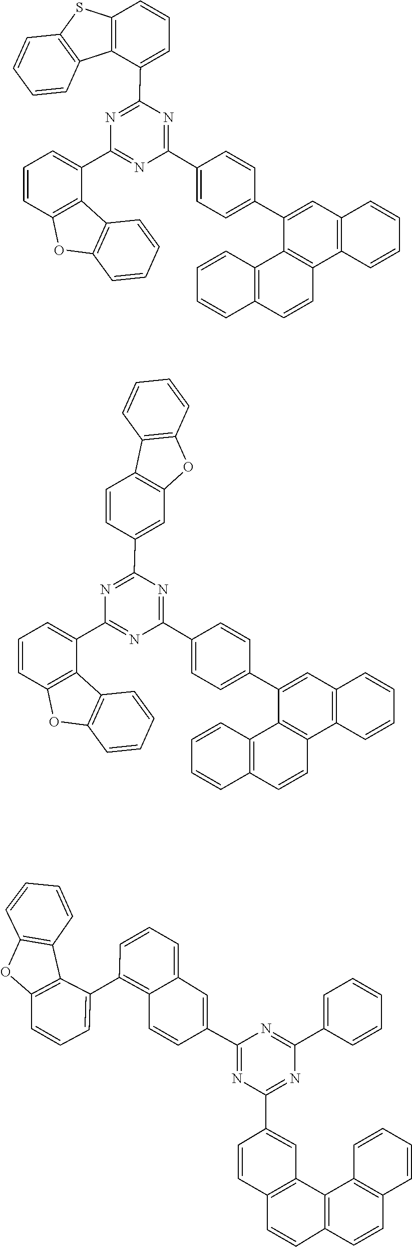 [SMILES: c1ccc2c(c1)cc(-c1ccc(-c3nc(-c4cccc5oc6ccccc6c45)nc(-c4cccc5sc6ccccc6c45)n3)cc1)c1c3ccccc3ccc21 ; c1ccc2c(c1)cc(-c1ccc(-c3nc(-c4ccc5c(c4)oc4ccccc45)nc(-c4cccc5oc6ccccc6c45)n3)cc1)c1c3ccccc3ccc21 ; c1ccc(-c2nc(-c3ccc4c(-c5cccc6oc7ccccc7c56)cccc4c3)nc(-c3ccc4ccc5ccc6ccccc6c5c4c3)n2)cc1]

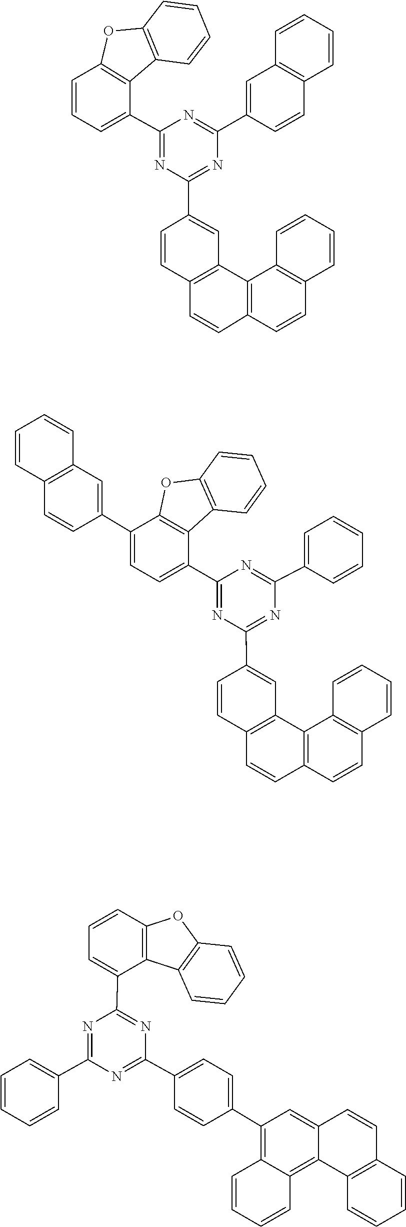 [SMILES: c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccc4ccc5ccc6ccccc6c5c4c3)nc(-c3ccc(-c4ccc5ccccc5c4)c4oc5ccccc5c34)n2)cc1 ; c1ccc(-c2nc(-c3ccc(-c4cc5ccc6ccccc6c5c5ccccc45)cc3)nc(-c3cccc4oc5ccccc5c34)n2)cc1]

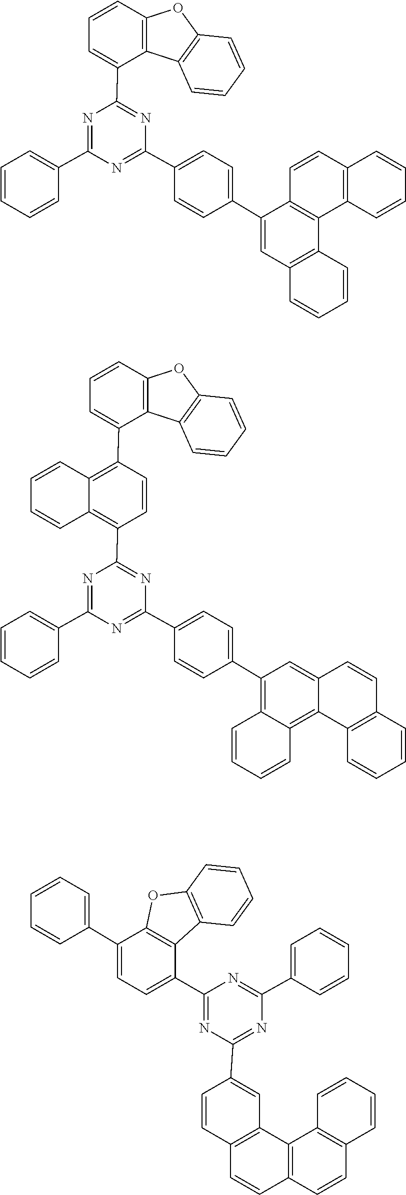 [SMILES: c1ccc(-c2nc(-c3ccc(-c4cc5ccccc5c5c4ccc4ccccc45)cc3)nc(-c3cccc4oc5ccccc5c34)n2)cc1 ; c1ccc(-c2nc(-c3ccc(-c4cc5ccc6ccccc6c5c5ccccc45)cc3)nc(-c3ccc(-c4cccc5oc6ccccc6c45)c4ccccc34)n2)cc1 ; c1ccc(-c2nc(-c3ccc4ccc5ccc6ccccc6c5c4c3)nc(-c3ccc(-c4ccccc4)c4oc5ccccc5c34)n2)cc1]

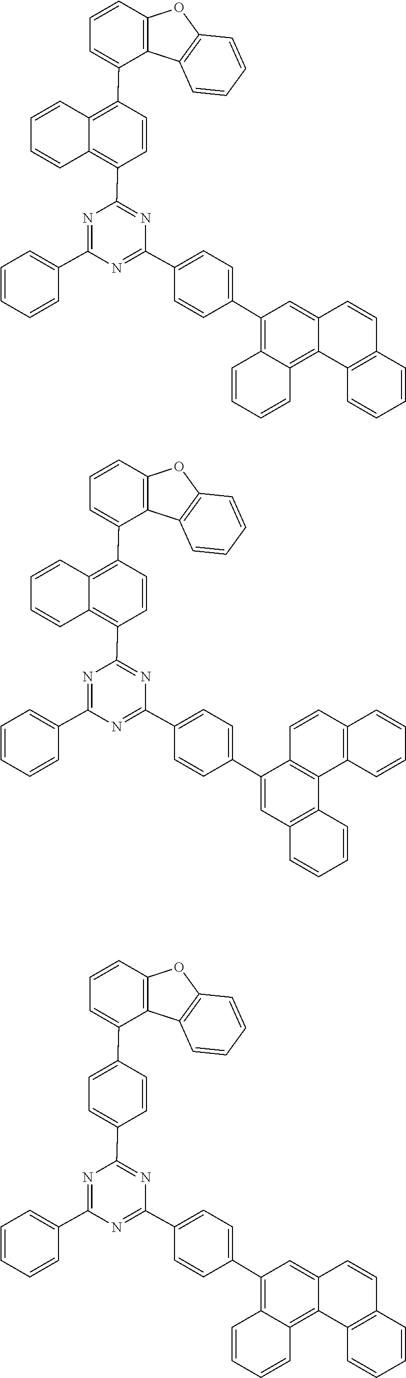 [SMILES: c1ccc(-c2nc(-c3ccc(-c4cc5ccc6ccccc6c5c5ccccc45)cc3)nc(-c3ccc(-c4cccc5oc6ccccc6c45)c4ccccc34)n2)cc1 ; c1ccc(-c2nc(-c3ccc(-c4cc5ccccc5c5c4ccc4ccccc45)cc3)nc(-c3ccc(-c4cccc5oc6ccccc6c45)c4ccccc34)n2)cc1 ; c1ccc(-c2nc(-c3ccc(-c4cc5ccc6ccccc6c5c5ccccc45)cc3)nc(-c3ccc(-c4cccc5oc6ccccc6c45)cc3)n2)cc1]

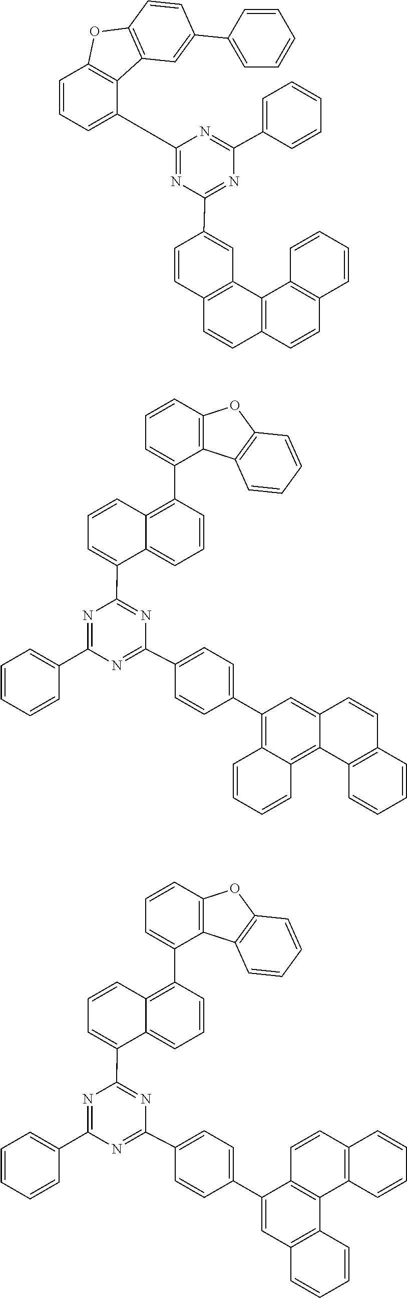 [SMILES: c1ccc(-c2ccc3oc4cccc(-c5nc(-c6ccccc6)nc(-c6ccc7ccc8ccc9ccccc9c8c7c6)n5)c4c3c2)cc1 ; c1ccc(-c2nc(-c3ccc(-c4cc5ccc6ccccc6c5c5ccccc45)cc3)nc(-c3cccc4c(-c5cccc6oc7ccccc7c56)cccc34)n2)cc1 ; c1ccc(-c2nc(-c3ccc(-c4cc5ccccc5c5c4ccc4ccccc45)cc3)nc(-c3cccc4c(-c5cccc6oc7ccccc7c56)cccc34)n2)cc1]

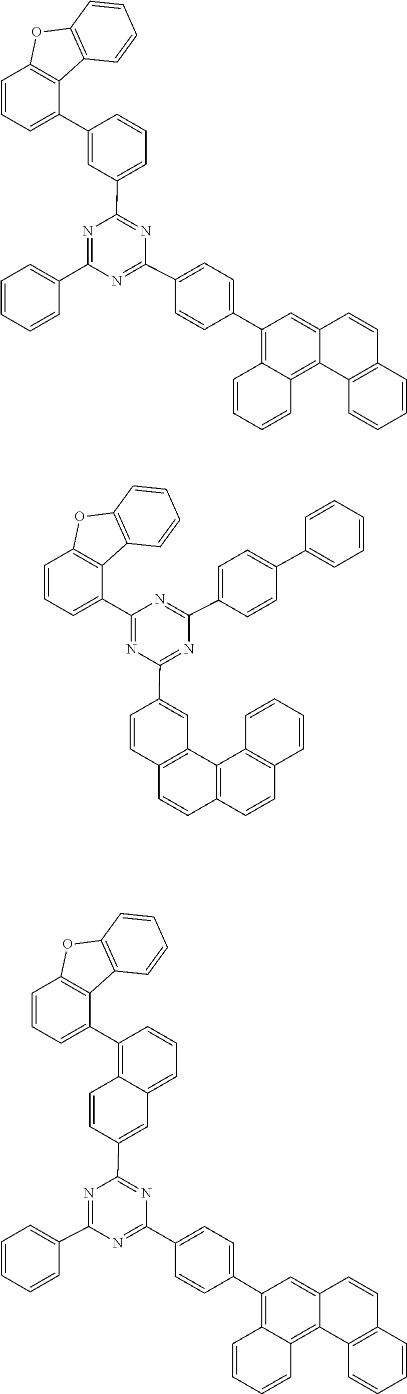 [SMILES: c1ccc(-c2nc(-c3ccc(-c4cc5ccc6ccccc6c5c5ccccc45)cc3)nc(-c3cccc(-c4cccc5oc6ccccc6c45)c3)n2)cc1 ; c1ccc(-c2ccc(-c3nc(-c4ccc5ccc6ccc7ccccc7c6c5c4)nc(-c4cccc5oc6ccccc6c45)n3)cc2)cc1 ; c1ccc(-c2nc(-c3ccc(-c4cc5ccc6ccccc6c5c5ccccc45)cc3)nc(-c3ccc4c(-c5cccc6oc7ccccc7c56)cccc4c3)n2)cc1]

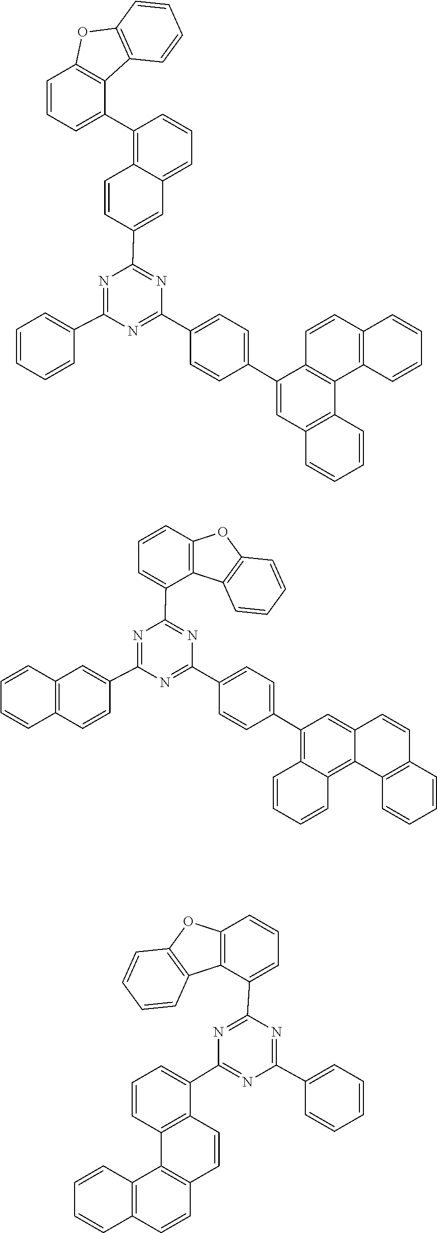 [SMILES: c1ccc(-c2nc(-c3ccc(-c4cc5ccccc5c5c4ccc4ccccc45)cc3)nc(-c3ccc4c(-c5cccc6oc7ccccc7c56)cccc4c3)n2)cc1 ; c1ccc2cc(-c3nc(-c4ccc(-c5cc6ccc7ccccc7c6c6ccccc56)cc4)nc(-c4cccc5oc6ccccc6c45)n3)ccc2c1 ; c1ccc(-c2nc(-c3cccc4c3ccc3ccc5ccccc5c34)nc(-c3cccc4oc5ccccc5c34)n2)cc1]

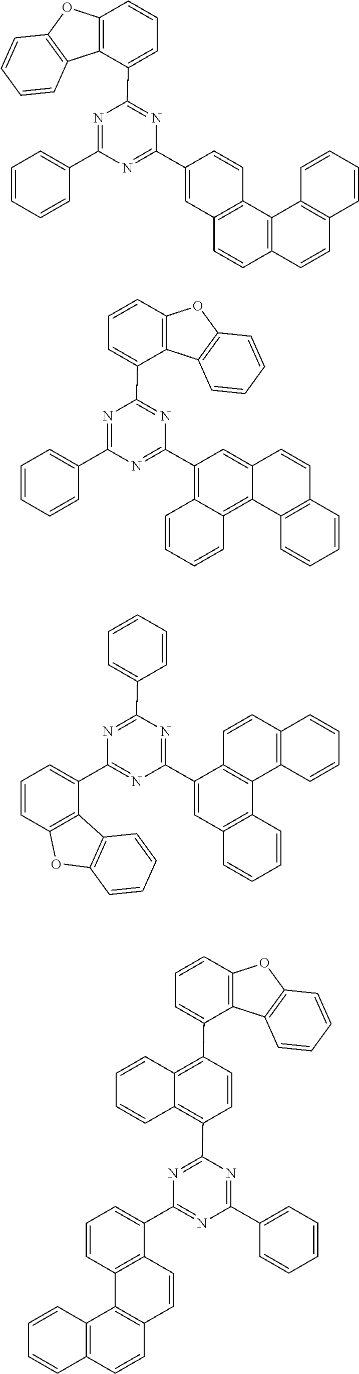 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3cc4ccc5ccccc5c4c4ccccc34)nc(-c3cccc4oc5ccccc5c34)n2)cc1 ; c1ccc(-c2nc(-c3cc4ccccc4c4c3ccc3ccccc34)nc(-c3cccc4oc5ccccc5c34)n2)cc1 ; c1ccc(-c2nc(-c3ccc(-c4cccc5oc6ccccc6c45)c4ccccc34)nc(-c3cccc4c3ccc3ccc5ccccc5c34)n2)cc1]

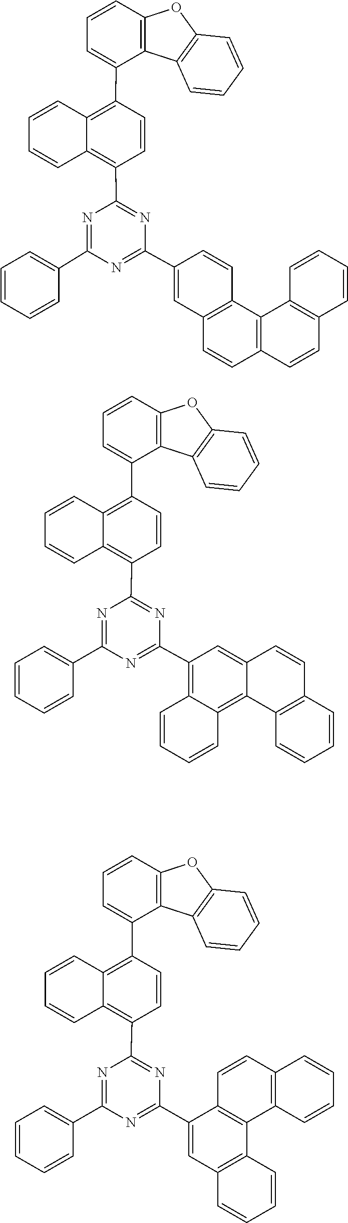 [SMILES: c1ccc(-c2nc(-c3ccc4c(ccc5ccc6ccccc6c54)c3)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3cc4ccc5ccccc5c4c4ccccc34)n2)cc1 ; c1ccc(-c2nc(-c3ccc(-c4cccc5oc6ccccc6c45)c4ccccc34)nc(-c3cc4ccccc4c4c3ccc3ccccc34)n2)cc1]

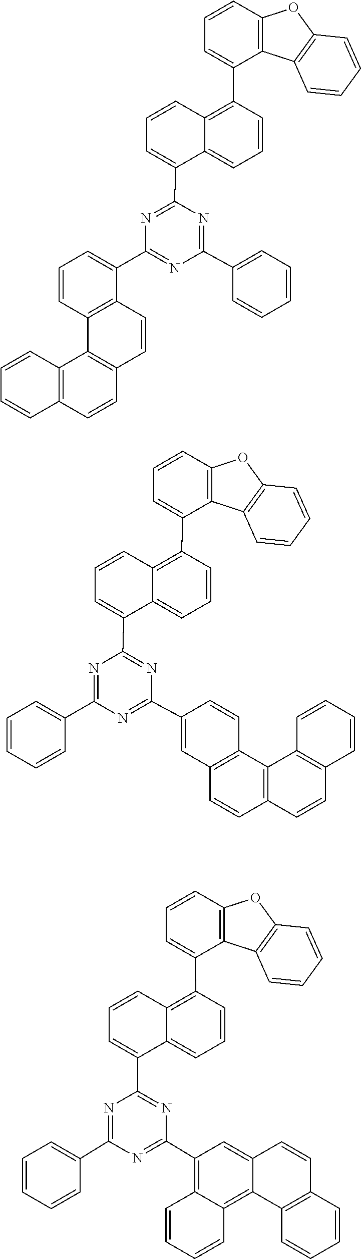 [SMILES: c1ccc(-c2nc(-c3cccc4c(-c5cccc6oc7ccccc7c56)cccc34)nc(-c3cccc4c3ccc3ccc5ccccc5c34)n2)cc1 ; c1ccc(-c2nc(-c3ccc4c(ccc5ccc6ccccc6c54)c3)nc(-c3cccc4c(-c5cccc6oc7ccccc7c56)cccc34)n2)cc1 ; c1ccc(-c2nc(-c3cccc4c(-c5cccc6oc7ccccc7c56)cccc34)nc(-c3cc4ccc5ccccc5c4c4ccccc34)n2)cc1]

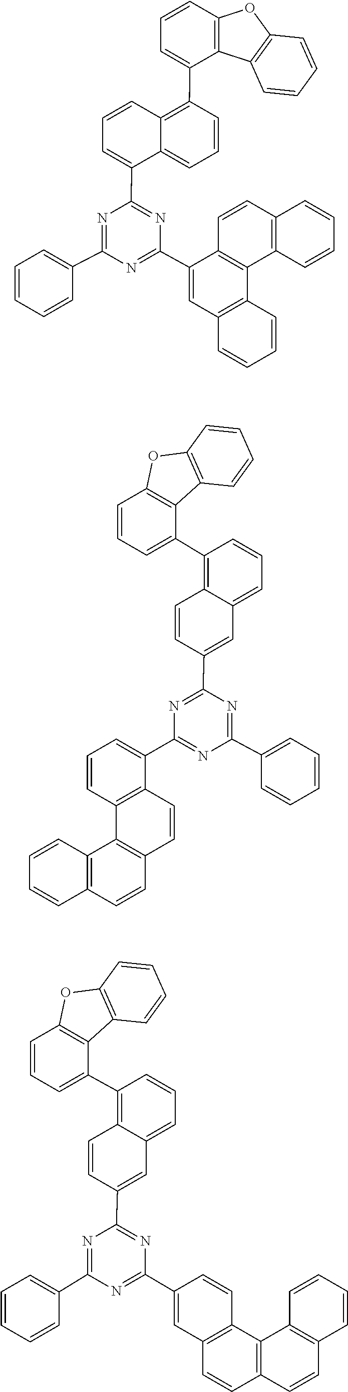 [SMILES: c1ccc(-c2nc(-c3cccc4c(-c5cccc6oc7ccccc7c56)cccc34)nc(-c3cc4ccccc4c4c3ccc3ccccc34)n2)cc1 ; c1ccc(-c2nc(-c3ccc4c(-c5cccc6oc7ccccc7c56)cccc4c3)nc(-c3cccc4c3ccc3ccc5ccccc5c34)n2)cc1 ; c1ccc(-c2nc(-c3ccc4c(-c5cccc6oc7ccccc7c56)cccc4c3)nc(-c3ccc4c(ccc5ccc6ccccc6c54)c3)n2)cc1]

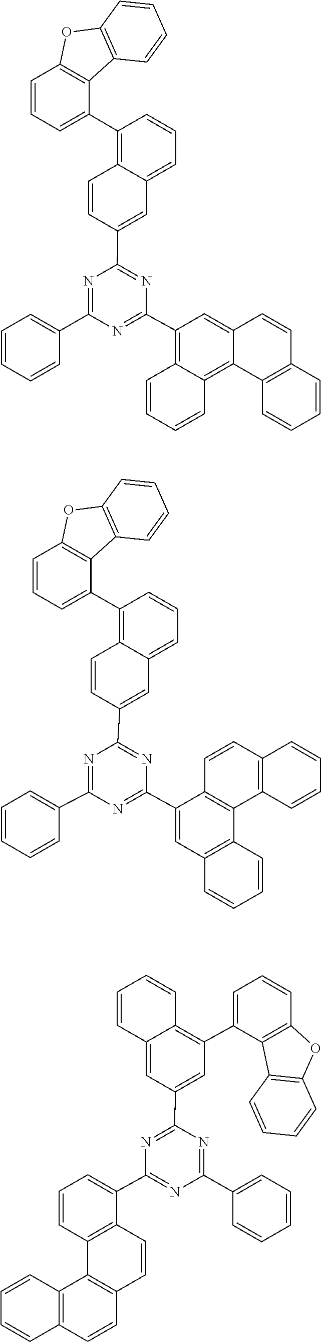 [SMILES: c1ccc(-c2nc(-c3ccc4c(-c5cccc6oc7ccccc7c56)cccc4c3)nc(-c3cc4ccc5ccccc5c4c4ccccc34)n2)cc1 ; c1ccc(-c2nc(-c3ccc4c(-c5cccc6oc7ccccc7c56)cccc4c3)nc(-c3cc4ccccc4c4c3ccc3ccccc34)n2)cc1 ; c1ccc(-c2nc(-c3cc(-c4cccc5oc6ccccc6c45)c4ccccc4c3)nc(-c3cccc4c3ccc3ccc5ccccc5c34)n2)cc1]

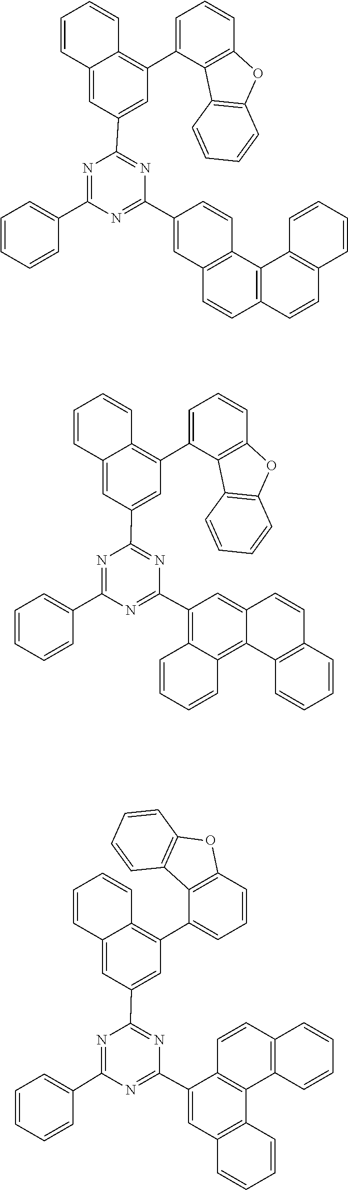 [SMILES: c1ccc(-c2nc(-c3cc(-c4cccc5oc6ccccc6c45)c4ccccc4c3)nc(-c3ccc4c(ccc5ccc6ccccc6c54)c3)n2)cc1 ; c1ccc(-c2nc(-c3cc(-c4cccc5oc6ccccc6c45)c4ccccc4c3)nc(-c3cc4ccc5ccccc5c4c4ccccc34)n2)cc1 ; c1ccc(-c2nc(-c3cc(-c4cccc5oc6ccccc6c45)c4ccccc4c3)nc(-c3cc4ccccc4c4c3ccc3ccccc34)n2)cc1]

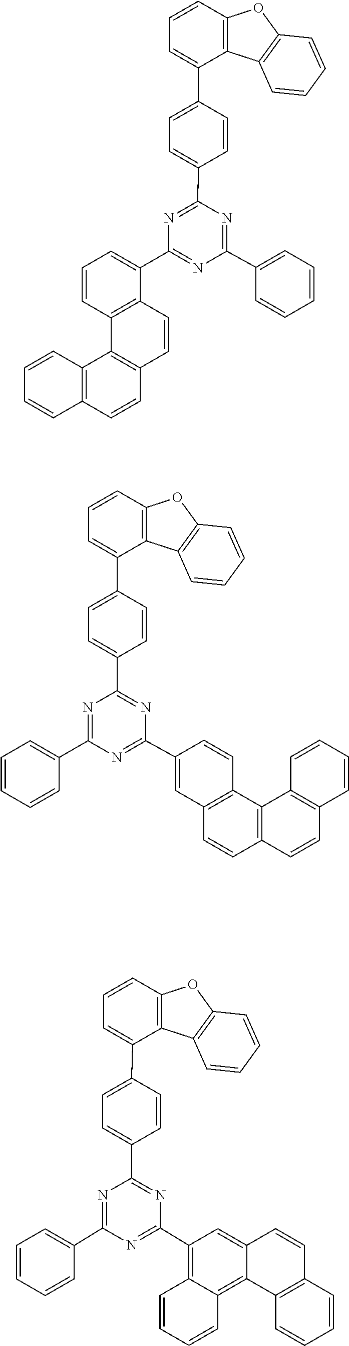 [SMILES: c1ccc(-c2nc(-c3ccc(-c4cccc5oc6ccccc6c45)cc3)nc(-c3cccc4c3ccc3ccc5ccccc5c34)n2)cc1 ; c1ccc(-c2nc(-c3ccc(-c4cccc5oc6ccccc6c45)cc3)nc(-c3ccc4c(ccc5ccc6ccccc6c54)c3)n2)cc1 ; c1ccc(-c2nc(-c3ccc(-c4cccc5oc6ccccc6c45)cc3)nc(-c3cc4ccc5ccccc5c4c4ccccc34)n2)cc1]

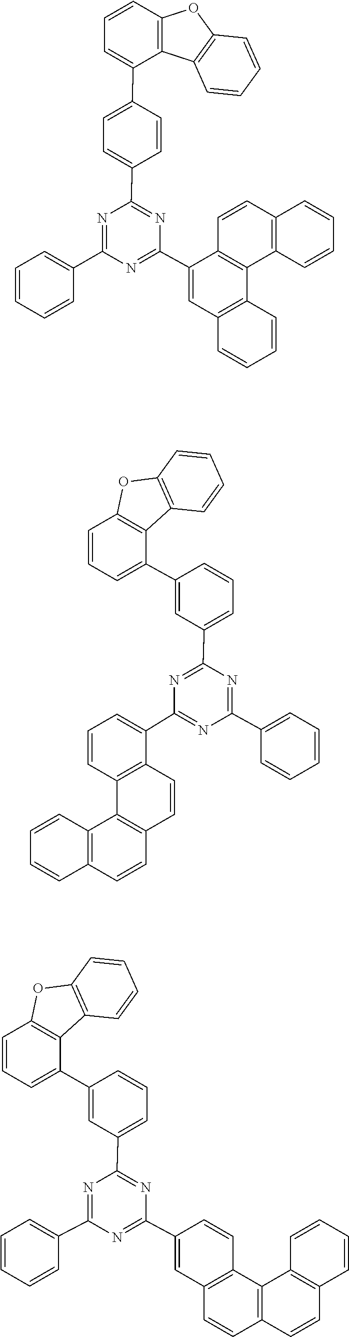 [SMILES: c1ccc(-c2nc(-c3ccc(-c4cccc5oc6ccccc6c45)cc3)nc(-c3cc4ccccc4c4c3ccc3ccccc34)n2)cc1 ; c1ccc(-c2nc(-c3cccc(-c4cccc5oc6ccccc6c45)c3)nc(-c3cccc4c3ccc3ccc5ccccc5c34)n2)cc1 ; c1ccc(-c2nc(-c3cccc(-c4cccc5oc6ccccc6c45)c3)nc(-c3ccc4c(ccc5ccc6ccccc6c54)c3)n2)cc1]

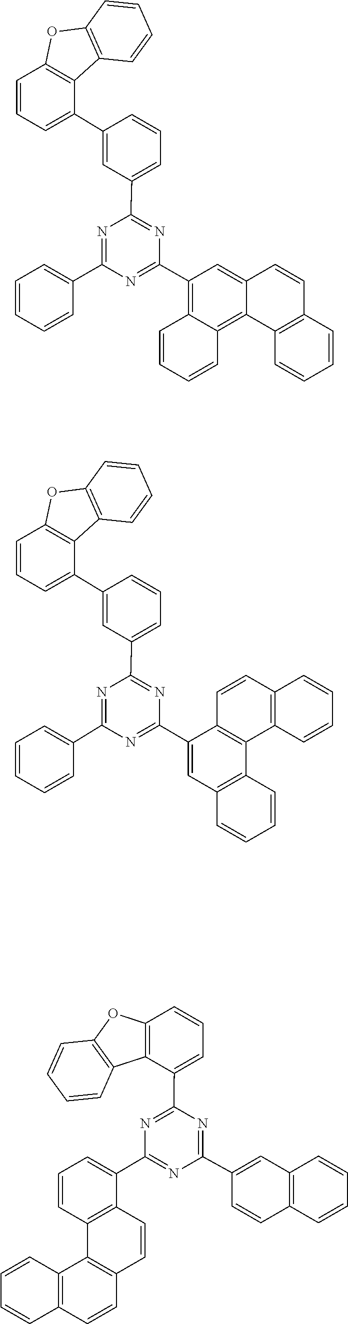 [SMILES: c1ccc(-c2nc(-c3cccc(-c4cccc5oc6ccccc6c45)c3)nc(-c3cc4ccc5ccccc5c4c4ccccc34)n2)cc1 ; c1ccc(-c2nc(-c3cccc(-c4cccc5oc6ccccc6c45)c3)nc(-c3cc4ccccc4c4c3ccc3ccccc34)n2)cc1 ; c1ccc2cc(-c3nc(-c4cccc5c4ccc4ccc6ccccc6c45)nc(-c4cccc5oc6ccccc6c45)n3)ccc2c1]

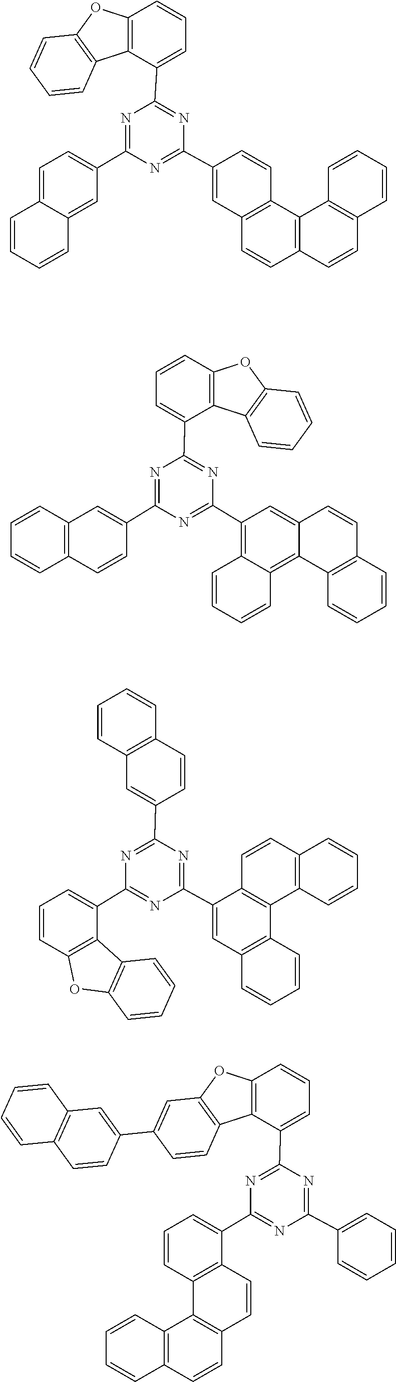 [SMILES: c1ccc2cc(-c3nc(-c4ccc5c(ccc6ccc7ccccc7c65)c4)nc(-c4cccc5oc6ccccc6c45)n3)ccc2c1 ; c1ccc2cc(-c3nc(-c4cc5ccc6ccccc6c5c5ccccc45)nc(-c4cccc5oc6ccccc6c45)n3)ccc2c1 ; c1ccc2cc(-c3nc(-c4cc5ccccc5c5c4ccc4ccccc45)nc(-c4cccc5oc6ccccc6c45)n3)ccc2c1 ; c1ccc(-c2nc(-c3cccc4c3ccc3ccc5ccccc5c34)nc(-c3cccc4oc5cc(-c6ccc7ccccc7c6)ccc5c34)n2)cc1]

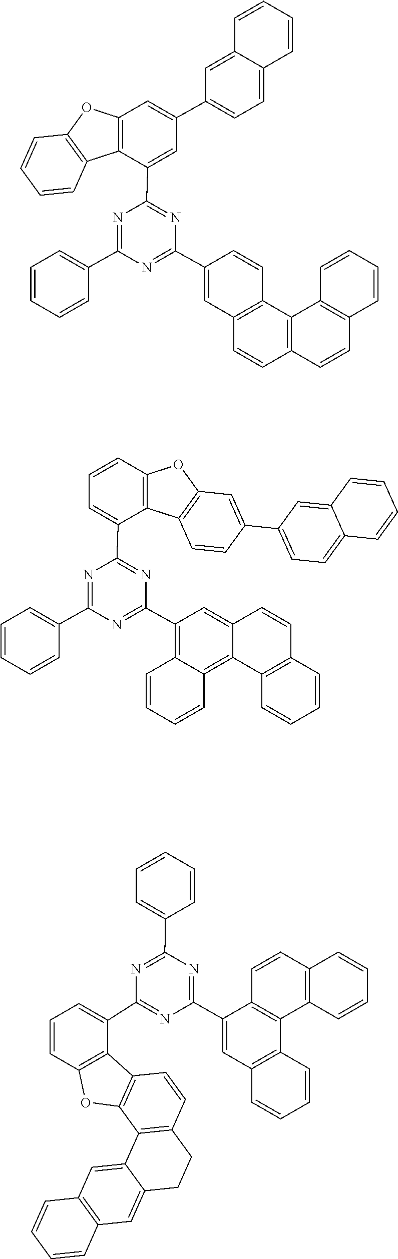 [SMILES: c1ccc(-c2nc(-c3ccc4c(ccc5ccc6ccccc6c54)c3)nc(-c3cc(-c4ccc5ccccc5c4)cc4oc5ccccc5c34)n2)cc1 ; c1ccc(-c2nc(-c3cc4ccc5ccccc5c4c4ccccc34)nc(-c3cccc4oc5cc(-c6ccc7ccccc7c6)ccc5c34)n2)cc1 ; c1ccc(-c2nc(-c3cc4ccccc4c4c3ccc3ccccc34)nc(-c3cccc4oc5c6c(ccc5c34)CCc3cc4ccccc4cc3-6)n2)cc1]

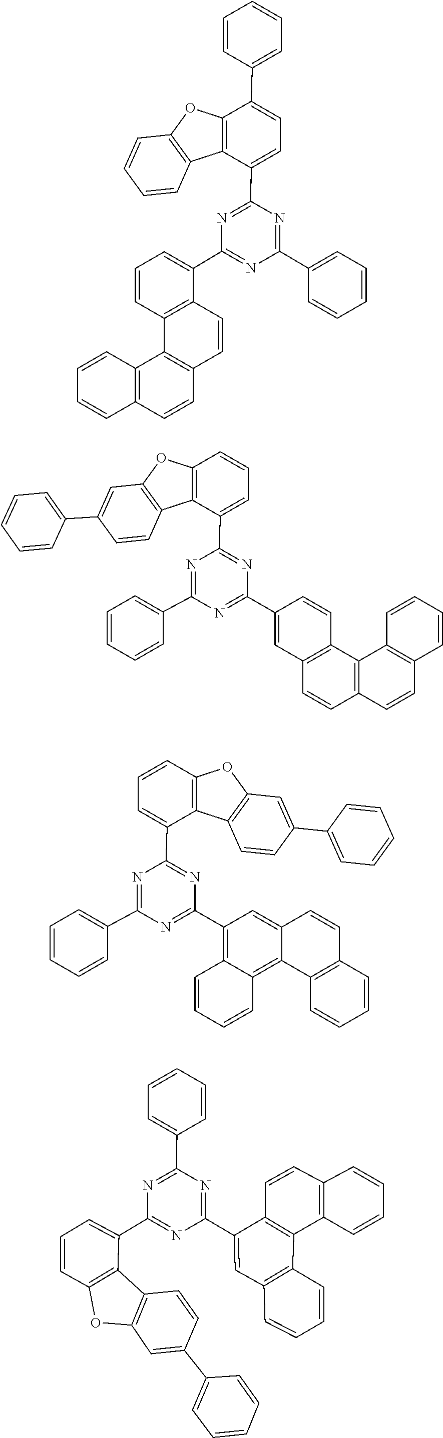

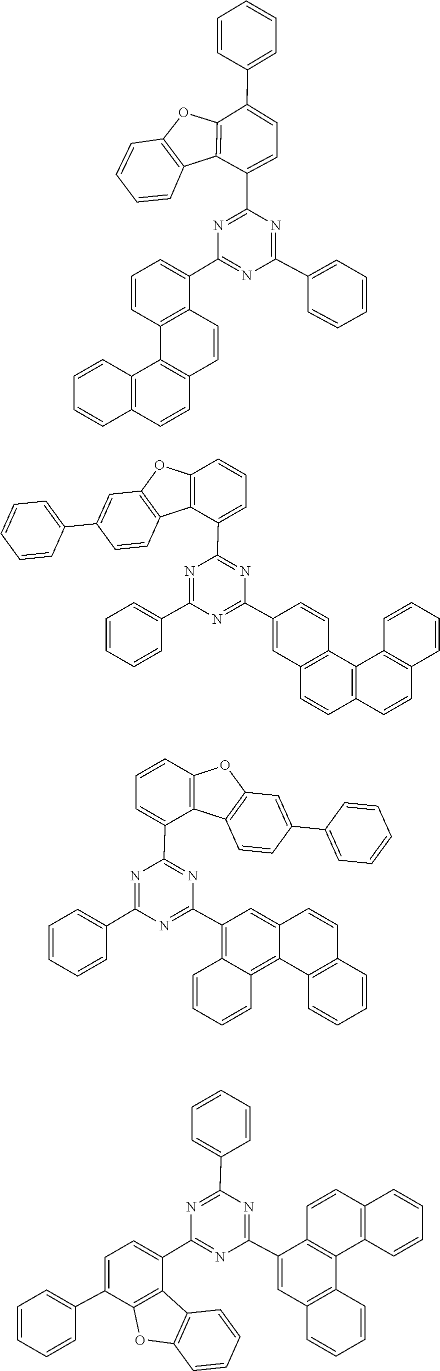

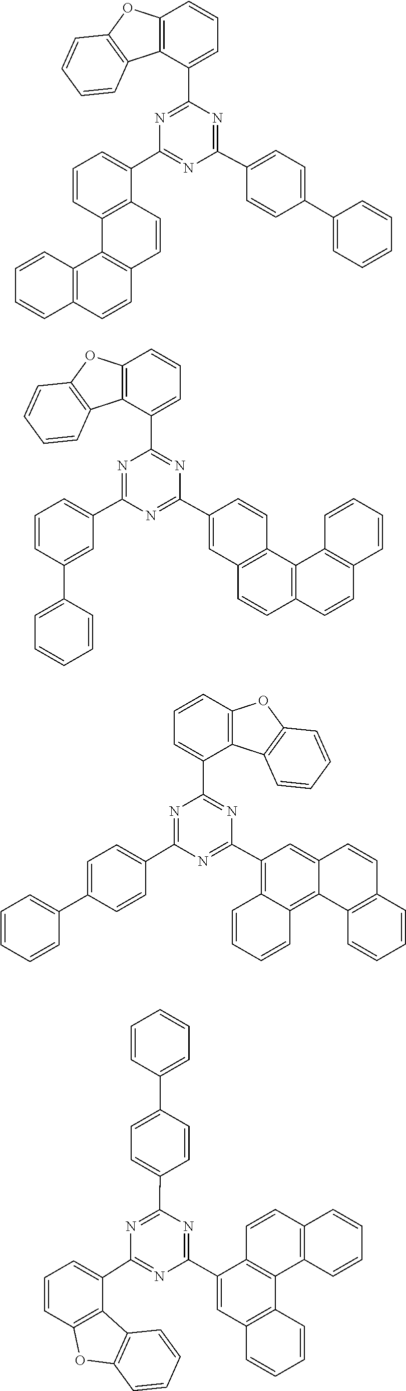

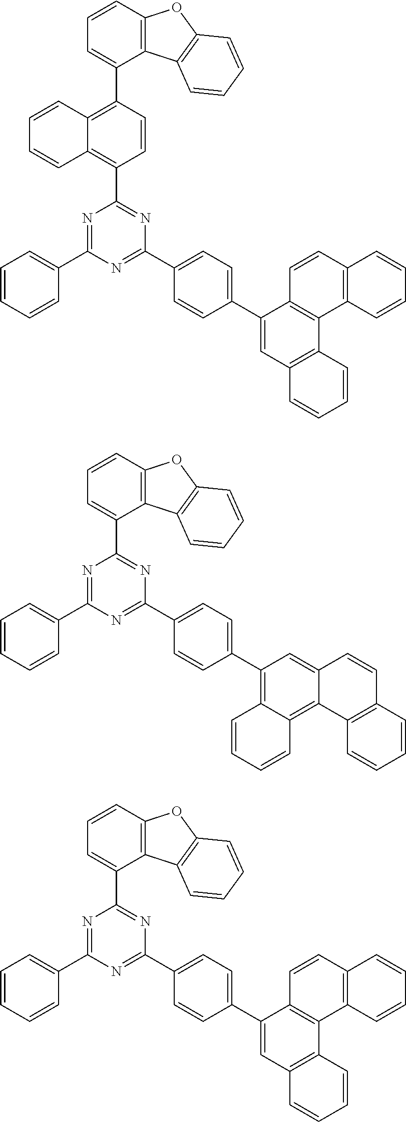 [SMILES: c1ccc(-c2nc(-c3ccc(-c4cc5ccccc5c5c4ccc4ccccc45)cc3)nc(-c3ccc(-c4cccc5oc6ccccc6c45)c4ccccc34)n2)cc1 ; c1ccc(-c2nc(-c3ccc(-c4cc5ccc6ccccc6c5c5ccccc45)cc3)nc(-c3cccc4oc5ccccc5c34)n2)cc1 ; c1ccc(-c2nc(-c3ccc(-c4cc5ccccc5c5c4ccc4ccccc45)cc3)nc(-c3cccc4oc5ccccc5c34)n2)cc1]

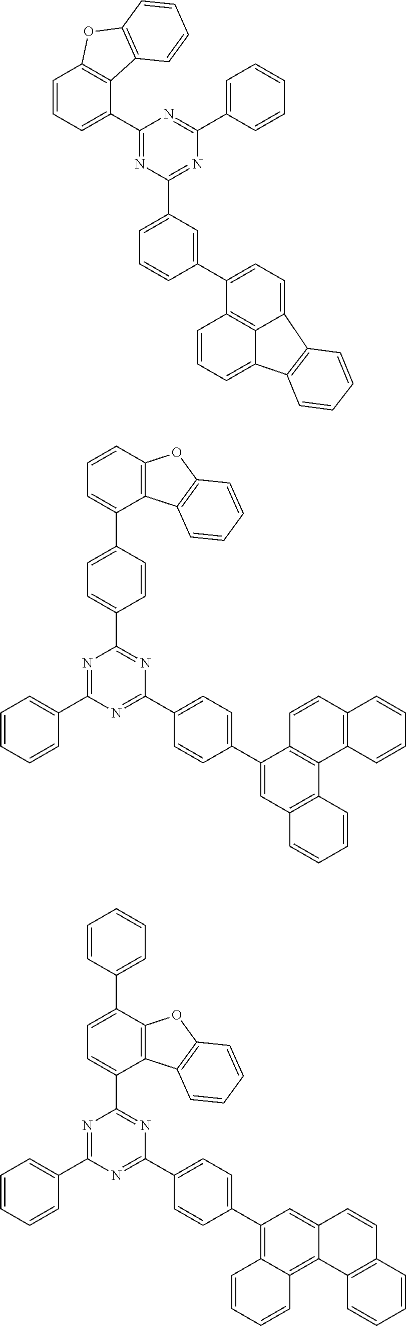 [SMILES: c1ccc(-c2nc(-c3cccc(-c4ccc5c6c(cccc46)-c4ccccc4-5)c3)nc(-c3cccc4oc5ccccc5c34)n2)cc1 ; c1ccc(-c2nc(-c3ccc(-c4cc5ccccc5c5c4ccc4ccccc45)cc3)nc(-c3ccc(-c4cccc5oc6ccccc6c45)cc3)n2)cc1 ; c1ccc(-c2nc(-c3ccc(-c4cc5ccc6ccccc6c5c5ccccc45)cc3)nc(-c3ccc(-c4ccccc4)c4oc5ccccc5c34)n2)cc1]

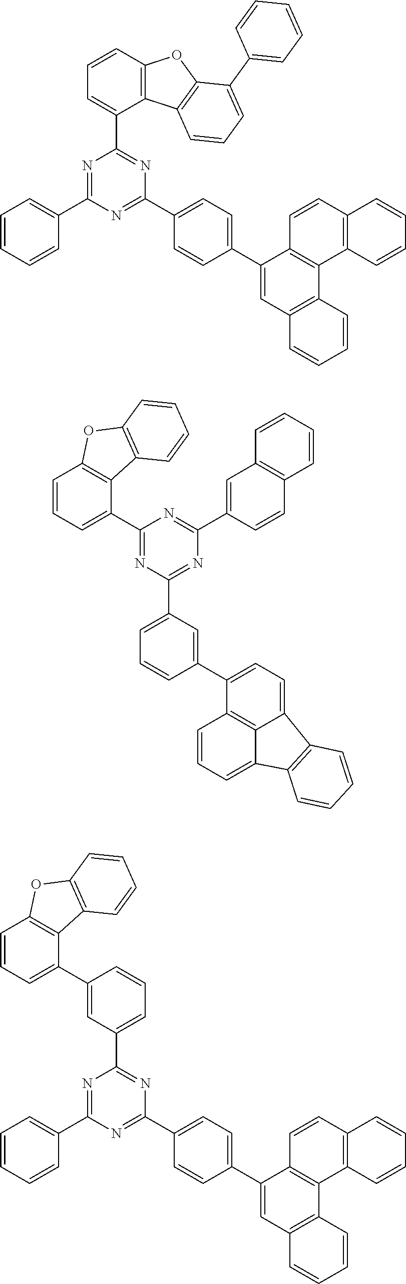 [SMILES: c1ccc(-c2nc(-c3ccc(-c4cc5ccccc5c5c4ccc4ccccc45)cc3)nc(-c3cccc4oc5c(-c6ccccc6)cccc5c34)n2)cc1 ; c1cc(-c2nc(-c3ccc4ccccc4c3)nc(-c3cccc4oc5ccccc5c34)n2)cc(-c2ccc3c4c(cccc24)-c2ccccc2-3)c1 ; c1ccc(-c2nc(-c3ccc(-c4cc5ccccc5c5c4ccc4ccccc45)cc3)nc(-c3cccc(-c4cccc5oc6ccccc6c45)c3)n2)cc1]

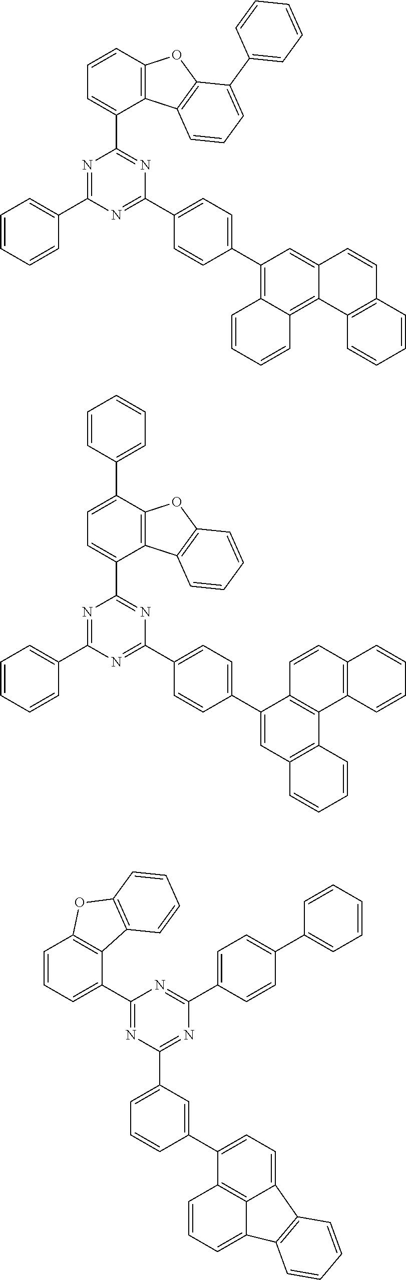 [SMILES: c1ccc(-c2nc(-c3ccc(-c4cc5ccc6ccccc6c5c5ccccc45)cc3)nc(-c3cccc4oc5c(-c6ccccc6)cccc5c34)n2)cc1 ; c1ccc(-c2nc(-c3ccc(-c4cc5ccccc5c5c4ccc4ccccc45)cc3)nc(-c3ccc(-c4ccccc4)c4oc5ccccc5c34)n2)cc1 ; c1ccc(-c2ccc(-c3nc(-c4cccc(-c5ccc6c7c(cccc57)-c5ccccc5-6)c4)nc(-c4cccc5oc6ccccc6c45)n3)cc2)cc1]

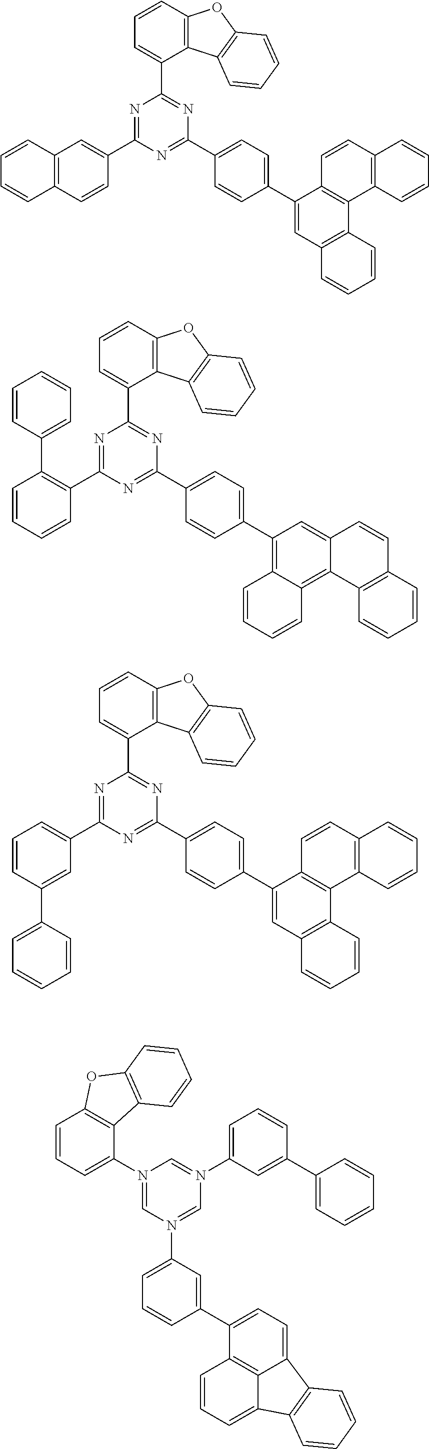

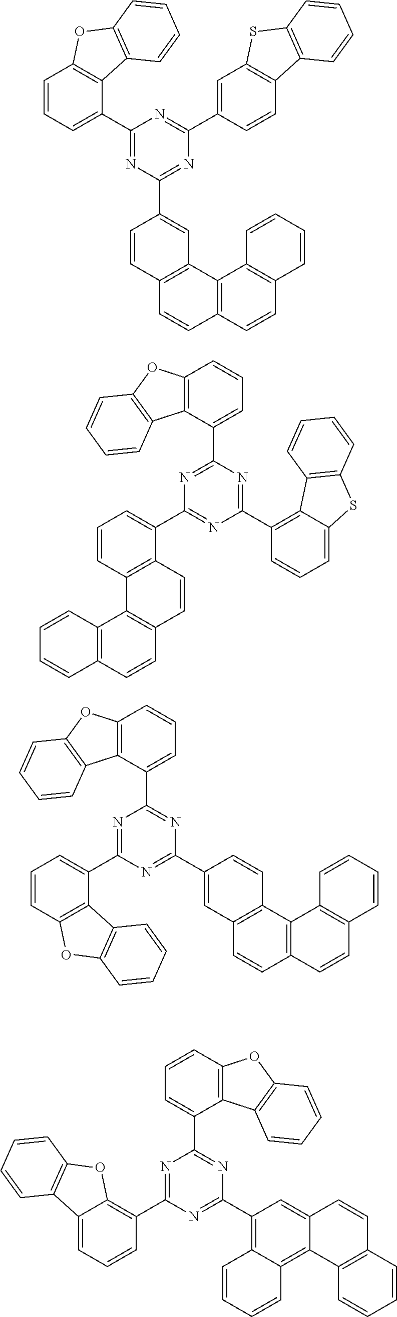

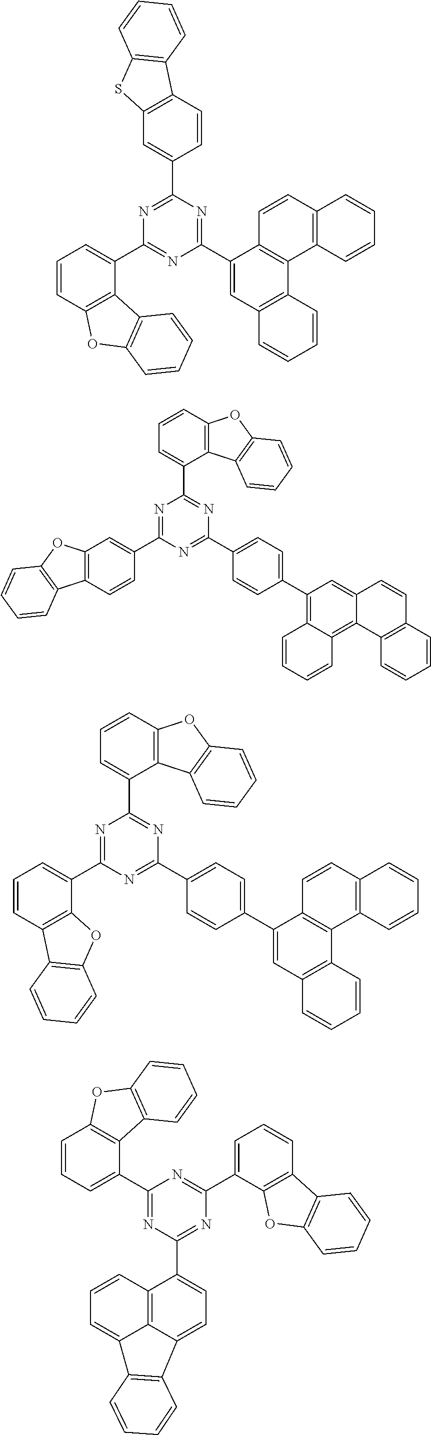

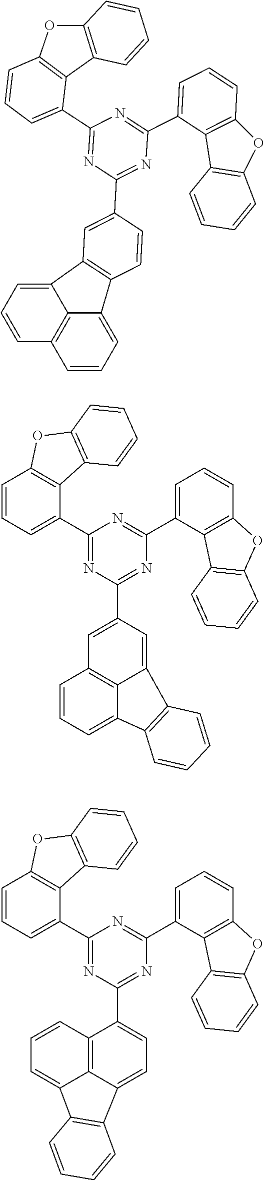

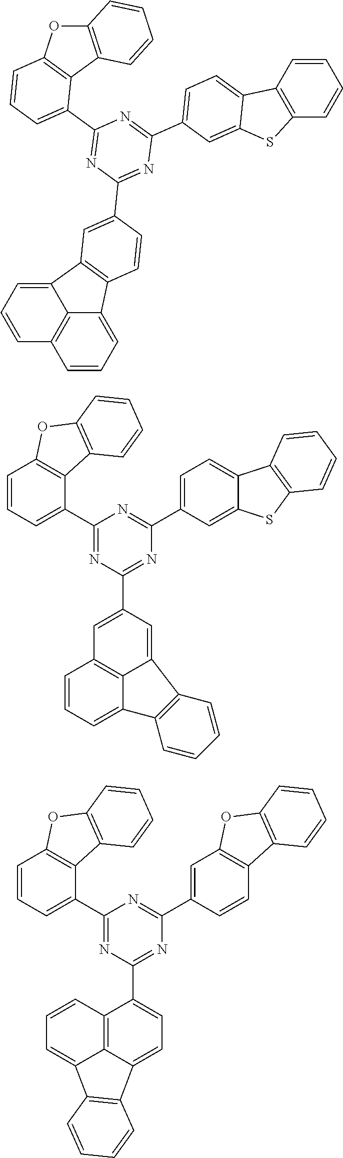

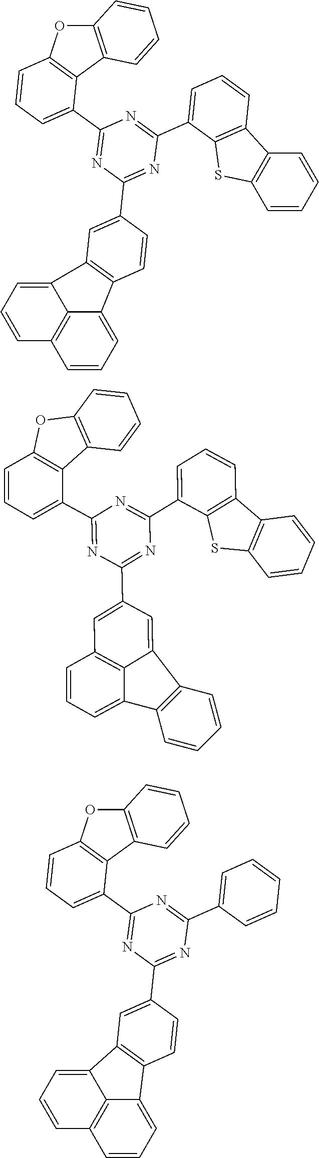

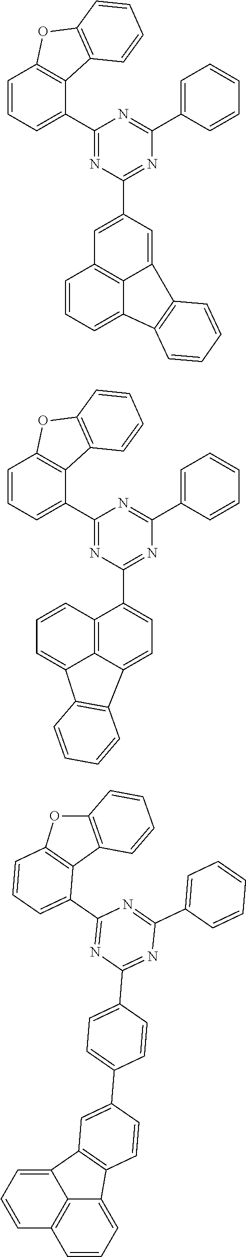

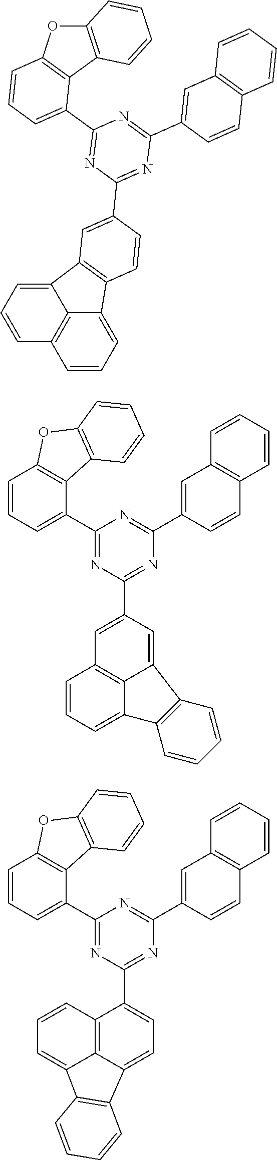

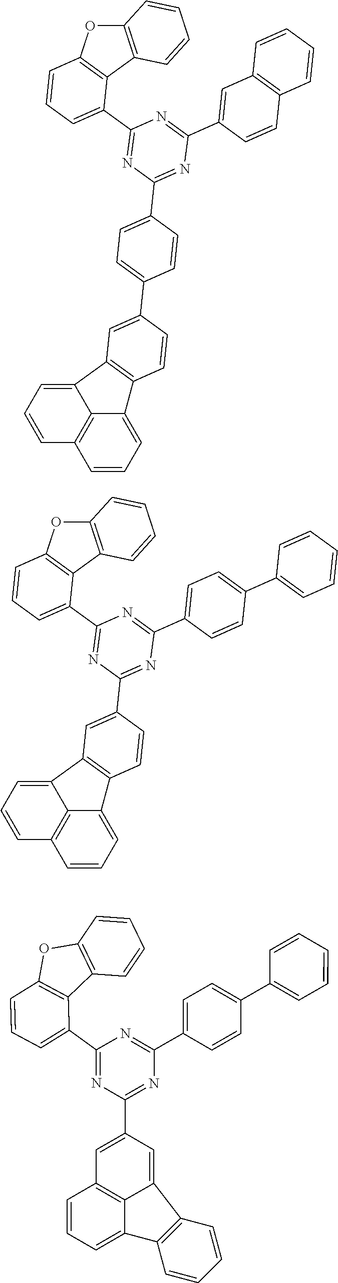

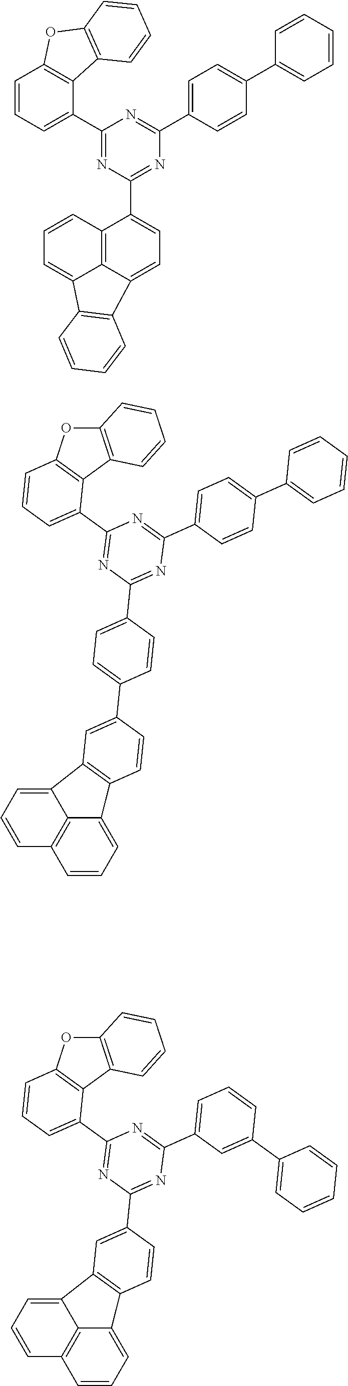

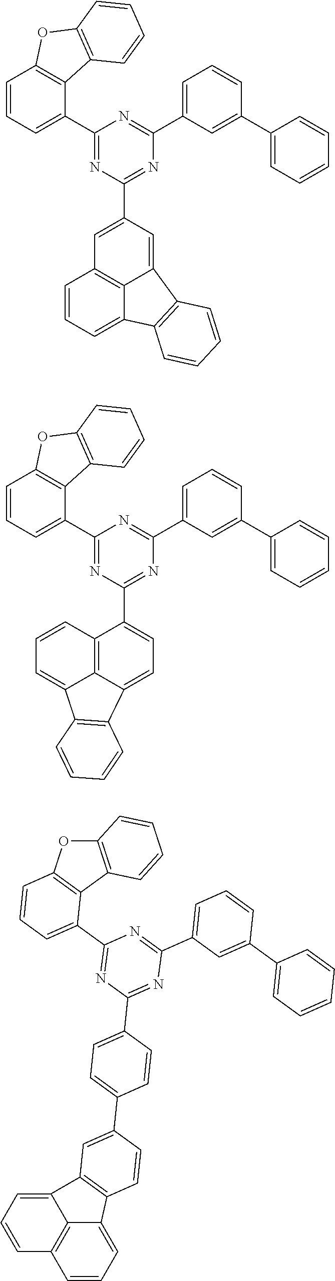

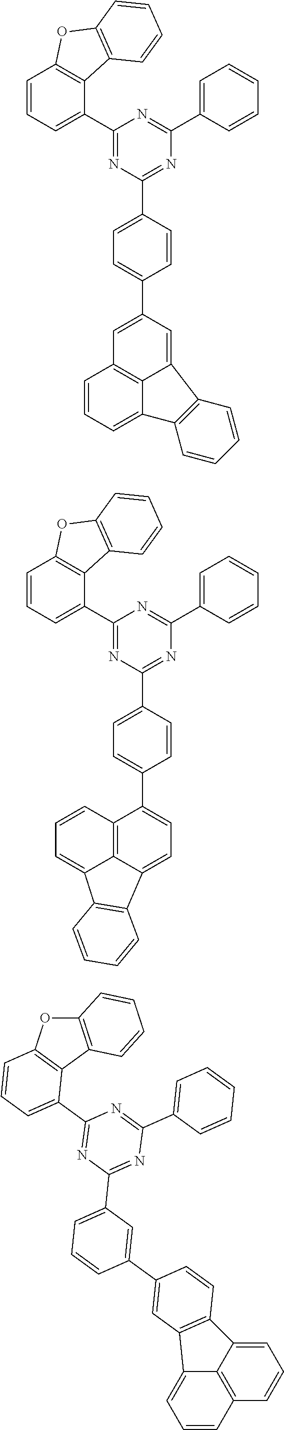

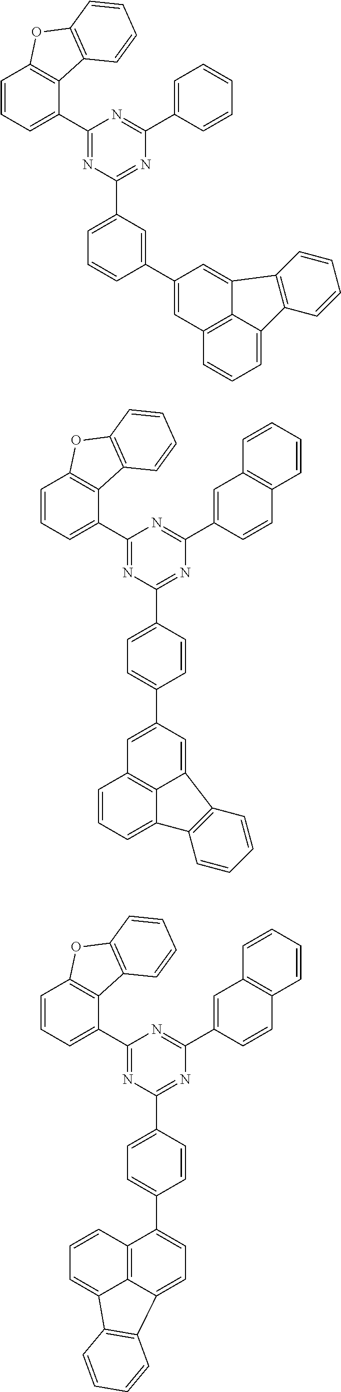 [SMILES: c1ccc(-c2nc(-c3cccc(-c4cc5c6c(cccc6c4)-c4ccccc4-5)c3)nc(-c3cccc4oc5ccccc5c34)n2)cc1 ; c1ccc2c(c1)-c1cccc3cc(-c4ccc(-c5nc(-c6ccc7ccccc7c6)nc(-c6cccc7oc8ccccc8c67)n5)cc4)cc-2c13 ; c1ccc2c(c1)-c1cccc3c(-c4ccc(-c5nc(-c6ccc7ccccc7c6)nc(-c6cccc7oc8ccccc8c67)n5)cc4)ccc-2c13]

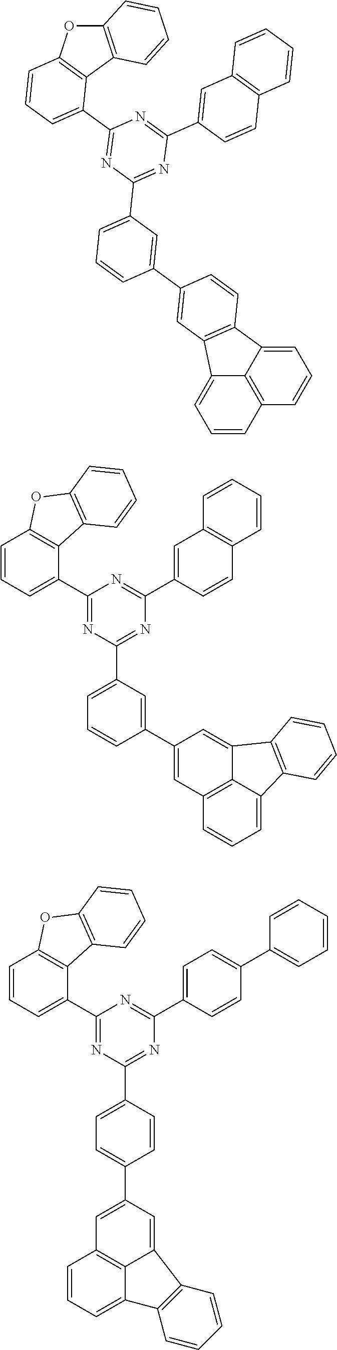 [SMILES: c1cc(-c2ccc3c(c2)-c2cccc4cccc-3c24)cc(-c2nc(-c3ccc4ccccc4c3)nc(-c3cccc4oc5ccccc5c34)n2)c1 ; c1cc(-c2cc3c4c(cccc4c2)-c2ccccc2-3)cc(-c2nc(-c3ccc4ccccc4c3)nc(-c3cccc4oc5ccccc5c34)n2)c1 ; c1ccc(-c2ccc(-c3nc(-c4ccc(-c5cc6c7c(cccc7c5)-c5ccccc5-6)cc4)nc(-c4cccc5oc6ccccc6c45)n3)cc2)cc1]

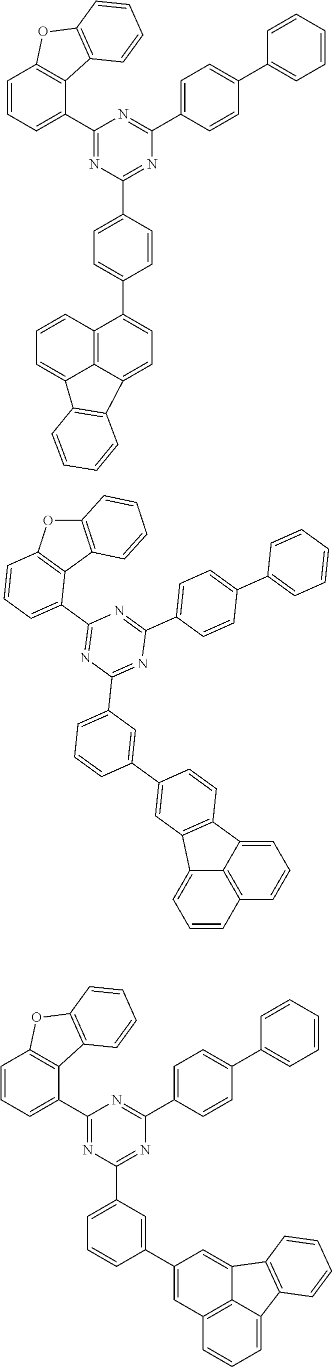

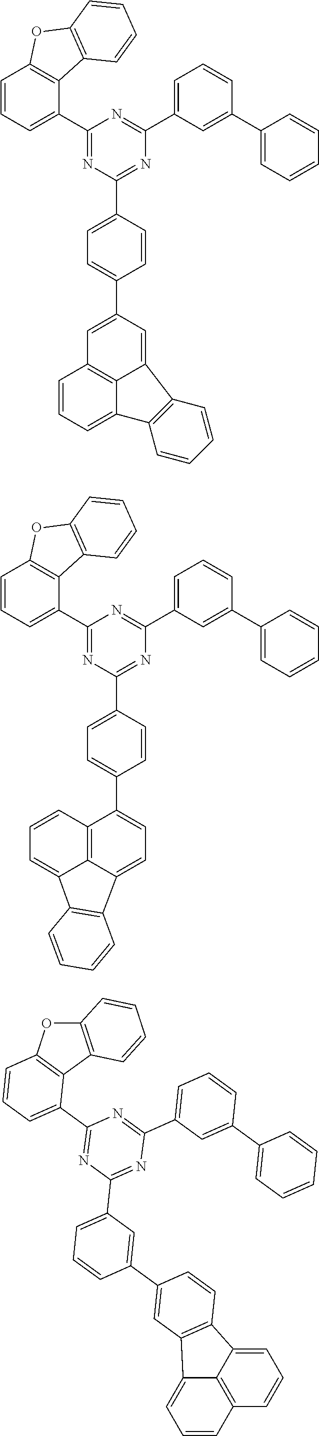

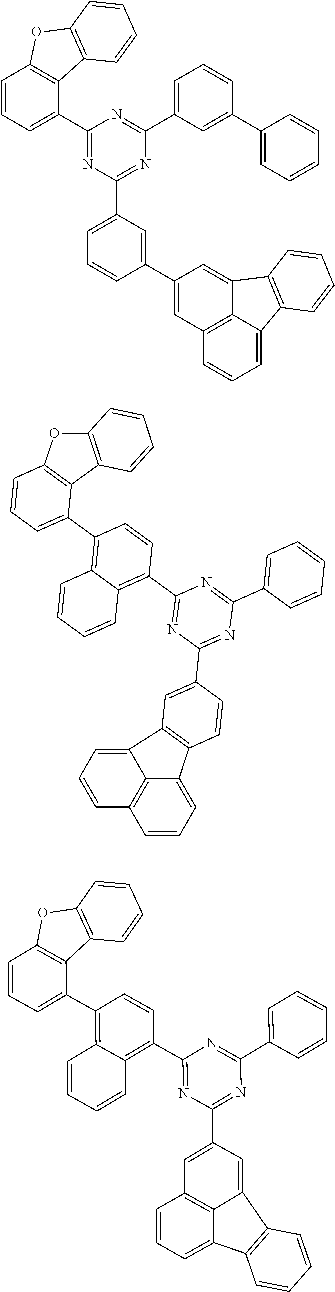 [SMILES: c1ccc(-c2cccc(-c3nc(-c4cccc(-c5cc6c7c(cccc7c5)-c5ccccc5-6)c4)nc(-c4cccc5oc6ccccc6c45)n3)c2)cc1 ; c1ccc(-c2nc(-c3ccc4c(c3)-c3cccc5cccc-4c35)nc(-c3ccc(-c4cccc5oc6ccccc6c45)c4ccccc34)n2)cc1 ; c1ccc(-c2nc(-c3cc4c5c(cccc5c3)-c3ccccc3-4)nc(-c3ccc(-c4cccc5oc6ccccc6c45)c4ccccc34)n2)cc1]

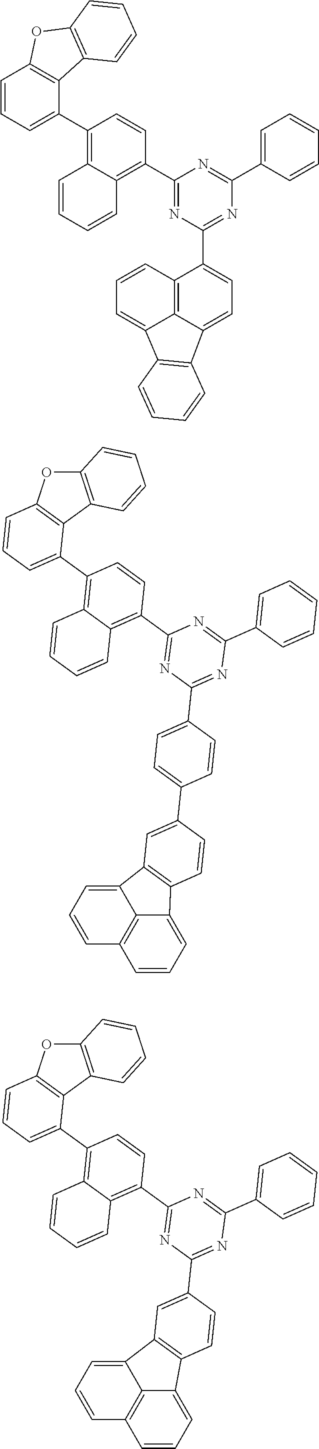

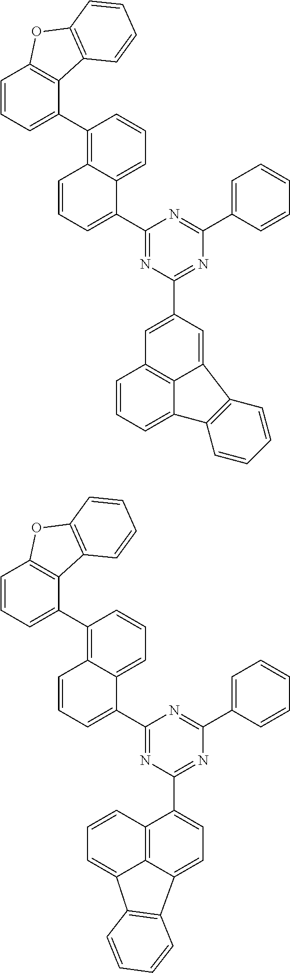 [SMILES: c1ccc(-c2nc(-c3cc4c5c(cccc5c3)-c3ccccc3-4)nc(-c3cccc4c(-c5cccc6oc7ccccc7c56)cccc34)n2)cc1 ; c1ccc(-c2nc(-c3cccc4c(-c5cccc6oc7ccccc7c56)cccc34)nc(-c3ccc4c5c(cccc35)-c3ccccc3-4)n2)cc1]

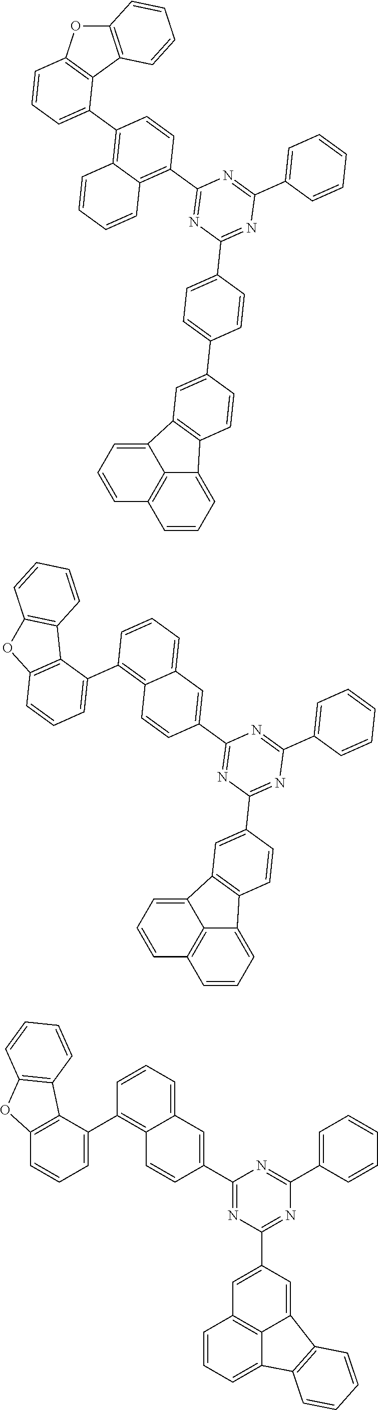 [SMILES: c1ccc(-c2nc(-c3ccc(-c4ccc5c(c4)-c4cccc6cccc-5c46)cc3)nc(-c3ccc(-c4cccc5oc6ccccc6c45)c4ccccc34)n2)cc1 ; c1ccc(-c2nc(-c3ccc4c(c3)-c3cccc5cccc-4c35)nc(-c3ccc4c(-c5cccc6oc7ccccc7c56)cccc4c3)n2)cc1 ; c1ccc(-c2nc(-c3ccc4c(-c5cccc6oc7ccccc7c56)cccc4c3)nc(-c3cc4c5c(cccc5c3)-c3ccccc3-4)n2)cc1]

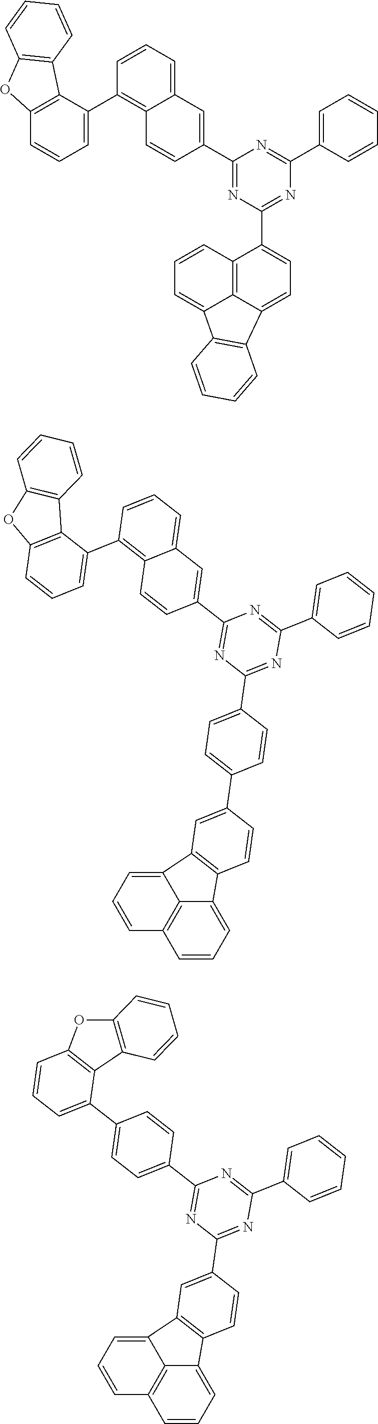

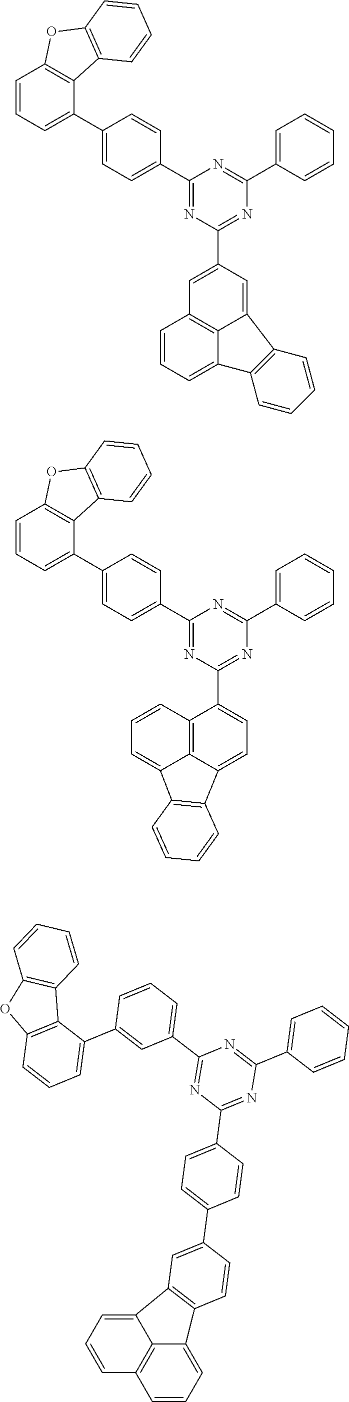 [SMILES: c1ccc(-c2nc(-c3ccc(-c4cccc5oc6ccccc6c45)cc3)nc(-c3cc4c5c(cccc5c3)-c3ccccc3-4)n2)cc1 ; c1ccc(-c2nc(-c3ccc(-c4cccc5oc6ccccc6c45)cc3)nc(-c3ccc4c5c(cccc35)-c3ccccc3-4)n2)cc1 ; c1ccc(-c2nc(-c3ccc(-c4ccc5c(c4)-c4cccc6cccc-5c46)cc3)nc(-c3cccc(-c4cccc5oc6ccccc6c45)c3)n2)cc1]

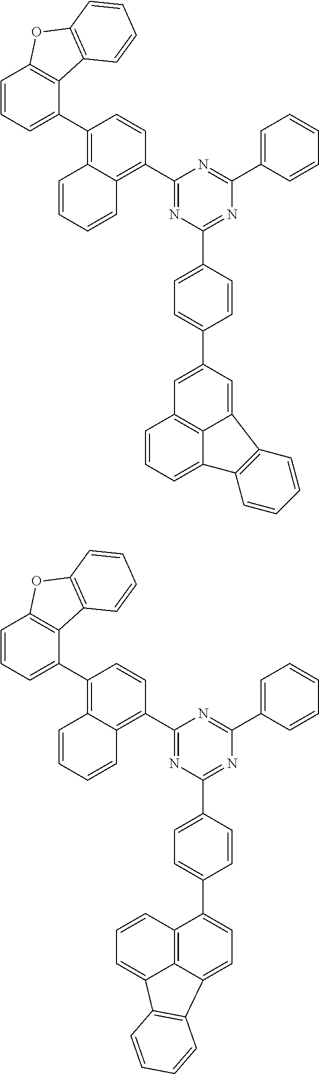 [SMILES: c1ccc(-c2nc(-c3ccc(-c4cc5c6c(cccc6c4)-c4ccccc4-5)cc3)nc(-c3ccc(-c4cccc5oc6ccccc6c45)c4ccccc34)n2)cc1 ; c1ccc(-c2nc(-c3ccc(-c4ccc5c6c(cccc46)-c4ccccc4-5)cc3)nc(-c3ccc(-c4cccc5oc6ccccc6c45)c4ccccc34)n2)cc1]

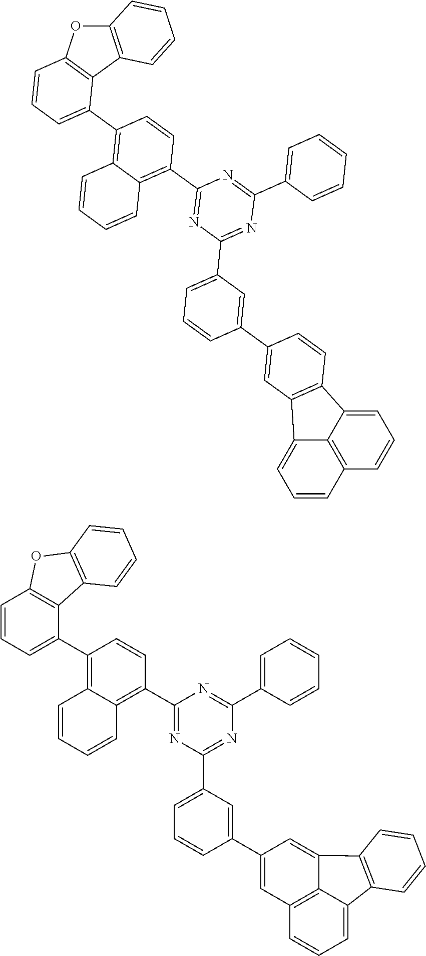 [SMILES: c1ccc(-c2nc(-c3cccc(-c4ccc5c(c4)-c4cccc6cccc-5c46)c3)nc(-c3ccc(-c4cccc5oc6ccccc6c45)c4ccccc34)n2)cc1 ; c1ccc(-c2nc(-c3cccc(-c4cc5c6c(cccc6c4)-c4ccccc4-5)c3)nc(-c3ccc(-c4cccc5oc6ccccc6c45)c4ccccc34)n2)cc1]

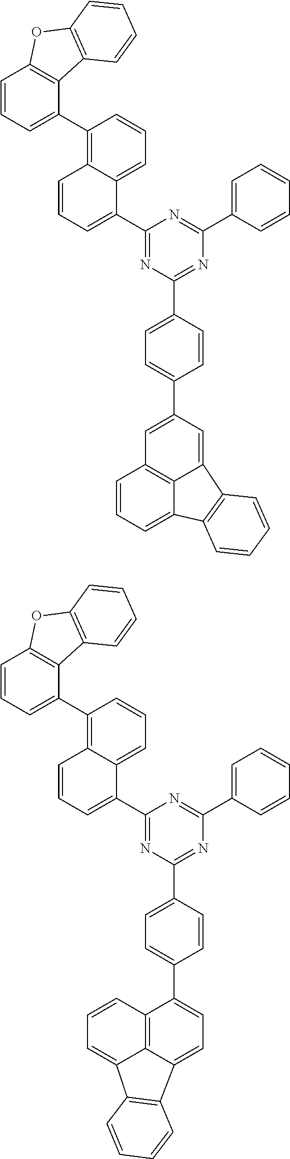 [SMILES: c1ccc(-c2nc(-c3ccc(-c4cc5c6c(cccc6c4)-c4ccccc4-5)cc3)nc(-c3cccc4c(-c5cccc6oc7ccccc7c56)cccc34)n2)cc1 ; c1ccc(-c2nc(-c3ccc(-c4ccc5c6c(cccc46)-c4ccccc4-5)cc3)nc(-c3cccc4c(-c5cccc6oc7ccccc7c56)cccc34)n2)cc1]

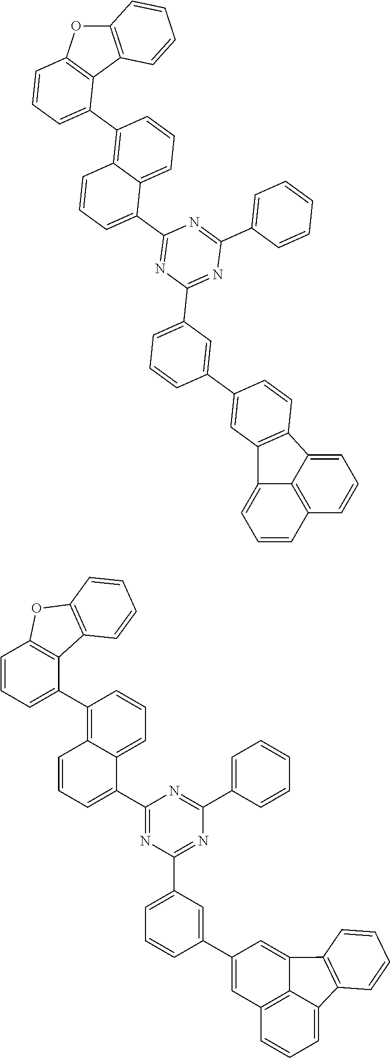 [SMILES: c1ccc(-c2nc(-c3cccc(-c4ccc5c(c4)-c4cccc6cccc-5c46)c3)nc(-c3cccc4c(-c5cccc6oc7ccccc7c56)cccc34)n2)cc1 ; c1ccc(-c2nc(-c3cccc(-c4cc5c6c(cccc6c4)-c4ccccc4-5)c3)nc(-c3cccc4c(-c5cccc6oc7ccccc7c56)cccc34)n2)cc1]

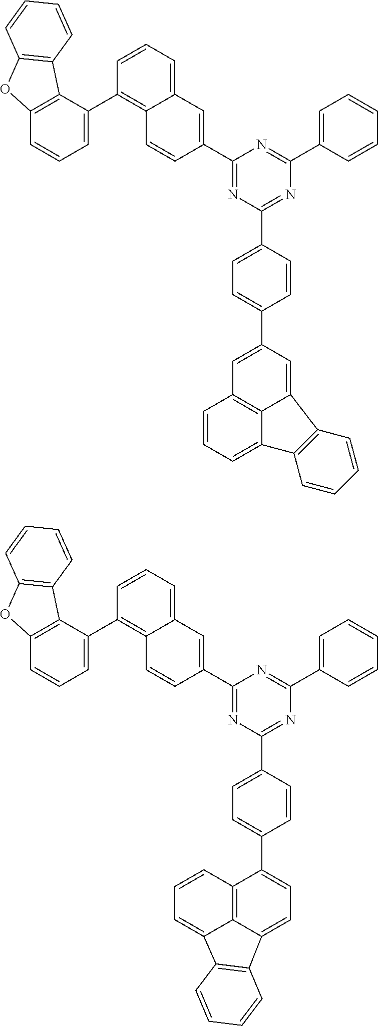 [SMILES: c1ccc(-c2nc(-c3ccc(-c4cc5c6c(cccc6c4)-c4ccccc4-5)cc3)nc(-c3ccc4c(-c5cccc6oc7ccccc7c56)cccc4c3)n2)cc1 ; c1ccc(-c2nc(-c3ccc(-c4ccc5c6c(cccc46)-c4ccccc4-5)cc3)nc(-c3ccc4c(-c5cccc6oc7ccccc7c56)cccc4c3)n2)cc1]

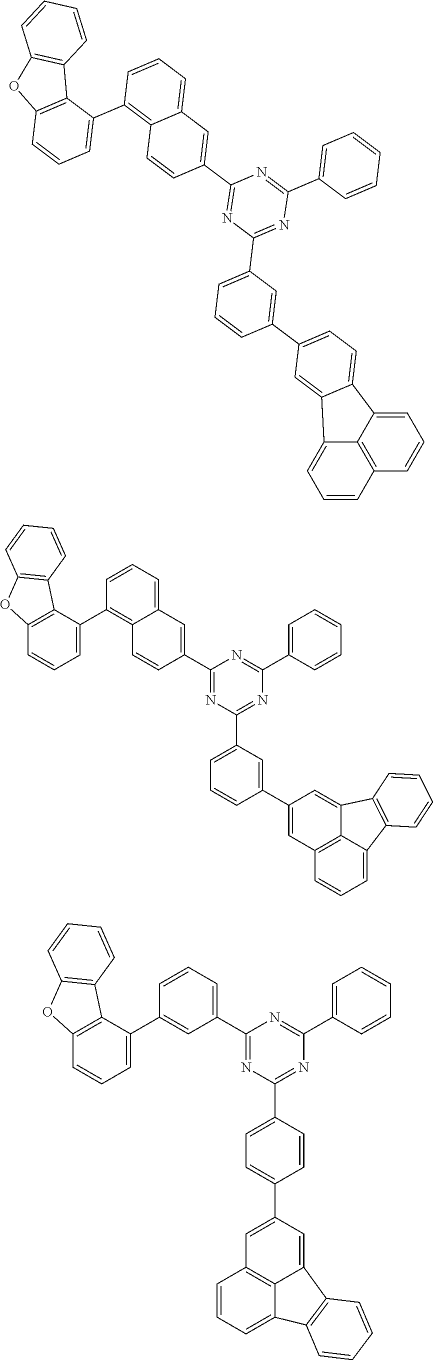 [SMILES: c1ccc(-c2nc(-c3cccc(-c4ccc5c(c4)-c4cccc6cccc-5c46)c3)nc(-c3ccc4c(-c5cccc6oc7ccccc7c56)cccc4c3)n2)cc1 ; c1ccc(-c2nc(-c3cccc(-c4cc5c6c(cccc6c4)-c4ccccc4-5)c3)nc(-c3ccc4c(-c5cccc6oc7ccccc7c56)cccc4c3)n2)cc1 ; c1ccc(-c2nc(-c3ccc(-c4cc5c6c(cccc6c4)-c4ccccc4-5)cc3)nc(-c3cccc(-c4cccc5oc6ccccc6c45)c3)n2)cc1]

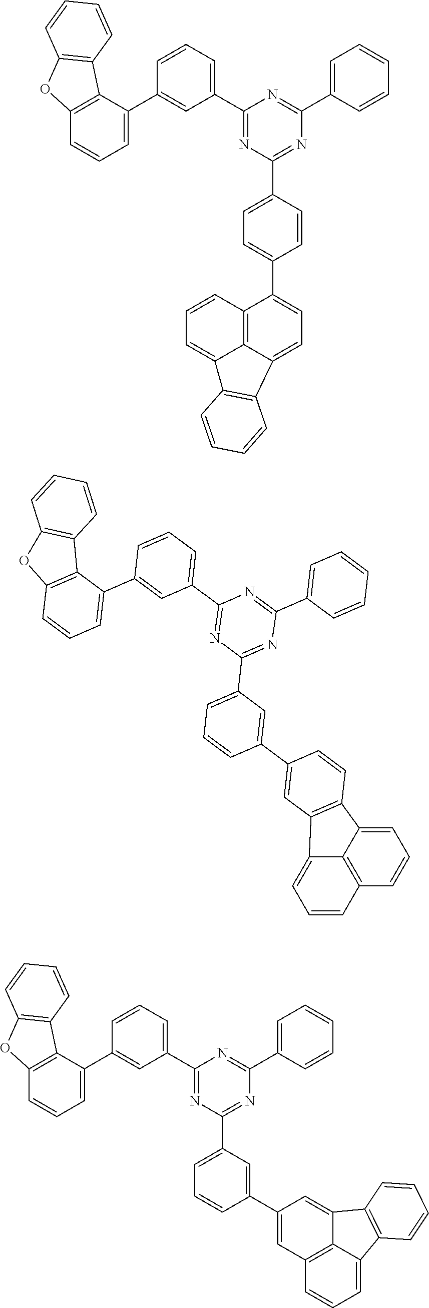 [SMILES: c1ccc(-c2nc(-c3ccc(-c4ccc5c6c(cccc46)-c4ccccc4-5)cc3)nc(-c3cccc(-c4cccc5oc6ccccc6c45)c3)n2)cc1 ; c1ccc(-c2nc(-c3cccc(-c4ccc5c(c4)-c4cccc6cccc-5c46)c3)nc(-c3cccc(-c4cccc5oc6ccccc6c45)c3)n2)cc1 ; c1ccc(-c2nc(-c3cccc(-c4cc5c6c(cccc6c4)-c4ccccc4-5)c3)nc(-c3cccc(-c4cccc5oc6ccccc6c45)c3)n2)cc1]

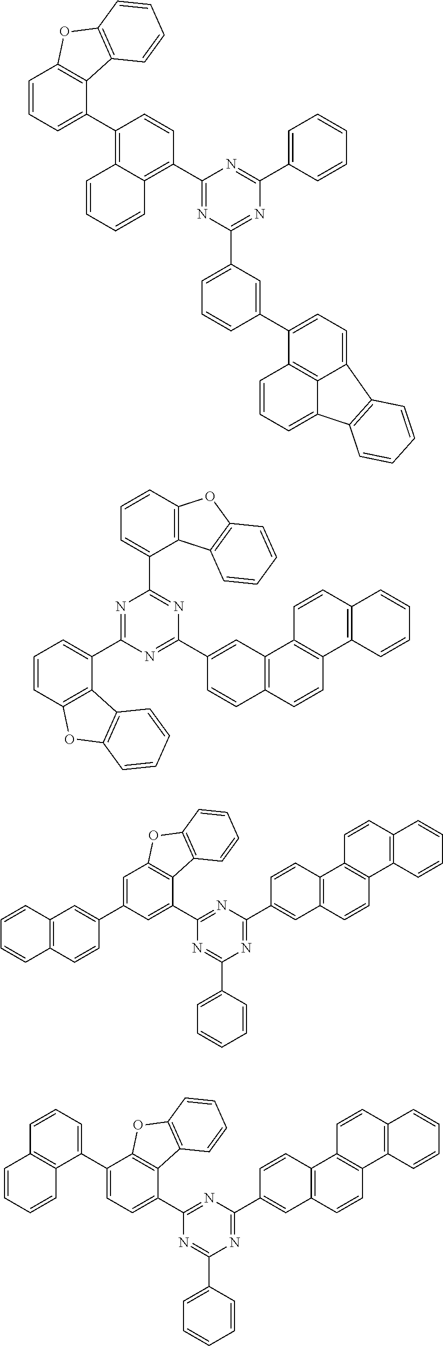 [SMILES: c1ccc(-c2nc(-c3cccc(-c4ccc5c6c(cccc46)-c4ccccc4-5)c3)nc(-c3ccc(-c4cccc5oc6ccccc6c45)c4ccccc34)n2)cc1 ; c1ccc2c(c1)ccc1c3cc(-c4nc(-c5cccc6oc7ccccc7c56)nc(-c5cccc6oc7ccccc7c56)n4)ccc3ccc21 ; c1ccc(-c2nc(-c3ccc4c(ccc5c6ccccc6ccc45)c3)nc(-c3cc(-c4ccc5ccccc5c4)cc4oc5ccccc5c34)n2)cc1 ; c1ccc(-c2nc(-c3ccc4c(ccc5c6ccccc6ccc45)c3)nc(-c3ccc(-c4cccc5ccccc45)c4oc5ccccc5c34)n2)cc1]

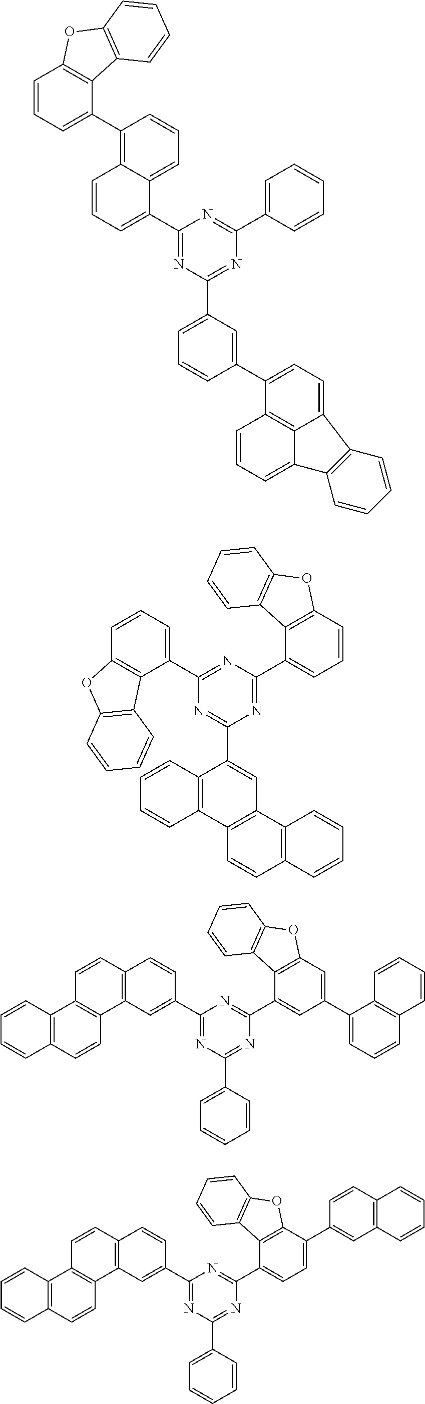

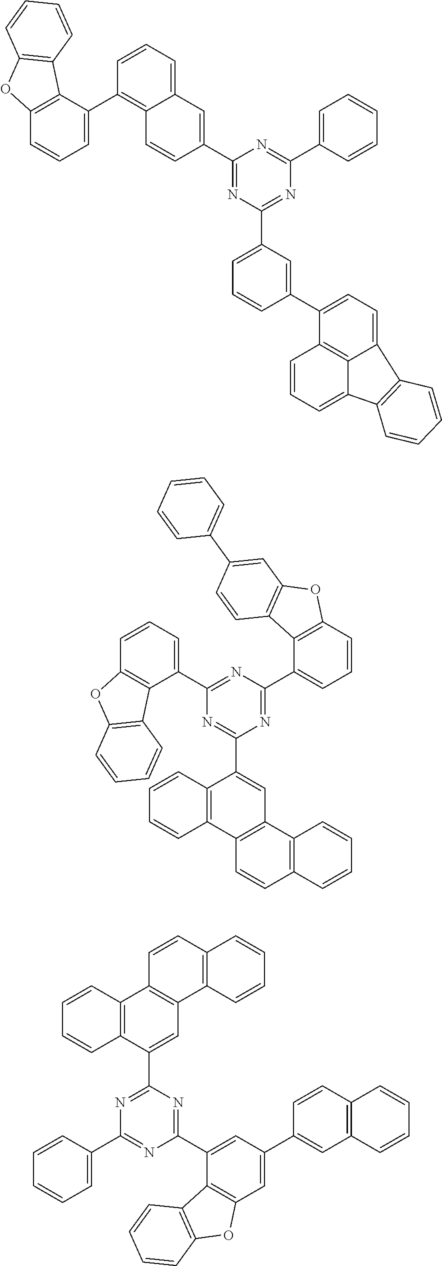 [SMILES: c1ccc(-c2nc(-c3cccc(-c4ccc5c6c(cccc46)-c4ccccc4-5)c3)nc(-c3ccc4c(-c5cccc6oc7ccccc7c56)cccc4c3)n2)cc1 ; c1ccc(-c2ccc3c(c2)oc2cccc(-c4nc(-c5cc6c7ccccc7ccc6c6ccccc56)nc(-c5cccc6oc7ccccc7c56)n4)c23)cc1 ; c1ccc(-c2nc(-c3cc4c5ccccc5ccc4c4ccccc34)nc(-c3cc(-c4ccc5ccccc5c4)cc4oc5ccccc5c34)n2)cc1]

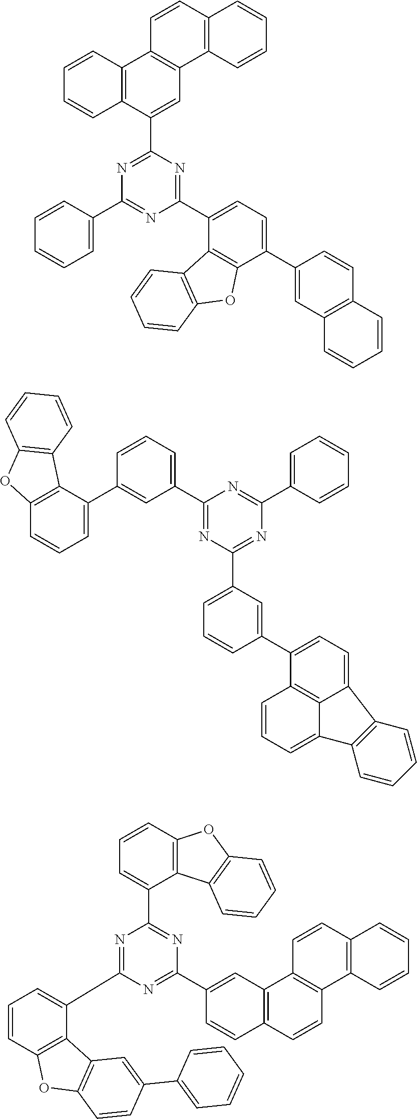 [SMILES: c1ccc(-c2nc(-c3cc4c5ccccc5ccc4c4ccccc34)nc(-c3ccc(-c4ccc5ccccc5c4)c4oc5ccccc5c34)n2)cc1 ; c1ccc(-c2nc(-c3cccc(-c4ccc5c6c(cccc46)-c4ccccc4-5)c3)nc(-c3cccc(-c4cccc5oc6ccccc6c45)c3)n2)cc1 ; c1ccc(-c2ccc3oc4cccc(-c5nc(-c6ccc7ccc8c9ccccc9ccc8c7c6)nc(-c6cccc7oc8ccccc8c67)n5)c4c3c2)cc1]

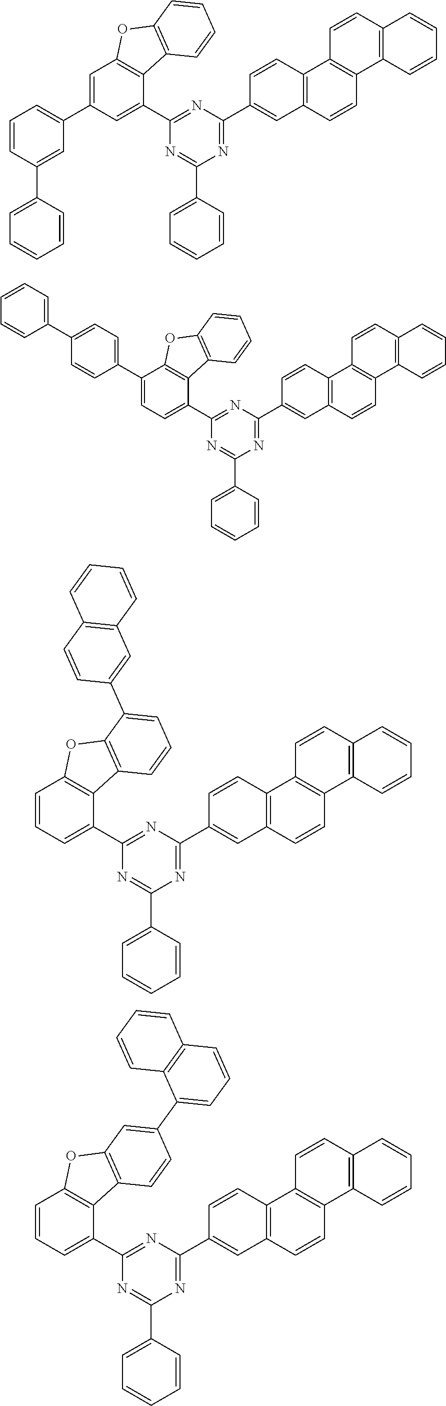

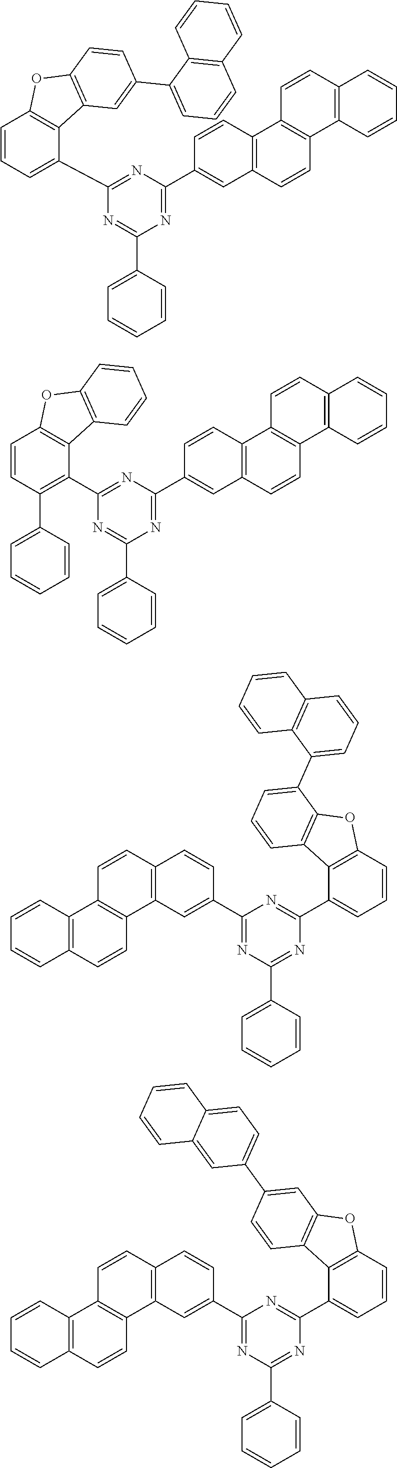

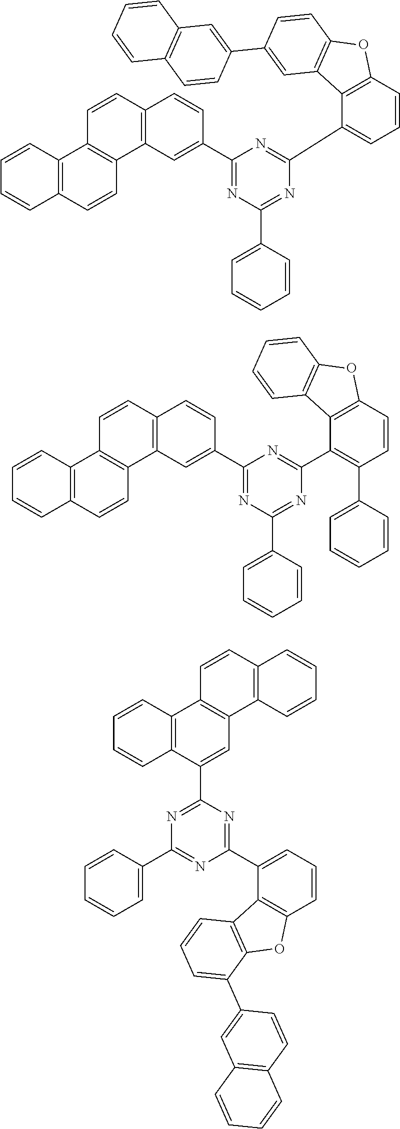

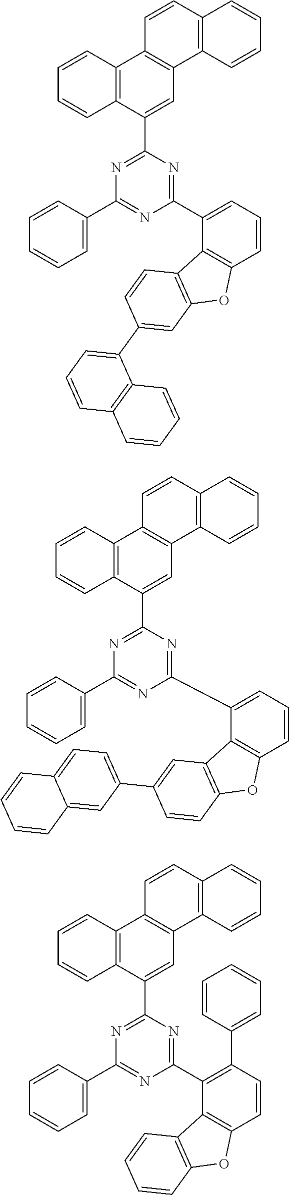

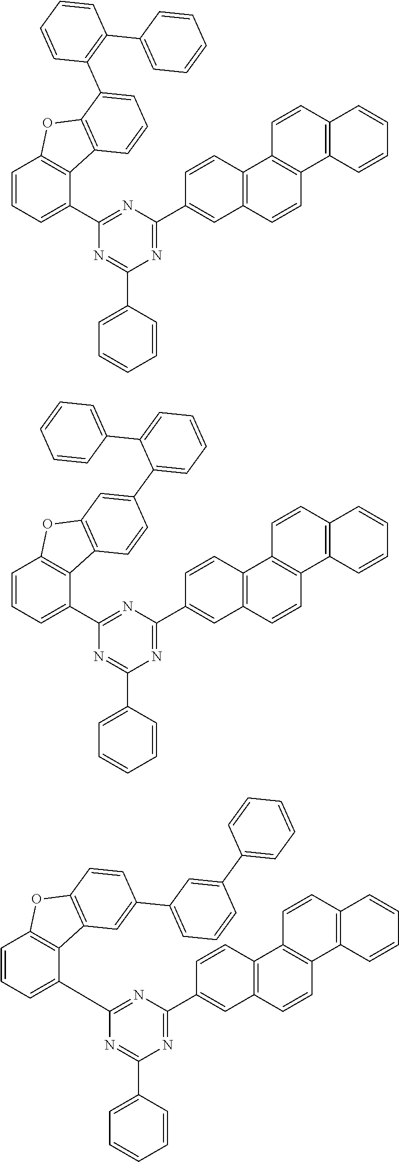 [SMILES: c1ccc(-c2nc(-c3ccc4c(ccc5c6ccccc6ccc45)c3)nc(-c3cccc4oc5c(-c6ccccc6-c6ccccc6)cccc5c34)n2)cc1 ; c1ccc(-c2nc(-c3ccc4c(ccc5c6ccccc6ccc45)c3)nc(-c3cccc4oc5cc(-c6ccccc6-c6ccccc6)ccc5c34)n2)cc1 ; c1ccc(-c2cccc(-c3ccc4oc5cccc(-c6nc(-c7ccccc7)nc(-c7ccc8c(ccc9c%10ccccc%10ccc89)c7)n6)c5c4c3)c2)cc1]

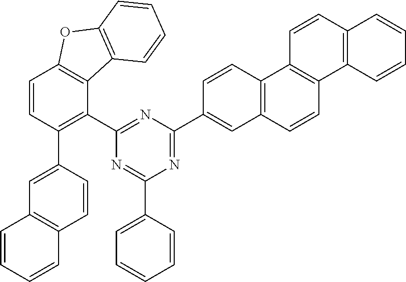 [SMILES: c1ccc(-c2nc(-c3ccc4c(ccc5c6ccccc6ccc45)c3)nc(-c3c(-c4ccc5ccccc5c4)ccc4oc5ccccc5c34)n2)cc1]

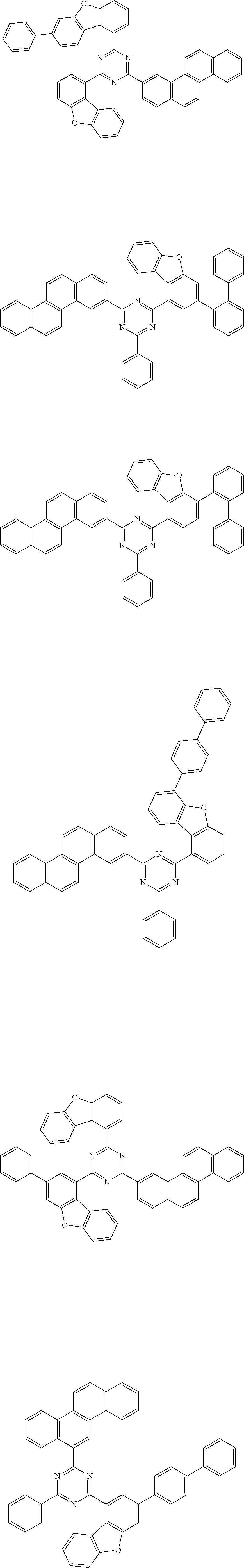

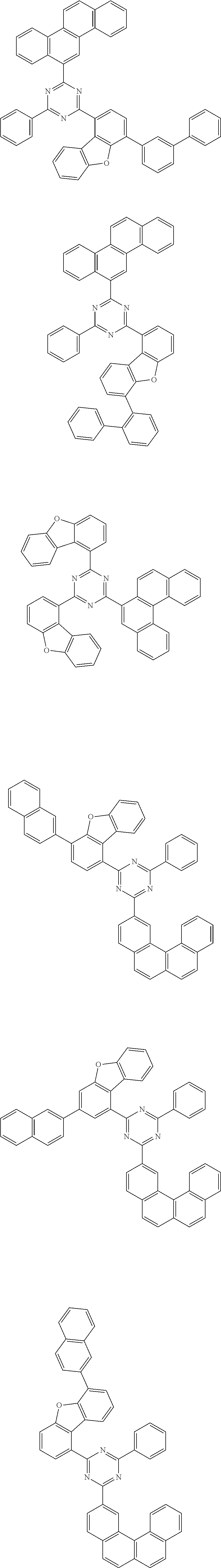

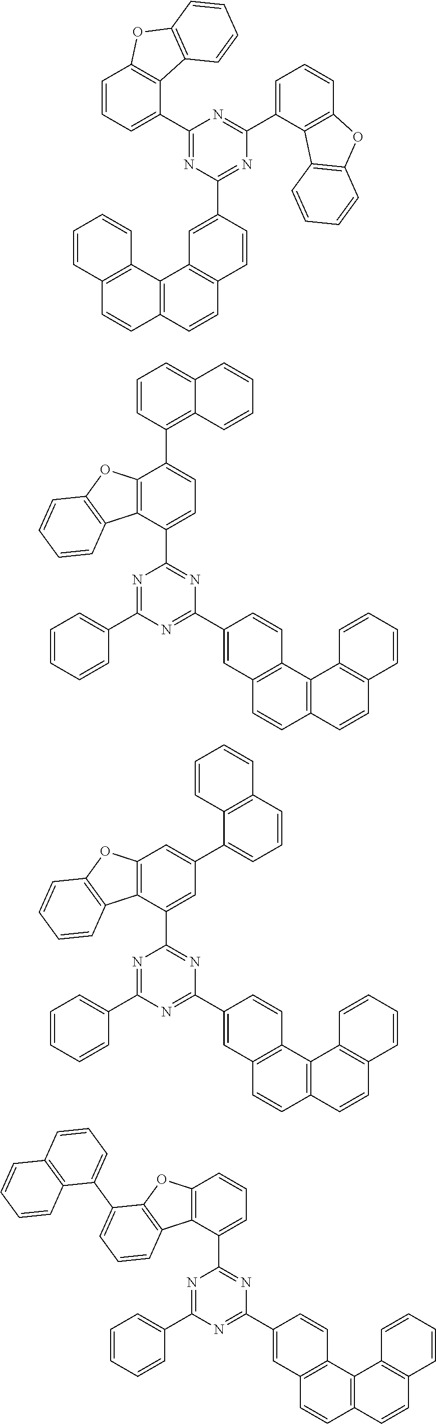

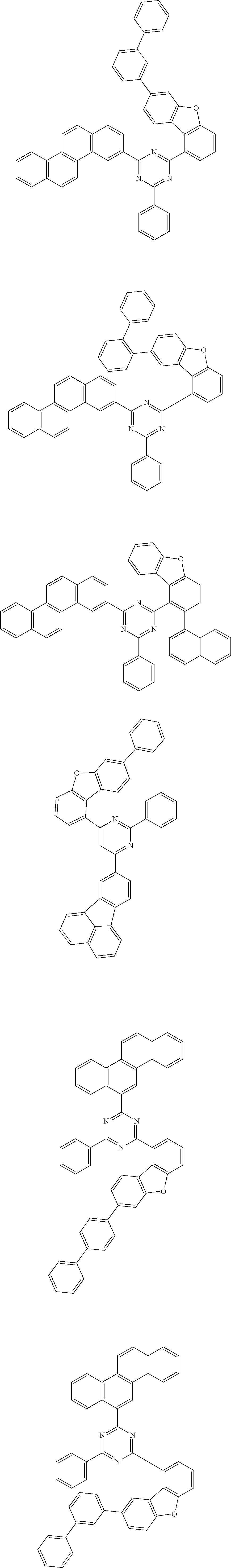

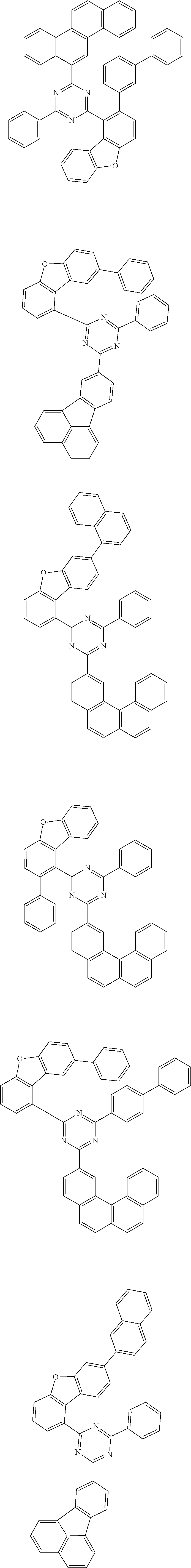

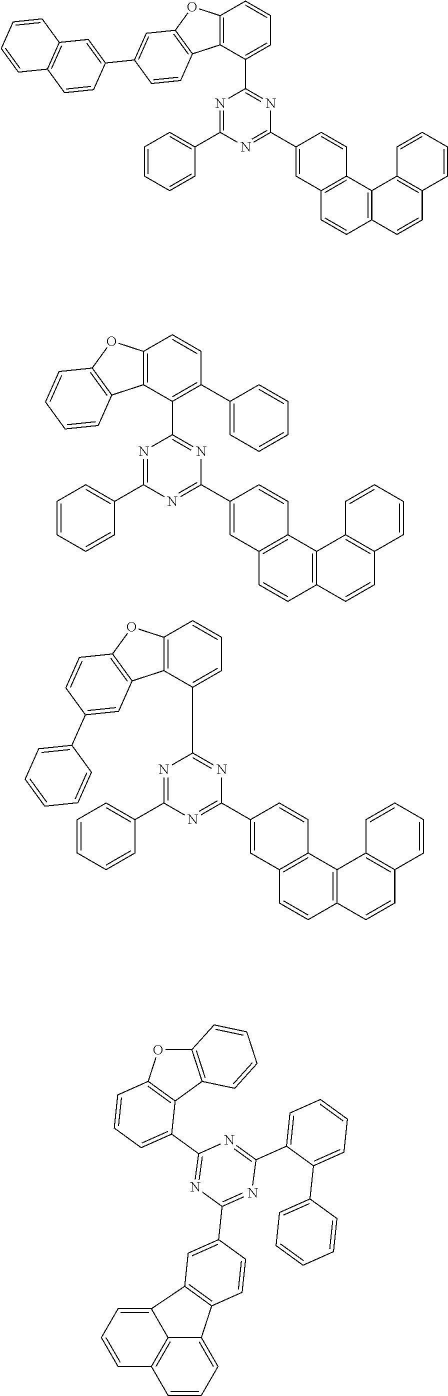 [SMILES: c1ccc(-c2nc(-c3ccc4c(ccc5ccc6ccccc6c54)c3)nc(-c3cccc4oc5cc(-c6ccc7ccccc7c6)ccc5c34)n2)cc1 ; c1ccc(-c2nc(-c3ccc4c(ccc5ccc6ccccc6c54)c3)nc(-c3c(-c4ccccc4)ccc4oc5ccccc5c34)n2)cc1 ; c1ccc(-c2ccc3oc4cccc(-c5nc(-c6ccccc6)nc(-c6ccc7c(ccc8ccc9ccccc9c87)c6)n5)c4c3c2)cc1 ; c1ccc(-c2ccccc2-c2nc(-c3ccc4c(c3)-c3cccc5cccc-4c35)nc(-c3cccc4oc5ccccc5c34)n2)cc1]

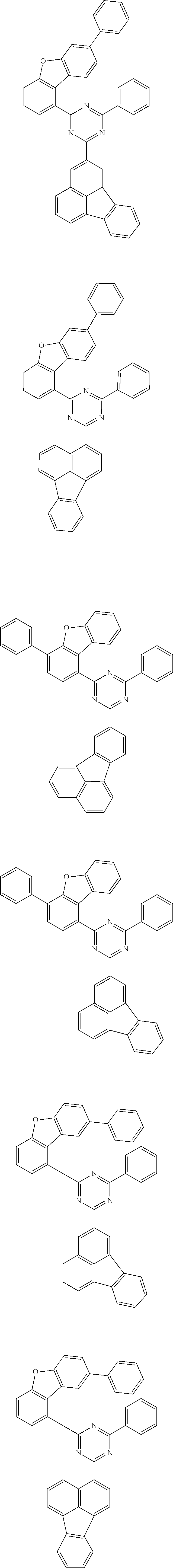

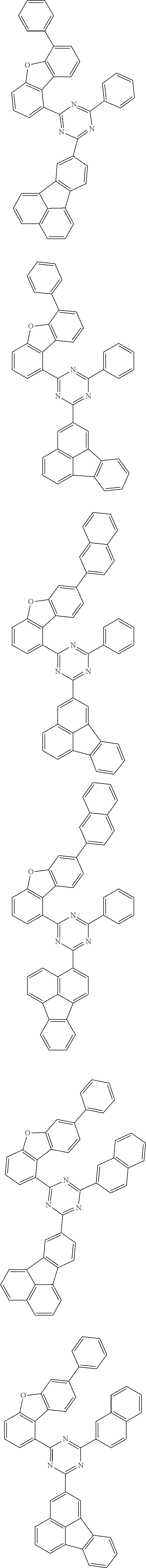

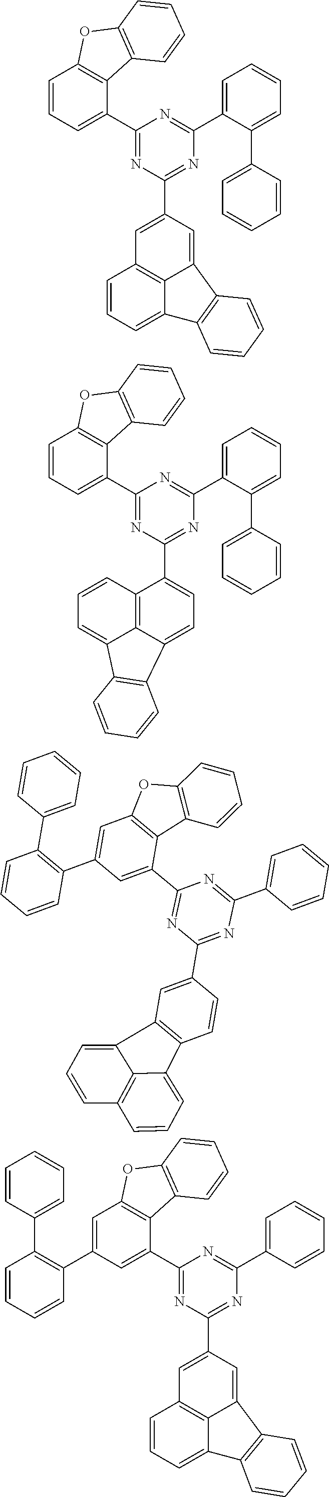

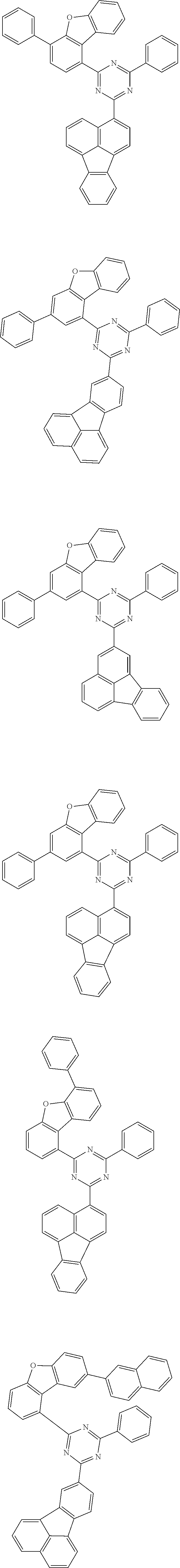

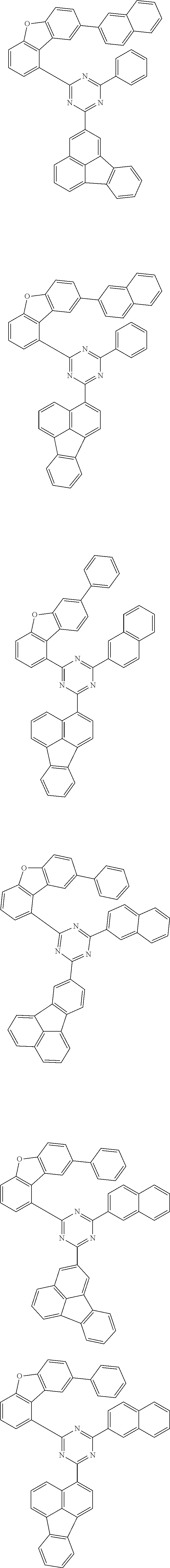

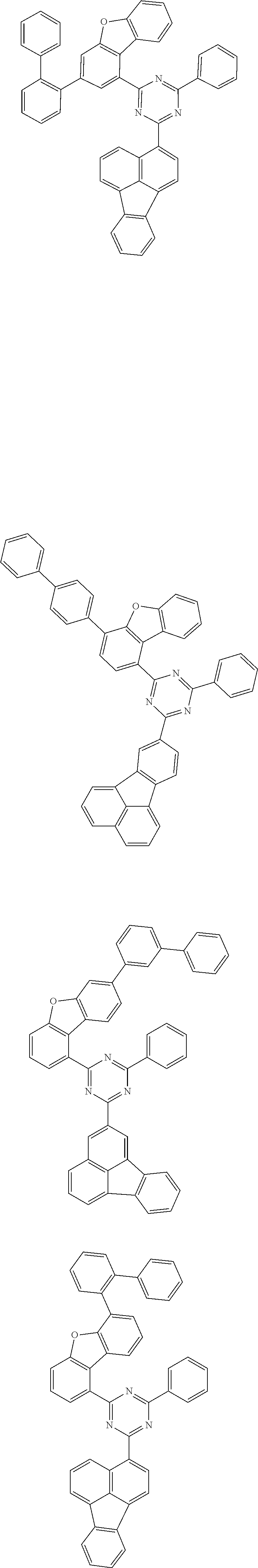 [SMILES: c1ccc(-c2nc(-c3ccc4c5c(cccc35)-c3ccccc3-4)nc(-c3cc(-c4ccccc4-c4ccccc4)cc4oc5ccccc5c34)n2)cc1 ; c1ccc(-c2ccc(-c3ccc(-c4nc(-c5ccccc5)nc(-c5ccc6c(c5)-c5cccc7cccc-6c57)n4)c4c3oc3ccccc34)cc2)cc1 ; c1ccc(-c2cccc(-c3ccc4c(c3)oc3cccc(-c5nc(-c6ccccc6)nc(-c6cc7c8c(cccc8c6)-c6ccccc6-7)n5)c34)c2)cc1 ; c1ccc(-c2nc(-c3ccc4c5c(cccc35)-c3ccccc3-4)nc(-c3cccc4oc5c(-c6ccccc6-c6ccccc6)cccc5c34)n2)cc1]

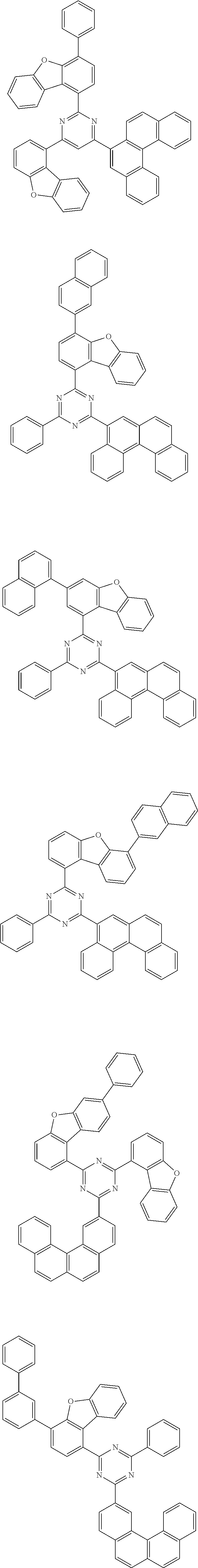

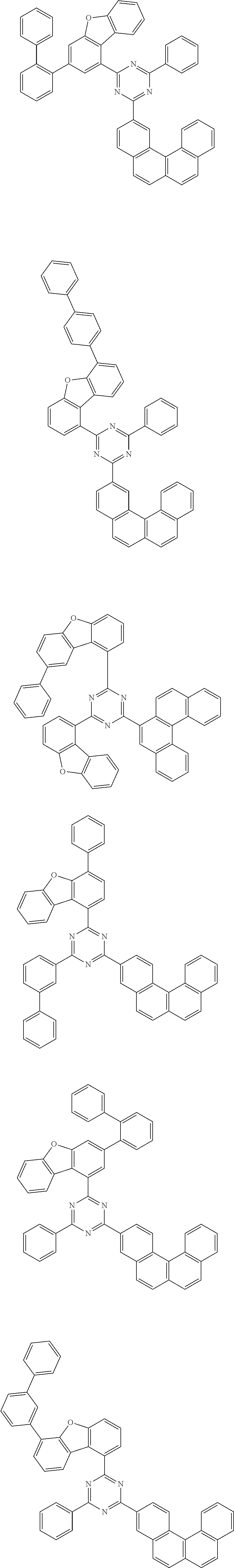

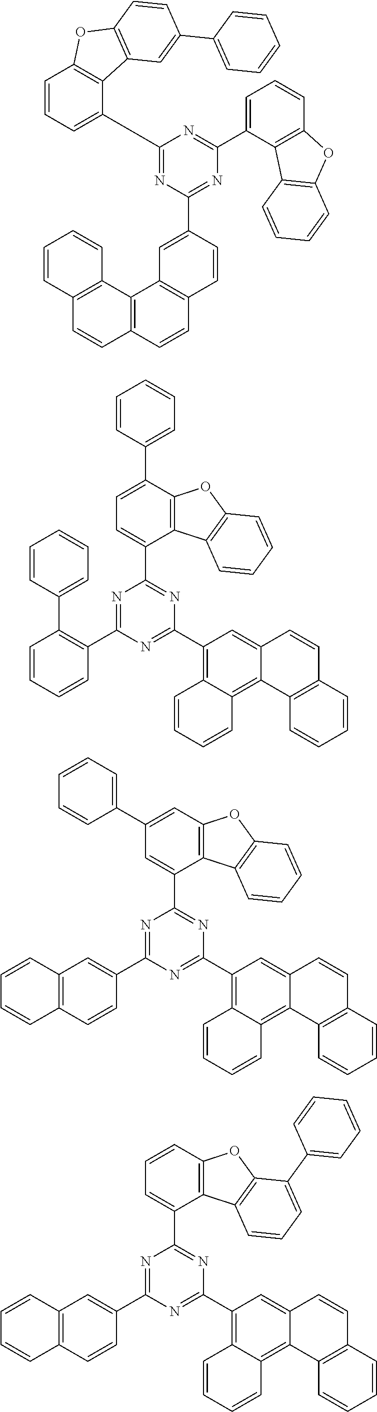

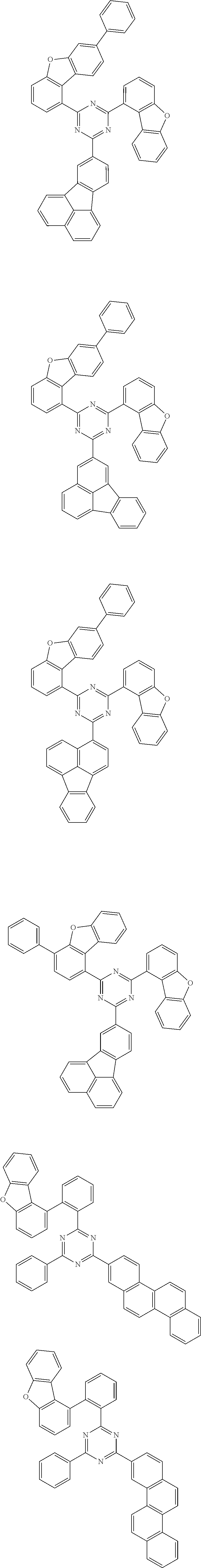

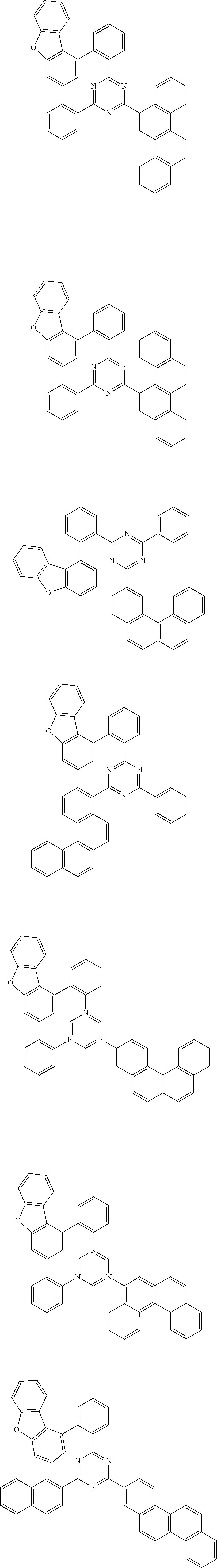

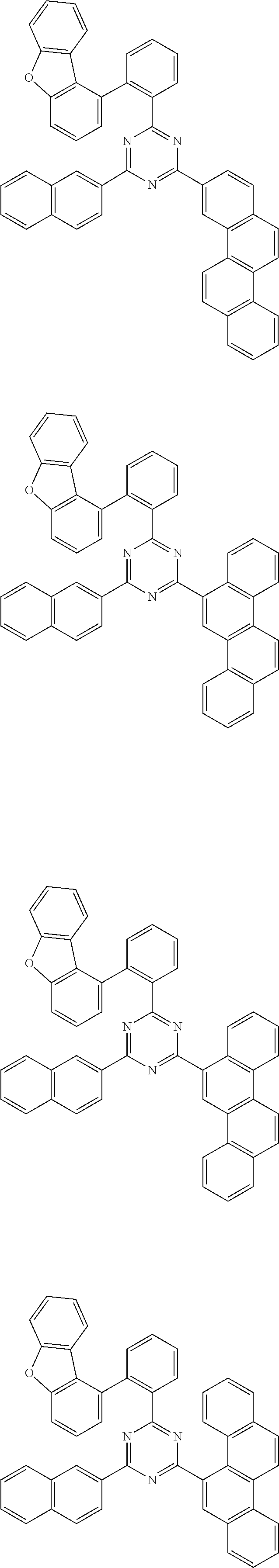 [SMILES: c1ccc(-c2cccc3oc4ccccc4c23)c(-c2nc(-c3ccc4ccccc4c3)nc(-c3ccc4ccc5c6ccccc6ccc5c4c3)n2)c1 ; c1ccc(-c2cccc3oc4ccccc4c23)c(-c2nc(-c3ccc4ccccc4c3)nc(-c3cc4c5ccccc5ccc4c4ccccc34)n2)c1 ; c1ccc(-c2cccc3oc4ccccc4c23)c(-c2nc(-c3ccc4ccccc4c3)nc(-c3cc4c5ccccc5ccc4c4ccccc34)n2)c1 ; c1ccc(-c2cccc3oc4ccccc4c23)c(-c2nc(-c3ccc4ccccc4c3)nc(-c3cc4ccccc4c4ccc5ccccc5c34)n2)c1]

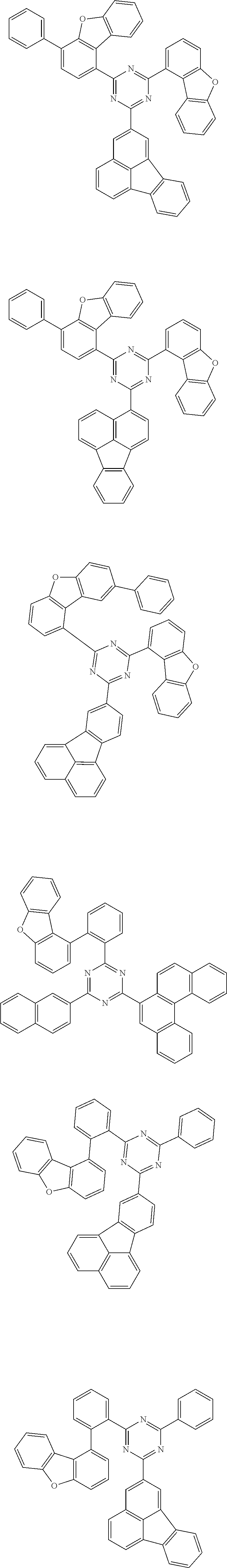

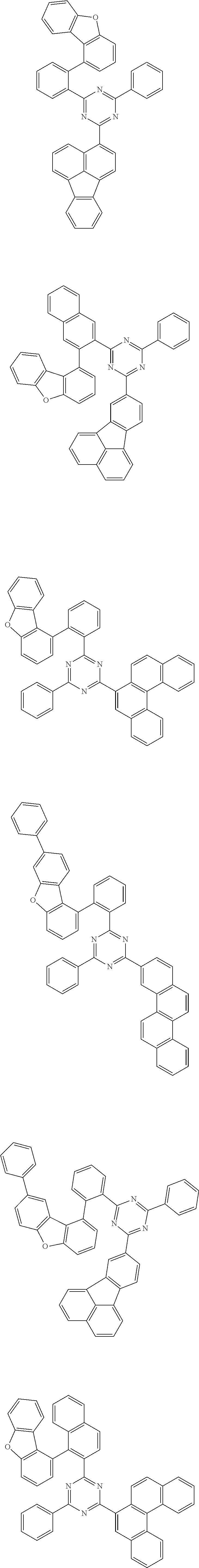

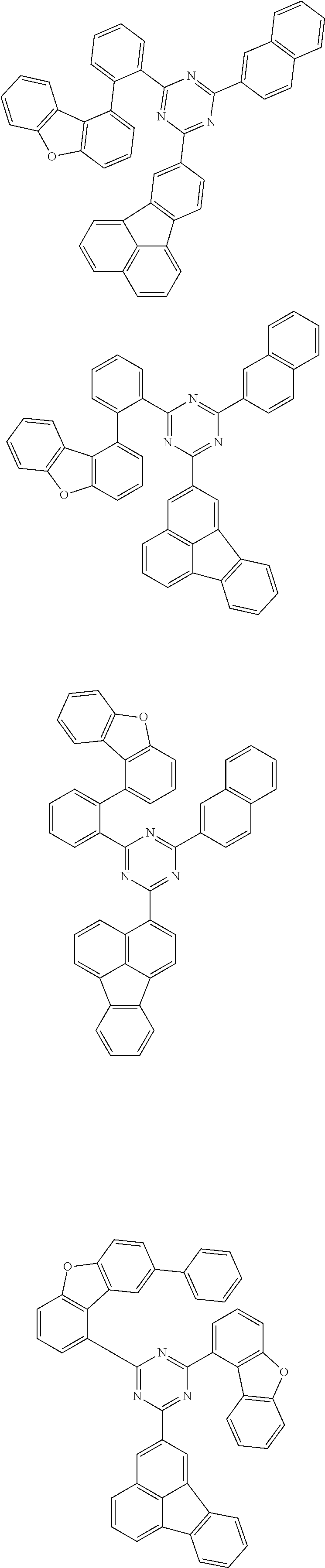 [SMILES: c1ccc(-c2cccc3oc4ccccc4c23)c(-c2nc(-c3ccc4c(c3)-c3cccc5cccc-4c35)nc(-c3ccc4ccccc4c3)n2)c1 ; c1ccc(-c2cccc3oc4ccccc4c23)c(-c2nc(-c3ccc4ccccc4c3)nc(-c3cc4c5c(cccc5c3)-c3ccccc3-4)n2)c1 ; c1ccc(-c2cccc3oc4ccccc4c23)c(-c2nc(-c3ccc4ccccc4c3)nc(-c3ccc4c5c(cccc35)-c3ccccc3-4)n2)c1 ; c1ccc(-c2ccc3oc4cccc(-c5nc(-c6cc7c8c(cccc8c6)-c6ccccc6-7)nc(-c6cccc7oc8ccccc8c67)n5)c4c3c2)cc1]

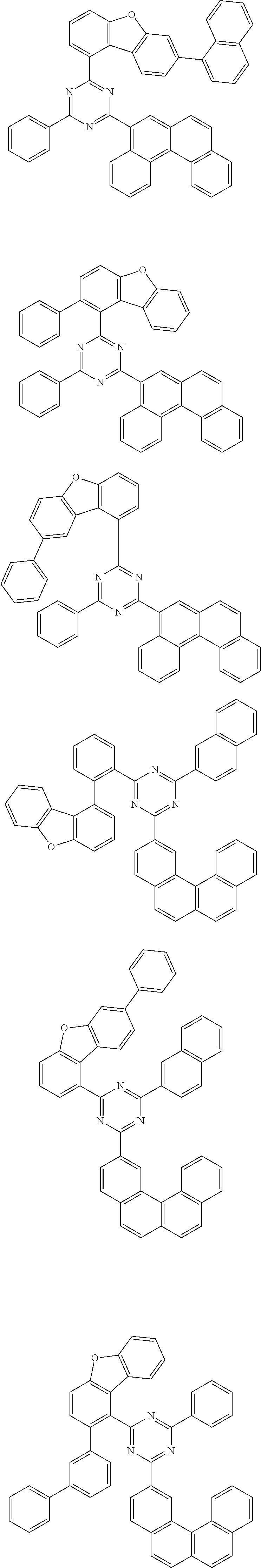

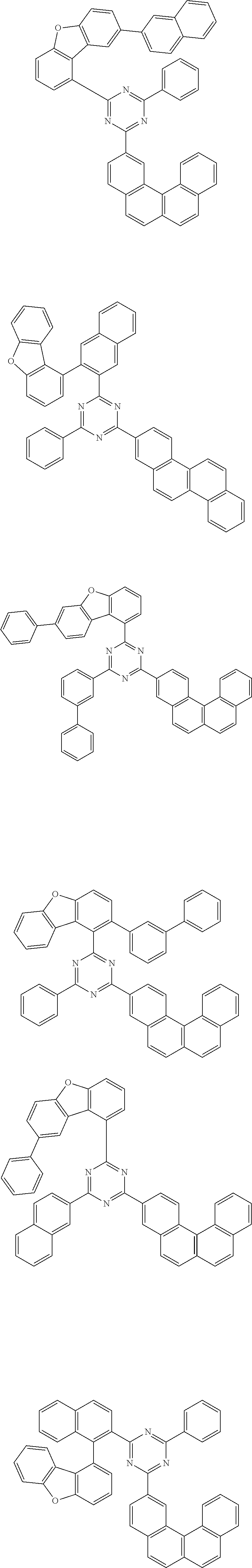

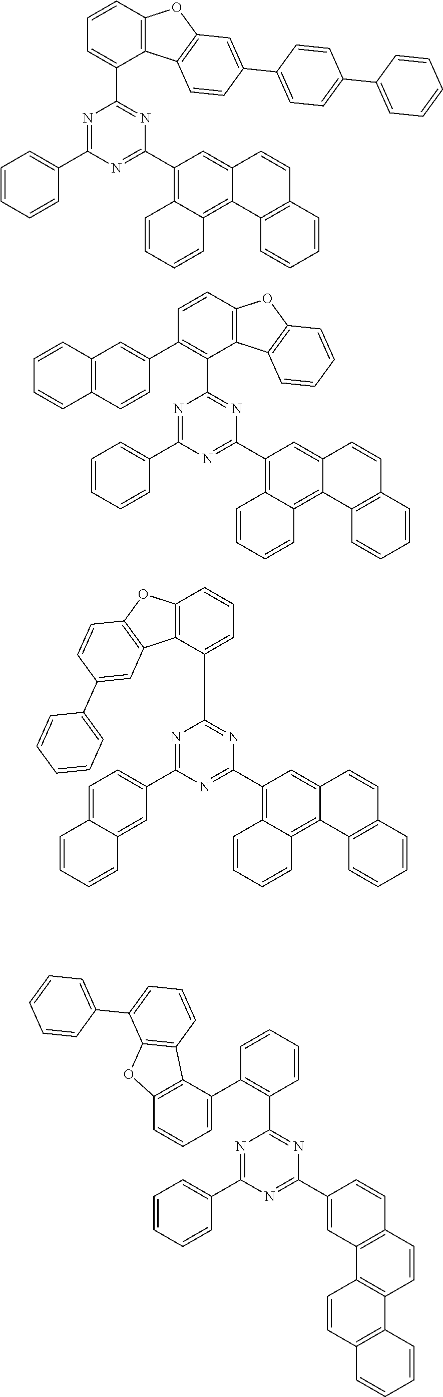

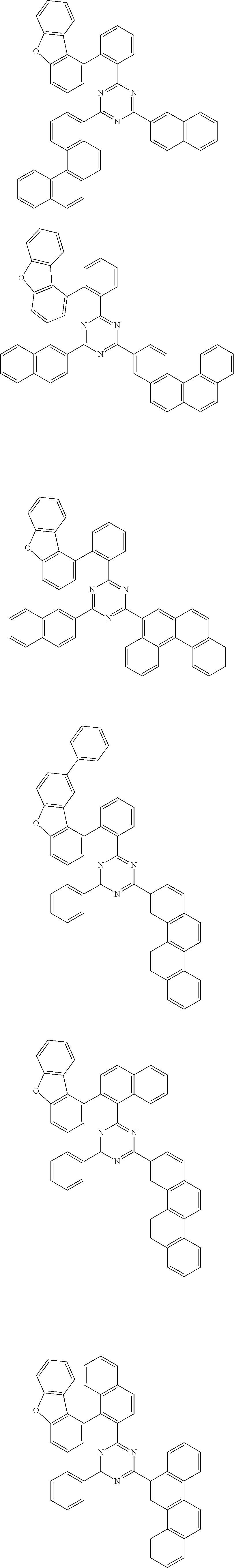

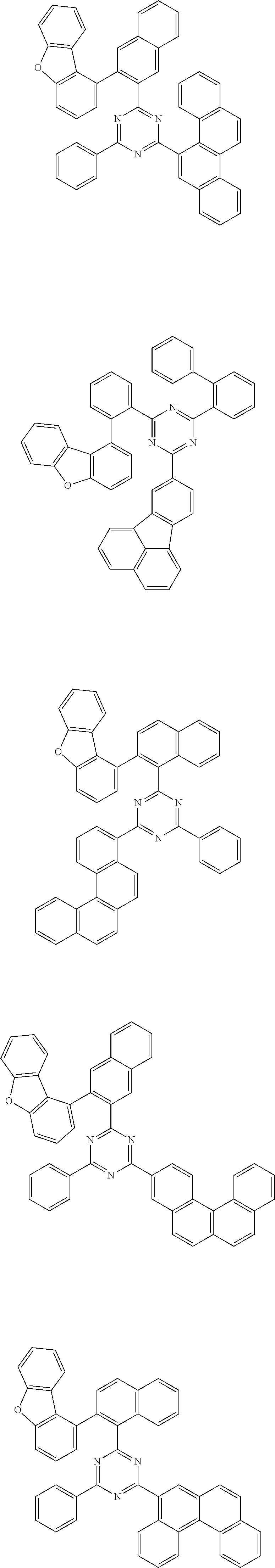 [SMILES: c1ccc(-c2nc(-c3cc4ccccc4cc3-c3cccc4oc5ccccc5c34)nc(-c3cc4ccccc4c4ccc5ccccc5c34)n2)cc1 ; c1ccc(-c2ccccc2-c2nc(-c3ccc4c(c3)-c3cccc5cccc-4c35)nc(-c3ccccc3-c3cccc4oc5ccccc5c34)n2)cc1 ; c1ccc(-c2nc(-c3c(-c4cccc5oc6ccccc6c45)ccc4ccccc34)nc(-c3cccc4c3ccc3ccc5ccccc5c34)n2)cc1 ; c1ccc(-c2nc(-c3ccc4c(ccc5ccc6ccccc6c54)c3)nc(-c3cc4ccccc4cc3-c3cccc4oc5ccccc5c34)n2)cc1 ; c1ccc(-c2nc(-c3c(-c4cccc5oc6ccccc6c45)ccc4ccccc34)nc(-c3cc4ccc5ccccc5c4c4ccccc34)n2)cc1]

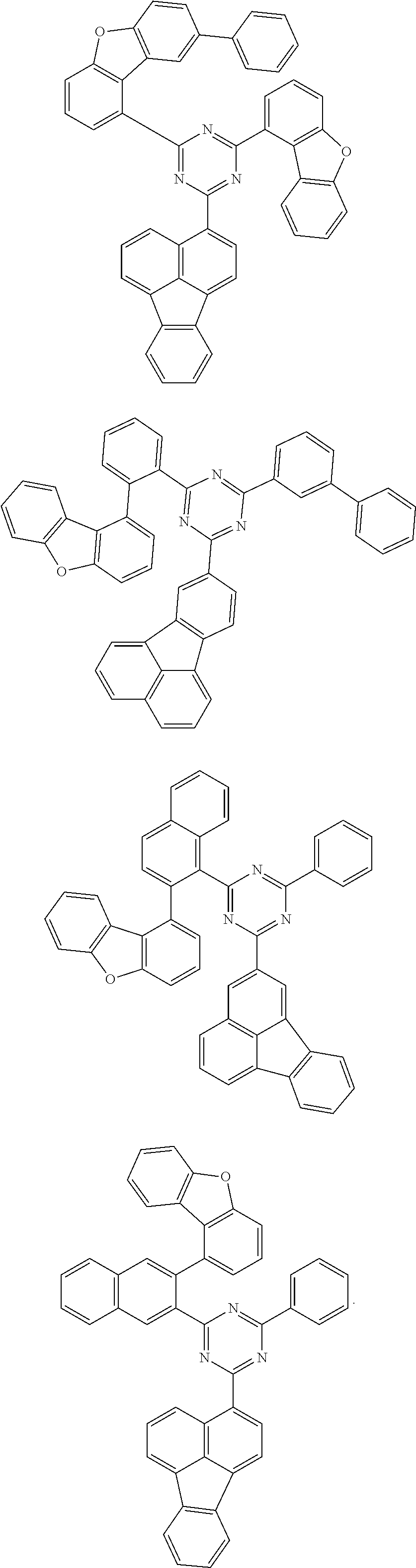

Meanwhile, the present disclosure provides a method for preparing the compound of Chemical Formula 1, as shown in the following Reaction Scheme 1 as an example.

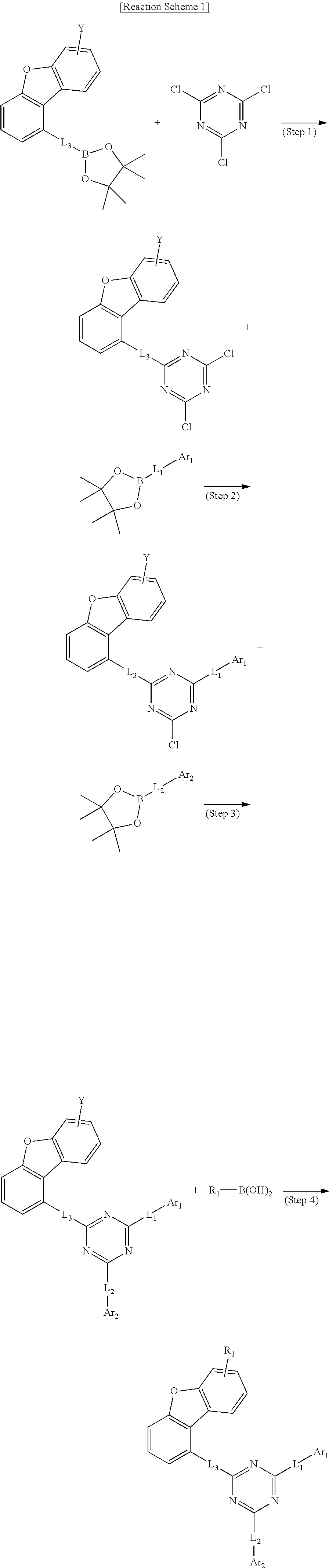

In Reaction Scheme 1, Y is halogen, preferably bromo or chloro. Further, the definitions of L₁, L₂, L₃, Ar₁, Ar₂ and R₁ in the Reaction Scheme 1 are the same as defined in Chemical Formula 1.

(Step 1), (Step 2) and (Step 3) can be performed by adding potassium carbonate and bis(tri-tert-butylphosphine)palladium(0) or tetrakis(triphenylphosphine)palladium(0) under a nitrogen atmosphere and a THF solvent, respectively. In addition, (Step 4) can be performed by adding potassium carbonate and bis(tri-tert-butylphosphine)palladium(0) under a nitrogen atmosphere and a THF solvent.

The above preparation method can be further embodied in Preparation Examples described hereinafter.

### (Organic Light Emitting Device)

In another embodiment of the present disclosure, provided is an organic light emitting device including the compound of Chemical Formula 1. In one example, the present disclosure provides an organic light emitting device comprising: a first electrode; a second electrode that is opposite to the first electrode; and one or more organic material layers that are between the first electrode and the second electrode, wherein one or more layers of the one or more organic material layers include the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, a hole blocking layer, an electron transport layer, an electron injection layer and the like as the organic material layer. However, the structure of the organic light emitting device is not limited thereto, and it can include a smaller number of organic layers.

Further, the organic layer can include a hole injection layer, a hole transport layer, or a layer that simultaneously performs hole injection and transport, wherein the hole injection layer, the hole transport layer, or the layer that simultaneously performs hole injection and transport can include the compound of Chemical Formula 1.

Further, the organic layer can include a light emitting layer, and the light emitting layer can include a compound of Chemical Formula 1.

Further, the organic layer can include a hole blocking layer, an electron transport layer, an electron injection layer, or a layer that simultaneously transports and injects electrons, wherein the hole blocking layer, the electron transport layer, the electron injection layer, or the layer that simultaneously performs hole injection and transport can include the compound of Chemical Formula 1.

Further, the organic layer can include a light emitting layer and an electron injection and transport layer, wherein the electron injection and transport layer can include the compound of Chemical Formula 1.

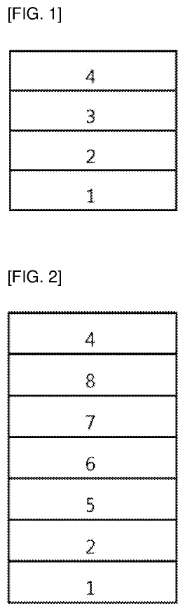

Further, the organic light emitting device according to the present disclosure can be a normal-type organic light emitting device in which an anode, one or more organic material layers, and a cathode are sequentially stacked on a substrate. Further, the organic light emitting device according to the present disclosure can be an inverted-type organic light emitting device in which a cathode, one or more organic material layers, and an anode are sequentially stacked on a substrate. For example, the structure of the organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a light emitting layer 7, an electron injection and transport layer 8, and a cathode 4. In such a structure, the compound of Chemical Formula 1 can be included in the light emitting layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking an anode, an organic material layer and a cathode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Wherein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive compound, a block copolymer in which a conjugate portion and a non-conjugate portion are present together, and the like, but are not limited thereto.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzothiazole and benzimidazole-based compound; a poly(p-phenylenevinylene) (PPV)-based polymer; a spiro compound; polyfluorene, rubrene, and the like, but are not limited thereto.

The electron blocking layer is a layer provided between the hole transport layer and the light emitting layer in order to prevent the electrons injected in the cathode from being transferred to the hole transport layer without being recombined in the light emitting layer, which can also be referred to as an electron inhibition layer or an electron stopping layer. The electron blocking layer is preferably a material having a smaller electron affinity than the electron transport layer. Preferably, the compound of Chemical Formula 1 can be included as a material for the electron blocking layer.

The light emitting layer can include a host material and a dopant material. As the host material, the above-mentioned compound of Chemical Formula 1 can be used. In addition, as the host material that can be further used, a fused aromatic ring derivative, a heterocycle-containing compound, or the like can be used. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Further, examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

For example, the dopant material of the present disclosure can include one of the following Dp-1 to Dp-38, but is not limited thereto.

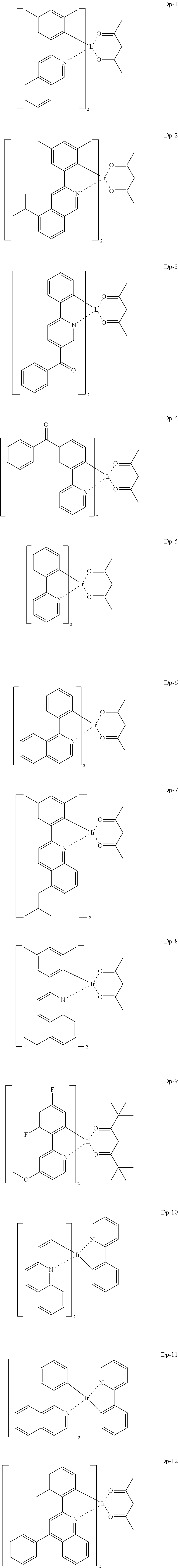

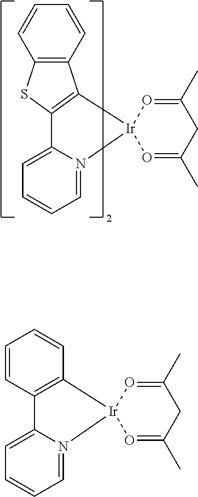

The electron transport layer is a layer which receives electrons from an electron injection layer and transports the electrons to a light emitting layer, and an electron transport material is suitably a material which can receive electrons well from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer can be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

On the other hand, in the present disclosure, the “electron injection and transport layer” is a layer that performs both the roles of the electron injection layer and the electron transport layer, and the materials that perform the roles of each layer can be used alone or in combination, without being limited thereto. Preferably, the compound of Chemical Formula 1 can be included as a material for the electron injection and transport layer.

The organic light emitting device according to the present disclosure can be a bottom emission device, a top emission device, or a double-sided light emitting device, and particularly, can be a bottom emission device that requires relatively high luminous efficiency.

In addition, the compound according to the present disclosure can be included in an organic solar cell or an organic transistor in addition to an organic light emitting device.

The preparation of the compound of Chemical Formula 1 and the organic light emitting device including the same will be specifically described in the following Examples. However, the following Examples are provided for illustrative purposes only, and are not intended to limit the scope of the present disclosure.

### EXAMPLES

### Example 1: Synthesis of Compound 1

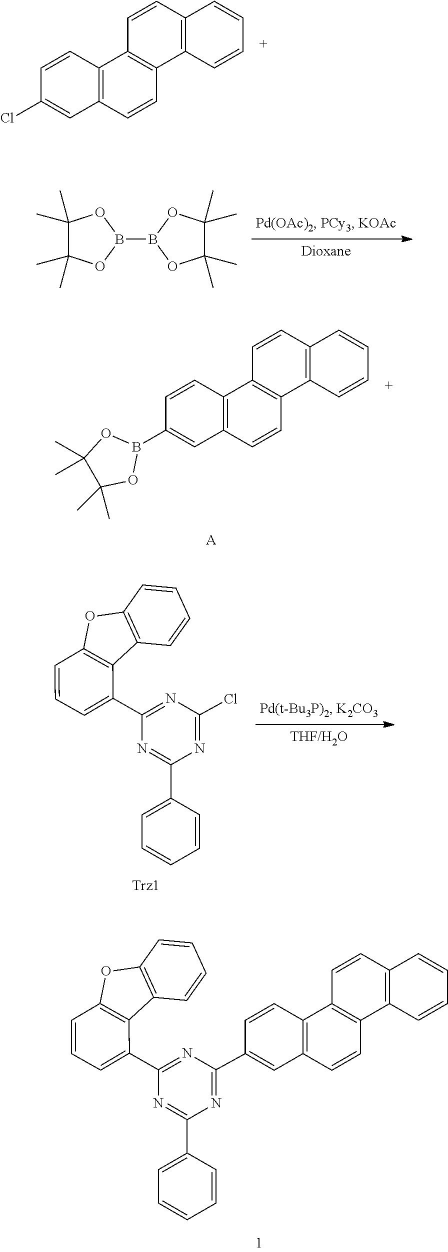

2-Chlorochrysene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone)palladium(0) (1 g, 1.7 mmol) and tricyclohexylphosphine (1 g, 3.4 mmol) were added. After reacting for 10 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 13.2 g of Compound A. (Yield: 66%, MS: [M+H]+=352)

Compound A (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17.2 g of Compound 1. (Yield: 74%, MS: [M+H]+=550)

### Example 2: Synthesis of Compound 2

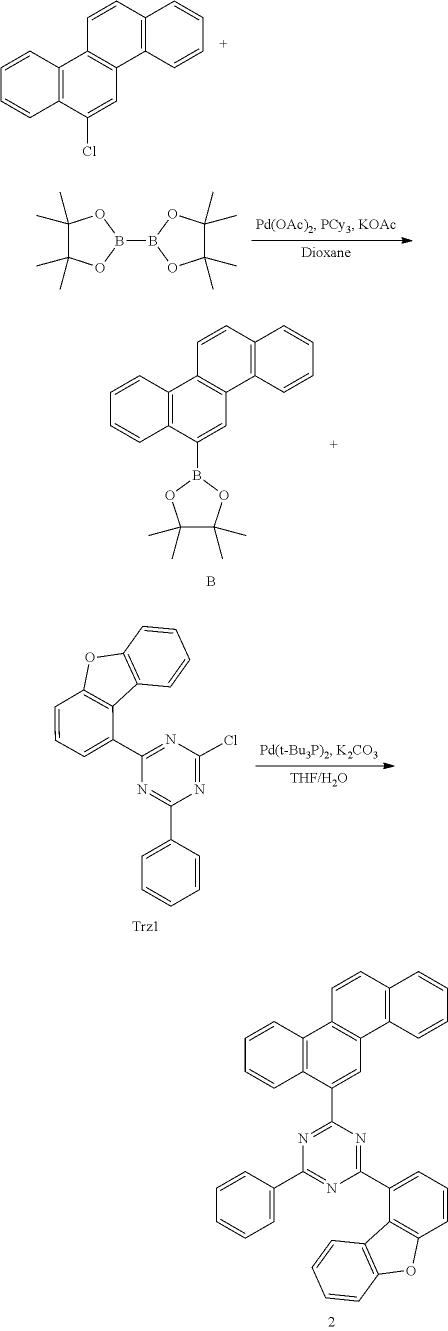

6-Chlorochrysene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone)palladium(0) (1 g, 1.7 mmol) and tricyclohexylphosphine (1 g, 3.4 mmol) were added. After reacting for 8 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 15.8 g of Compound B. (Yield: 79%, MS: [M+H]+=352)

Compound B (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 14.2 g of Compound 2. (Yield: 61%, MS: [M+H]+=550)

### Example 3: Synthesis of Compound 3

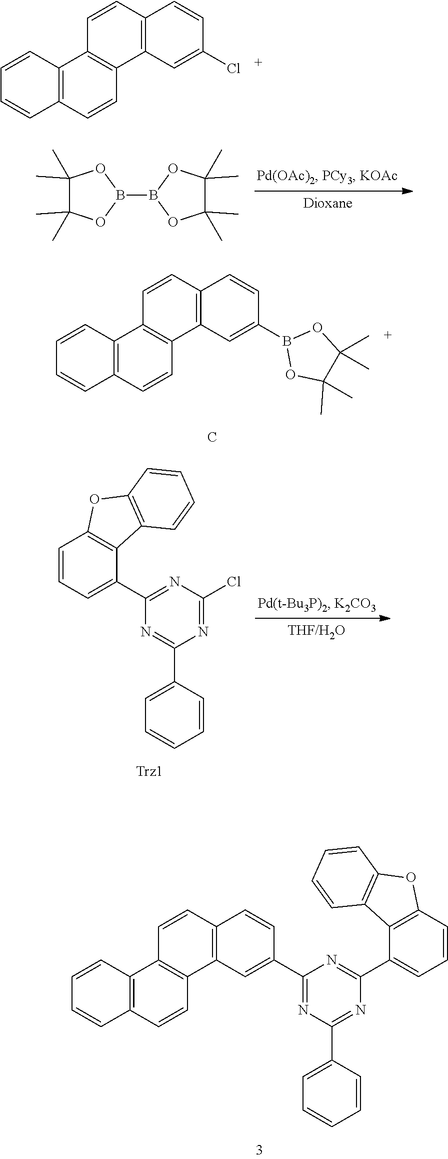

3-Chlorochrysene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone)palladium(0) (1 g, 1.7 mmol) and tricyclohexylphosphine (1 g, 3.4 mmol) were added. After reacting for 7 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.4 g of Compound C. (Yield: 62%, MS: [M+H]+=352)

Compound C (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 15.8 g of Compound 3. (Yield: 68%, MS: [M+H]+=550)

### Example 4: Synthesis of Compound 4

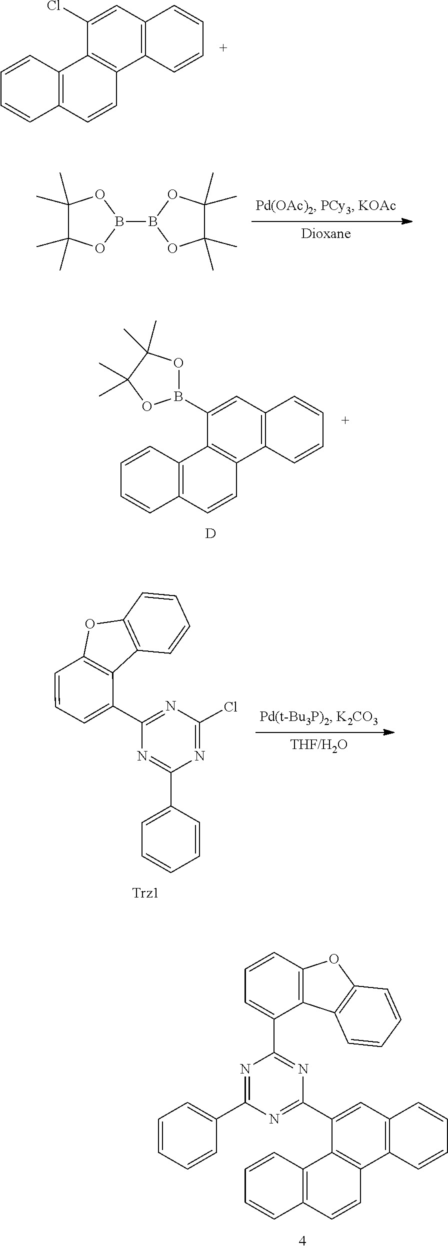

5-Chlorochrysene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone)palladium(0) (1 g, 1.7 mmol) and tricyclohexylphosphine (1 g, 3.4 mmol) were added. After reacting for 7 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 15.2 g of Compound D. (Yield: 76%, MS: [M+H]+=352)

Compound D (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17 g of Compound 4. (Yield: 73%, MS: [M+H]+=550)

### Example 5: Synthesis of Compound 5

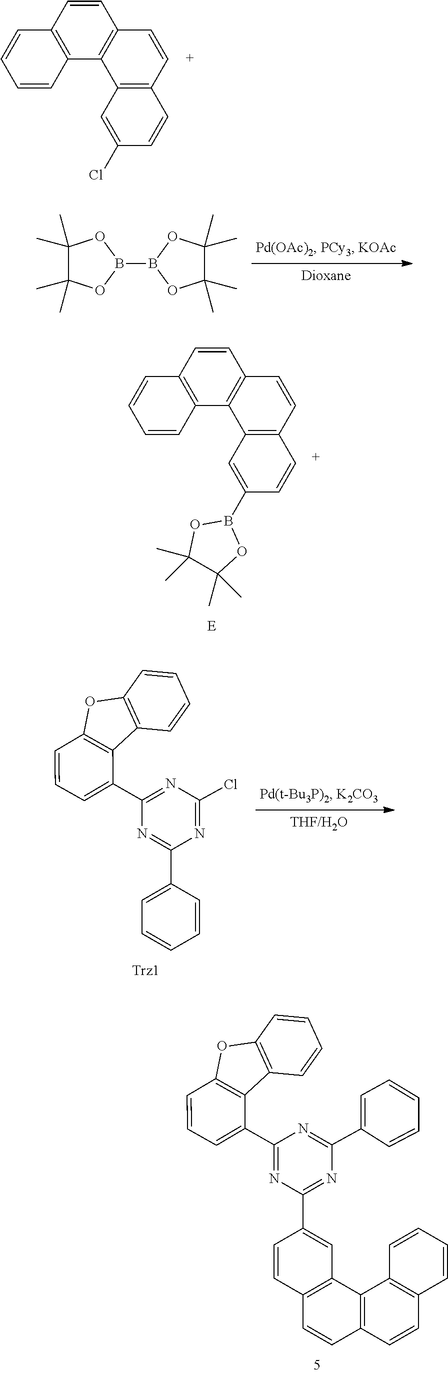

2-Chlorobenzo[c]phenanthrene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone)palladium(0) (1 g, 1.7 mmol) and tricyclohexylphosphine (1 g, 3.4 mmol) were added. After reacting for 7 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 15 g of Compound E. (Yield: 75%, MS: [M+H]+=352)

Compound E (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18.4 g of Compound 5. (Yield: 79%, MS: [M+H]+=550)

### Example 6: Synthesis of Compound 6

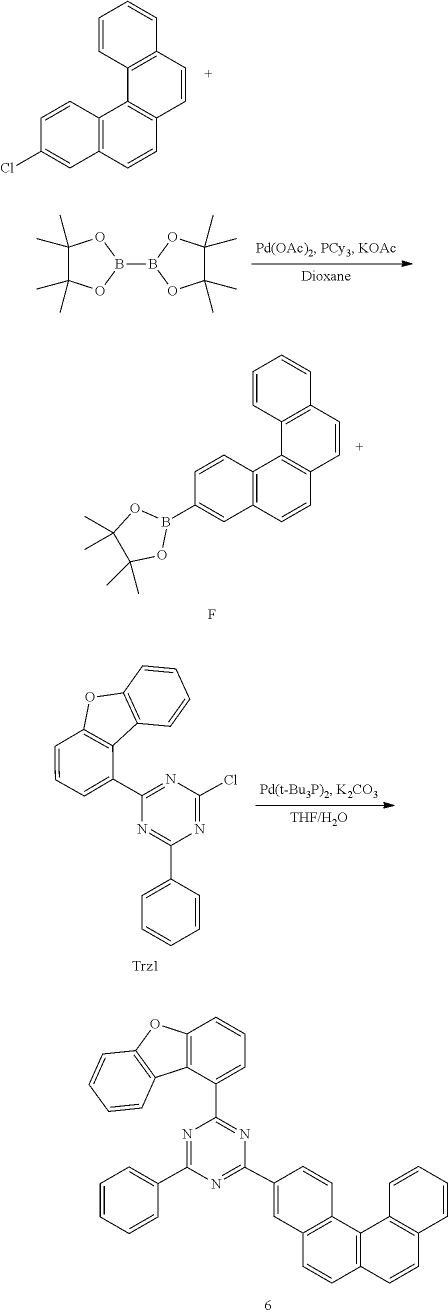

3-Chlorobenzo[c]phenanthrene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone) palladium (0) (1 g, 1.7 mmol) and tricyclohexylphosphine (1 g, 3.4 mmol) were added. After reacting for 6 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 14.2 g of Compound F. (Yield: 71%, MS: [M+H]+=352)

Compound F (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17.7 g of Compound 6. (Yield: 76%, MS: [M+H]+=550)

### Example 7: Synthesis of Compound 7

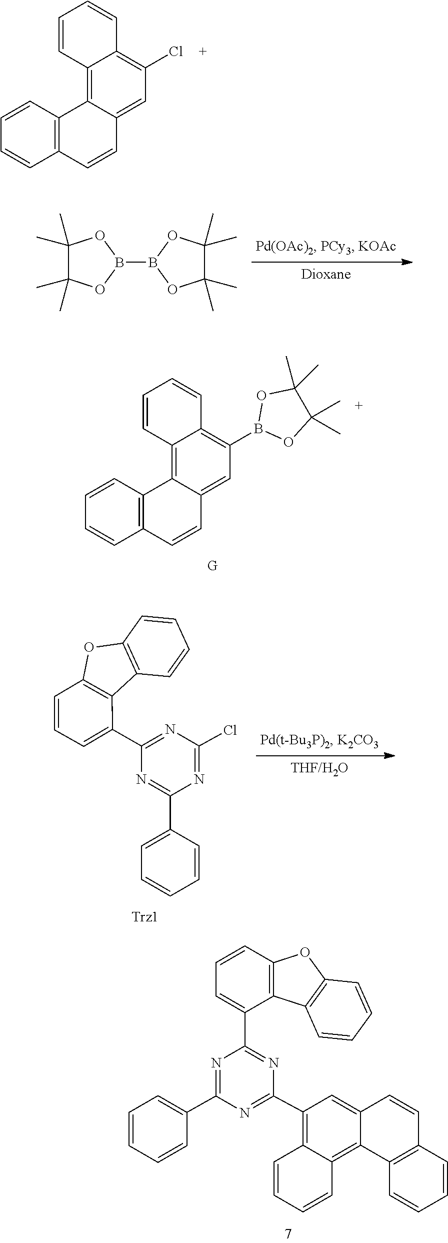

5-Chlorobenzo[c]phenanthrene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone) palladium (0) (1 g, 1.7 mmol) and tricyclohexylphosphine (1 g, 3.4 mmol) were added. After reacting for 5 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.8 g of Compound G. (Yield: 64%, MS: [M+H]+=352)

Compound G (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 14.9 g of Compound 7. (Yield: 64%, MS: [M+H]+=550)

### Example 8: Synthesis of Compound 8

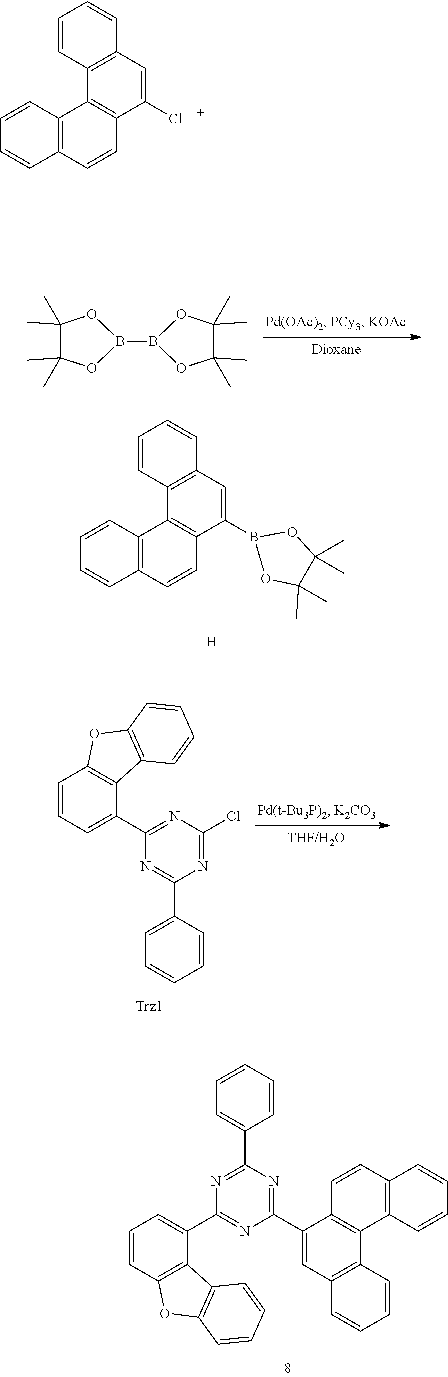

6-Chlorobenzo[c]phenanthrene (15 g, 57.1 mmol) and bis(pinacolato)diboron (15.9 g, 62.8 mmol) were added to 300 ml of 1,4-dioxane under a nitrogen atmosphere, and the mixture was stirred under reflux. Then, potassium acetate (8.4 g, 85.6 mmol) was added thereto, sufficiently stirred, and then bis(dibenzylideneacetone) palladium (0) (1 g, 1.7 mmol) and tricyclohexylphosphine (1 g, 3.4 mmol) were added. After reacting for 5 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 14.6 g of Compound H. (Yield: 73%, MS: [M+H]+=352)

Compound H (15 g, 42.3 mmol) and Trz1 (15.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 14.4 g of Compound 8. (Yield: 62%, MS: [M+H]+=550)

### Example 9: Synthesis of Compound 9

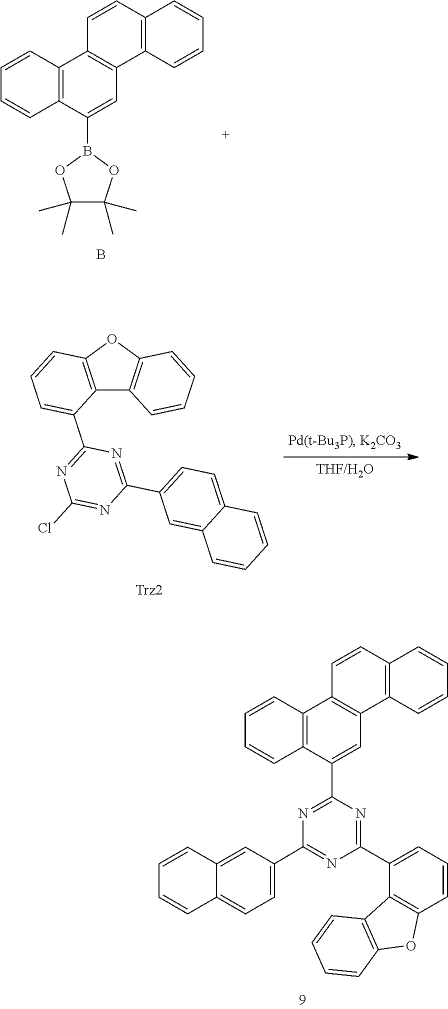

Compound B (15 g, 42.3 mmol) and Trz2 (18.1 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 16.2 g of Compound 9. (Yield: 64%, MS: [M+H]+=600)

### Example 10: Synthesis of Compound 10

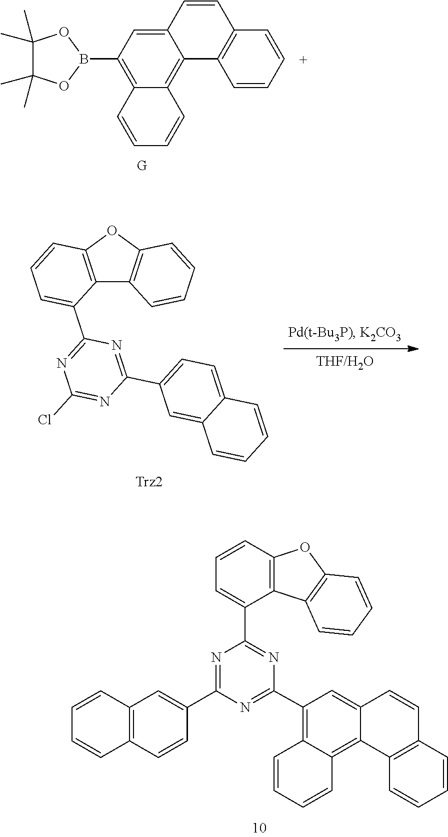

Compound G (15 g, 42.3 mmol) and Trz2 (18.1 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine)palladium(0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 16.2 g of Compound 10. (Yield: 64%, MS: [M+H]+=600)

### Example 11: Synthesis of Compound 11

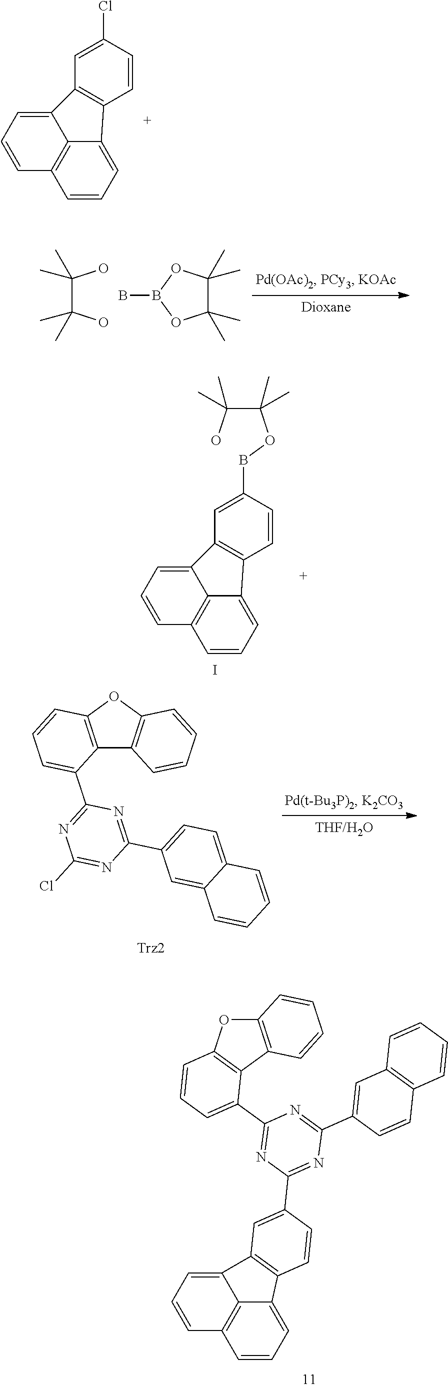

8-Chlorofluoranthene (15 g, 63.4 mmol) and bis(pinacolato)diboron (17.7 g, 69.7 mmol) were added to 300 ml of 1,4-dioxane under a nitrogen atmosphere, and the mixture was stirred under reflux. Then, potassium acetate (9.3 g, 95.1 mmol) was added thereto, sufficiently stirred, and then bis(dibenzylideneacetone) palladium (0) (1.1 g, 1.9 mmol) and tricyclohexylphosphine (1.1 g, 3.8 mmol) were added. After reacting for 8 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 15 g of Compound I. (Yield: 72%, MS: [M+H]+=329)

Compound I (15 g, 45.7 mmol) and Trz2 (19.6 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17.6 g of Compound 11. (Yield: 67%, MS: [M+H]+=574)

### Example 12: Synthesis of Compound 12

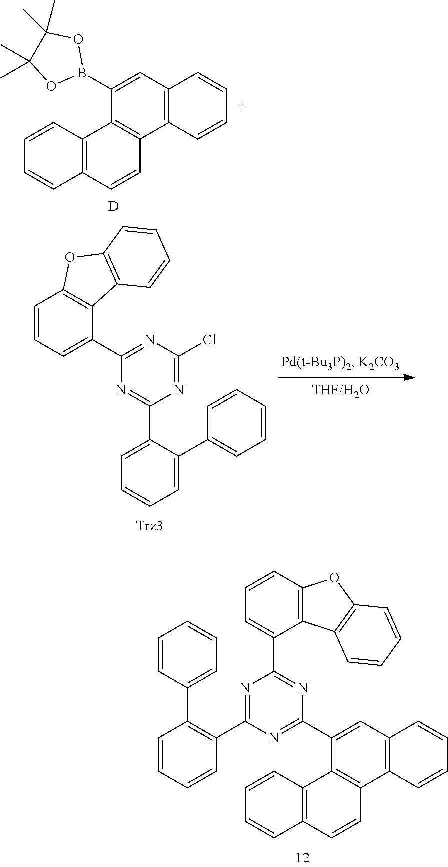

Compound D (15 g, 42.3 mmol) and Trz3 (19.3 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 20.6 g of Compound 12. (Yield: 78%, MS: [M+H]+=626)

### Example 13: Synthesis of Compound 13

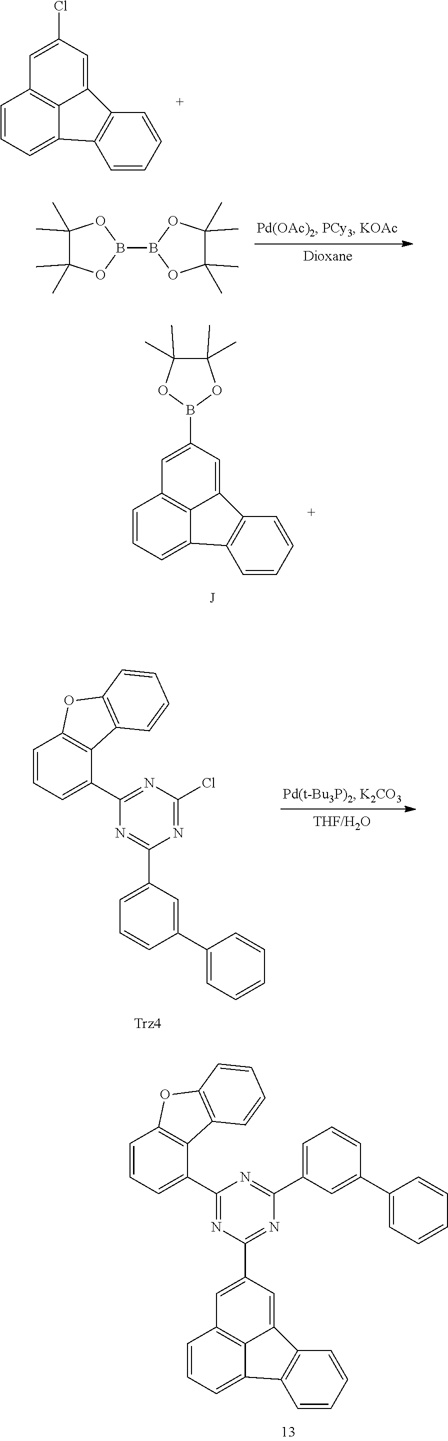

2-Chlorofluoranthene (15 g, 63.4 mmol) and bis(pinacolato)diboron (17.7 g, 69.7 mmol) were added to 300 ml of 1,4-dioxane under a nitrogen atmosphere, and the mixture was stirred under reflux. Then, potassium acetate (9.3 g, 95.1 mmol) was added thereto, sufficiently stirred, and then bis(dibenzylideneacetone) palladium (0) (1.1 g, 1.9 mmol) and tricyclohexylphosphine (1.1 g, 3.8 mmol) were added. After reacting for 9 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 14.3 g of Compound J. (Yield: 69%, MS: [M+H]+=329)

Compound J (15 g, 45.7 mmol) and Trz4 (20.8 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 16.7 g of Compound 13. (Yield: 61%, MS: [M+H]+=600)

### Example 14: Synthesis of Compound 14

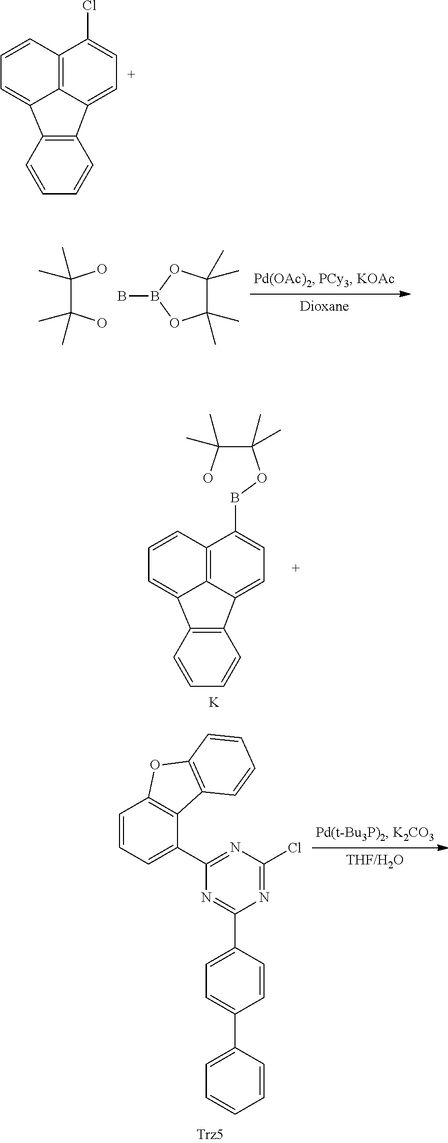

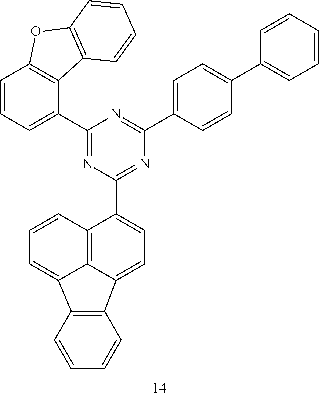 [SMILES: c1ccc(-c2ccc(-c3nc(-c4ccc5c6c(cccc46)-c4ccccc4-5)nc(-c4cccc5oc6ccccc6c45)n3)cc2)cc1]

3-Chlorofluoranthene (15 g, 63.4 mmol) and bis(pinacolato)diboron (17.7 g, 69.7 mmol) were added to 300 ml of 1,4-dioxane under a nitrogen atmosphere, and the mixture was stirred under reflux. Then, potassium acetate (9.3 g, 95.1 mmol) was added thereto, sufficiently stirred, and then bis(dibenzylideneacetone) palladium (0) (1.1 g, 1.9 mmol) and tricyclohexylphosphine (1.1 g, 3.8 mmol) were added. After reacting for 9 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 15.2 g of Compound K. (Yield: 73%, MS: [M+H]+=329)

Compound K (15 g, 45.7 mmol) and Trz5 (20.8 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17.3 g of Compound 14. (Yield: 63%, MS: [M+H]+=600)

### Example 15: Synthesis of Compound 15

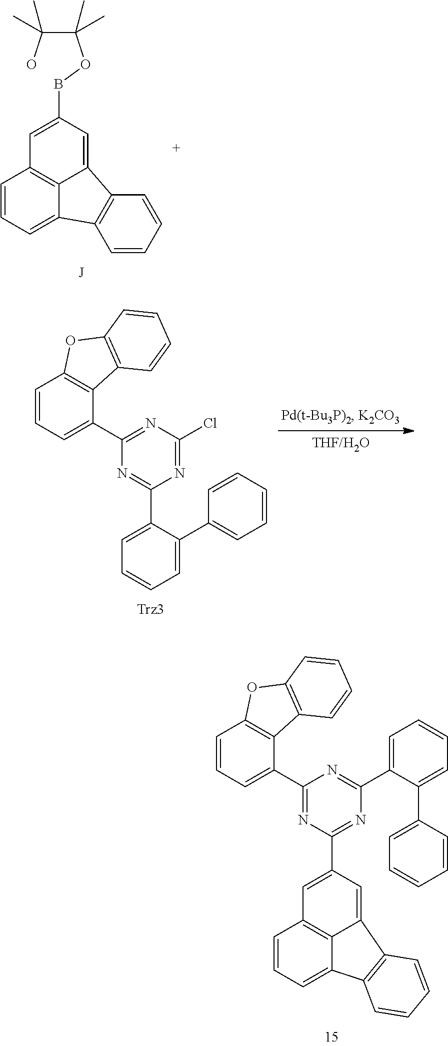

Compound J (15 g, 45.7 mmol) and Trz3 (20.8 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 21.9 g of Compound 15. (Yield: 80%, MS: [M+H]+=600)

### Example 16: Synthesis of Compound 16

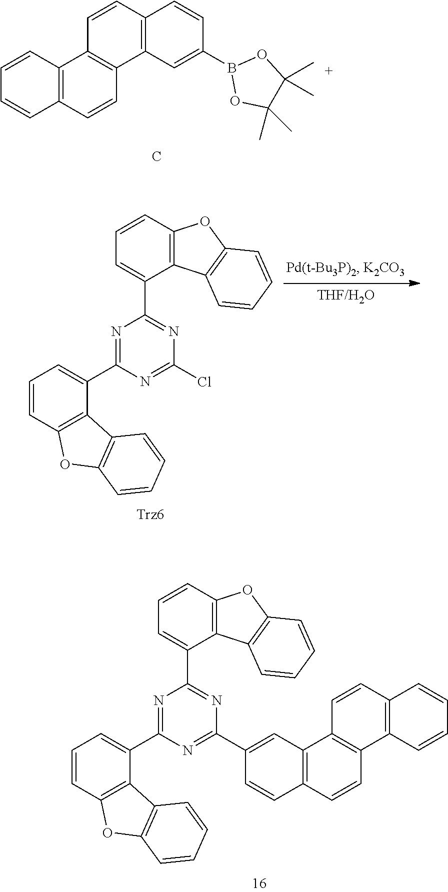

Compound C (15 g, 42.3 mmol) and Trz6 (19.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 20.8 g of Compound 16. (Yield: 77%, MS: [M+H]+=640)

### Example 17: Synthesis of Compound 17

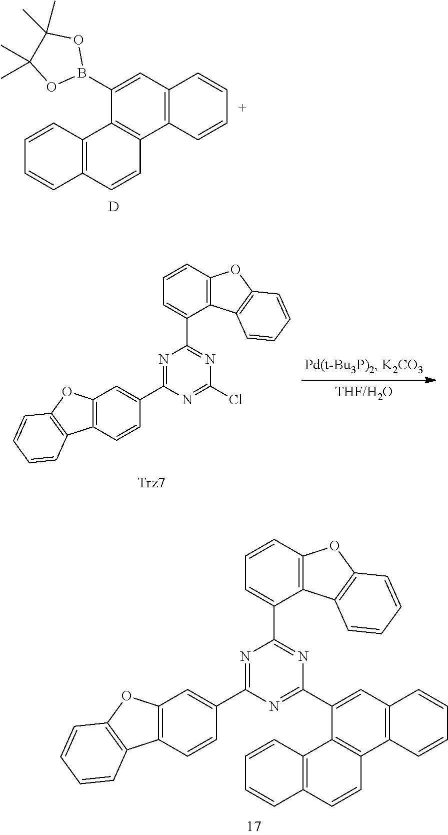

Compound D (15 g, 42.3 mmol) and Trz7 (19.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18.4 g of Compound 17. (Yield: 68%, MS: [M+H]+=640)

### Example 18: Synthesis of Compound 18

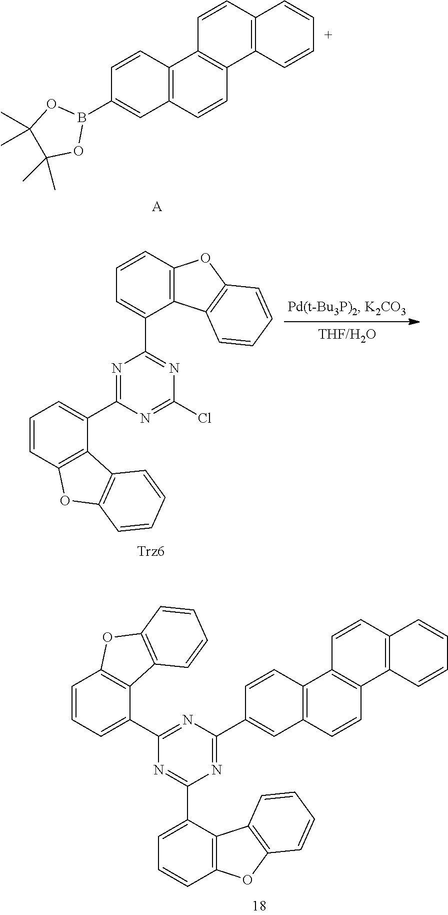

Compound A (15 g, 42.3 mmol) and Trz6 (19.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 21.4 g of Compound 18. (Yield: 79%, MS: [M+H]+=640)

### Example 19: Synthesis of Compound 19

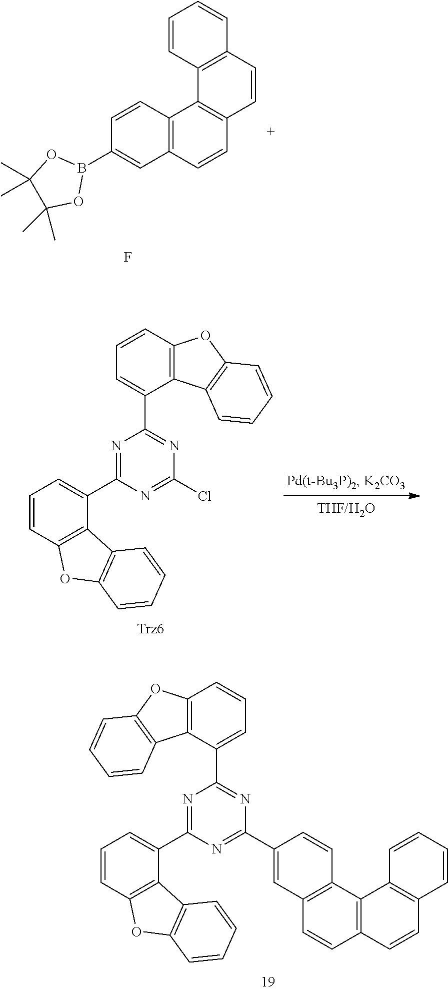

Compound F (15 g, 42.3 mmol) and Trz6 (19.9 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 19.2 g of Compound 19. (Yield: 71%, MS: [M+H]+=640)

### Example 20: Synthesis of Compound 20

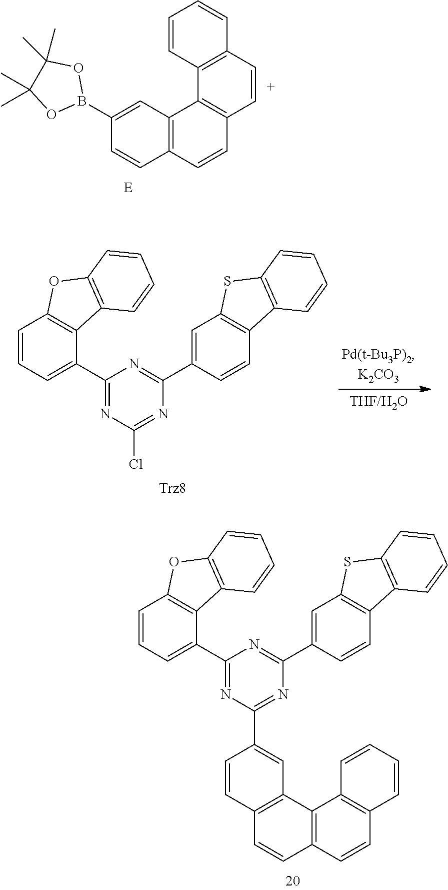 [SMILES: CC1(C)OB(c2ccc3ccc4ccc5ccccc5c4c3c2)OC1(C)C ; Clc1nc(-c2ccc3c(c2)sc2ccccc23)nc(-c2cccc3oc4ccccc4c23)n1 ; c1ccc2c(c1)ccc1ccc3ccc(-c4nc(-c5ccc6c(c5)sc5ccccc56)nc(-c5cccc6oc7ccccc7c56)n4)cc3c12]

Compound E (15 g, 42.3 mmol) and Trz8 (20.6 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18.3 g of Compound 20. (Yield: 66%, MS: [M+H]+=656)

### Example 21: Synthesis of Compound 21

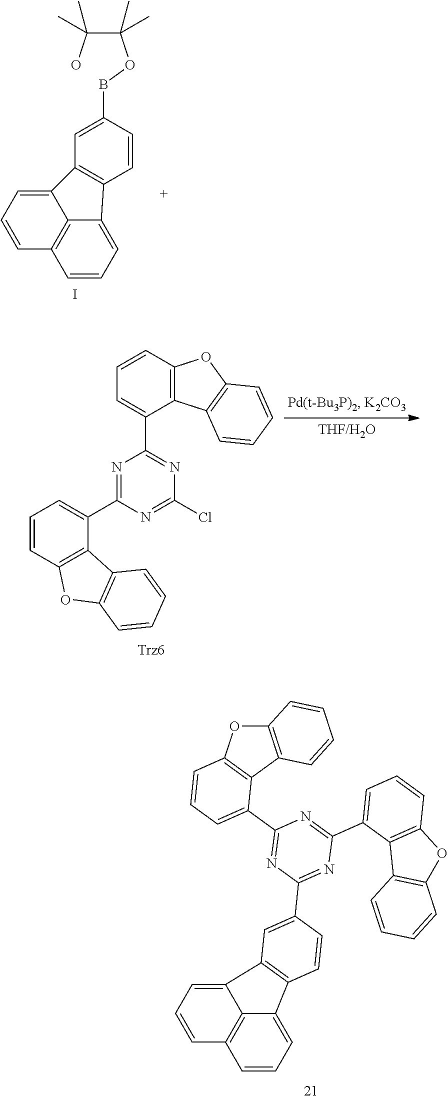

Compound I (15 g, 45.7 mmol) and Trz6 (21.5 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 22.4 g of Compound 21. (Yield: 80%, MS: [M+H]+=614)

### Example 22: Synthesis of Compound 22

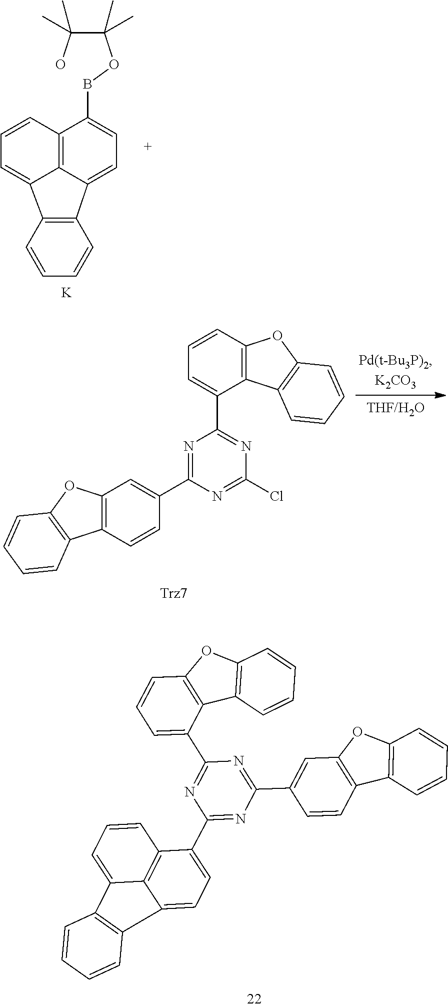

Compound K (15 g, 45.7 mmol) and Trz7 (21.5 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 20.5 g of Compound 22. (Yield: 73%, MS: [M+H]+=614)

### Example 23: Synthesis of Compound 23

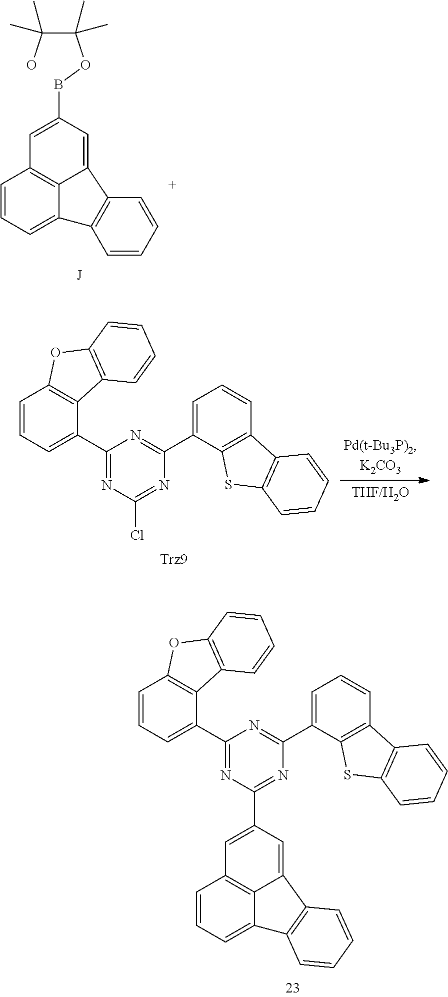

Compound J (15 g, 45.7 mmol) and Trz9 (22.3 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 19.8 g of Compound 23. (Yield: 69%, MS: [M+H]+=630)

### Example 24: Synthesis of Compound 24

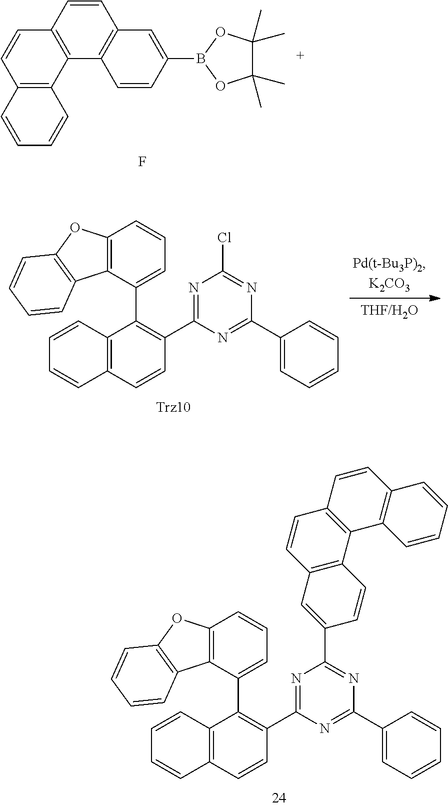

Compound F (15 g, 42.3 mmol) and Trz10 (21.5 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 22.6 g of Compound 24. (Yield: 79%, MS: [M+H]+=676)

### Example 25: Synthesis of Compound 25

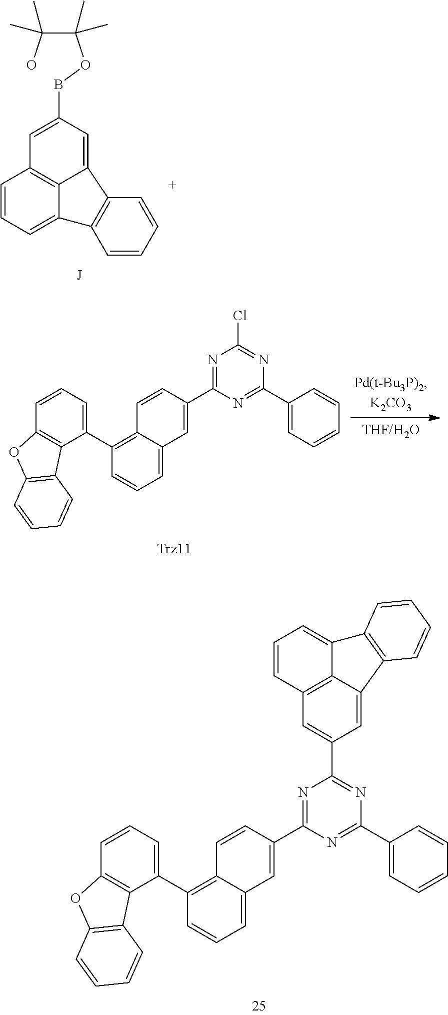

Compound J (15 g, 45.7 mmol) and Trz11 (23.2 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18.1 g of Compound 25. (Yield: 61%, MS: [M+H]+=650)

### Example 26: Synthesis of Compound 26

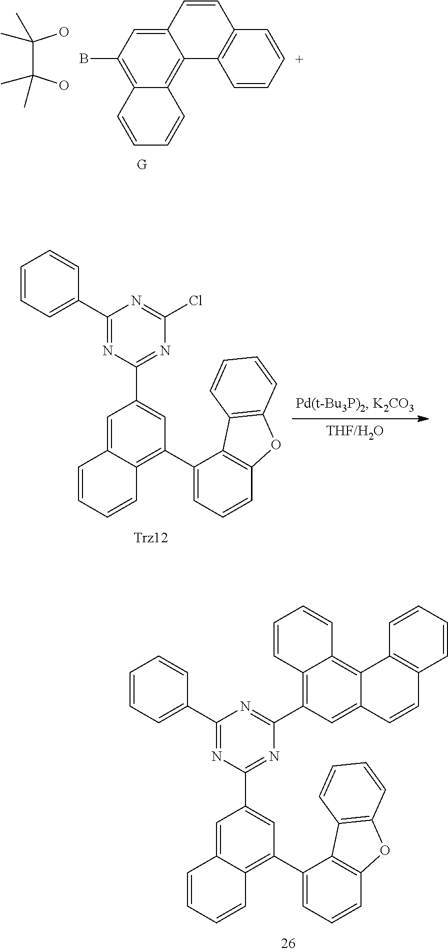

Compound G (15 g, 42.3 mmol) and Trz12 (21.5 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 21.4 g of Compound 26. (Yield: 75%, MS: [M+H]+=676)

### Example 27: Synthesis of Compound 27

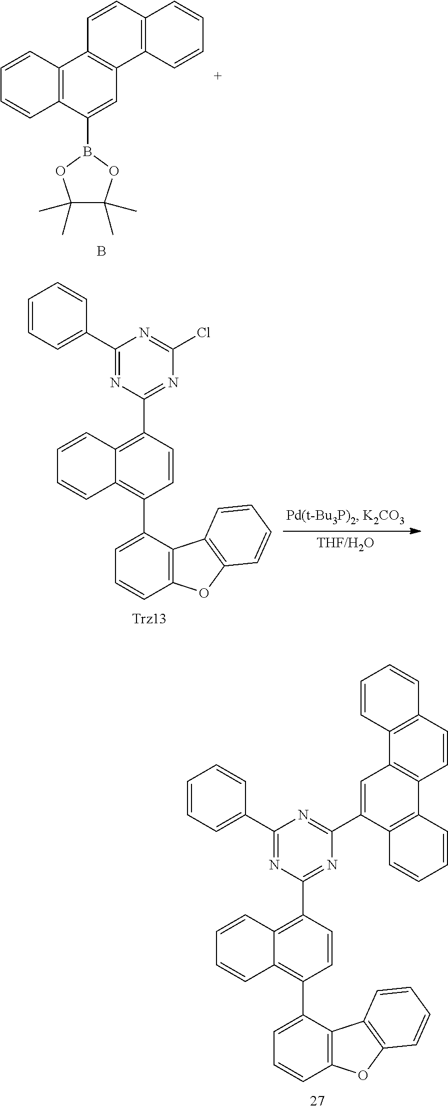

Compound B (15 g, 42.3 mmol) and Trz13 (21.5 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 19.4 g of Compound 27. (Yield: 68%, MS: [M+H]+=676)

### Example 28: Synthesis of Compound 28

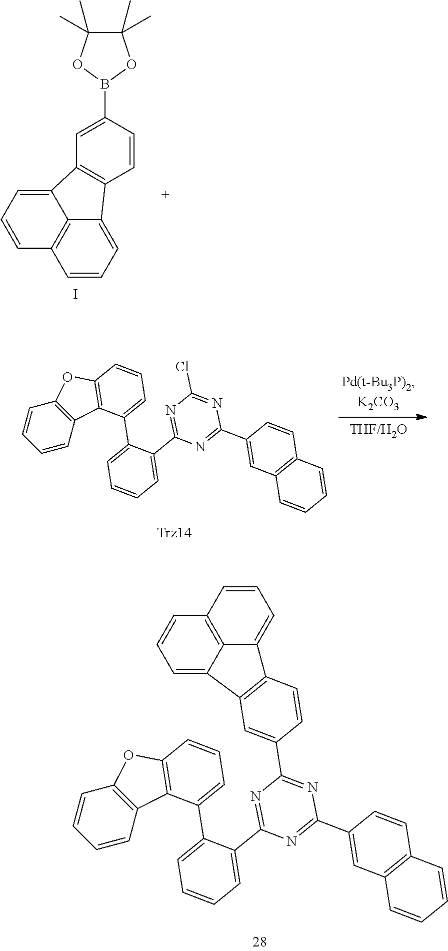

Compound I (15 g, 45.7 mmol) and Trz14 (23.2 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 23.4 g of Compound 28. (Yield: 79%, MS: [M+H]+=650)

### Example 29: Synthesis of Compound 29

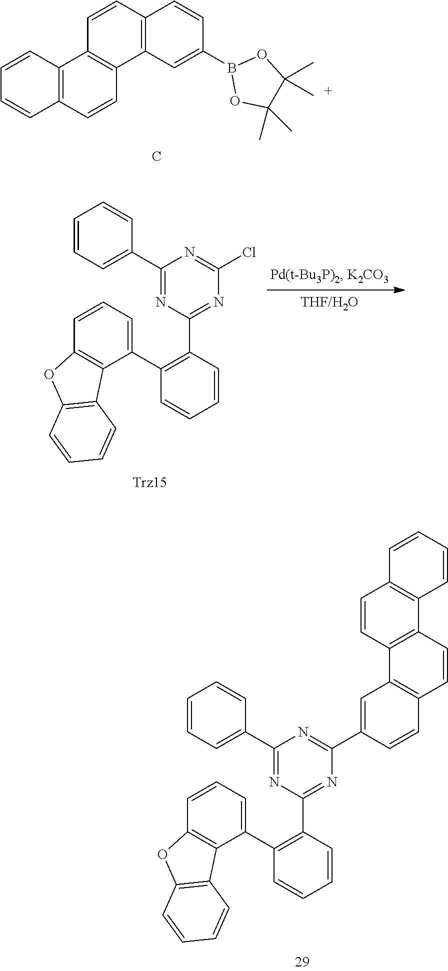

Compound C (15 g, 42.3 mmol) and Trz15 (19.3 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 19.1 g of Compound 29. (Yield: 72%, MS: [M+H]+=626)

### Example 30: Synthesis of Compound 30

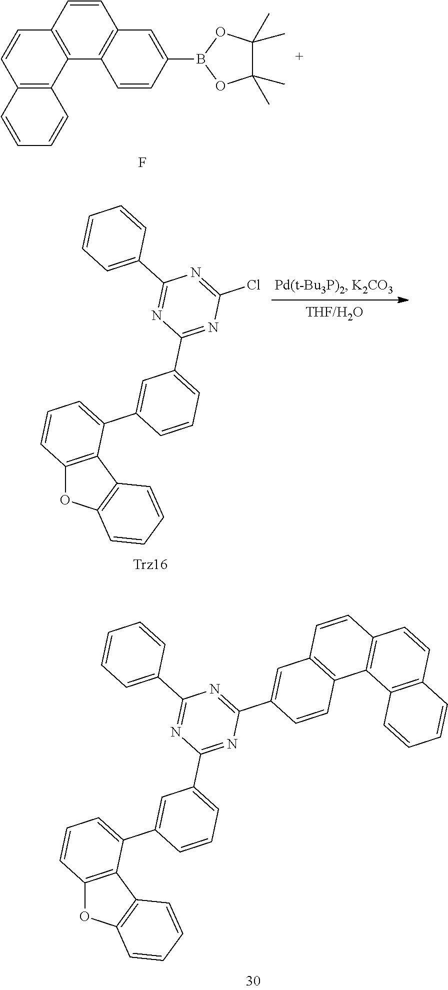

Compound F (15 g, 42.3 mmol) and Trz16 (19.3 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18 g of Compound 30. (Yield: 68%, MS: [M+H]+=626)

### Example 31: Synthesis of Compound 31

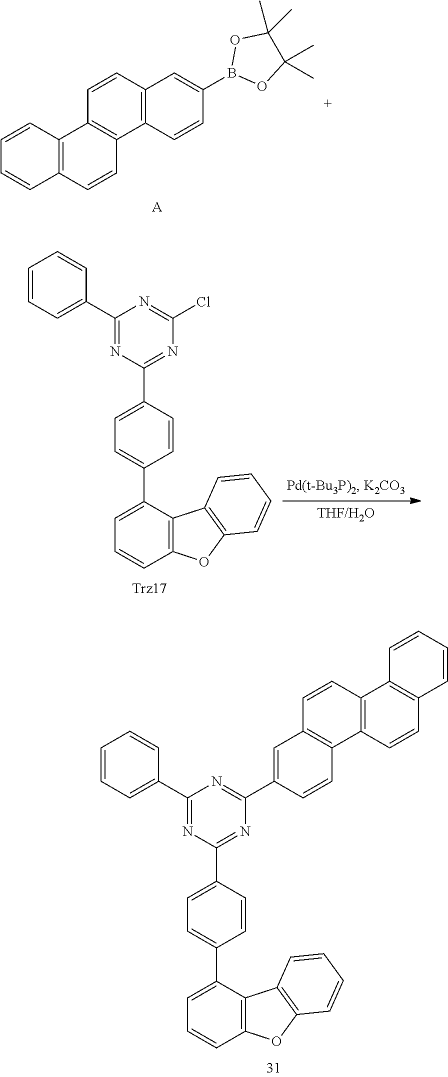

Compound A (15 g, 42.3 mmol) and Trz17 (19.3 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 20.6 g of Compound 31. (Yield: 78%, MS: [M+H]+=626)

### Example 32: Synthesis of Compound 32

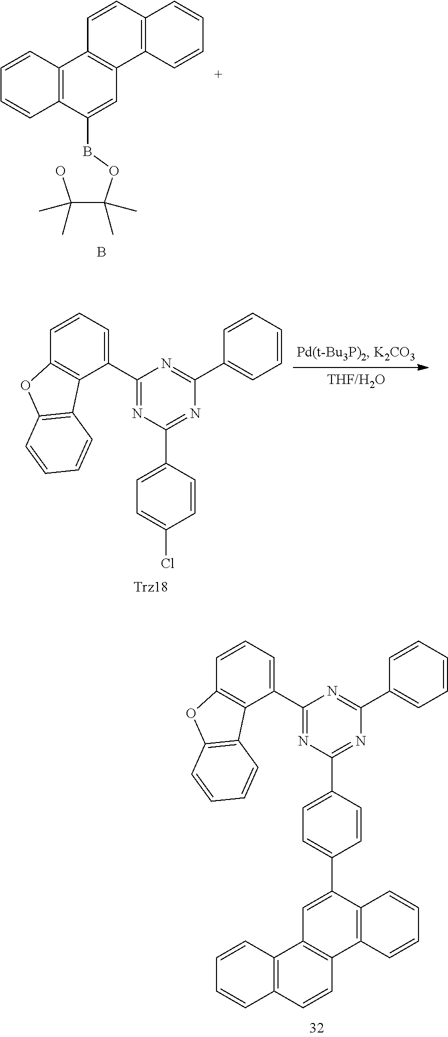

Compound B (15 g, 42.3 mmol) and Trz18 (19.3 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18.3 g of Compound 32. (Yield: 69%, MS: [M+H]+=626)

### Example 33: Synthesis of Compound 33

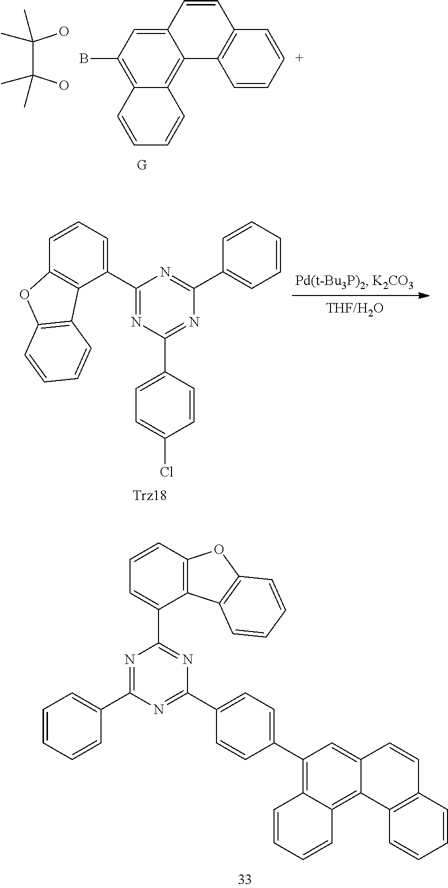

Compound G (15 g, 42.3 mmol) and Trz18 (19.3 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 20.1 g of Compound 33. (Yield: 76%, MS: [M+H]+=626)

### Example 34: Synthesis of Compound 34

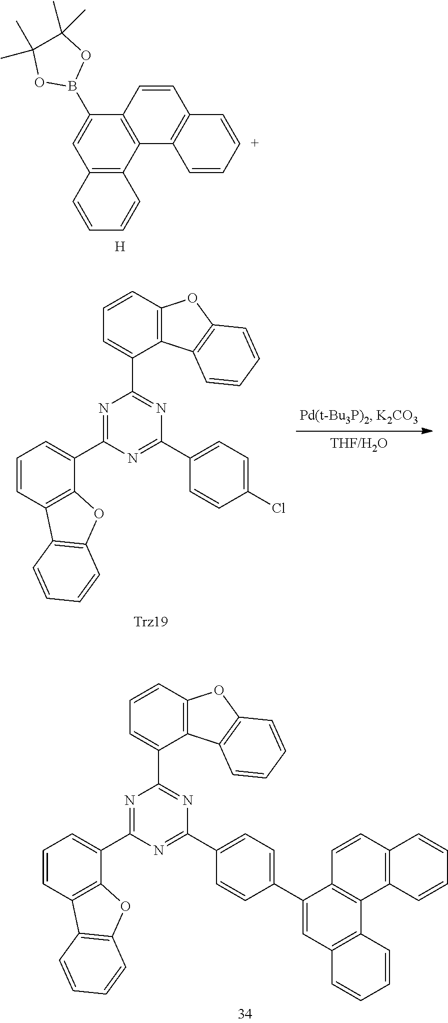

Compound H (15 g, 42.3 mmol) and Trz19 (23.3 g, 44.5 mmol) were added to 300 ml of THE under a nitrogen atmosphere, and the mixture was stirred and refluxed. Then, potassium carbonate (17.6 g, 127 mmol) was dissolved in 53 ml of water and added thereto, and the mixture was sufficiently stirred and then bis(tri-tert-butylphosphine) palladium (0) (0.2 g, 0.4 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18.2 g of Compound 34. (Yield: 60%, MS: [M+H]+=716)

### Example 35: Synthesis of Compound 35

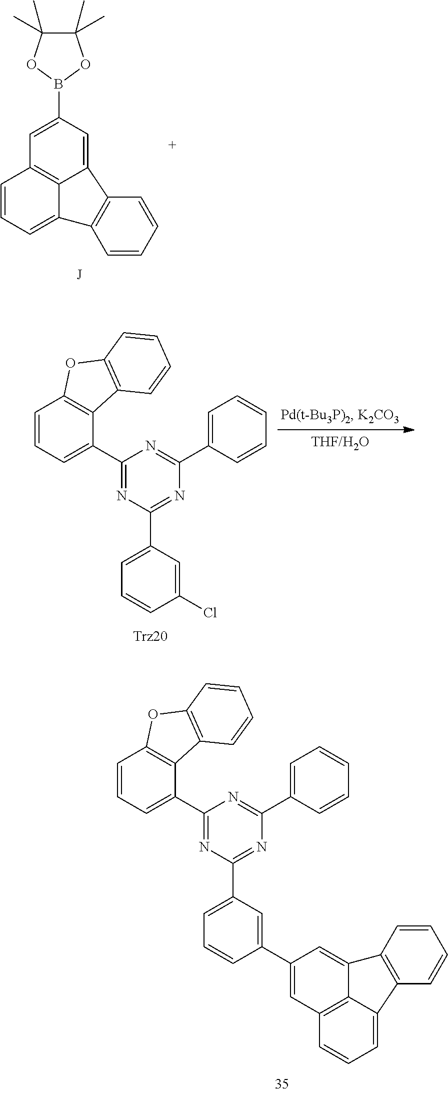 [SMILES: CC1(C)OB(c2cc3c4c(cccc4c2)-c2ccccc2-3)OC1(C)C ; Clc1cccc(-c2nc(-c3ccccc3)nc(-c3cccc4oc5ccccc5c34)n2)c1 ; c1ccc(-c2nc(-c3cccc(-c4cc5c6c(cccc6c4)-c4ccccc4-5)c3)nc(-c3cccc4oc5ccccc5c34)n2)cc1]

Compound J (15 g, 45.7 mmol) and Trz20 (20.8 g, 48 mmol) were added to 300 ml of THE under a nitrogen atmosphere, and the mixture was stirred and refluxed. Then, potassium carbonate (18.9 g, 137.1 mmol) was dissolved in 57 ml of water and added thereto, and the mixture was sufficiently stirred and then bis(tri-tert-butylphosphine) palladium (0) (0.2 g, 0.5 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 19.2 g of Compound 35. (Yield: 70%, MS: [M+H]+=600)

### Example 36: Synthesis of Compound 36

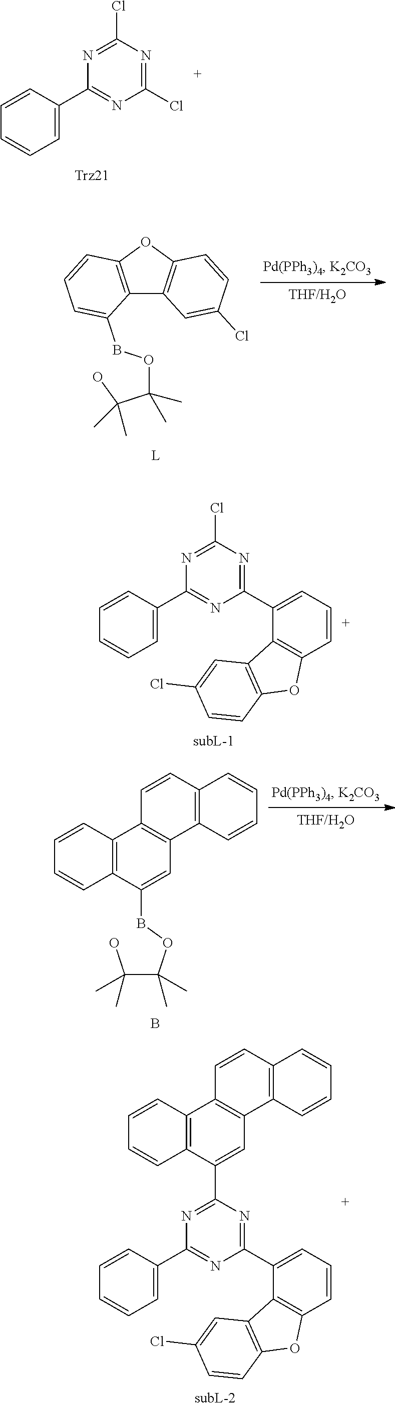

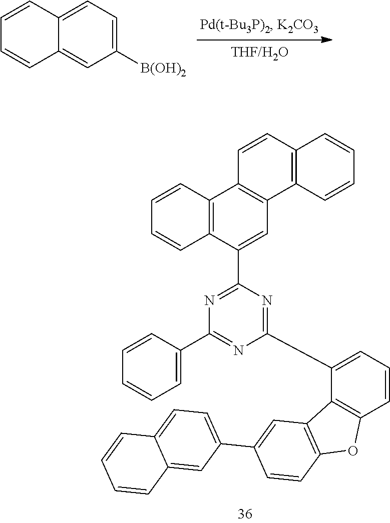 [SMILES: OB(O)c1ccc2ccccc2c1 ; c1ccc(-c2nc(-c3cc4c5ccccc5ccc4c4ccccc34)nc(-c3cccc4oc5ccc(-c6ccc7ccccc7c6)cc5c34)n2)cc1]

Trz21 (15 g, 66.4 mmol) and Compound L (22.9 g, 69.7 mmol) were added to 300 ml of THE under a nitrogen atmosphere, and the mixture was stirred and refluxed. Then, potassium carbonate (27.5 g, 199.1 mmol) was dissolved in 83 ml of water and added thereto, and the mixture was sufficiently stirred and then tetrakis(triphenylphosphine)palladium(0) (0.8 g, 0.7 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 16.6 g of subL-1. (Yield: 64%, MS: [M+H]+=392)

subL-1 (15 g, 38.2 mmol) and Compound B (14.2 g, 40.2 mmol) were added to 300 ml of THE under a nitrogen atmosphere, and the mixture was stirred and refluxed. Then, potassium carbonate (15.9 g, 114.7 mmol) was dissolved in 48 ml of water and added thereto, and the mixture was sufficiently stirred and then tetrakis(triphenylphosphine) palladium (0) (0.4 g, 0.4 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 13.8 g of subL-2. (Yield: 62%, MS: [M+H]+=584)

subL-2 (15 g, 25.7 mmol) and naphthalen-2-ylboronic acid (4.6 g, 27 mmol) were added to 300 ml of THE under a nitrogen atmosphere, and the mixture was stirred and refluxed. Then, potassium carbonate (10.6 g, 77 mmol) was dissolved in 32 ml of water and added thereto, and the mixture was sufficiently stirred and then bis(tri-tert-butylphosphine)palladium(0) (0.1 g, 0.3 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.1 g of Compound 36. (Yield: 70%, MS: [M+H]+=676)

### Example 37: Synthesis of Compound 37

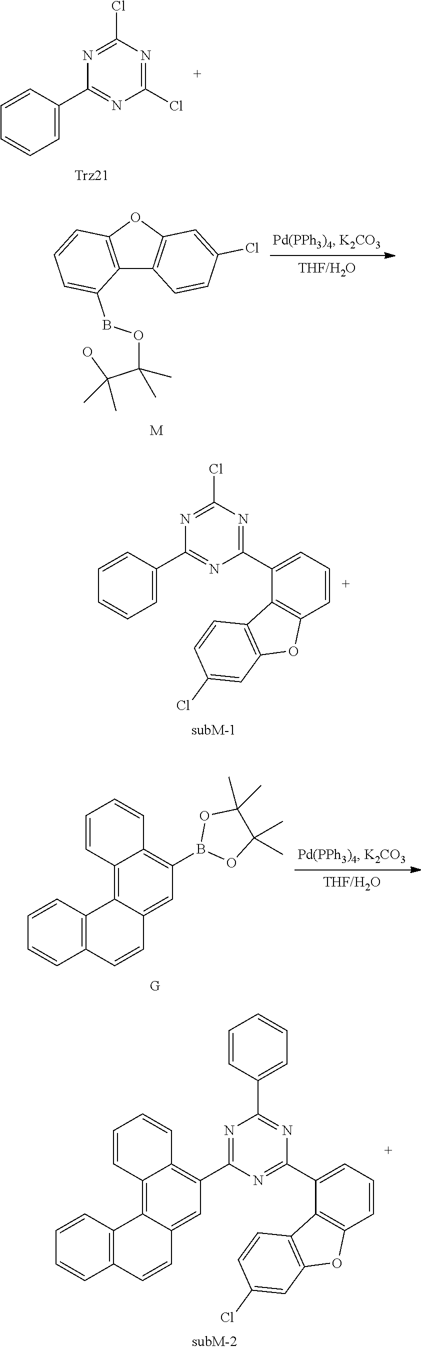

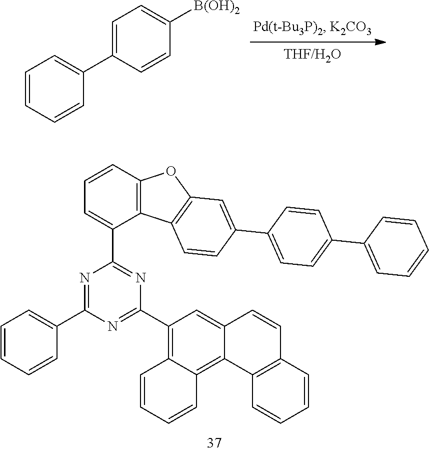 [SMILES: OB(O)c1ccc(-c2ccccc2)cc1 ; c1ccc(-c2ccc(-c3ccc4c(c3)oc3cccc(-c5nc(-c6ccccc6)nc(-c6cc7ccc8ccccc8c7c7ccccc67)n5)c34)cc2)cc1]

Trz21 (15 g, 66.4 mmol) and Compound M (22.9 g, 69.7 mmol) were added to 300 ml of THE under a nitrogen atmosphere, and the mixture was stirred and refluxed. Then, potassium carbonate (27.5 g, 199.1 mmol) was dissolved in 83 ml of water and added thereto, and the mixture was sufficiently stirred and then tetrakis(triphenylphosphine) palladium (0) (0.8 g, 0.7 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 18.4 g of subM-1. (Yield: 71%, MS: [M+H]+=392)

subM-1 (15 g, 38.2 mmol) and Compound G (14.2 g, 40.2 mmol) were added to 300 ml of THE under a nitrogen atmosphere, and the mixture was stirred and refluxed. Then, potassium carbonate (15.9 g, 114.7 mmol) was dissolved in 48 ml of water and added thereto, and the mixture was sufficiently stirred and then tetrakis(triphenylphosphine) palladium (0) (0.4 g, 0.4 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 14.5 g of subM-2. (Yield: 65%, MS: [M+H]+=584)

subM-2 (15 g, 25.7 mmol) and [1,1′-biphenyl]-4-ylboronic acid (5.3 g, 27 mmol) were added to 300 ml of THE under a nitrogen atmosphere, and the mixture was stirred and refluxed. Then, potassium carbonate (10.7 g, 77.2 mmol) was dissolved in 32 ml of water and added thereto, and the mixture was sufficiently stirred and then bis(tri-tert-butylphosphine)palladium(0) (0.1 g, 0.3 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 11.9 g of Compound 37. (Yield: 66%, MS: [M+H]+=702)

### Example 38: Synthesis of Compound 38

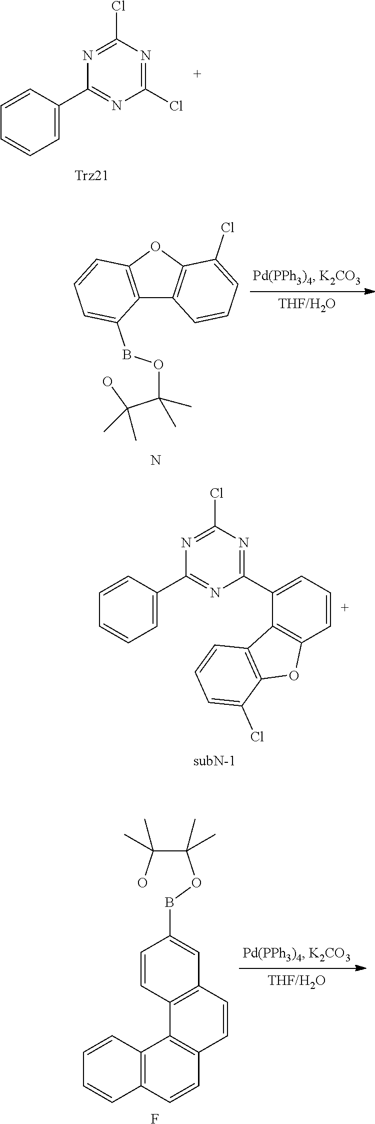

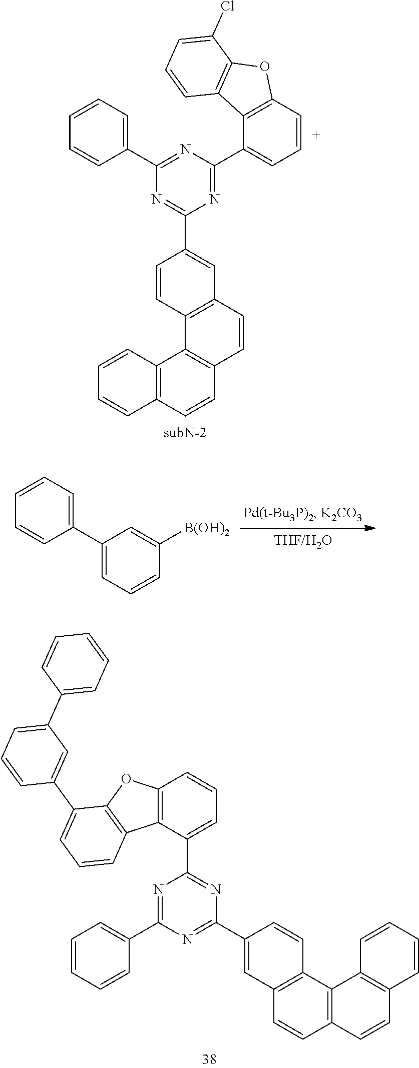

Trz21 (15 g, 66.4 mmol) and Compound N (22.9 g, 69.7 mmol) were added to 300 ml of THE under a nitrogen atmosphere, and the mixture was stirred and refluxed. Then, potassium carbonate (27.5 g, 199.1 mmol) was dissolved in 83 ml of water and added thereto, and the mixture was sufficiently stirred and then tetrakis(triphenylphosphine) palladium (0) (0.8 g, 0.7 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17.1 g of subN-1. (Yield: 66%, MS: [M+H]+=392)

subN-1 (15 g, 38.2 mmol) and Compound F (14.2 g, 40.2 mmol) were added to 300 ml of THE under a nitrogen atmosphere, and the mixture was stirred and refluxed. Then, potassium carbonate (15.9 g, 114.7 mmol) was dissolved in 48 ml of water and added thereto, and the mixture was sufficiently stirred and then tetrakis(triphenylphosphine) palladium (0) (0.4 g, 0.4 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 13.6 g of subN-2. (Yield: 61%, MS: [M+H]+=584)

subN-2(15 g, 25.7 mmol) and [1,1′-biphenyl]-3-ylboronic acid (5.3 g, 27 mmol) were added to 300 ml of THE under a nitrogen atmosphere, and the mixture was stirred and refluxed. Then, potassium carbonate (10.7 g, 77.2 mmol) was dissolved in 32 ml of water and added thereto, and the mixture was sufficiently stirred and then bis(tri-tert-butylphosphine)palladium(0) (0.1 g, 0.3 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.1 g of Compound 38. (Yield: 67%, MS: [M+H]+=702)

### Example 39: Synthesis of Compound 39

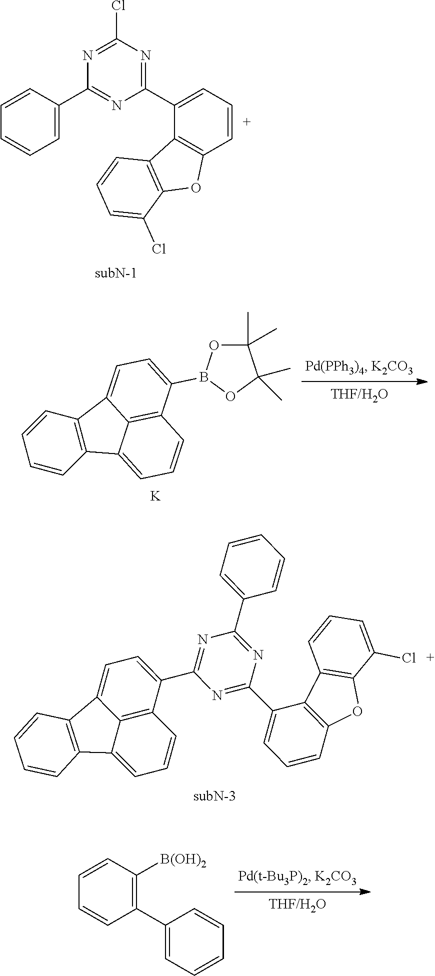

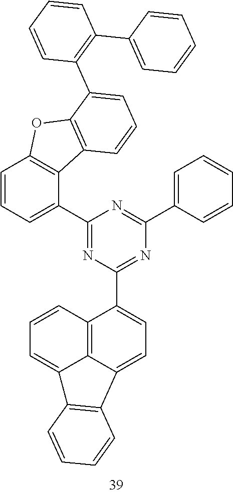 [SMILES: c1ccc(-c2nc(-c3ccc4c5c(cccc35)-c3ccccc3-4)nc(-c3cccc4oc5c(-c6ccccc6-c6ccccc6)cccc5c34)n2)cc1]

subN-1 (15 g, 38.2 mmol) and Compound K (13.2 g, 40.2 mmol) were added to 300 ml of THE under a nitrogen atmosphere, and the mixture was stirred and refluxed. Then, potassium carbonate (15.9 g, 114.7 mmol) was dissolved in 48 ml of water and added thereto, and the mixture was sufficiently stirred and then tetrakis(triphenylphosphine) palladium (0) (0.4 g, 0.4 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17 g of subN-3. (Yield: 80%, MS: [M+H]+=558)

subN-3 (15 g, 26.9 mmol) and [1,1′-biphenyl]-2-ylboronic acid (5.6 g, 28.2 mmol) were added to 300 ml of THE under a nitrogen atmosphere, and the mixture was stirred and refluxed. Then, potassium carbonate (11.1 g, 80.6 mmol) was dissolved in 33 ml of water and added thereto, and the mixture was sufficiently stirred and then bis(tri-tert-butylphosphine)palladium(0) (0.1 g, 0.3 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 13.2 g of Compound 39. (Yield: 73%, MS: [M+H]+=676)

### Example 40: Synthesis of Compound 40

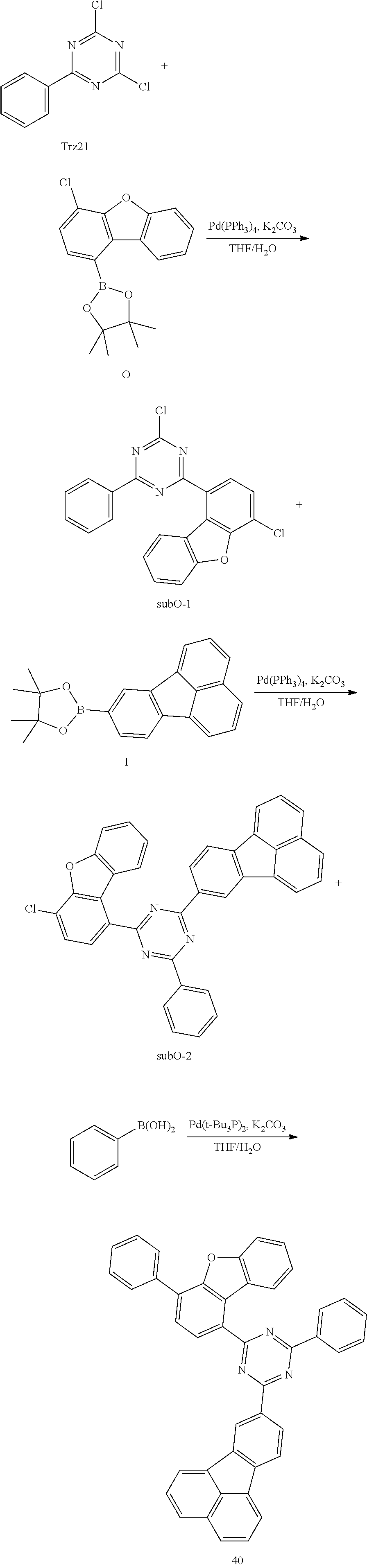

Trz21 (15 g, 66.4 mmol) and Compound O (22.9 g, 69.7 mmol) were added to 300 ml of THE under a nitrogen atmosphere, and the mixture was stirred and refluxed. Then, potassium carbonate (27.5 g, 199.1 mmol) was dissolved in 83 ml of water and added thereto, and the mixture was sufficiently stirred and then tetrakis(triphenylphosphine)palladium (0) (0.8 g, 0.7 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17.9 g of subO-1. (Yield: 69%, MS: [M+H]+=392)

subO-1 (15 g, 38.2 mmol) and Compound I (13.2 g, 40.2 mmol) were added to 300 ml of THE under a nitrogen atmosphere, and the mixture was stirred and refluxed. Then, potassium carbonate (15.9 g, 114.7 mmol) was dissolved in 48 ml of water and added thereto, and the mixture was sufficiently stirred and then tetrakis(triphenylphosphine) palladium (0) (0.4 g, 0.4 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 13 g of subO-2. (Yield: 61%, MS: [M+H]+=558)

subO-2 (15 g, 26.9 mmol) and phenylboronic acid (3.4 g, 28.2 mmol) were added to 300 ml of THE under a nitrogen atmosphere, and the mixture was stirred and refluxed. Then, potassium carbonate (11.1 g, 80.6 mmol) was dissolved in 33 ml of water and added thereto, and the mixture was sufficiently stirred and then bis(tri-tert-butylphosphine)palladium(0) (0.1 g, 0.3 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.4 g of Compound 40. (Yield: 77%, MS: [M+H]+=600)

### Example 41: Synthesis of Compound 41

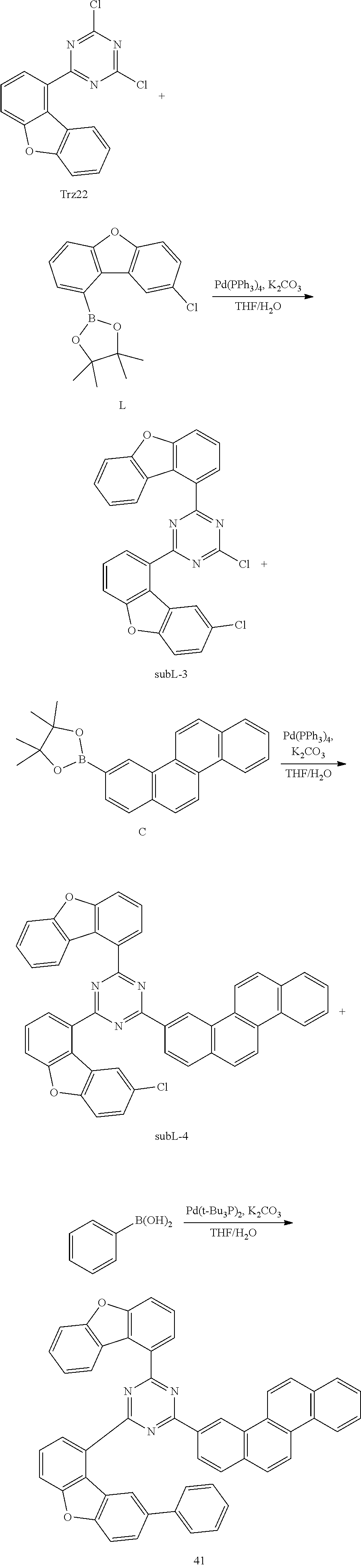

Trz22 (15 g, 47.4 mmol) and Compound L (16.4 g, 49.8 mmol) were added to 300 ml of THE under a nitrogen atmosphere, and the mixture was stirred and refluxed. Then, potassium carbonate (19.7 g, 142.3 mmol) was dissolved in 59 ml of water and added thereto, and the mixture was sufficiently stirred and then tetrakis(triphenylphosphine) palladium (0) (0.5 g, 0.5 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 17.1 g of subL-3. (Yield: 75%, MS: [M+H]+=482)

subL-3 (15 g, 31.1 mmol) and Compound C (11.6 g, 32.7 mmol) were added to 300 ml of THE under a nitrogen atmosphere, and the mixture was stirred and refluxed. Then, potassium carbonate (12.9 g, 93.3 mmol) was dissolved in 39 ml of water and added thereto, and the mixture was sufficiently stirred, and then tetrakis(triphenylphosphine) palladium (0) (0.4 g, 0.3 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.6 g of subL-4. (Yield: 60%, MS: [M+H]+=674)

subL-4 (15 g, 22.2 mmol) and phenylboronic acid (2.8 g, 23.4 mmol) were added to 300 ml of THE under a nitrogen atmosphere, and the mixture was stirred and refluxed. Then, potassium carbonate (9.2 g, 66.7 mmol) was dissolved in 28 ml of water and added thereto, and the mixture was sufficiently stirred and then bis(tri-tert-butylphosphine)palladium(0) (0.1 g, 0.2 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 10.7 g of Compound 41. (Yield: 67%, MS: [M+H]+=716)

### Example 42: Synthesis of Compound 42

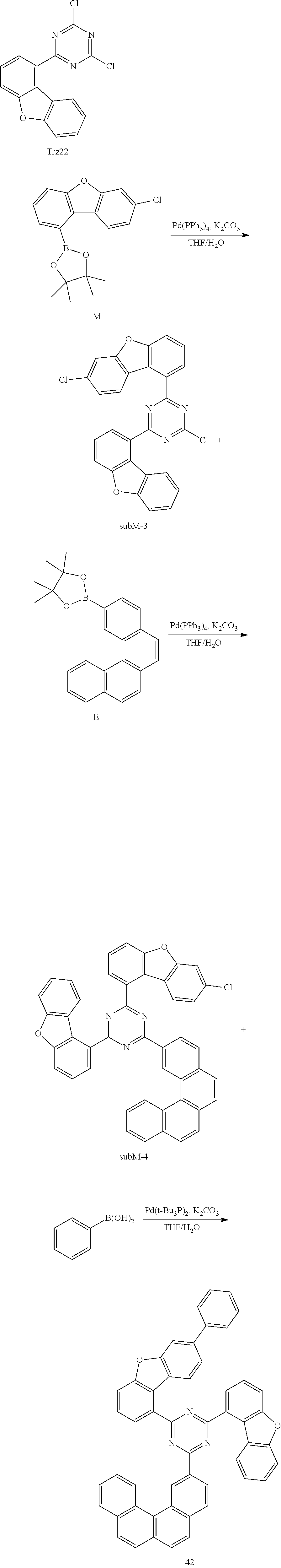

Trz22 (15 g, 47.4 mmol) and Compound M (16.4 g, 49.8 mmol) were added to 300 ml of THE under a nitrogen atmosphere, and the mixture was stirred and refluxed. Then, potassium carbonate (19.7 g, 142.3 mmol) was dissolved in 59 ml of water and added thereto, and the mixture was sufficiently stirred, and then tetrakis(triphenylphosphine) palladium (0) (0.5 g, 0.5 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 16.2 g of subM-3. (Yield: 71%, MS: [M+H]+=482)

subM-3(15 g, 31.1 mmol) and Compound E (11.6 g, 32.7 mmol) were added to 300 ml of THE under a nitrogen atmosphere, and the mixture was stirred and refluxed. Then, potassium carbonate (12.9 g, 93.3 mmol) was dissolved in 39 ml of water and added thereto, and the mixture was sufficiently stirred and then tetrakis(triphenylphosphine) palladium (0) (0.4 g, 0.3 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.6 g of subM-4. (Yield: 60%, MS: [M+H]+=674)

subM-4 (15 g, 22.2 mmol) and phenylboronic acid (2.8 g, 23.4 mmol) were added to 300 ml of THE under a nitrogen atmosphere, and the mixture was stirred and refluxed. Then, potassium carbonate (9.2 g, 66.7 mmol) was dissolved in 28 ml of water and added thereto, and the mixture was sufficiently stirred and then bis(tri-tert-butylphosphine)palladium(0) (0.1 g, 0.2 mmol) was added. After reacting for 12 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 11 g of Compound 42. (Yield: 69%, MS: [M+H]+=716)

### Example 43: Synthesis of Compound 43

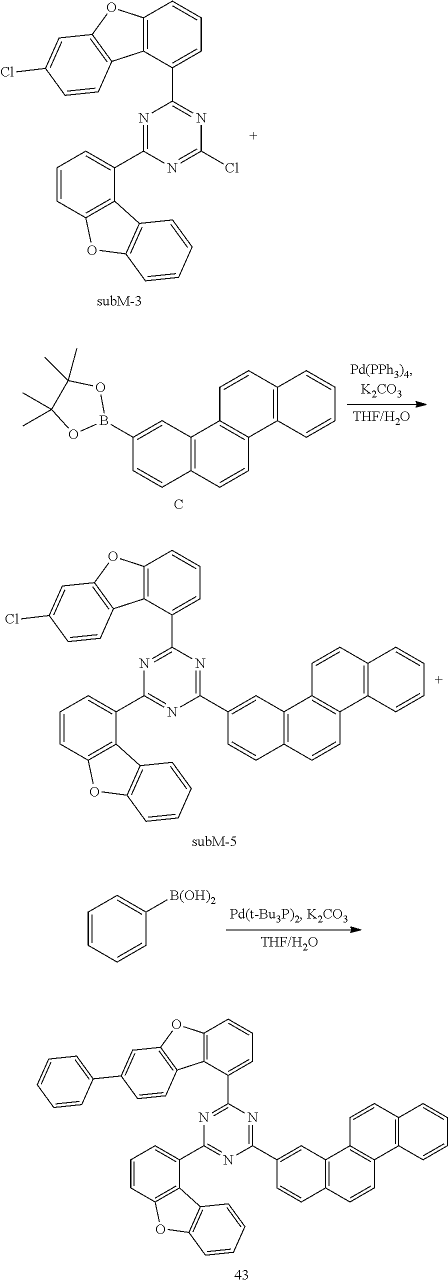

subM-3(15 g, 31.1 mmol) and Compound C (11.6 g, 32.7 mmol) were added to 300 ml of THE under a nitrogen atmosphere, and the mixture was stirred and refluxed. Then, potassium carbonate (12.9 g, 93.3 mmol) was dissolved in 39 ml of water and added thereto, and the mixture was sufficiently stirred, and then tetrakis(triphenylphosphine) palladium (0) (0.4 g, 0.3 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.6 g of subM-5. (Yield: 60%, MS: [M+H]+=674)

subM-5 (15 g, 22.2 mmol) and phenylboronic acid (2.8 g, 23.4 mmol) were added to 300 ml of THE under a nitrogen atmosphere, and the mixture was stirred and refluxed. Then, potassium carbonate (9.2 g, 66.7 mmol) was dissolved in 28 ml of water and added thereto, and the mixture was sufficiently stirred, and then bis(tri-tert-butylphosphine)palladium(0) (0.1 g, 0.2 mmol) was added. After reacting for 10 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 11.3 g of Compound 43. (Yield: 71%, MS: [M+H]+=716)

### Example 44: Synthesis of Compound 44

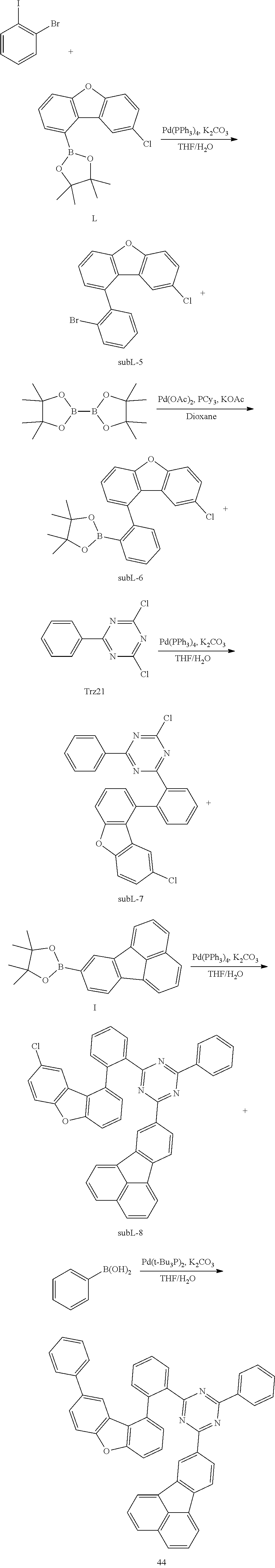

1-Bromo-2-iodobenzene (15 g, 53 mmol) and Compound L (18.3 g, 55.7 mmol) were added to 300 ml of THE under a nitrogen atmosphere, and the mixture was stirred and refluxed. Then, potassium carbonate (22 g, 159.1 mmol) was dissolved in 66 ml of water and added thereto, and the mixture was sufficiently stirred, and then tetrakis(triphenylphosphine) palladium (0) (0.6 g, 0.5 mmol) was added. After reacting for 11 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 11.9 g of subL-5. (Yield: 63%, MS: [M+H]+=357)

subL-5 (15 g, 41.9 mmol) and bis(pinacolato)diboron (11.7 g, 46.1 mmol) were added to 300 ml of 1,4-dioxane under a nitrogen atmosphere, and the mixture was stirred under reflux. Then, potassium acetate (6.2 g, 62.9 mmol) was added thereto, sufficiently stirred, and then bis(dibenzylideneacetone) palladium (0) (0.7 g, 1.3 mmol) and tricyclohexylphosphine (0.7 g, 2.5 mmol) were added. After reacting for 9 hours, the reaction mixture was cooled to room temperature, the organic layer was separated using chloroform and water, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 13.2 g of subL-6. (Yield: 78%, MS: [M+H]+=405)

subL-6 (15 g, 37.1 mmol) and Trz21 (8.8 g, 38.9 mmol) were added to 300 ml of THE under a nitrogen atmosphere, and the mixture was stirred and refluxed. Then, potassium carbonate (15.4 g, 111.2 mmol) was dissolved in 46 ml of water and added thereto, and the mixture was sufficiently stirred, and then tetrakis(triphenylphosphine) palladium (0) (0.4 g, 0.4 mmol) was added. After reacting for 8 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.6 g of subL-7. (Yield: 73%, MS: [M+H]+=468)

subL-7 (15 g, 32 mmol) and Compound I (11 g, 33.6 mmol) were added to 300 ml of THE under a nitrogen atmosphere, and the mixture was stirred and refluxed. Then, potassium carbonate (13.3 g, 96.1 mmol) was dissolved in 40 ml of water and added thereto, and the mixture was sufficiently stirred, and then tetrakis(triphenylphosphine) palladium (0) (0.4 g, 0.3 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 13 g of subL-8. (Yield: 64%, MS: [M+H]+=634)

subL-8 (15 g, 23.7 mmol) and phenylboronic acid (3 g, 24.8 mmol) were added to 300 ml of THE under a nitrogen atmosphere, and the mixture was stirred and refluxed. Then, potassium carbonate (9.8 g, 71 mmol) was dissolved in 29 ml of water and added thereto, and the mixture was sufficiently stirred and then bis(tri-tert-butylphosphine)palladium(0) (0.1 g, 0.2 mmol) was added. After reacting for 9 hours, the reaction mixture was cooled to room temperature, the organic layer and the aqueous layer were separated, and then the organic layer was distilled. This was dissolved again in chloroform, washed twice with water, and the organic layer was separated. Anhydrous magnesium sulfate was added, and the mixture was stirred, filtered, and the filtrate was distilled under reduced pressure. The concentrated compound was purified by silica gel column chromatography to prepare 12.8 g of Compound 44. (Yield: 80%, MS: [M+H]+=676)

### EXPERIMENTAL EXAMPLES

### Experimental Example 1

A glass substrate on which ITO (indium tin oxide) was coated as a thin film to a thickness of 1,000 Å was put into distilled water in which a detergent was dissolved, and ultrasonically cleaned. At this time, a product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. The substrate was cleaned for 5 minutes using oxygen plasma and then transferred to a vacuum depositor.

On the ITO transparent electrode thus prepared, the following compound HI-1 was formed in a thickness of 1150 Å as a hole injection layer, but the following compound A-1 was p-doped at a concentration of 1.5 wt. %. The following compound HT-1 was vacuum deposited on the hole injection layer to form a hole transport layer with a film thickness of 800 Å. Then, the following compound EB-1 was vacuum deposited on the hole transport layer to a film thickness of 150 Å to form an electron blocking layer.

Then, Compound 1 as a host and the following Compound Dp-7 as a dopant were vacuum deposited in a weight ratio of 98:2 on the EB-1 deposited film to form a red light emitting layer with a film thickness of 400 Å. The following compound HB-1 was vacuum deposited on the light emitting layer to a film thickness of 30 Å to form a hole blocking layer.

Then, the following compound ET-1 and the following compound LiQ were vacuum deposited in a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

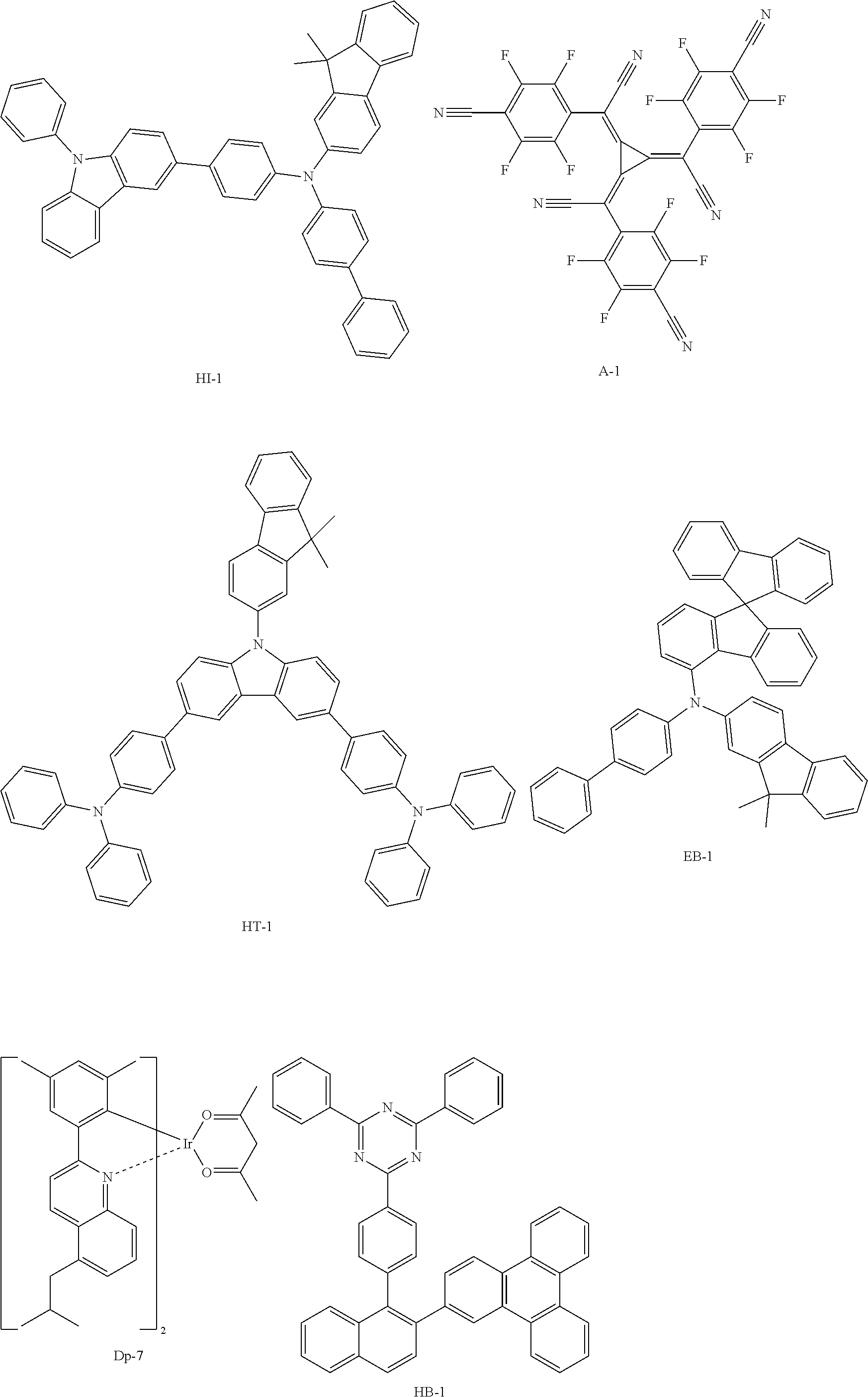

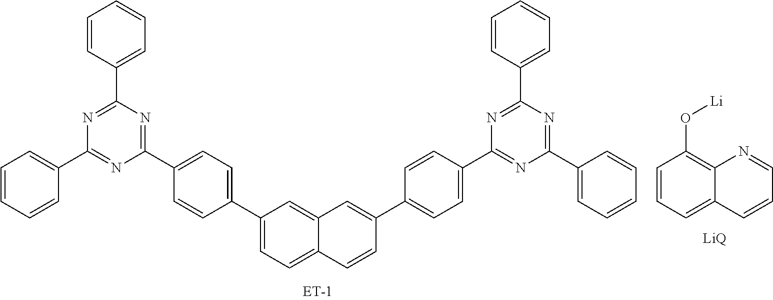

In the above-mentioned processes, the deposition rates of the organic materials were maintained at 0.4˜0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Experimental Examples 2 to 44

An organic light emitting device was manufactured in the same manner as in Experimental Example 1, except that the compound of Chemical Formula 1 as shown in Tables 1 and 2 was used as a host in the organic light emitting device of Experimental Example 1.

### Comparative Experimental Examples 1 to 14

An organic light emitting device was manufactured in the same manner as in Experimental Example 1, except that Comparative Compounds B-1 to B-14 as shown in Table 3 were used as hosts in the organic light emitting device of Experimental Example 1.

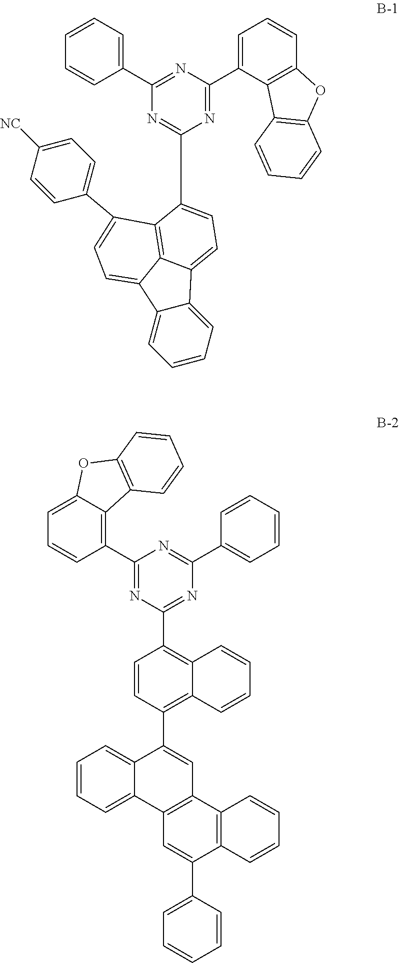 [SMILES: N#Cc1ccc(-c2ccc3c4c(ccc(-c5nc(-c6ccccc6)nc(-c6cccc7oc8ccccc8c67)n5)c24)-c2ccccc2-3)cc1 ; c1ccc(-c2nc(-c3ccc(-c4cc5c6ccccc6c(-c6ccccc6)cc5c5ccccc45)c4ccccc34)nc(-c3cccc4oc5ccccc5c34)n2)cc1]

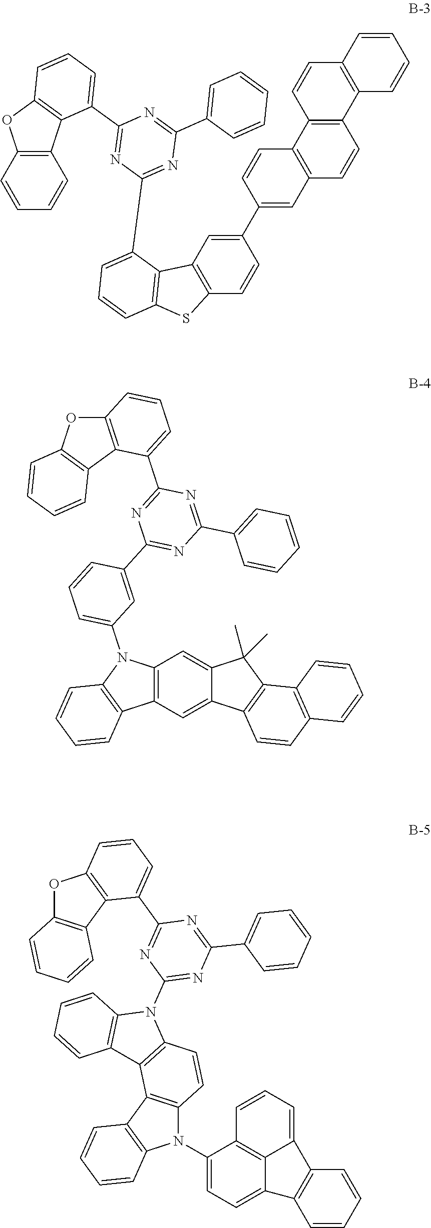 [SMILES: c1ccc(-c2nc(-c3cccc4oc5ccccc5c34)nc(-c3cccc4sc5ccc(-c6ccc7c(ccc8c9ccccc9ccc78)c6)cc5c34)n2)cc1 ; CC1(C)c2cc3c(cc2-c2ccc4ccccc4c21)c1ccccc1n3-c1cccc(-c2nc(-c3ccccc3)nc(-c3cccc4oc5ccccc5c34)n2)c1 ; c1ccc(-c2nc(-c3cccc4oc5ccccc5c34)nc(-n3c4ccccc4c4c5c6ccccc6n(-c6ccc7c8c(cccc68)-c6ccccc6-7)c5ccc43)n2)cc1]

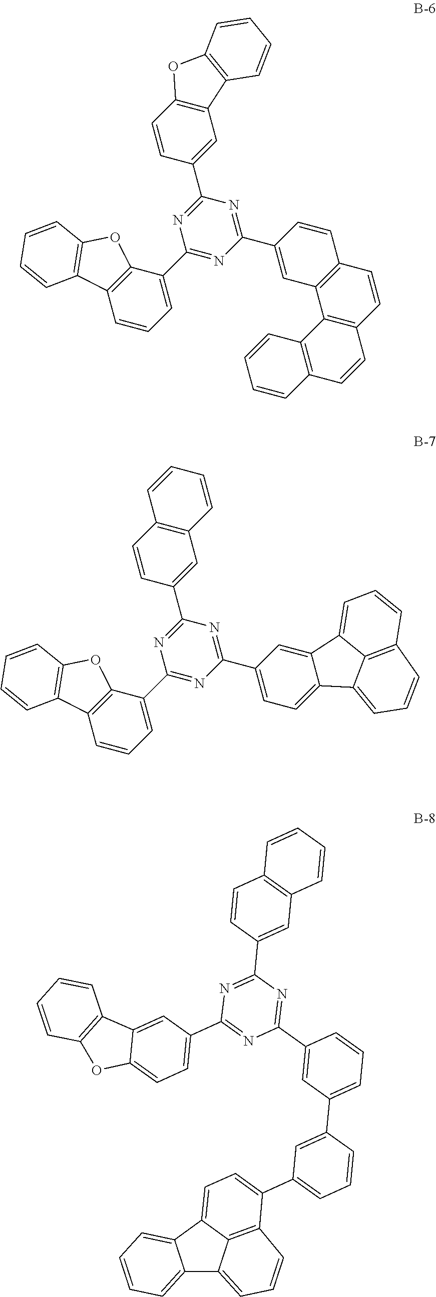 [SMILES: c1ccc2c(c1)ccc1ccc3ccc(-c4nc(-c5ccc6oc7ccccc7c6c5)nc(-c5cccc6c5oc5ccccc56)n4)cc3c12 ; c1ccc2cc(-c3nc(-c4ccc5c(c4)-c4cccc6cccc-5c46)nc(-c4cccc5c4oc4ccccc45)n3)ccc2c1 ; c1cc(-c2cccc(-c3ccc4c5c(cccc35)-c3ccccc3-4)c2)cc(-c2nc(-c3ccc4ccccc4c3)nc(-c3ccc4oc5ccccc5c4c3)n2)c1]

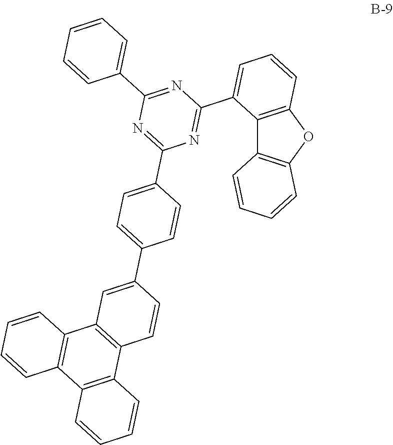 [SMILES: c1ccc(-c2nc(-c3ccc(-c4ccc5c6ccccc6c6ccccc6c5c4)cc3)nc(-c3cccc4oc5ccccc5c34)n2)cc1]

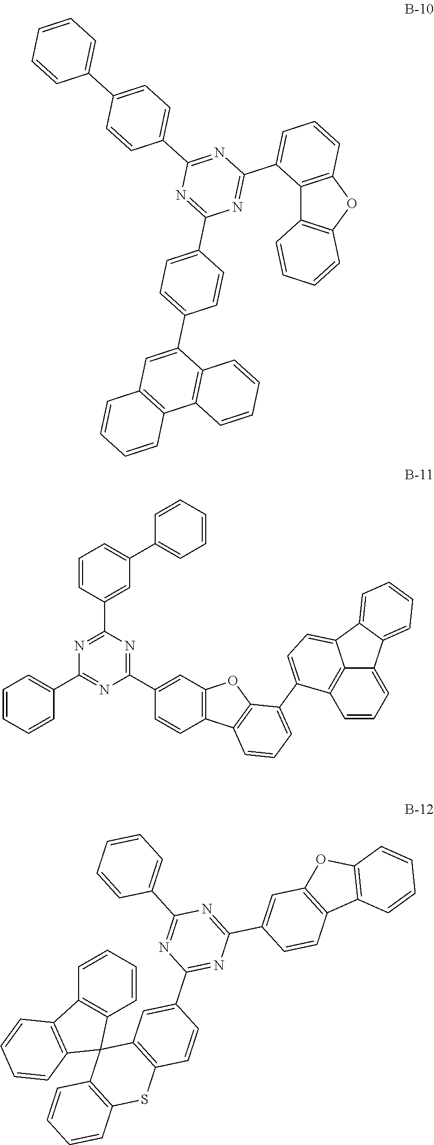 [SMILES: c1ccc(-c2ccc(-c3nc(-c4ccc(-c5cc6ccccc6c6ccccc56)cc4)nc(-c4cccc5oc6ccccc6c45)n3)cc2)cc1 ; c1ccc(-c2cccc(-c3nc(-c4ccccc4)nc(-c4ccc5c(c4)oc4c(-c6ccc7c8c(cccc68)-c6ccccc6-7)cccc45)n3)c2)cc1 ; c1ccc(-c2nc(-c3ccc4c(c3)C3(c5ccccc5S4)c4ccccc4-c4ccccc43)nc(-c3ccc4c(c3)oc3ccccc34)n2)cc1]

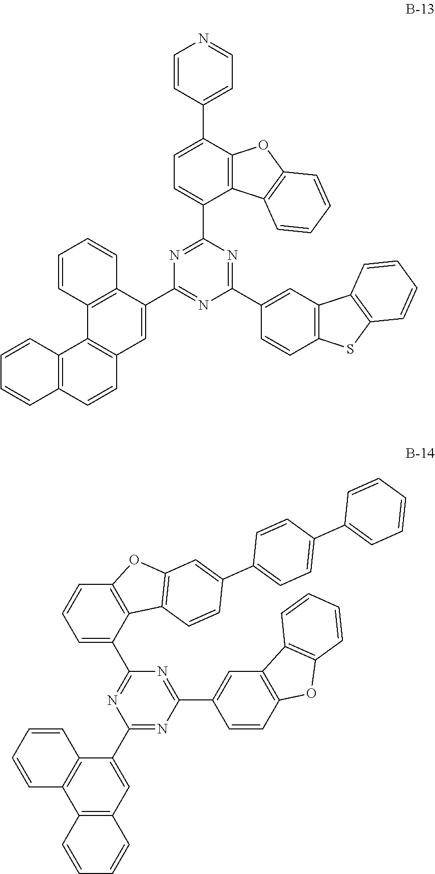 [SMILES: c1ccc2c(c1)ccc1cc(-c3nc(-c4ccc5sc6ccccc6c5c4)nc(-c4ccc(-c5ccncc5)c5oc6ccccc6c45)n3)c3ccccc3c12 ; c1ccc(-c2ccc(-c3ccc4c(c3)oc3cccc(-c5nc(-c6ccc7oc8ccccc8c7c6)nc(-c6cc7ccccc7c7ccccc67)n5)c34)cc2)cc1]

The driving voltage and efficiency were measured (15 mA/cm²) by applying a current to the organic light emitting devices manufactured in Examples 1 to 44 and Comparative Examples 1 to 14, and the results are shown in Tables 1 to 3 below. Lifetime T95 means the time required for the luminance to be reduced to 95% of the initial luminance (6000 nit).

TABLE 1

| | | Driving | Efficiency | Lifetime | Luminescent |
|---|---|---|---|---|---|
| Category | Host | voltage (V) | (cd/A) | T95 (hr) | color |
| Experimental | Compound1 | 4.11 | 17.11 | 105 | Red |
| Example 1 | | | | | |
| Experimental | Compound2 | 4.04 | 17.00 | 106 | Red |
| Example 2 | | | | | |
| Experimental | Compound3 | 4.16 | 18.37 | 117 | Red |
| Example 3 | | | | | |
| Experimental | Compound4 | 4.20 | 18.20 | 127 | Red |
| Example 4 | | | | | |
| Experimental | Compound5 | 4.03 | 18.05 | 123 | Red |
| Example 5 | | | | | |
| Experimental | Compound6 | 4.05 | 17.67 | 120 | Red |
| Example 6 | | | | | |
| Experimental | Compound7 | 4.09 | 18.30 | 123 | Red |
| Example 7 | | | | | |
| Experimental | Compound8 | 4.01 | 17.92 | 121 | Red |
| Example 8 | | | | | |
| Experimental | Compound9 | 4.17 | 16.95 | 114 | Red |
| Example 9 | | | | | |
| Experimental | Compound10 | 4.18 | 16.20 | 128 | Red |
| Example 10 | | | | | |
| Experimental | Compound11 | 4.36 | 17.92 | 142 | Red |
| Example 11 | | | | | |
| Experimental | Compound12 | 4.15 | 16.54 | 108 | Red |
| Example 12 | | | | | |
| Experimental | Compound13 | 4.24 | 17.21 | 139 | Red |
| Example 13 | | | | | |
| Experimental | Compound14 | 4.28 | 17.03 | 132 | Red |
| Example 14 | | | | | |
| Experimental | Compound15 | 4.33 | 17.23 | 149 | Red |
| Example 15 | | | | | |
| Experimental | Compound16 | 4.07 | 16.88 | 162 | Red |
| Example 16 | | | | | |
| Experimental | Compound17 | 4.05 | 16.82 | 151 | Red |
| Example 17 | | | | | |
| Experimental | Compound18 | 4.12 | 17.32 | 166 | Red |
| Example 18 | | | | | |
| Experimental | Compound19 | 4.00 | 17.49 | 153 | Red |
| Example 19 | | | | | |
| Experimental | Compound20 | 4.11 | 16.79 | 155 | Red |
| Example 20 | | | | | |
| Experimental | Compound21 | 4.04 | 16.85 | 156 | Red |
| Example 21 | | | | | |
| Experimental | Compound22 | 4.06 | 16.00 | 161 | Red |
| Example 22 | | | | | |

TABLE 2

| | | Driving | | Lifetime | Luminescent |
|---|---|---|---|---|---|
| Category | Host | voltage (V) | Efficiency(cd/A) | T95 (hr) | color |
| Experimental | Compound23 | 4.13 | 16.79 | 153 | Red |
| Example 23 | | | | | |
| Experimental | Compound24 | 4.38 | 15.94 | 116 | Red |
| Example 24 | | | | | |
| Experimental | Compound25 | 4.32 | 16.42 | 136 | Red |
| Example 25 | | | | | |
| Experimental | Compound26 | 4.37 | 16.31 | 117 | Red |
| Example 26 | | | | | |
| Experimental | Compound27 | 4.21 | 15.86 | 138 | Red |
| Example 27 | | | | | |
| Experimental | Compound28 | 4.02 | 18.79 | 105 | Red |
| Example 28 | | | | | |
| Experimental | Compound29 | 4.08 | 18.97 | 131 | Red |
| Example 29 | | | | | |
| Experimental | Compound30 | 4.17 | 15.54 | 128 | Red |
| Example 30 | | | | | |
| Experimental | Compound31 | 4.22 | 16.68 | 117 | Red |
| Example 31 | | | | | |
| Experimental | Compound32 | 4.29 | 15.08 | 128 | Red |
| Example 32 | | | | | |
| Experimental | Compound33 | 4.12 | 16.43 | 129 | Red |
| Example 33 | | | | | |
| Experimental | Compound34 | 4.11 | 16.83 | 123 | Red |
| Example 34 | | | | | |
| Experimental | Compound35 | 4.14 | 15.66 | 117 | Red |
| Example 35 | | | | | |
| Experimental | Compound36 | 4.30 | 16.27 | 137 | Red |
| Example 36 | | | | | |
| Experimental | Compound37 | 4.43 | 17.20 | 140 | Red |
| Example 37 | | | | | |
| Experimental | Compound38 | 4.18 | 17.94 | 159 | Red |
| Example 38 | | | | | |
| Experimental | Compound39 | 4.41 | 17.04 | 130 | Red |
| Example 39 | | | | | |
| Experimental | Compound40 | 4.24 | 17.48 | 148 | Red |
| Example 40 | | | | | |
| Experimental | Compound41 | 4.30 | 17.54 | 139 | Red |
| Example 41 | | | | | |
| Experimental | Compound42 | 4.25 | 17.38 | 152 | Red |
| Example 42 | | | | | |
| Experimental | Compound43 | 4.16 | 17.47 | 144 | Red |
| Example 43 | | | | | |
| Experimental | Compound44 | 4.22 | 17.32 | 133 | Red |
| Example 44 | | | | | |

TABLE 3

| | | Driving | Efficiency | Lifetime | Luminescent |
|---|---|---|---|---|---|
| Category | Host | voltage (V) | (cd/A) | T95 (hr) | color |
| Comparative | CompoundB-1 | 4.91 | 7.30 | 43 | Red |
| Experimental | | | | | |
| Example 1 | | | | | |
| Comparative | CompoundB-2 | 4.65 | 13.87 | 97 | Red |
| Experimental | | | | | |
| Example 2 | | | | | |
| Comparative | CompoundB-3 | 4.65 | 13.93 | 85 | Red |
| Experimental | | | | | |
| Example 3 | | | | | |
| Comparative | CompoundB-4 | 4.79 | 9.62 | 80 | Red |
| Experimental | | | | | |
| Example 4 | | | | | |
| Comparative | CompoundB-5 | 4.65 | 13.87 | 92 | Red |
| Experimental | | | | | |
| Example 5 | | | | | |
| Comparative | CompoundB-6 | 4.86 | 9.70 | 62 | Red |
| Experimental | | | | | |
| Example 6 | | | | | |
| Comparative | CompoundB-7 | 4.63 | 10.06 | 76 | Red |
| Experimental | | | | | |
| Example 7 | | | | | |
| Comparative | CompoundB-8 | 4.83 | 9.38 | 52 | Red |
| Experimental | | | | | |
| Example 8 | | | | | |
| Comparative | CompoundB-9 | 4.57 | 13.77 | 93 | Red |
| Experimental | | | | | |
| Example 9 | | | | | |
| Comparative | CompoundB-10 | 4.62 | 13.13 | 84 | Red |
| Experimental | | | | | |
| Example 10 | | | | | |
| Comparative | CompoundB-11 | 4.71 | 12.47 | 78 | Red |
| Experimental | | | | | |
| Example 11 | | | | | |
| Comparative | CompoundB-12 | 4.79 | 10.72 | 60 | Red |
| Experimental | | | | | |
| Example 12 | | | | | |
| Comparative | CompoundB-13 | 4.83 | 10.48 | 55 | Red |
| Experimental | | | | | |
| Example 13 | | | | | |
| Comparative | CompoundB-14 | 4.69 | 13.88 | 99 | Red |
| Experimental | | | | | |
| Example 14 | | | | | |

When a current was applied to the organic light emitting devices manufactured in Examples 1 to 44 and Comparative Experimental Examples 1 to 14, the results shown in Tables 1 to 3 were obtained. The red organic light emitting device of Experimental Example 1 used a material that has been widely used in the past, and has a structure using Compound [EB-1] as an electron blocking layer and Dp-7 as a red dopant. It can be confirmed that when the compound of Chemical Formula 1 of the present disclosure is used as a red light emitting layer, the driving voltage is decreased and the efficiency and lifetime are increased as compared with Comparative Experimental Examples, as shown in Tables 1 and 2. Furthermore, when the compounds B-1 to B-14 of Comparative Experimental Examples were used as the red light emitting layer, the driving voltage is increased and the efficiency and lifetime are decreased as compared with the compounds of Experimental Examples, as shown in Table 3.

From the above results, it can be inferred that the reason why the driving voltage is improved and the efficiency and lifetime are increased is that when the compounds of the present disclosure are used, energy transfer to the red dopant in the red light emitting layer is made more favorable as compared with the compounds of the Comparative Examples. Therefore, it can be confirmed that since the compounds of the present disclosure achieve a more stable balance in the light emitting layer than the compounds of the Comparative Examples, electrons and holes combine to form excitons, which is greatly increased in efficiency and lifetime. In conclusion, it was confirmed that when the compounds of the present disclosure are used as host for the red light emitting layer, the driving voltage, luminous efficiency, and lifetime characteristics of the organic light emitting device can be improved.

[Description of symbols]

| | |
|---|---|
| 1: substrate | 2: anode |
| 3: light emitting layer | 4: cathode |
| 5: hole injection layer | 6: hole transport layer |
| 7: light emitting layer | 8: electron injection and transport layer |

1. A compound of Chemical Formula 1: 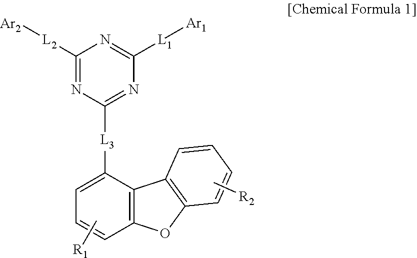
wherein, in Chemical Formula 1:
Ar₁ is unsubstituted benzophenanthrenyl, chrysenyl, or fluoranthenyl;
Ar₂ is a substituted or unsubstituted C₆₋₆₀ aryl, or a substituted or unsubstituted C₆₋₆₀ heteroaryl containing any one or more selected from the group consisting of N, O and S;
L₁ is a direct bond or a substituted or unsubstituted C₆₋₆₀ arylene;
L₂ is a direct bond or a substituted or unsubstituted C₆₋₆₀ arylene;
L₃ is a direct bond or a substituted or unsubstituted C₆₋₆₀ arylene; and
R₁ and R₂ are each independently hydrogen, deuterium, a substituted or unsubstituted C₁₋₁₂ alkyl, or a substituted or unsubstituted C₆₋₁₄ aryl.

2. The compound of claim 1, wherein:
L₁ is a direct bond or phenylene.

3. The compound of claim 1, wherein:
L₂ is a direct bond or phenylene.

4. The compound of claim 1, wherein:
L₃ is a direct bond, or phenylene, or naphthylene.

5. The compound of claim 1, wherein:
Ar₂ is phenyl, biphenyl, naphthyl, dibenzofuranyl, or dibenzothiophenyl.

6. The compound of claim 1, wherein:
R₁ and R₂ are each independently hydrogen, deuterium, phenyl, biphenyl or naphthyl.

7. The compound of claim 1, wherein:
the compound of Chemical Formula 1 is any one compound selected from the group consisting of:

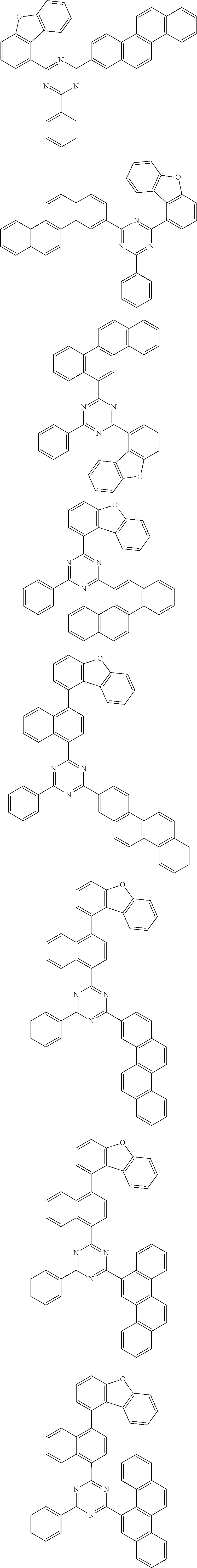

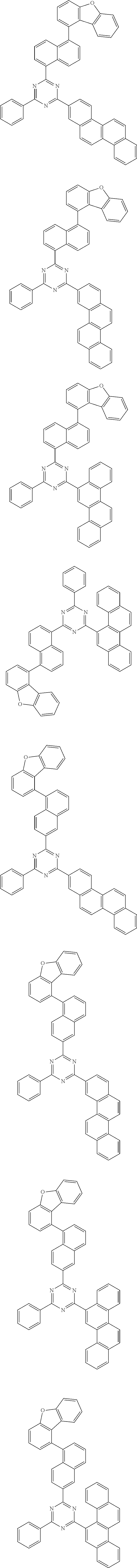

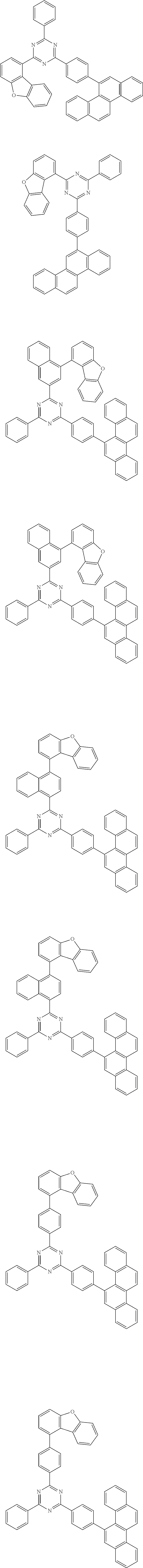

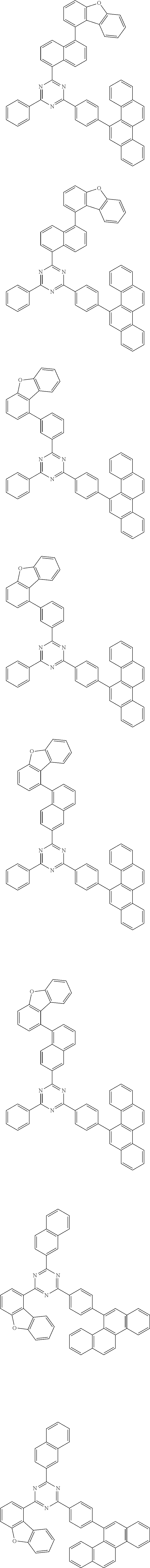

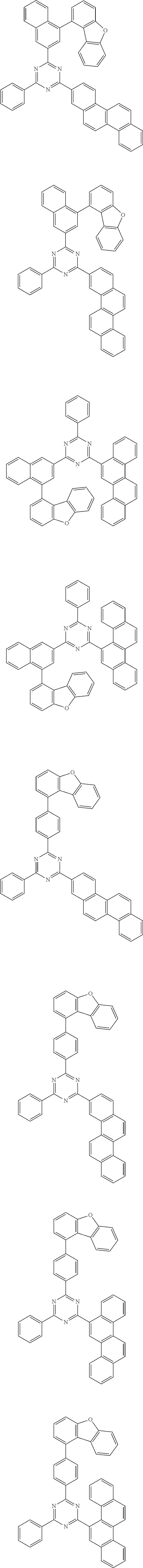

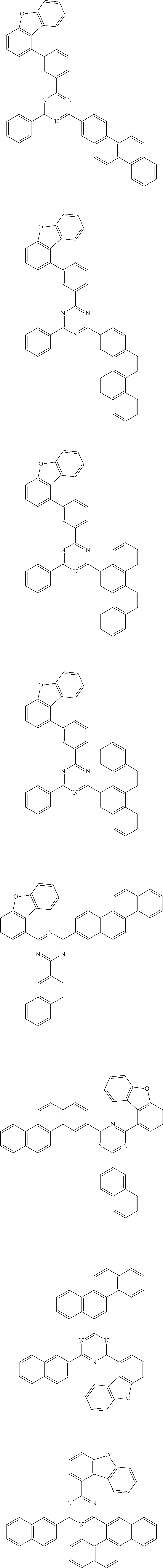

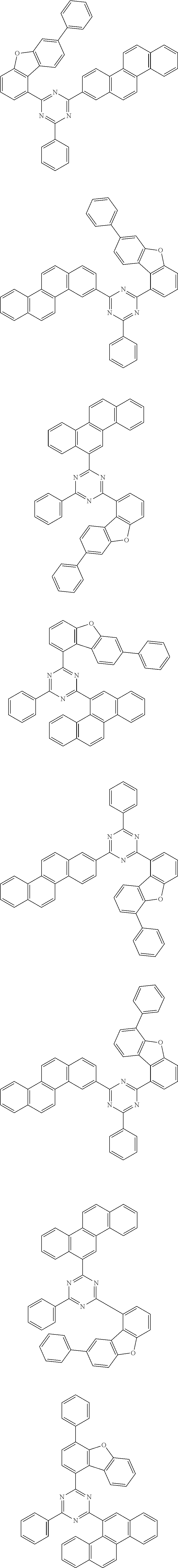

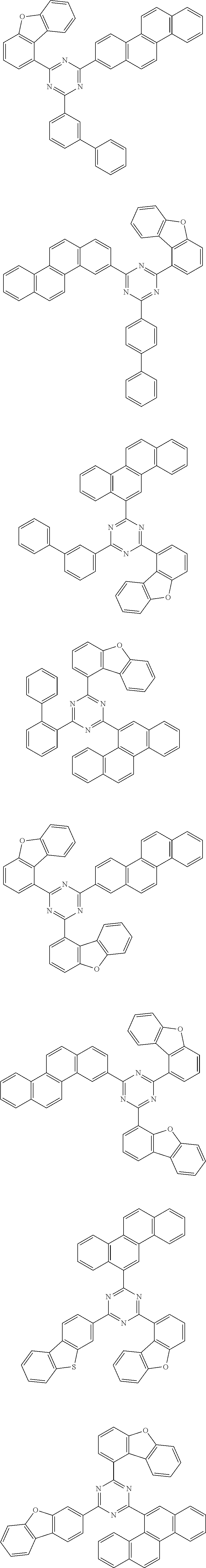

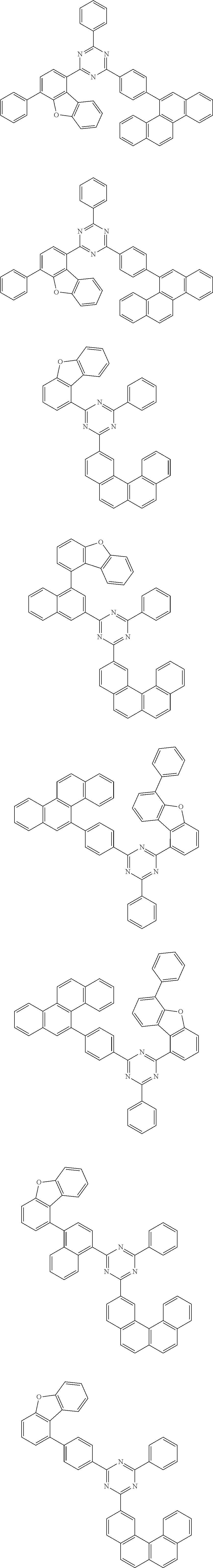

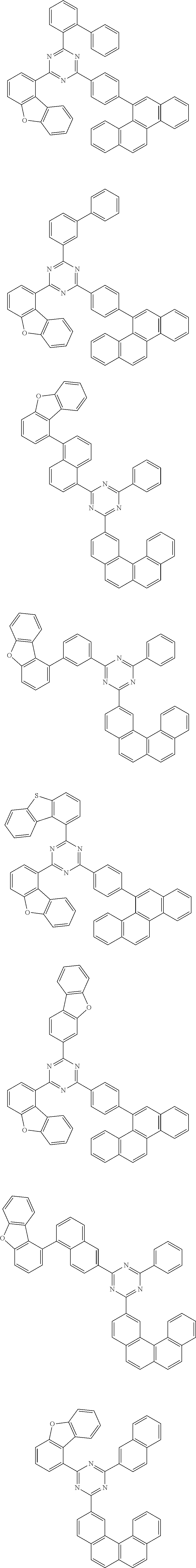

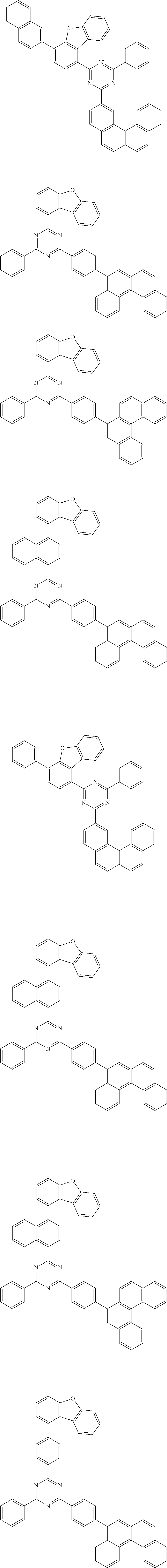

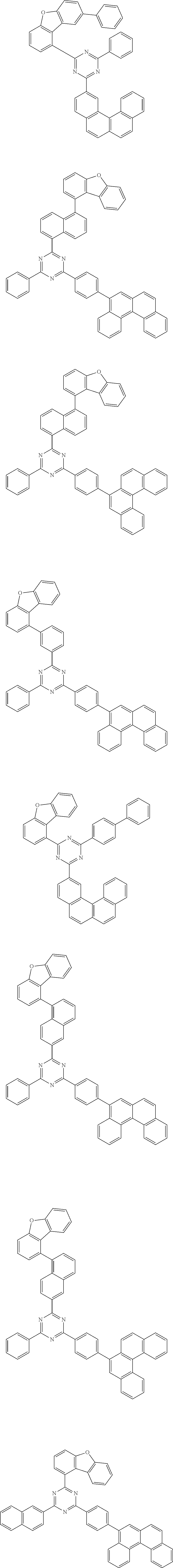

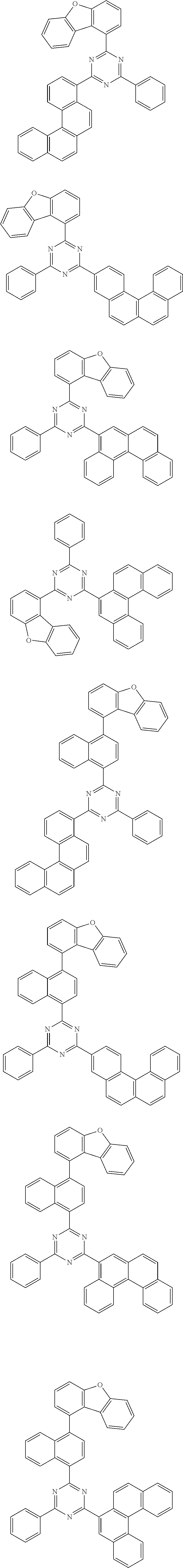

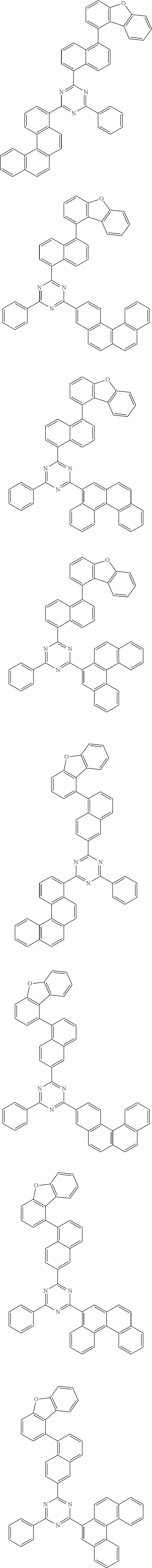

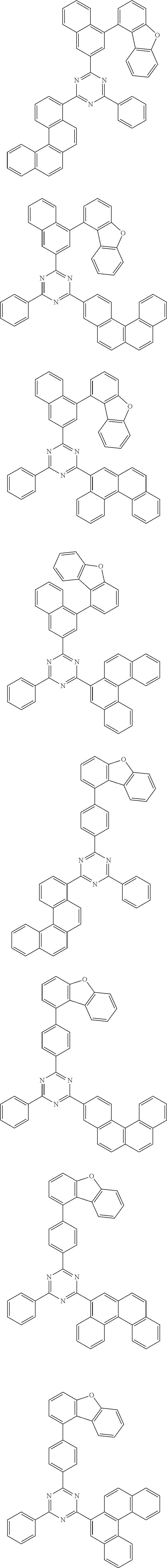

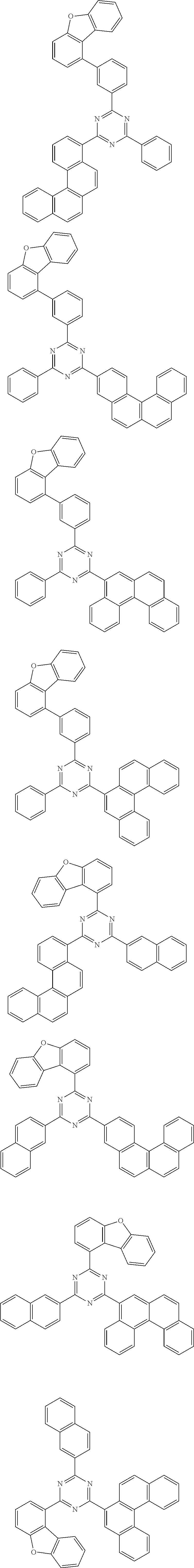

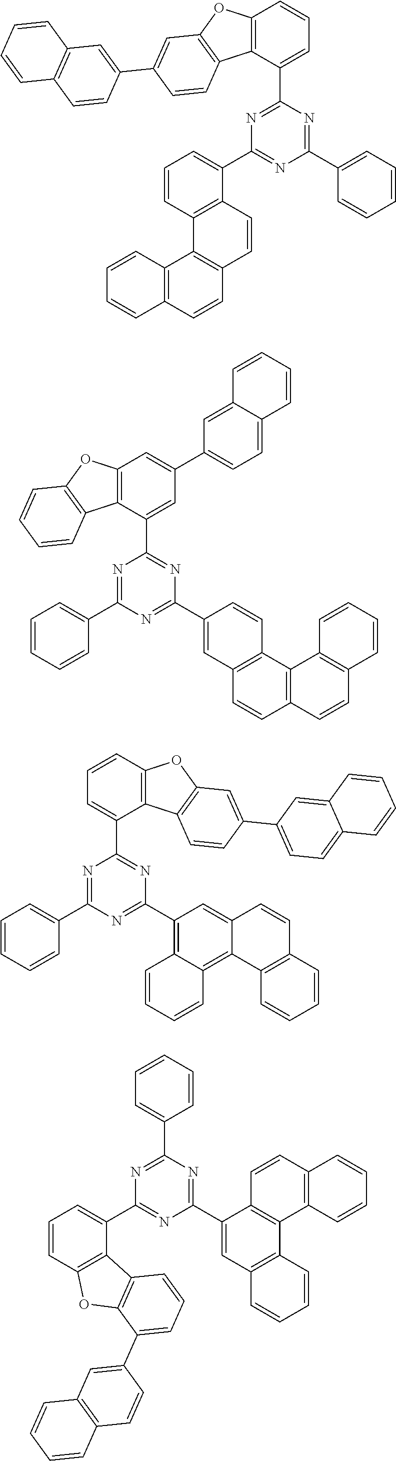

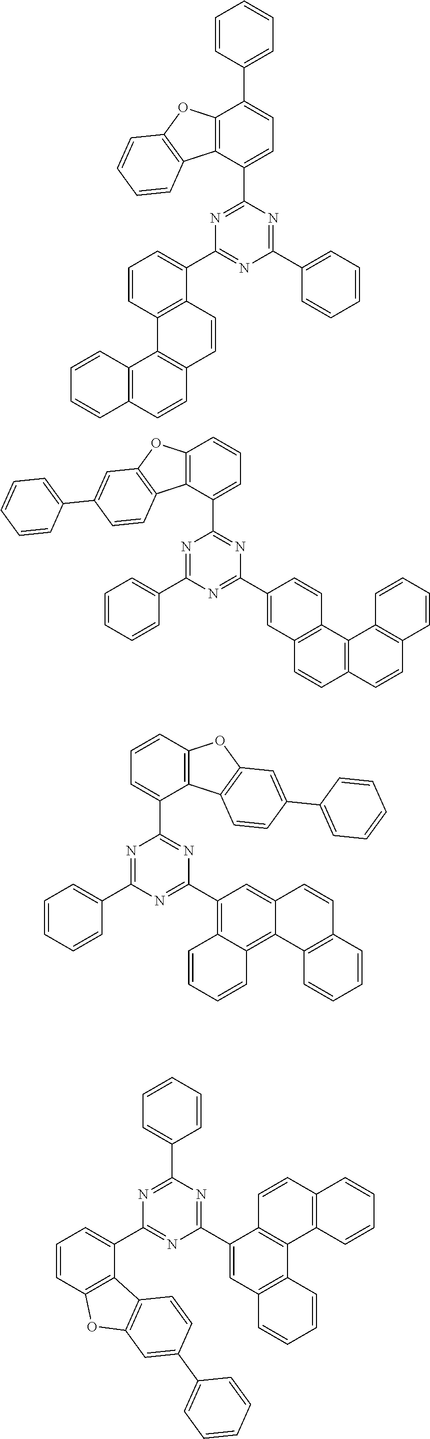

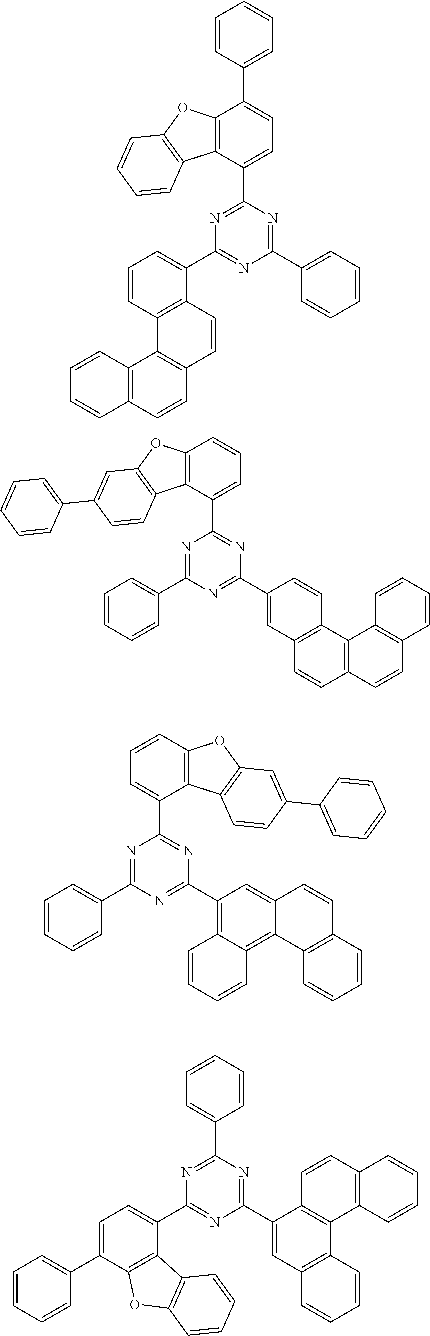

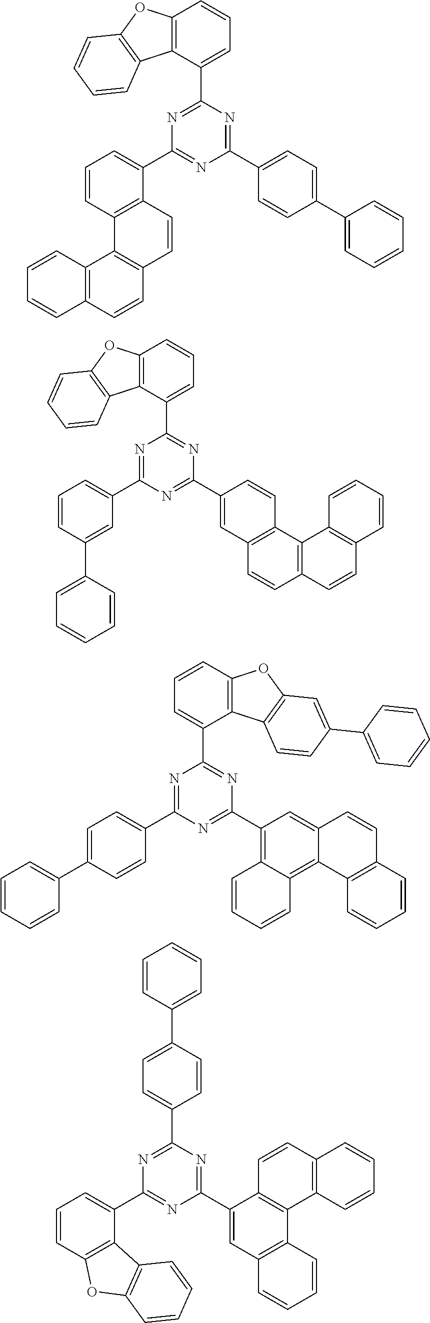

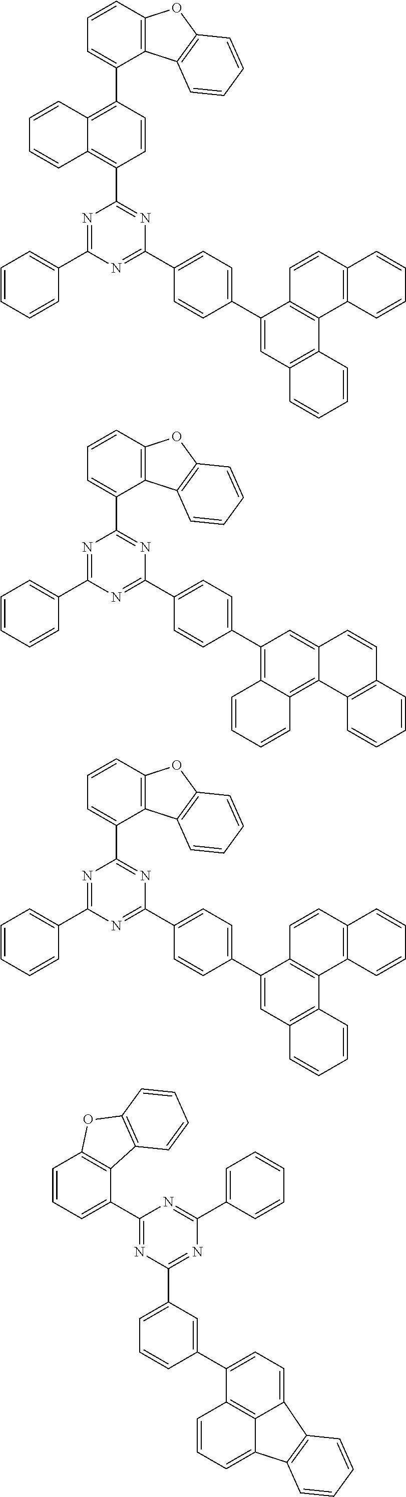 [SMILES: c1ccc(-c2nc(-c3ccc(-c4cc5ccccc5c5c4ccc4ccccc45)cc3)nc(-c3ccc(-c4cccc5oc6ccccc6c45)c4ccccc34)n2)cc1 ; c1ccc(-c2nc(-c3ccc(-c4cc5ccc6ccccc6c5c5ccccc45)cc3)nc(-c3cccc4oc5ccccc5c34)n2)cc1 ; c1ccc(-c2nc(-c3ccc(-c4cc5ccccc5c5c4ccc4ccccc45)cc3)nc(-c3cccc4oc5ccccc5c34)n2)cc1 ; c1ccc(-c2nc(-c3cccc(-c4ccc5c6c(cccc46)-c4ccccc4-5)c3)nc(-c3cccc4oc5ccccc5c34)n2)cc1]

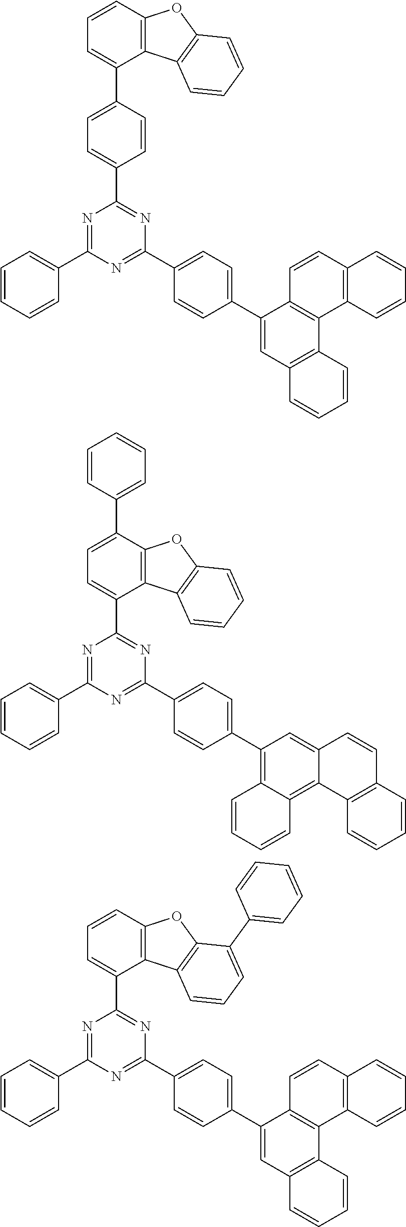

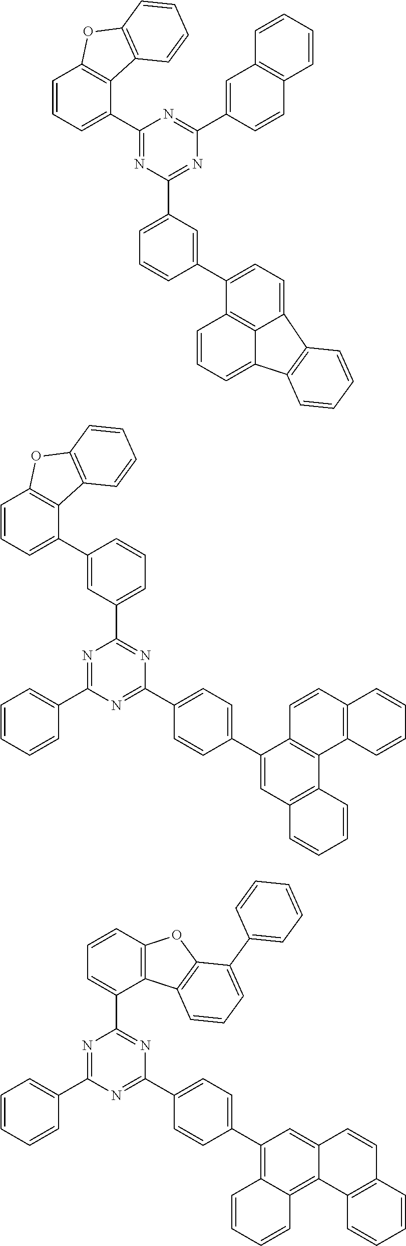

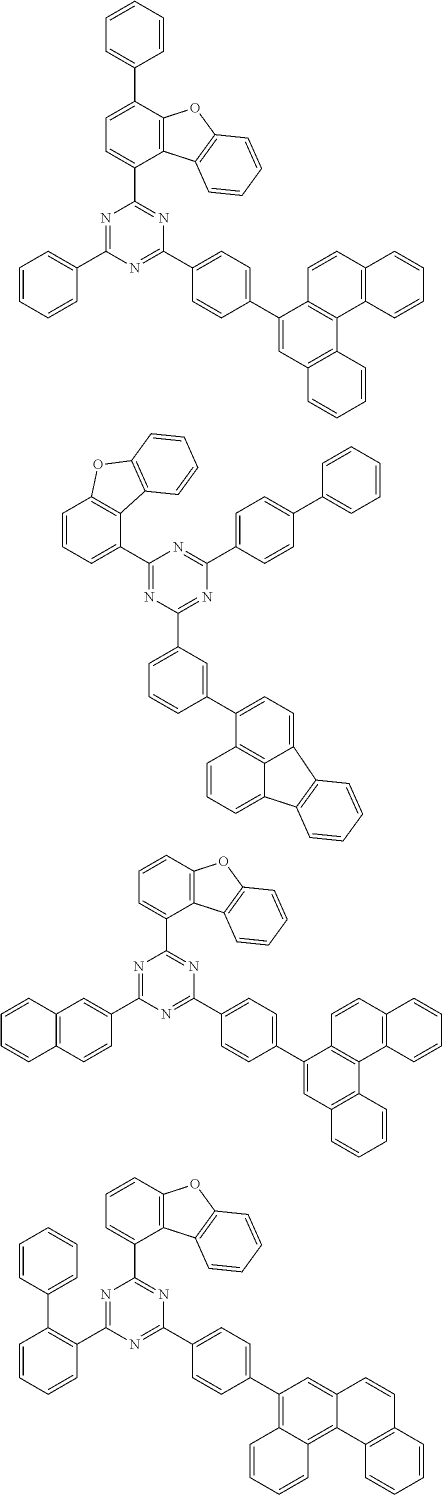

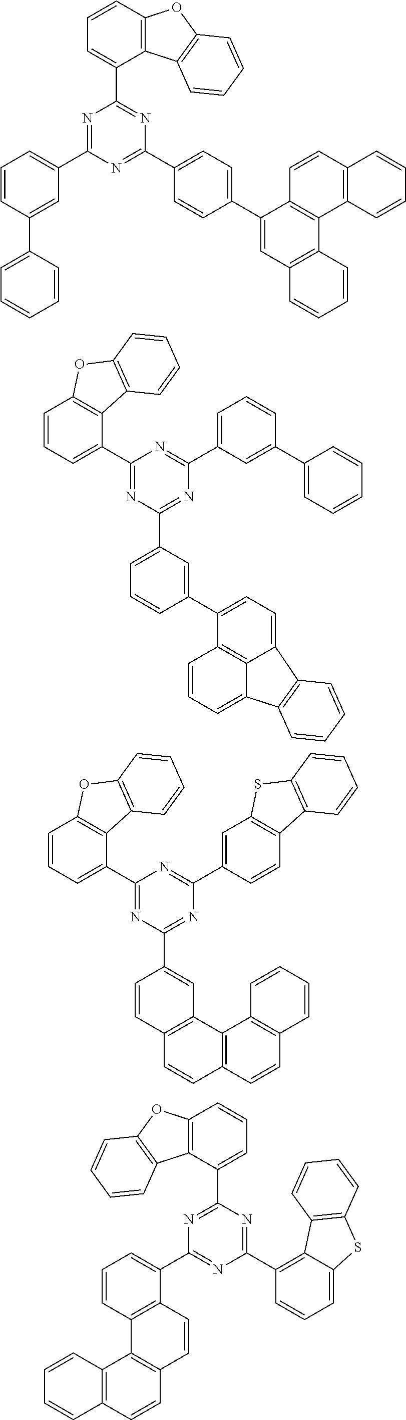

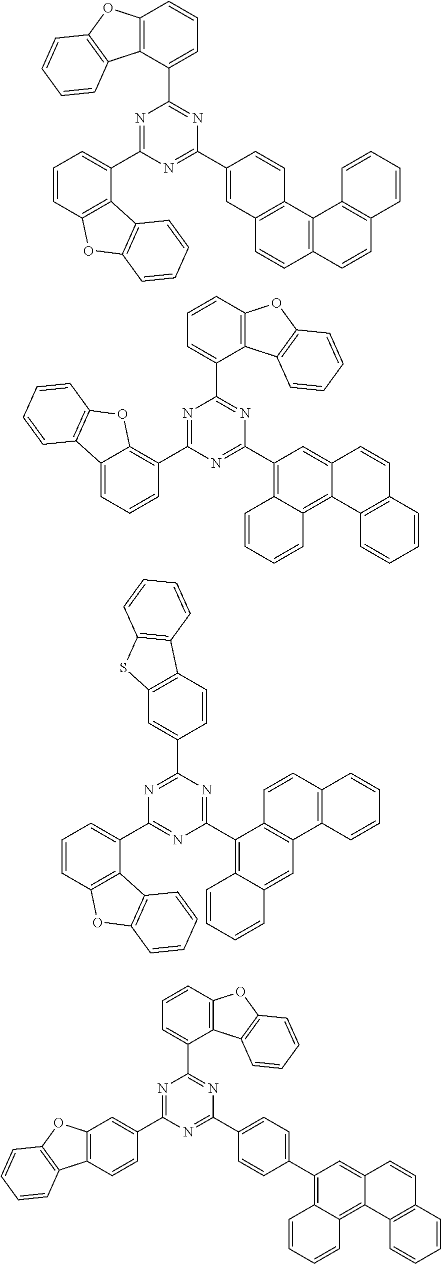

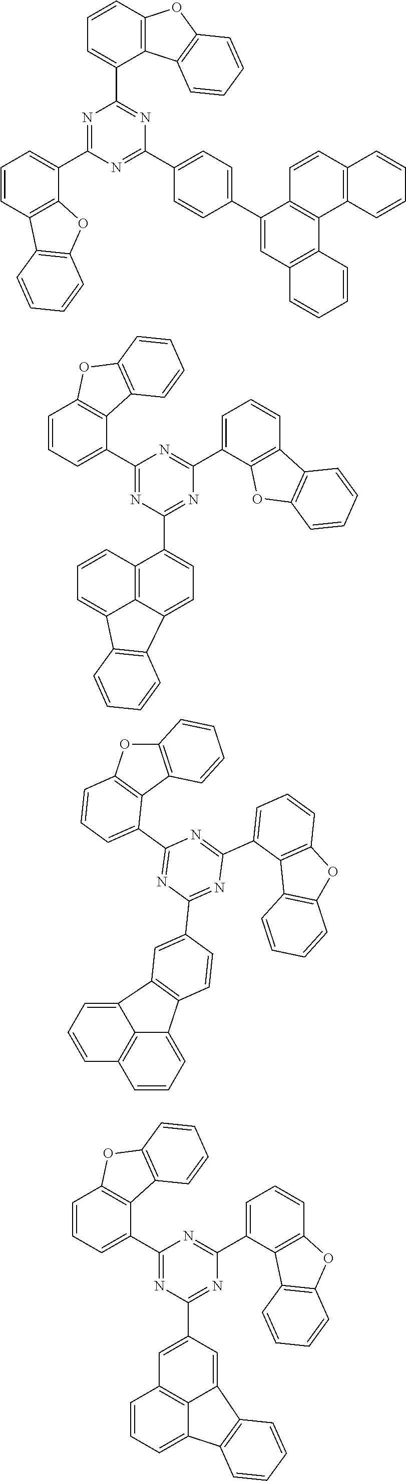

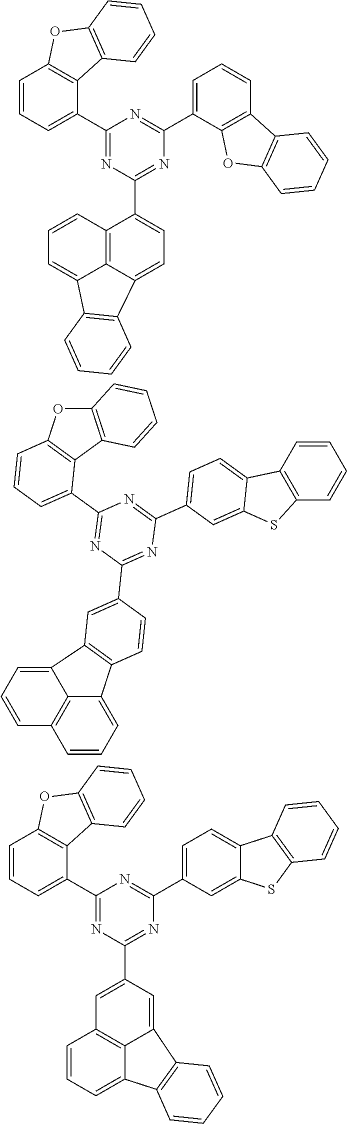

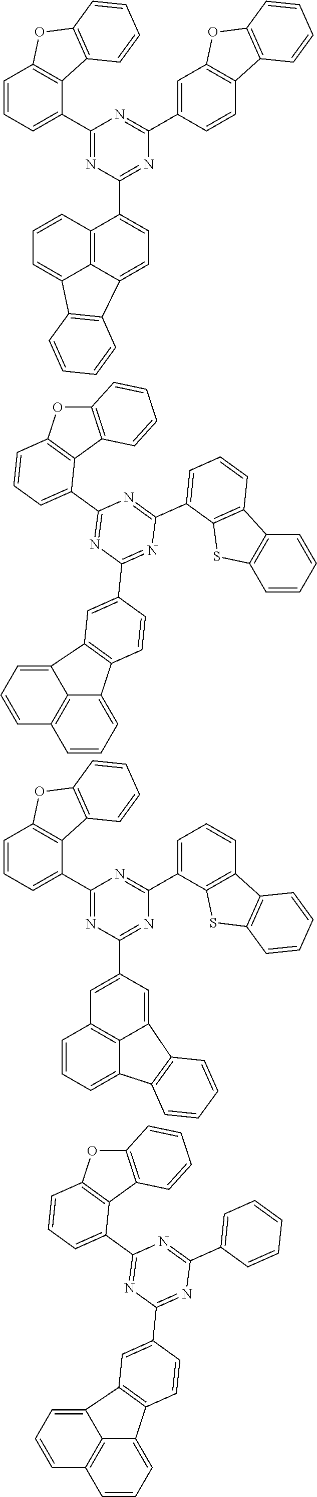

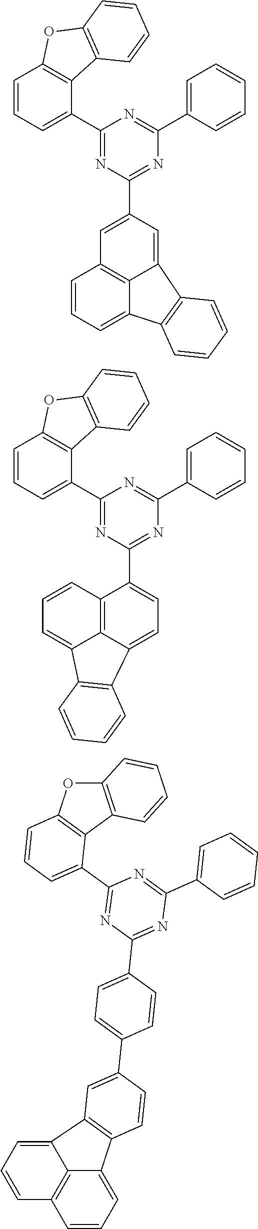

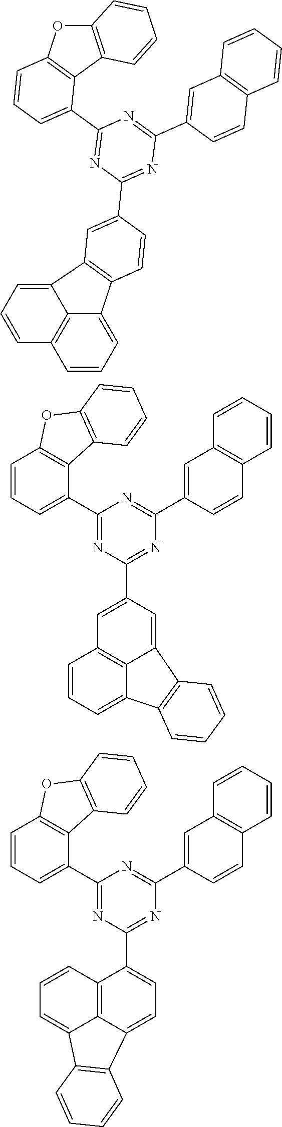

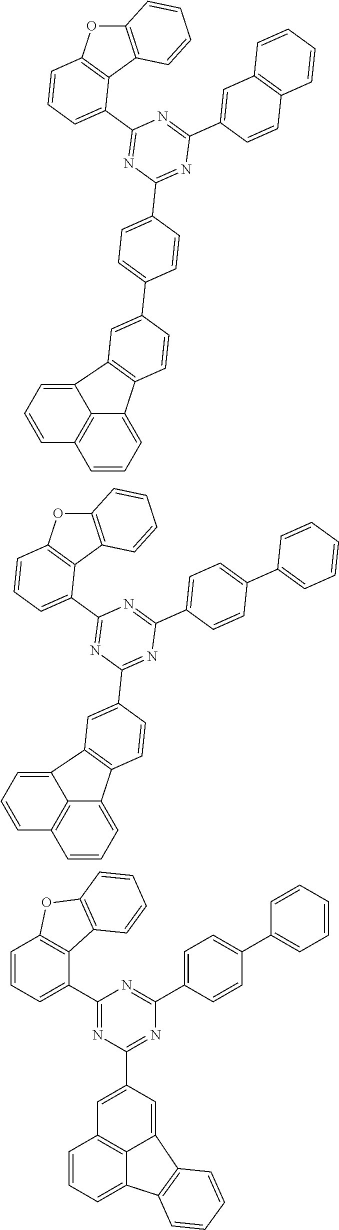

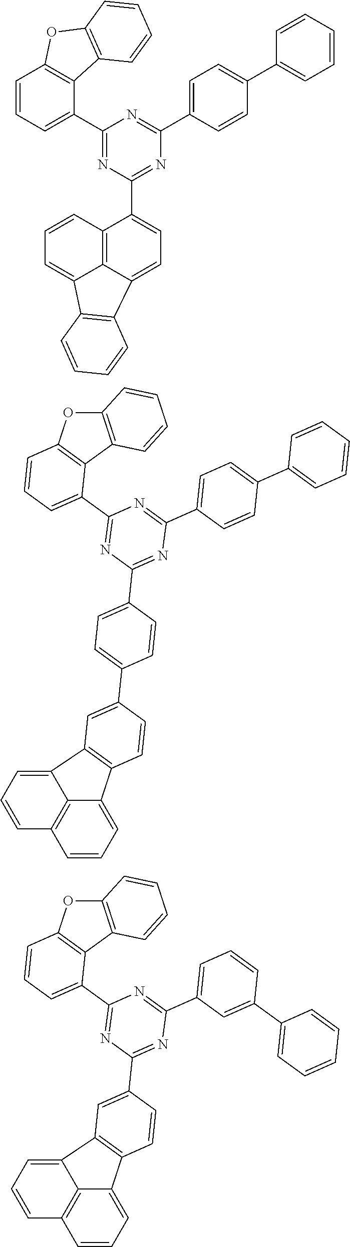

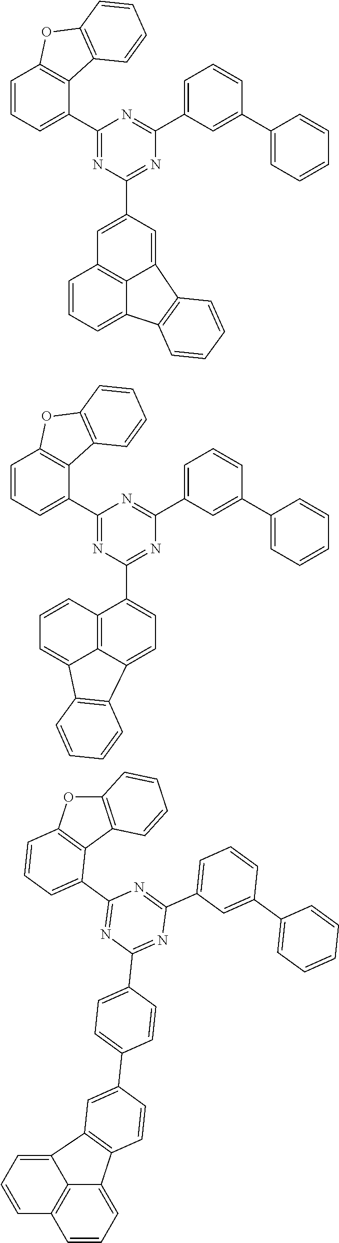

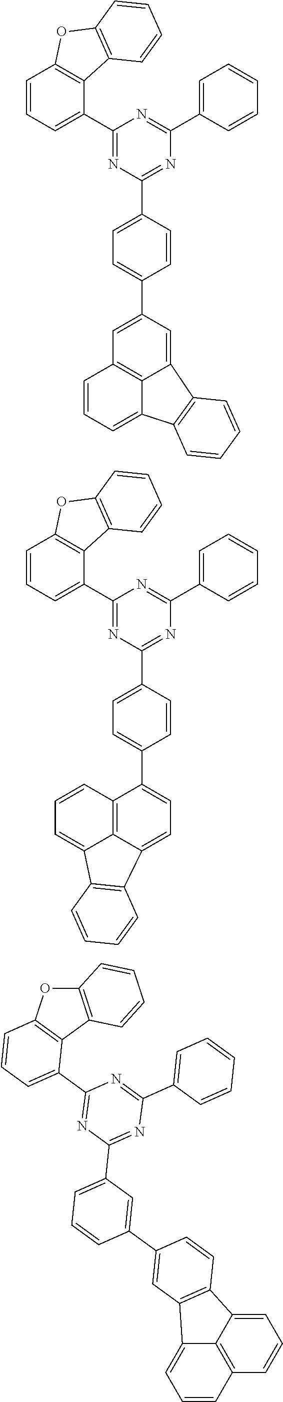

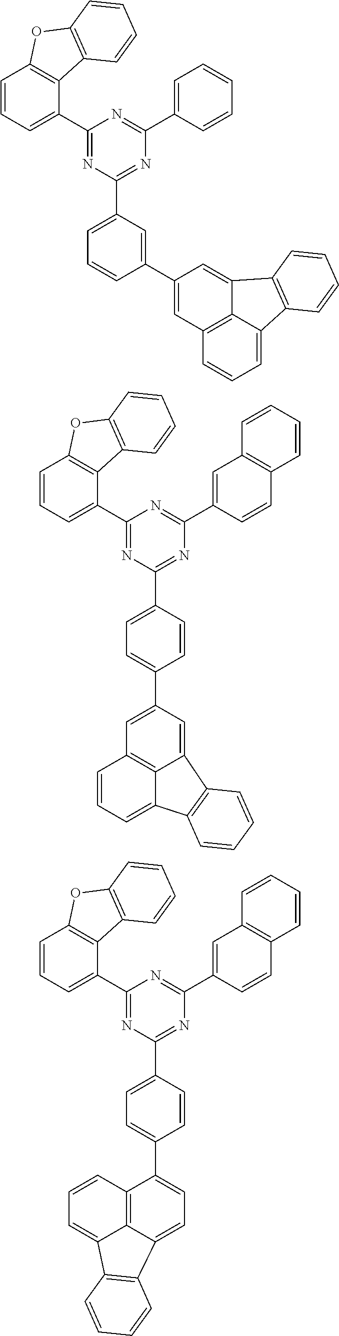

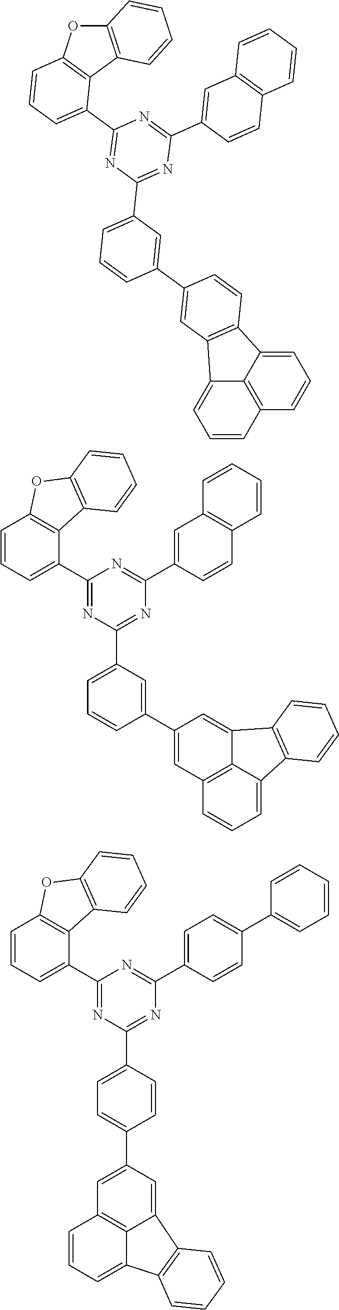

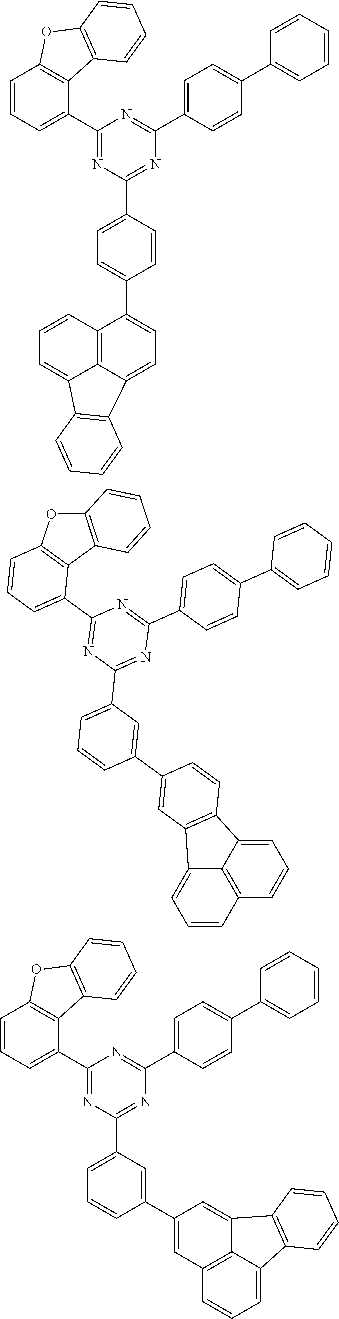

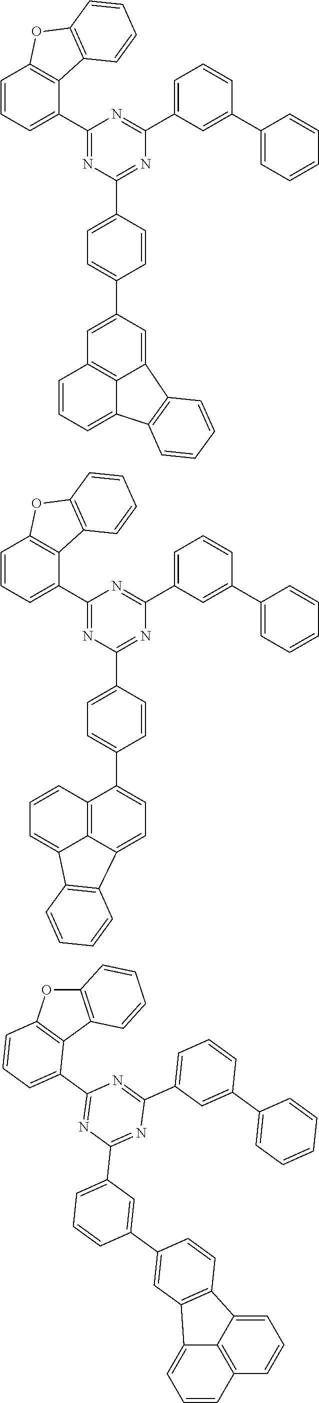

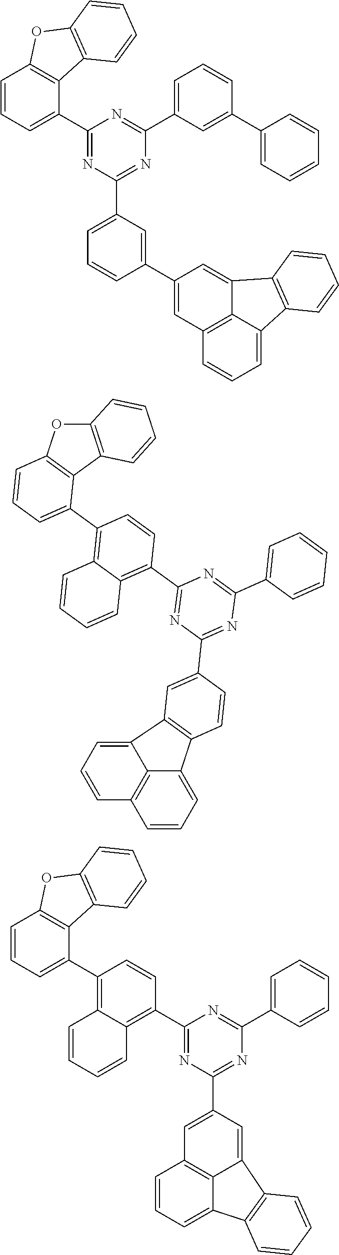

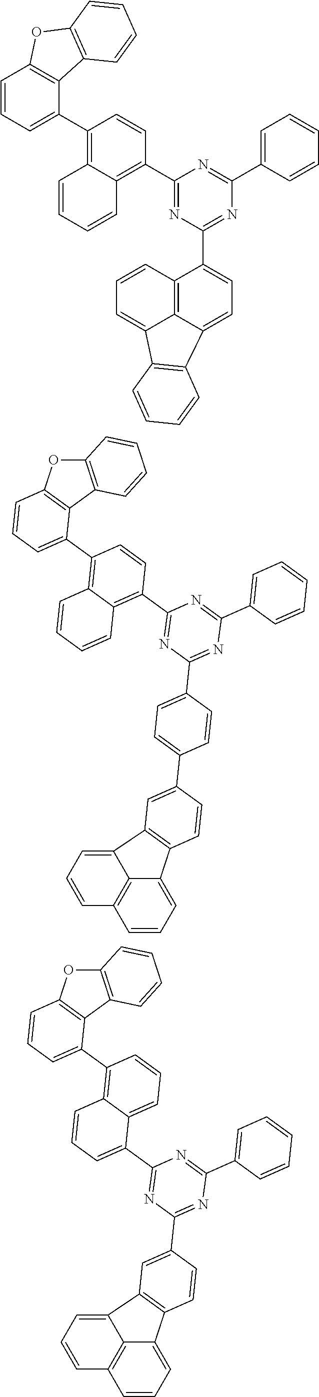

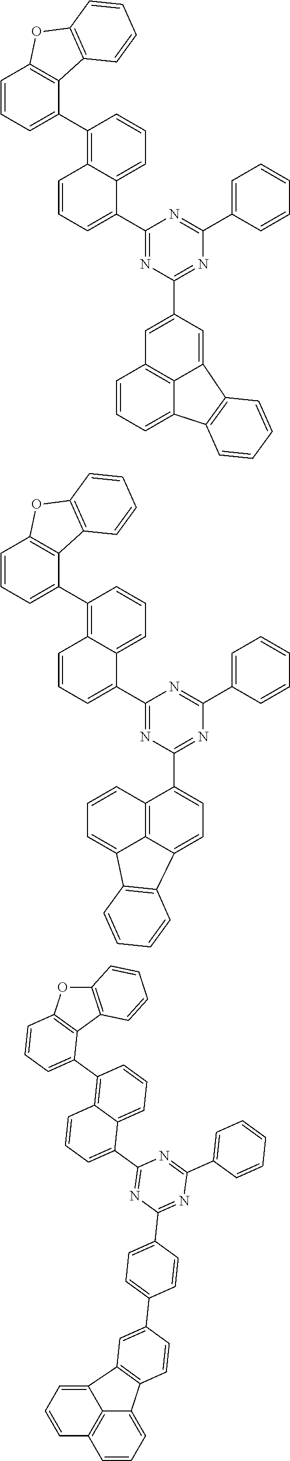

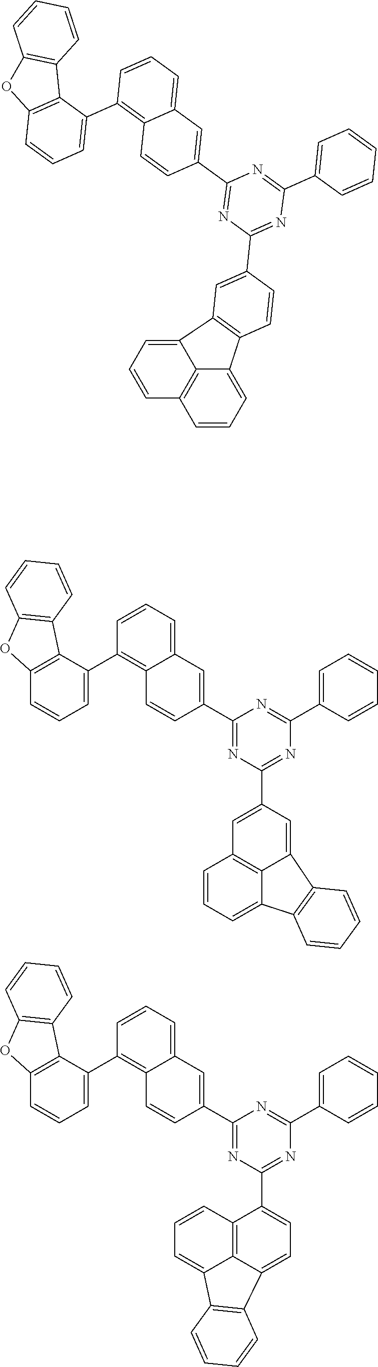 [SMILES: c1ccc(-c2nc(-c3ccc4c(c3)-c3cccc5cccc-4c35)nc(-c3ccc4c(-c5cccc6oc7ccccc7c56)cccc4c3)n2)cc1 ; c1ccc(-c2nc(-c3ccc4c(-c5cccc6oc7ccccc7c56)cccc4c3)nc(-c3cc4c5c(cccc5c3)-c3ccccc3-4)n2)cc1 ; c1ccc(-c2nc(-c3ccc4c(-c5cccc6oc7ccccc7c56)cccc4c3)nc(-c3ccc4c5c(cccc35)-c3ccccc3-4)n2)cc1]

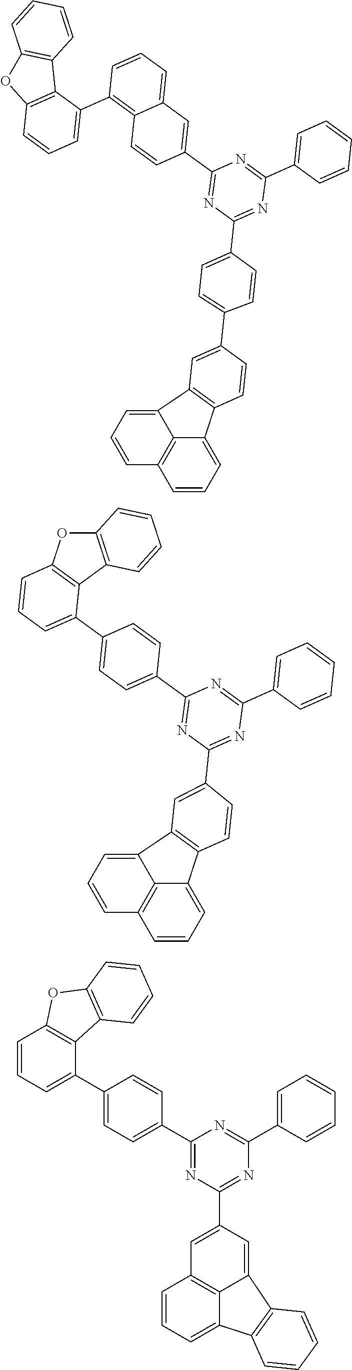

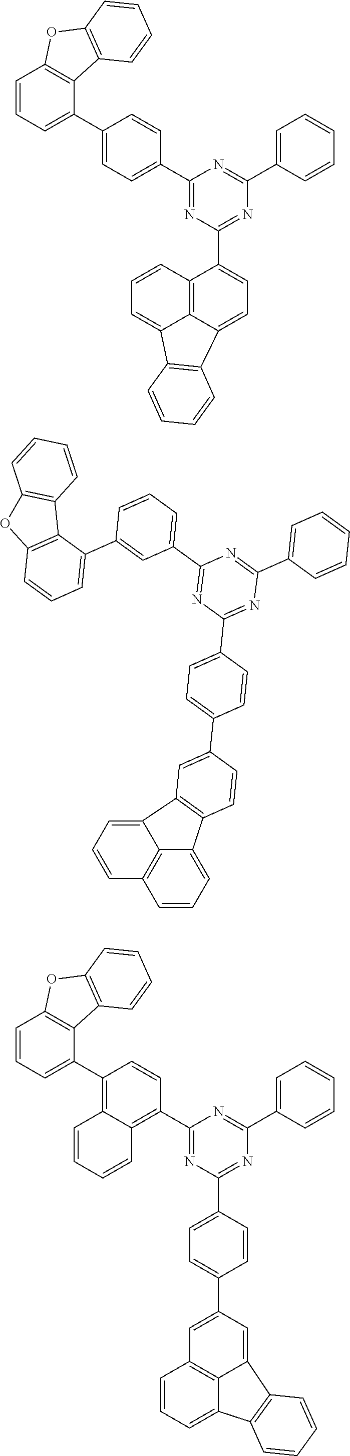 [SMILES: c1ccc(-c2nc(-c3ccc(-c4cccc5oc6ccccc6c45)cc3)nc(-c3ccc4c5c(cccc35)-c3ccccc3-4)n2)cc1 ; c1ccc(-c2nc(-c3ccc(-c4ccc5c(c4)-c4cccc6cccc-5c46)cc3)nc(-c3cccc(-c4cccc5oc6ccccc6c45)c3)n2)cc1 ; c1ccc(-c2nc(-c3ccc(-c4cc5c6c(cccc6c4)-c4ccccc4-5)cc3)nc(-c3ccc(-c4cccc5oc6ccccc6c45)c4ccccc34)n2)cc1]

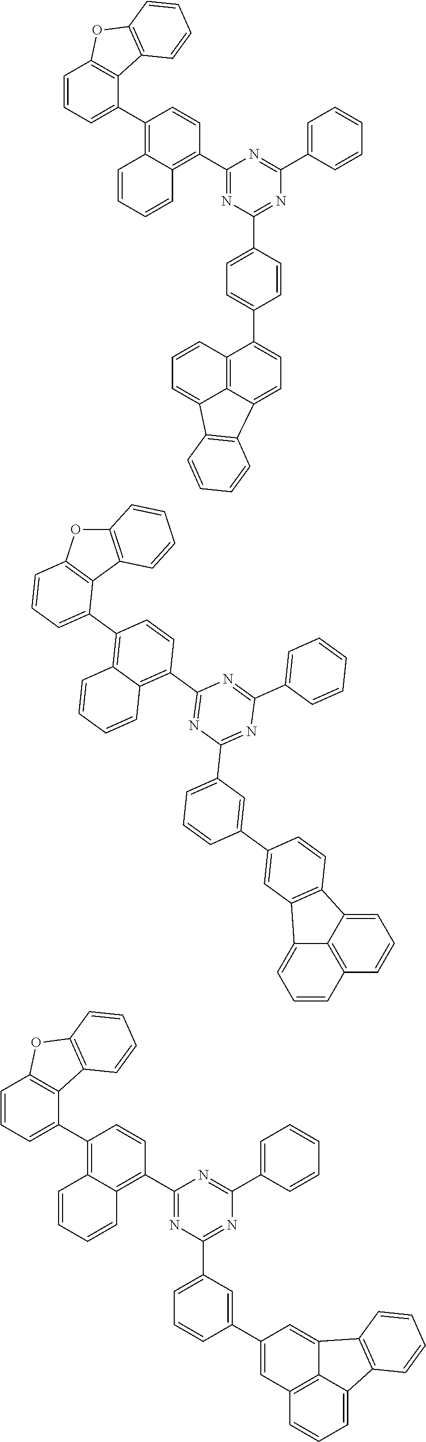

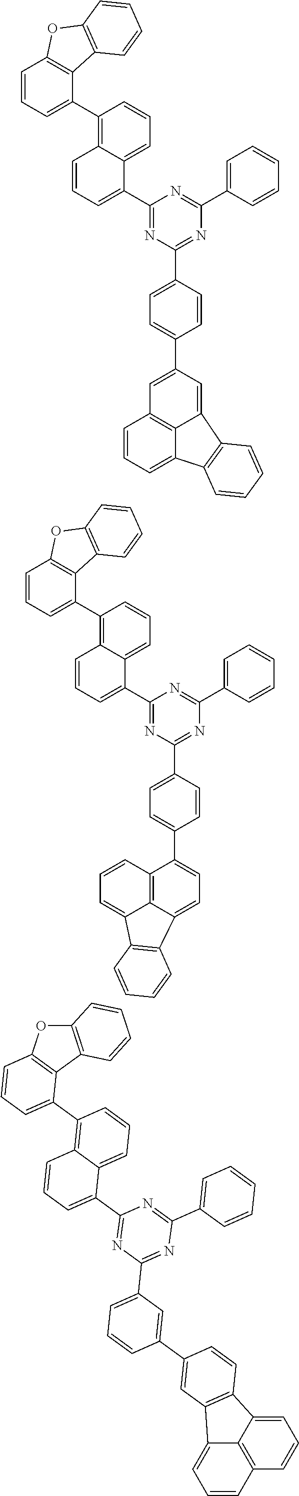

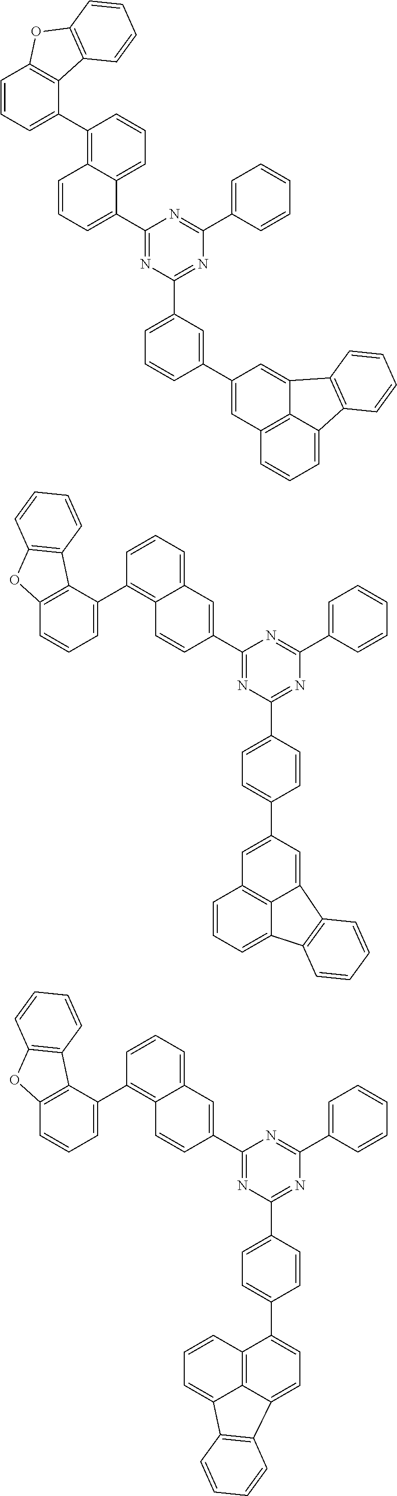 [SMILES: c1ccc(-c2nc(-c3cccc(-c4cc5c6c(cccc6c4)-c4ccccc4-5)c3)nc(-c3cccc4c(-c5cccc6oc7ccccc7c56)cccc34)n2)cc1 ; c1ccc(-c2nc(-c3ccc(-c4cc5c6c(cccc6c4)-c4ccccc4-5)cc3)nc(-c3ccc4c(-c5cccc6oc7ccccc7c56)cccc4c3)n2)cc1 ; c1ccc(-c2nc(-c3ccc(-c4ccc5c6c(cccc46)-c4ccccc4-5)cc3)nc(-c3ccc4c(-c5cccc6oc7ccccc7c56)cccc4c3)n2)cc1]

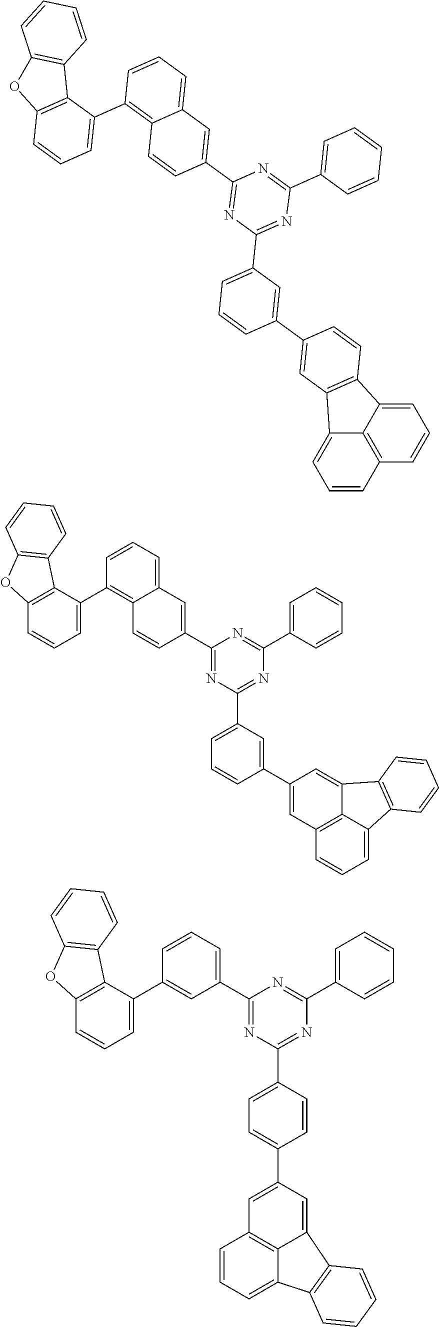 [SMILES: c1ccc(-c2nc(-c3cccc(-c4ccc5c(c4)-c4cccc6cccc-5c46)c3)nc(-c3ccc4c(-c5cccc6oc7ccccc7c56)cccc4c3)n2)cc1 ; c1ccc(-c2nc(-c3cccc(-c4cc5c6c(cccc6c4)-c4ccccc4-5)c3)nc(-c3ccc4c(-c5cccc6oc7ccccc7c56)cccc4c3)n2)cc1 ; c1ccc(-c2nc(-c3ccc(-c4cc5c6c(cccc6c4)-c4ccccc4-5)cc3)nc(-c3cccc(-c4cccc5oc6ccccc6c45)c3)n2)cc1]

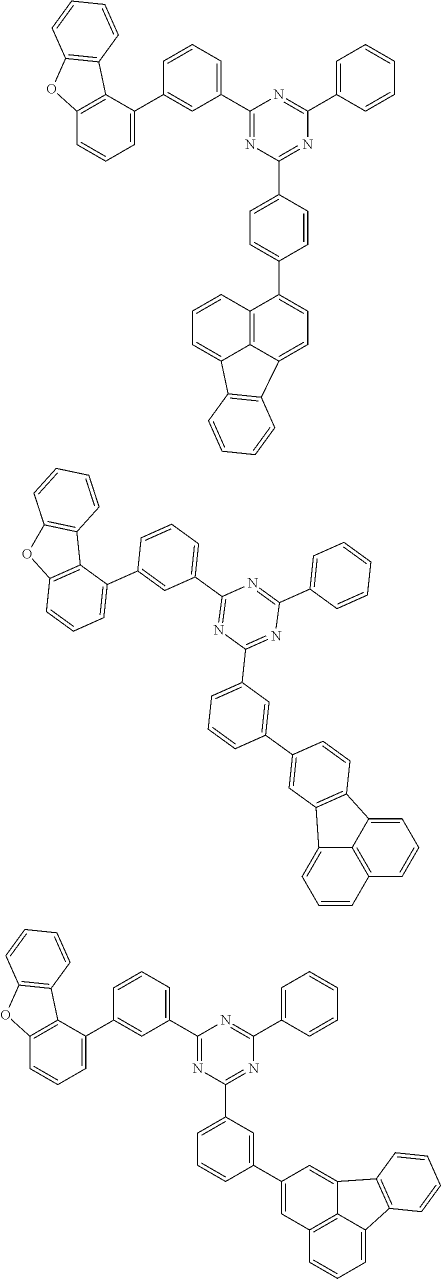 [SMILES: c1ccc(-c2nc(-c3ccc(-c4ccc5c6c(cccc46)-c4ccccc4-5)cc3)nc(-c3cccc(-c4cccc5oc6ccccc6c45)c3)n2)cc1 ; c1ccc(-c2nc(-c3cccc(-c4ccc5c(c4)-c4cccc6cccc-5c46)c3)nc(-c3cccc(-c4cccc5oc6ccccc6c45)c3)n2)cc1 ; c1ccc(-c2nc(-c3cccc(-c4cc5c6c(cccc6c4)-c4ccccc4-5)c3)nc(-c3cccc(-c4cccc5oc6ccccc6c45)c3)n2)cc1]

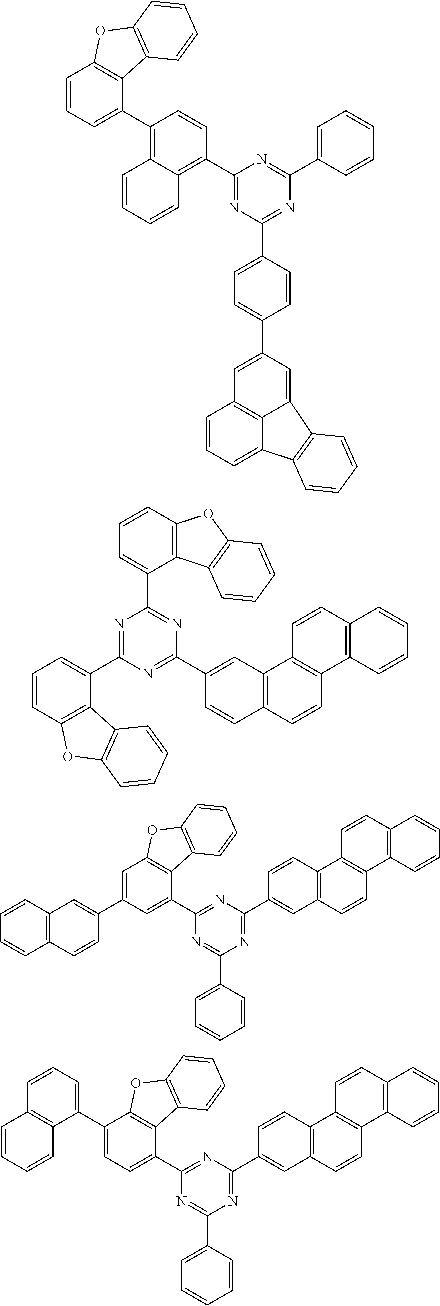

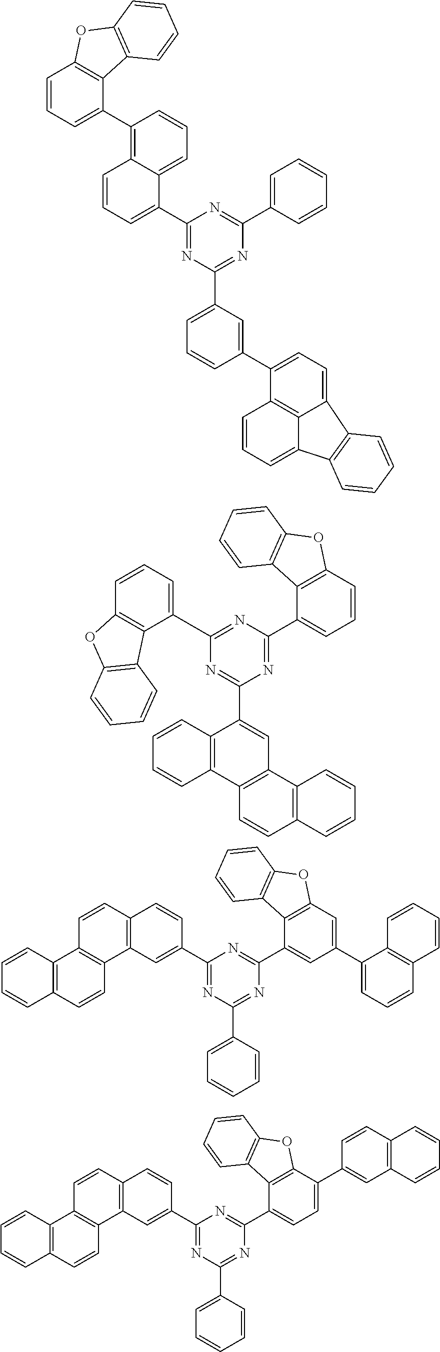

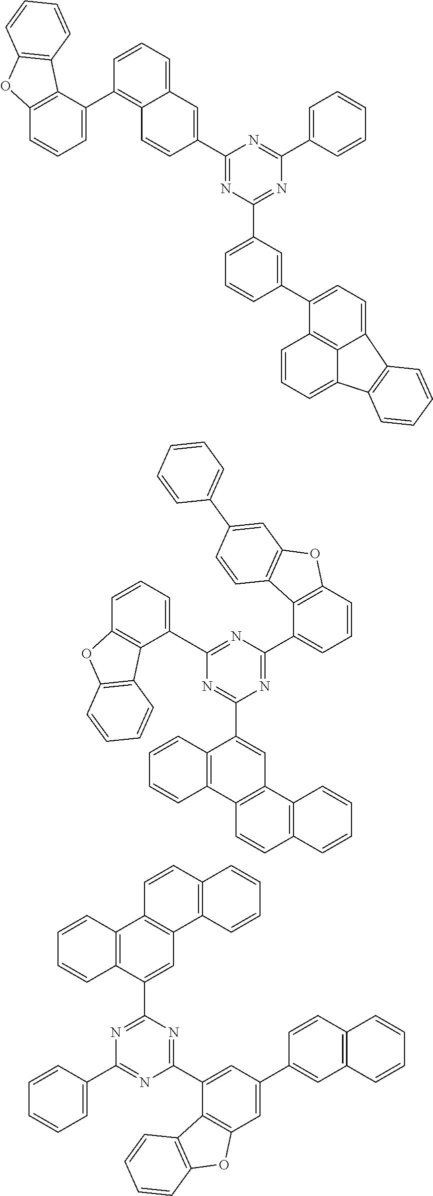

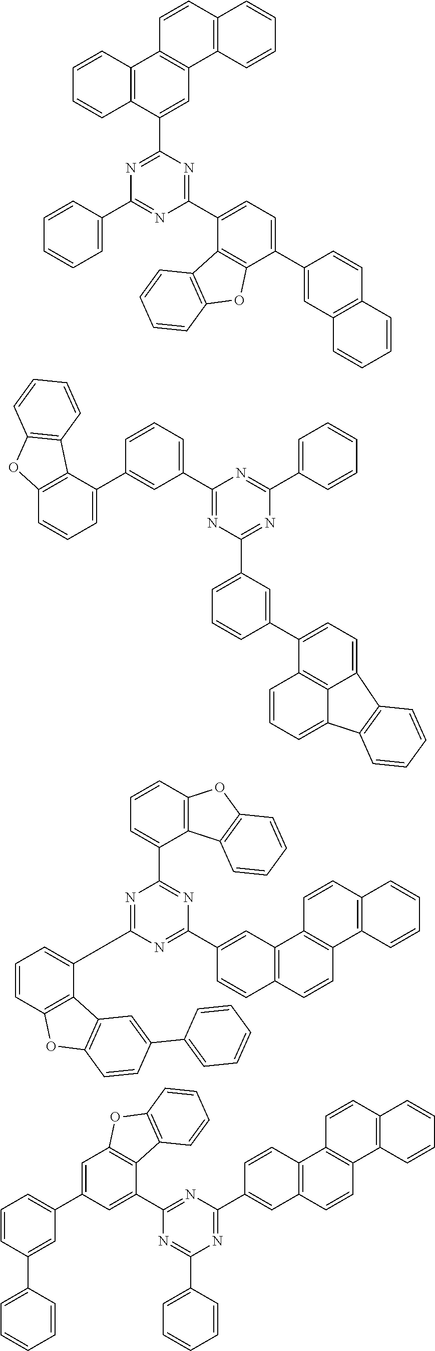

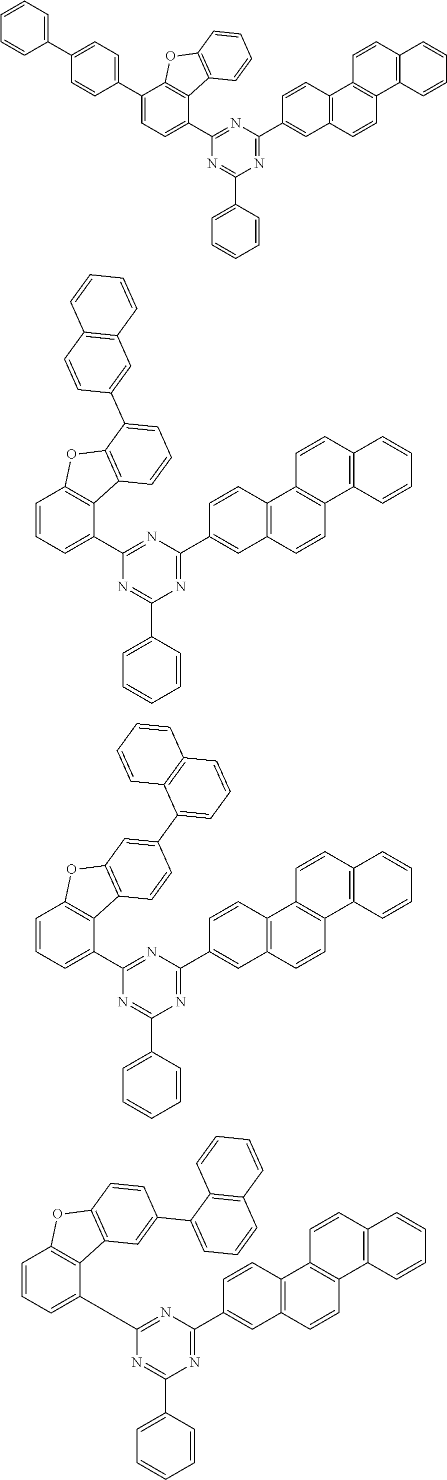

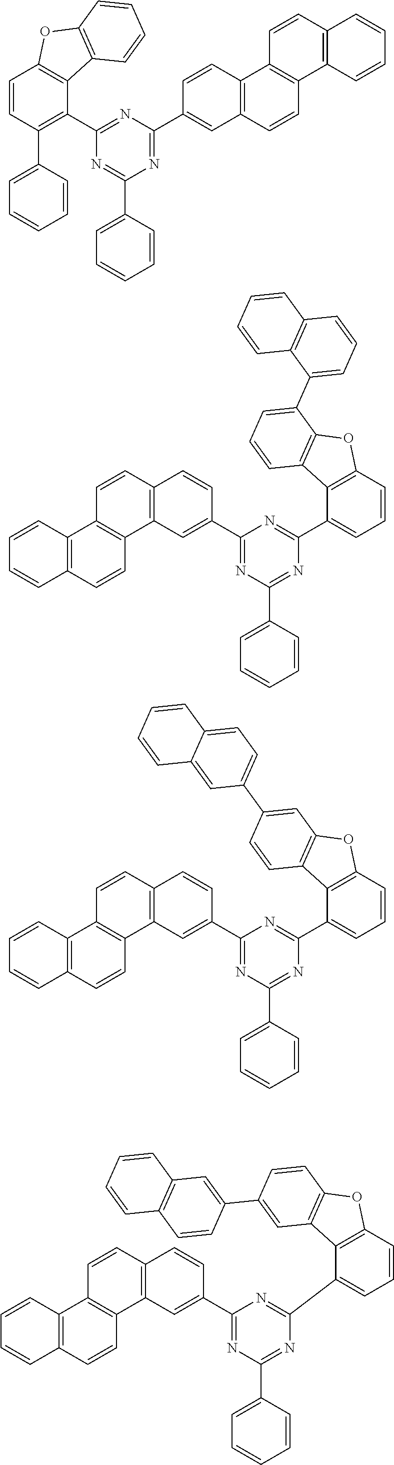 [SMILES: c1ccc(-c2nc(-c3ccc4c(ccc5c6ccccc6ccc45)c3)nc(-c3c(-c4ccccc4)ccc4oc5ccccc5c34)n2)cc1 ; c1ccc(-c2nc(-c3ccc4ccc5c6ccccc6ccc5c4c3)nc(-c3cccc4oc5c(-c6cccc7ccccc67)cccc5c34)n2)cc1 ; c1ccc(-c2nc(-c3ccc4ccc5c6ccccc6ccc5c4c3)nc(-c3cccc4oc5cc(-c6ccc7ccccc7c6)ccc5c34)n2)cc1 ; c1ccc(-c2nc(-c3ccc4ccc5c6ccccc6ccc5c4c3)nc(-c3cccc4oc5ccc(-c6ccc7ccccc7c6)cc5c34)n2)cc1]

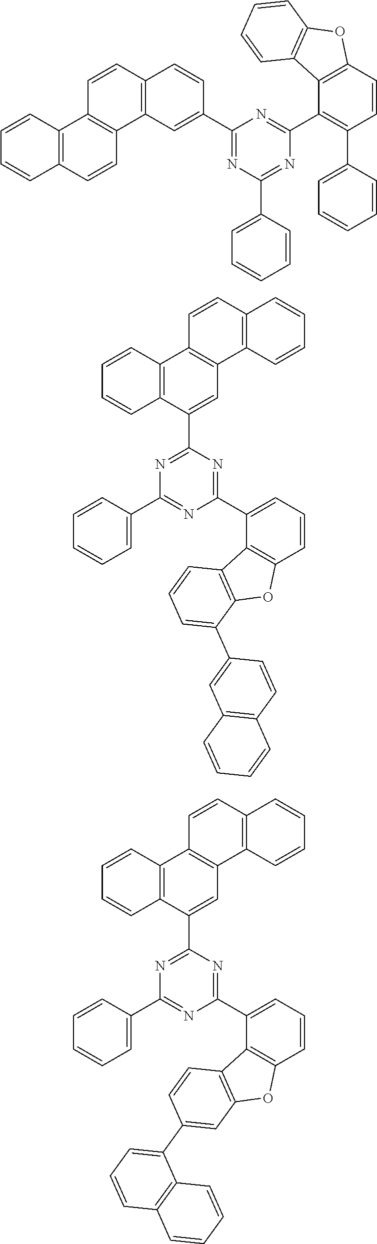

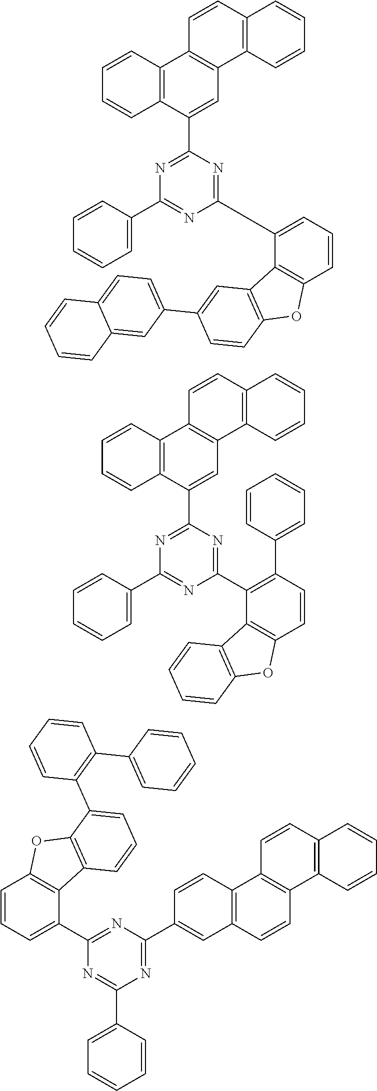

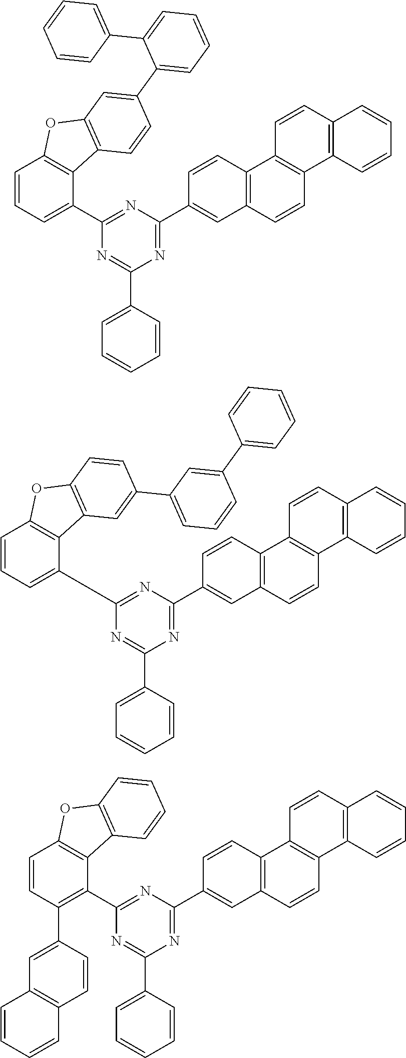

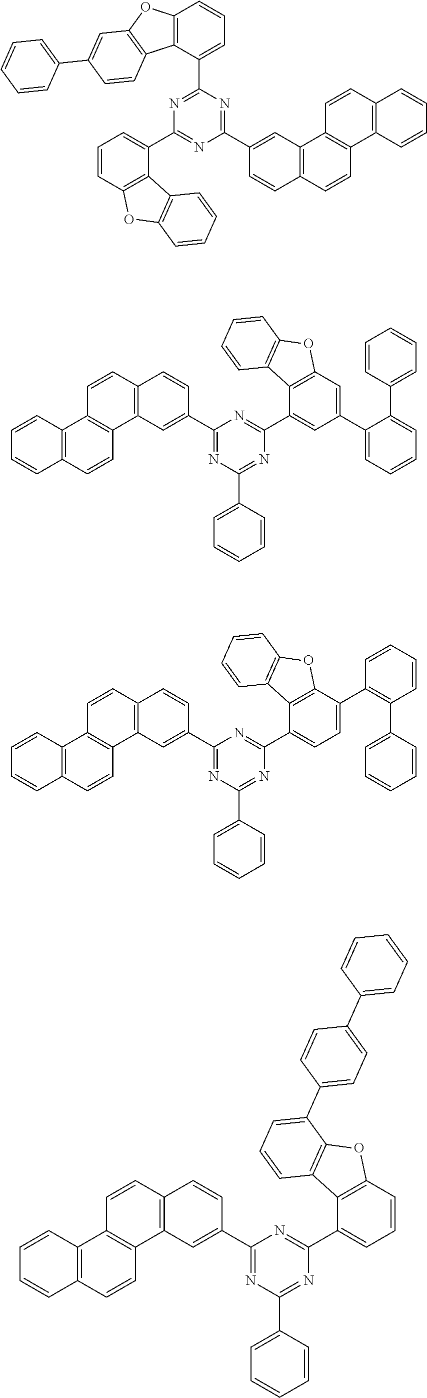 [SMILES: c1ccc(-c2ccc3c(c2)oc2cccc(-c4nc(-c5ccc6ccc7c8ccccc8ccc7c6c5)nc(-c5cccc6oc7ccccc7c56)n4)c23)cc1 ; c1ccc(-c2nc(-c3ccc4ccc5c6ccccc6ccc5c4c3)nc(-c3cc(-c4ccccc4-c4ccccc4)cc4oc5ccccc5c34)n2)cc1 ; c1ccc(-c2nc(-c3ccc4ccc5c6ccccc6ccc5c4c3)nc(-c3ccc(-c4ccccc4-c4ccccc4)c4oc5ccccc5c34)n2)cc1 ; c1ccc(-c2ccc(-c3cccc4c3oc3cccc(-c5nc(-c6ccccc6)nc(-c6ccc7ccc8c9ccccc9ccc8c7c6)n5)c34)cc2)cc1]

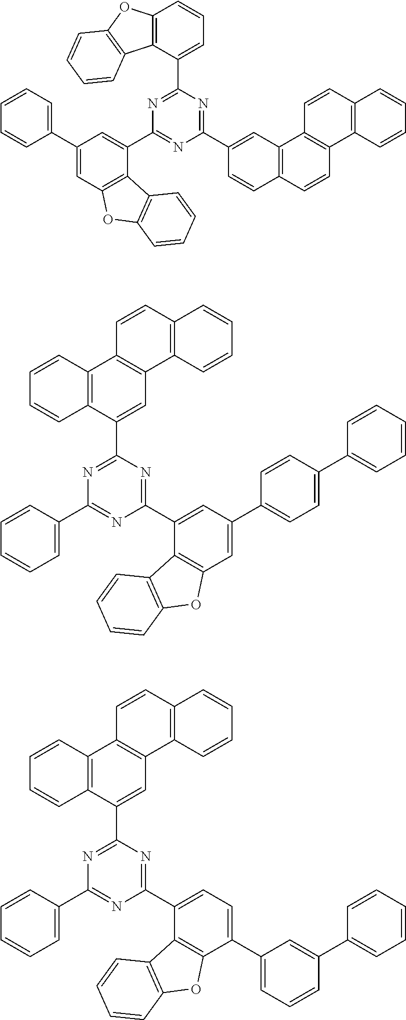 [SMILES: c1ccc(-c2cc(-c3nc(-c4ccc5ccc6c7ccccc7ccc6c5c4)nc(-c4cccc5oc6ccccc6c45)n3)c3c(c2)oc2ccccc23)cc1 ; c1ccc(-c2ccc(-c3cc(-c4nc(-c5ccccc5)nc(-c5cc6c7ccccc7ccc6c6ccccc56)n4)c4c(c3)oc3ccccc34)cc2)cc1 ; c1ccc(-c2cccc(-c3ccc(-c4nc(-c5ccccc5)nc(-c5cc6c7ccccc7ccc6c6ccccc56)n4)c4c3oc3ccccc34)c2)cc1]

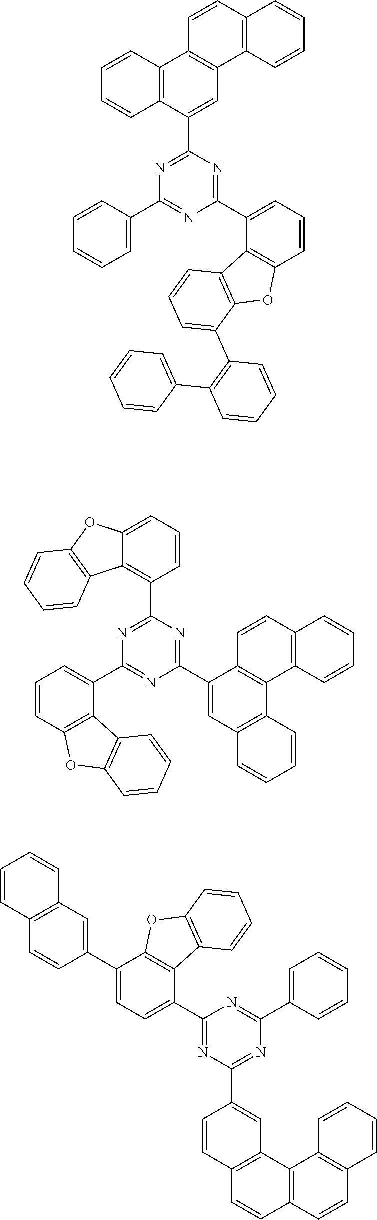 [SMILES: c1ccc(-c2nc(-c3cc4c5ccccc5ccc4c4ccccc34)nc(-c3cccc4oc5c(-c6ccccc6-c6ccccc6)cccc5c34)n2)cc1 ; c1ccc2c(c1)ccc1c(-c3nc(-c4cccc5oc6ccccc6c45)nc(-c4cccc5oc6ccccc6c45)n3)cc3ccccc3c12 ; c1ccc(-c2nc(-c3ccc4ccc5ccc6ccccc6c5c4c3)nc(-c3ccc(-c4ccc5ccccc5c4)c4oc5ccccc5c34)n2)cc1]

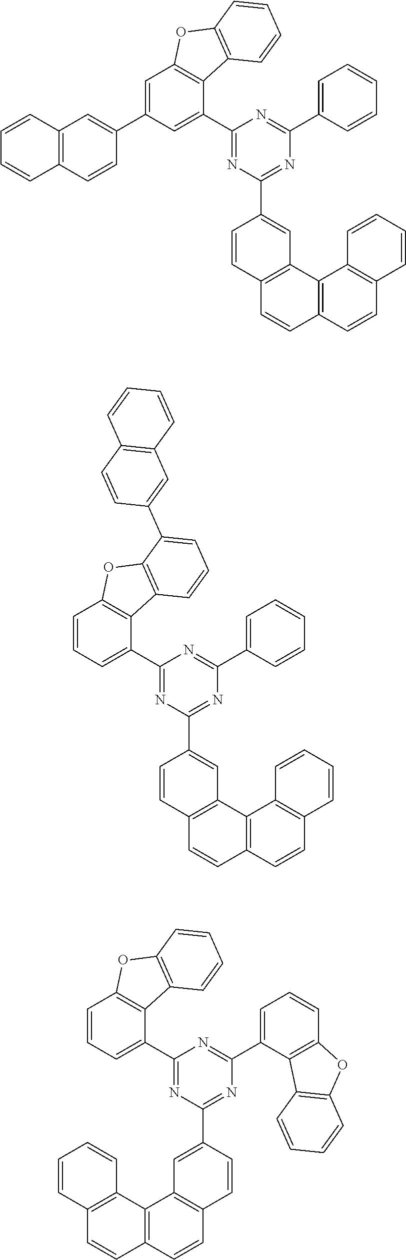 [SMILES: c1ccc(-c2nc(-c3ccc4ccc5ccc6ccccc6c5c4c3)nc(-c3cc(-c4ccc5ccccc5c4)cc4oc5ccccc5c34)n2)cc1 ; c1ccc(-c2nc(-c3ccc4ccc5ccc6ccccc6c5c4c3)nc(-c3cccc4oc5c(-c6ccc7ccccc7c6)cccc5c34)n2)cc1 ; c1ccc2c(c1)ccc1ccc3ccc(-c4nc(-c5cccc6oc7ccccc7c56)nc(-c5cccc6oc7ccccc7c56)n4)cc3c12]

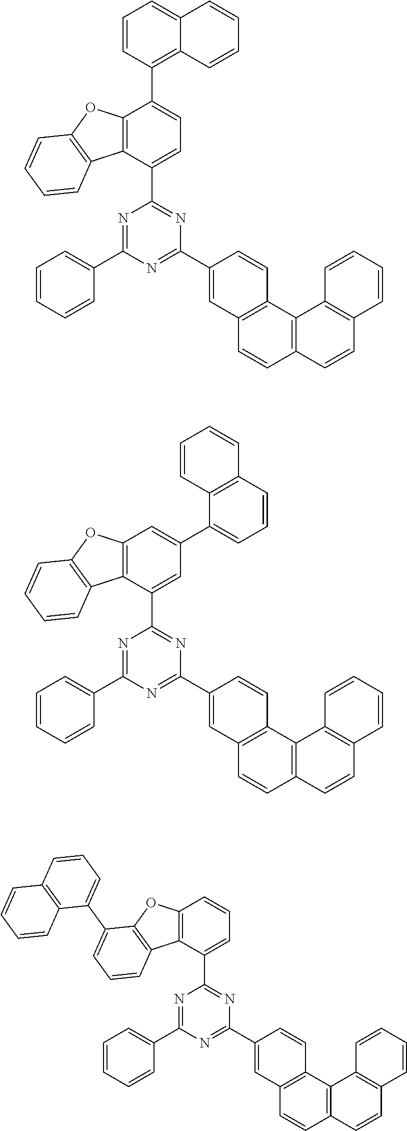 [SMILES: c1ccc(-c2nc(-c3ccc4c(ccc5ccc6ccccc6c54)c3)nc(-c3ccc(-c4cccc5ccccc45)c4oc5ccccc5c34)n2)cc1 ; c1ccc(-c2nc(-c3ccc4c(ccc5ccc6ccccc6c54)c3)nc(-c3cc(-c4cccc5ccccc45)cc4oc5ccccc5c34)n2)cc1 ; c1ccc(-c2nc(-c3ccc4c(ccc5ccc6ccccc6c54)c3)nc(-c3cccc4oc5c(-c6cccc7ccccc67)cccc5c34)n2)cc1]

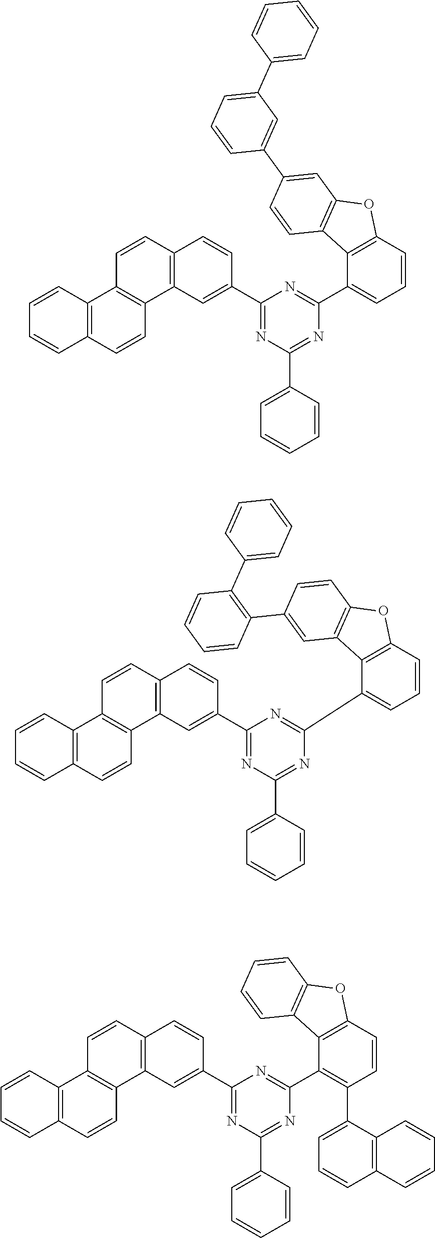 [SMILES: c1ccc(-c2cccc(-c3ccc4c(c3)oc3cccc(-c5nc(-c6ccccc6)nc(-c6ccc7ccc8c9ccccc9ccc8c7c6)n5)c34)c2)cc1 ; c1ccc(-c2nc(-c3ccc4ccc5c6ccccc6ccc5c4c3)nc(-c3cccc4oc5ccc(-c6ccccc6-c6ccccc6)cc5c34)n2)cc1 ; c1ccc(-c2nc(-c3ccc4ccc5c6ccccc6ccc5c4c3)nc(-c3c(-c4cccc5ccccc45)ccc4oc5ccccc5c34)n2)cc1]

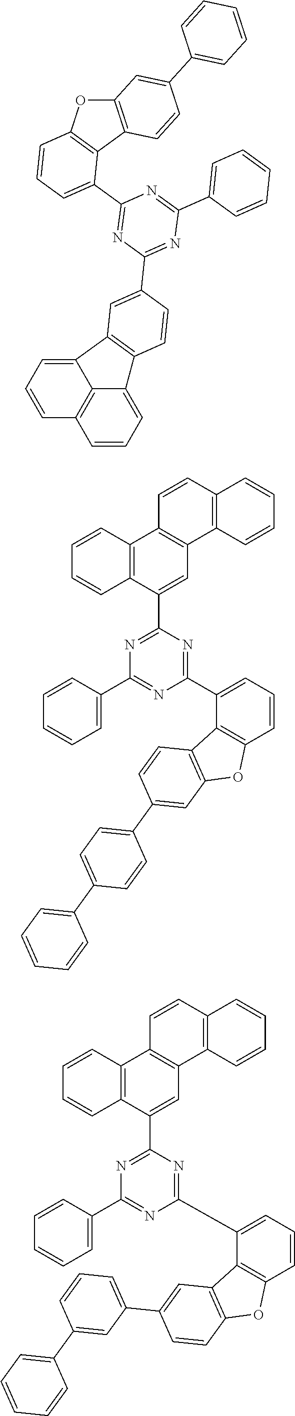 [SMILES: c1ccc(-c2ccc3c(c2)oc2cccc(-c4nc(-c5ccccc5)nc(-c5ccc6c(c5)-c5cccc7cccc-6c57)n4)c23)cc1 ; c1ccc(-c2ccc(-c3ccc4c(c3)oc3cccc(-c5nc(-c6ccccc6)nc(-c6cc7c8ccccc8ccc7c7ccccc67)n5)c34)cc2)cc1 ; c1ccc(-c2cccc(-c3ccc4oc5cccc(-c6nc(-c7ccccc7)nc(-c7cc8c9ccccc9ccc8c8ccccc78)n6)c5c4c3)c2)cc1]

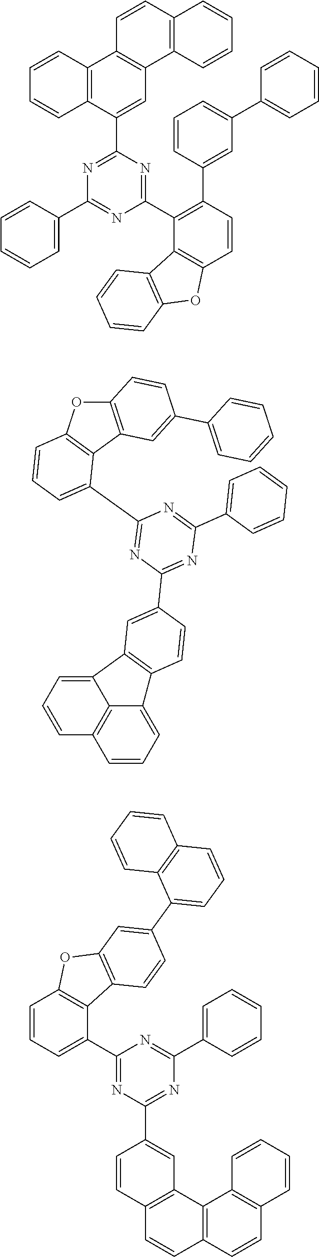 [SMILES: c1ccc(-c2cccc(-c3ccc4oc5ccccc5c4c3-c3nc(-c4ccccc4)nc(-c4cc5c6ccccc6ccc5c5ccccc45)n3)c2)cc1 ; c1ccc(-c2ccc3oc4cccc(-c5nc(-c6ccccc6)nc(-c6ccc7c(c6)-c6cccc8cccc-7c68)n5)c4c3c2)cc1 ; c1ccc(-c2nc(-c3ccc4ccc5ccc6ccccc6c5c4c3)nc(-c3cccc4oc5cc(-c6cccc7ccccc67)ccc5c34)n2)cc1]

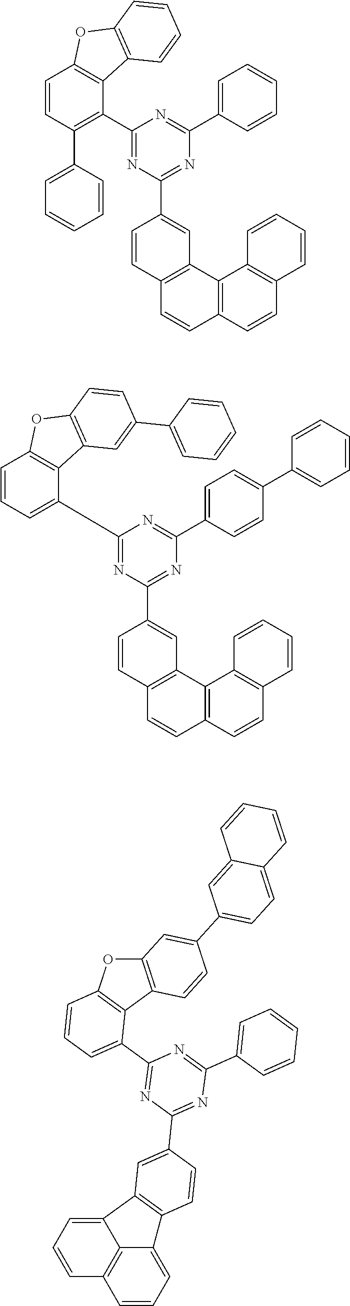 [SMILES: c1ccc(-c2nc(-c3ccc4ccc5ccc6ccccc6c5c4c3)nc(-c3c(-c4ccccc4)ccc4oc5ccccc5c34)n2)cc1 ; c1ccc(-c2ccc(-c3nc(-c4ccc5ccc6ccc7ccccc7c6c5c4)nc(-c4cccc5oc6ccc(-c7ccccc7)cc6c45)n3)cc2)cc1 ; c1ccc(-c2nc(-c3ccc4c(c3)-c3cccc5cccc-4c35)nc(-c3cccc4oc5cc(-c6ccc7ccccc7c6)ccc5c34)n2)cc1]

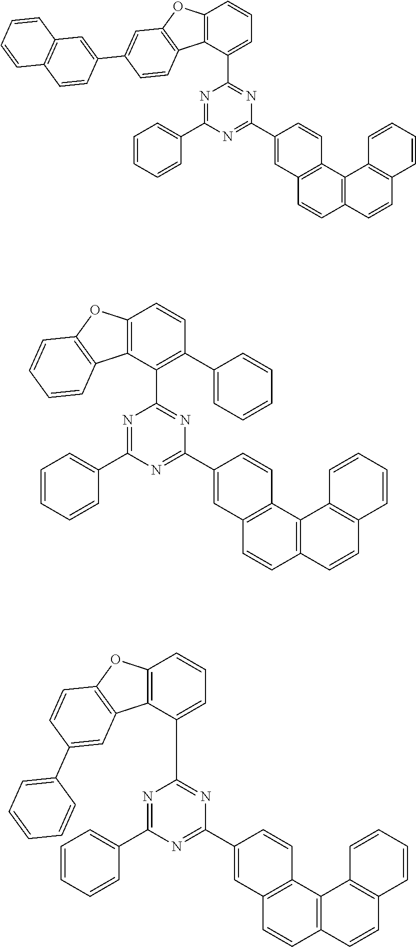 [SMILES: c1ccc(-c2nc(-c3ccc4c(ccc5ccc6ccccc6c54)c3)nc(-c3cccc4oc5cc(-c6ccc7ccccc7c6)ccc5c34)n2)cc1 ; c1ccc(-c2nc(-c3ccc4c(ccc5ccc6ccccc6c54)c3)nc(-c3c(-c4ccccc4)ccc4oc5ccccc5c34)n2)cc1 ; c1ccc(-c2ccc3oc4cccc(-c5nc(-c6ccccc6)nc(-c6ccc7c(ccc8ccc9ccccc9c87)c6)n5)c4c3c2)cc1]

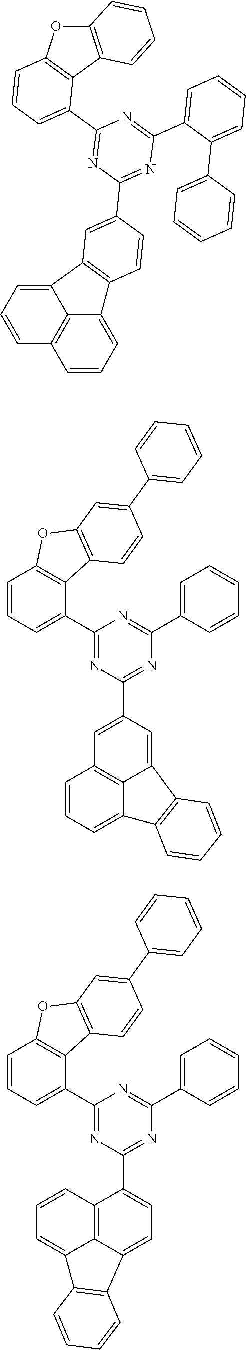

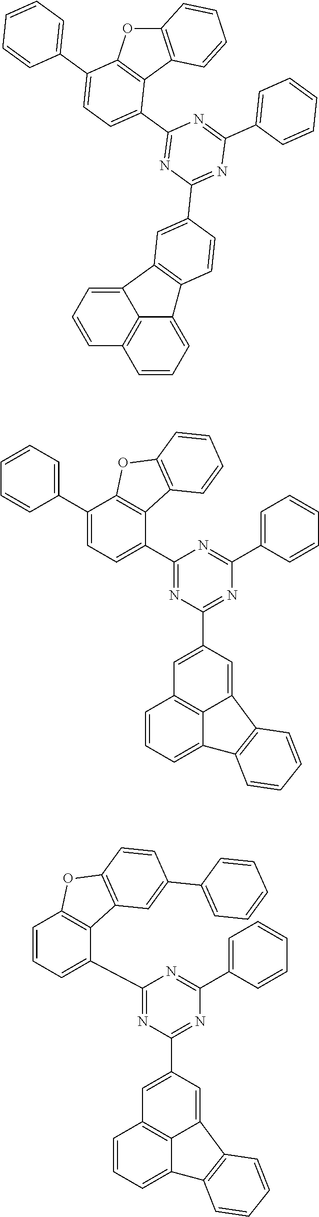 [SMILES: c1ccc(-c2nc(-c3ccc4c(c3)-c3cccc5cccc-4c35)nc(-c3ccc(-c4ccccc4)c4oc5ccccc5c34)n2)cc1 ; c1ccc(-c2nc(-c3cc4c5c(cccc5c3)-c3ccccc3-4)nc(-c3ccc(-c4ccccc4)c4oc5ccccc5c34)n2)cc1 ; c1ccc(-c2ccc3oc4cccc(-c5nc(-c6ccccc6)nc(-c6cc7c8c(cccc8c6)-c6ccccc6-7)n5)c4c3c2)cc1]

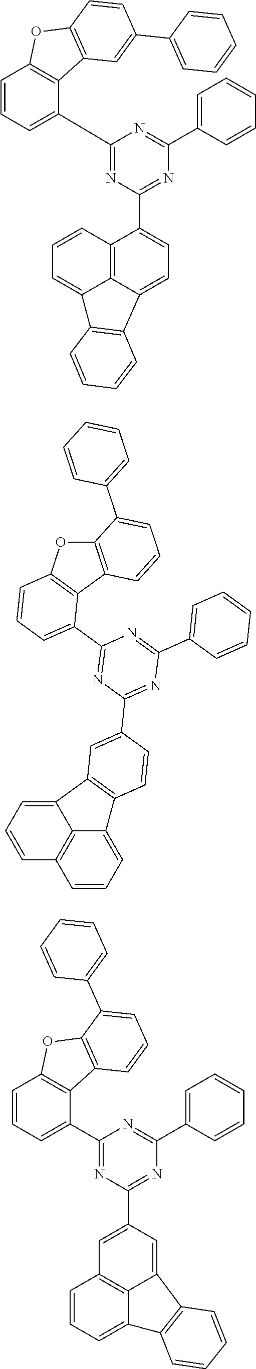

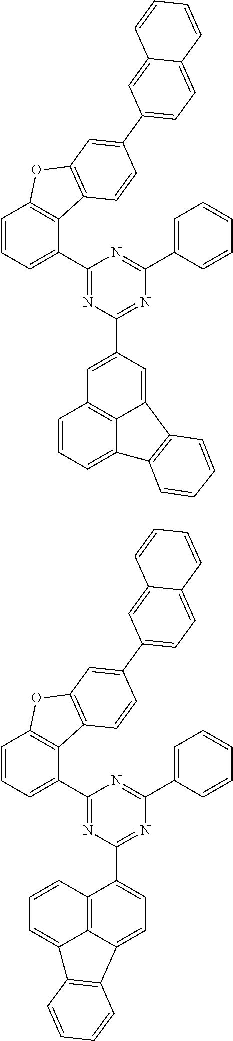

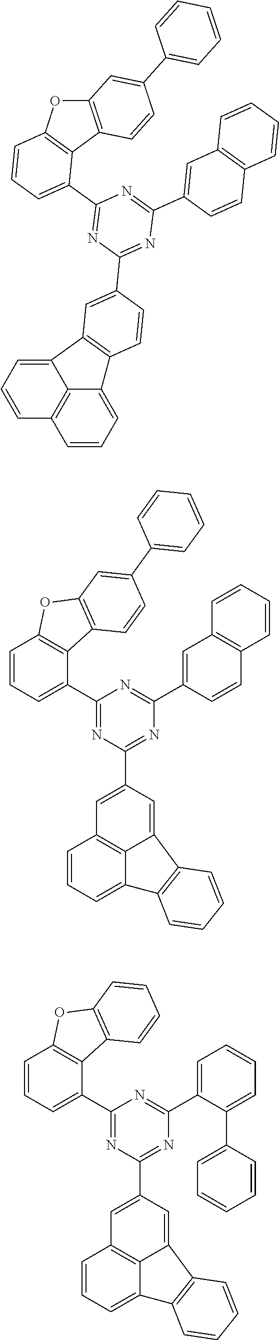 [SMILES: c1ccc(-c2ccc3c(c2)oc2cccc(-c4nc(-c5ccc6c(c5)-c5cccc7cccc-6c57)nc(-c5ccc6ccccc6c5)n4)c23)cc1 ; c1ccc(-c2ccc3c(c2)oc2cccc(-c4nc(-c5ccc6ccccc6c5)nc(-c5cc6c7c(cccc7c5)-c5ccccc5-6)n4)c23)cc1 ; c1ccc(-c2ccccc2-c2nc(-c3cc4c5c(cccc5c3)-c3ccccc3-4)nc(-c3cccc4oc5ccccc5c34)n2)cc1]

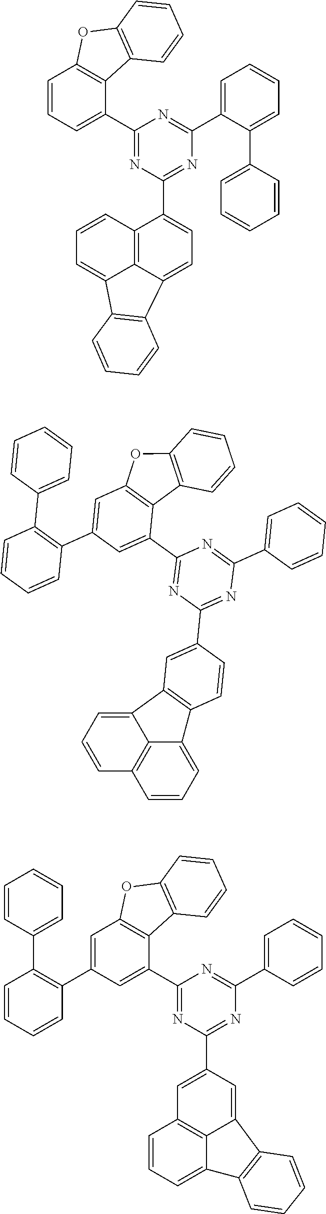 [SMILES: c1ccc(-c2ccccc2-c2nc(-c3ccc4c5c(cccc35)-c3ccccc3-4)nc(-c3cccc4oc5ccccc5c34)n2)cc1 ; c1ccc(-c2nc(-c3ccc4c(c3)-c3cccc5cccc-4c35)nc(-c3cc(-c4ccccc4-c4ccccc4)cc4oc5ccccc5c34)n2)cc1 ; c1ccc(-c2nc(-c3cc4c5c(cccc5c3)-c3ccccc3-4)nc(-c3cc(-c4ccccc4-c4ccccc4)cc4oc5ccccc5c34)n2)cc1]

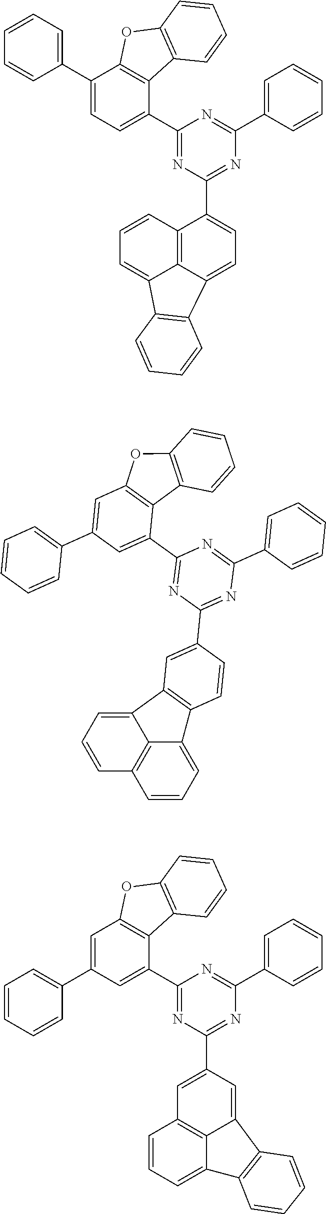 [SMILES: c1ccc(-c2nc(-c3ccc4c5c(cccc35)-c3ccccc3-4)nc(-c3ccc(-c4ccccc4)c4oc5ccccc5c34)n2)cc1 ; c1ccc(-c2cc(-c3nc(-c4ccccc4)nc(-c4ccc5c(c4)-c4cccc6cccc-5c46)n3)c3c(c2)oc2ccccc23)cc1 ; c1ccc(-c2cc(-c3nc(-c4ccccc4)nc(-c4cc5c6c(cccc6c4)-c4ccccc4-5)n3)c3c(c2)oc2ccccc23)cc1]

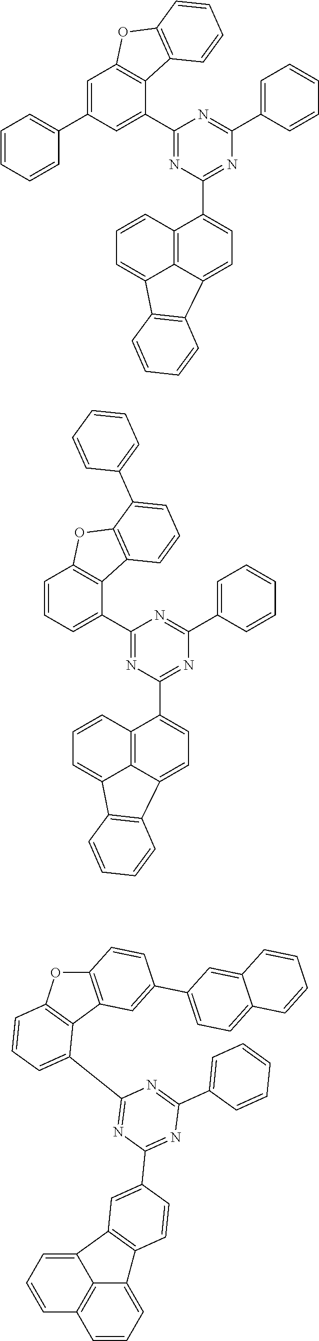 [SMILES: c1ccc(-c2cc(-c3nc(-c4ccccc4)nc(-c4ccc5c6c(cccc46)-c4ccccc4-5)n3)c3c(c2)oc2ccccc23)cc1 ; c1ccc(-c2nc(-c3ccc4c5c(cccc35)-c3ccccc3-4)nc(-c3cccc4oc5c(-c6ccccc6)cccc5c34)n2)cc1 ; c1ccc(-c2nc(-c3ccc4c(c3)-c3cccc5cccc-4c35)nc(-c3cccc4oc5ccc(-c6ccc7ccccc7c6)cc5c34)n2)cc1]

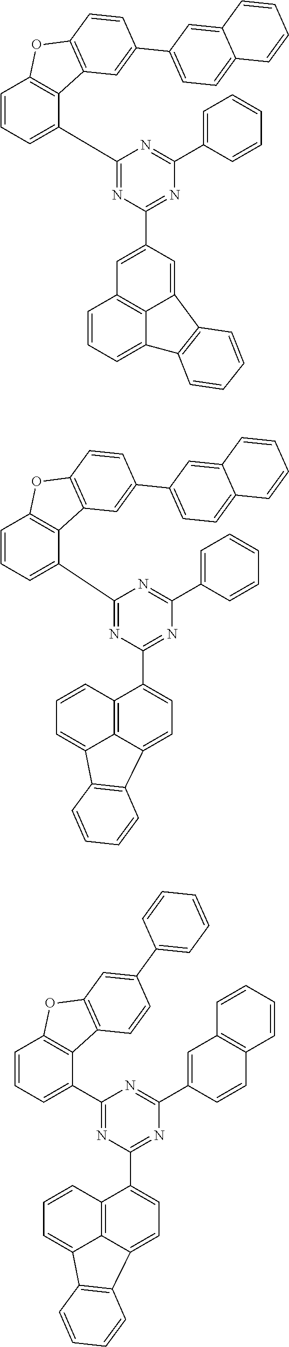 [SMILES: c1ccc(-c2nc(-c3cc4c5c(cccc5c3)-c3ccccc3-4)nc(-c3cccc4oc5ccc(-c6ccc7ccccc7c6)cc5c34)n2)cc1 ; c1ccc(-c2nc(-c3ccc4c5c(cccc35)-c3ccccc3-4)nc(-c3cccc4oc5ccc(-c6ccc7ccccc7c6)cc5c34)n2)cc1 ; c1ccc(-c2ccc3c(c2)oc2cccc(-c4nc(-c5ccc6ccccc6c5)nc(-c5ccc6c7c(cccc57)-c5ccccc5-6)n4)c23)cc1]

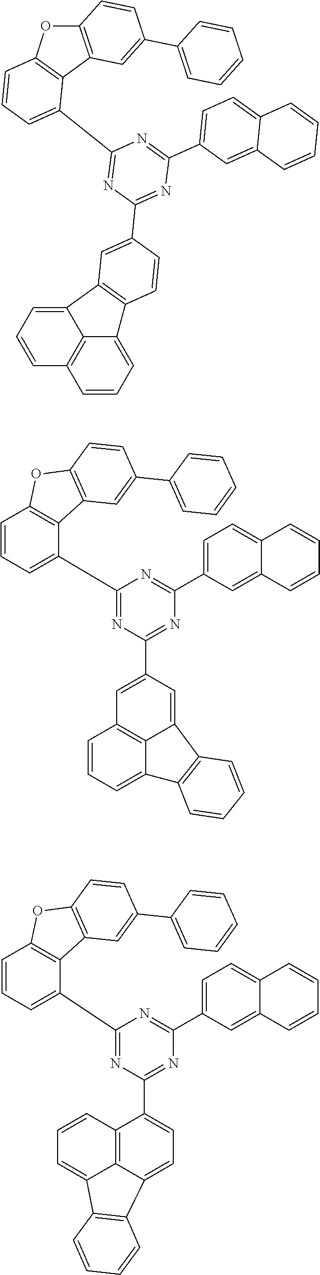 [SMILES: c1ccc(-c2ccc3oc4cccc(-c5nc(-c6ccc7c(c6)-c6cccc8cccc-7c68)nc(-c6ccc7ccccc7c6)n5)c4c3c2)cc1 ; c1ccc(-c2ccc3oc4cccc(-c5nc(-c6ccc7ccccc7c6)nc(-c6cc7c8c(cccc8c6)-c6ccccc6-7)n5)c4c3c2)cc1 ; c1ccc(-c2ccc3oc4cccc(-c5nc(-c6ccc7ccccc7c6)nc(-c6ccc7c8c(cccc68)-c6ccccc6-7)n5)c4c3c2)cc1]

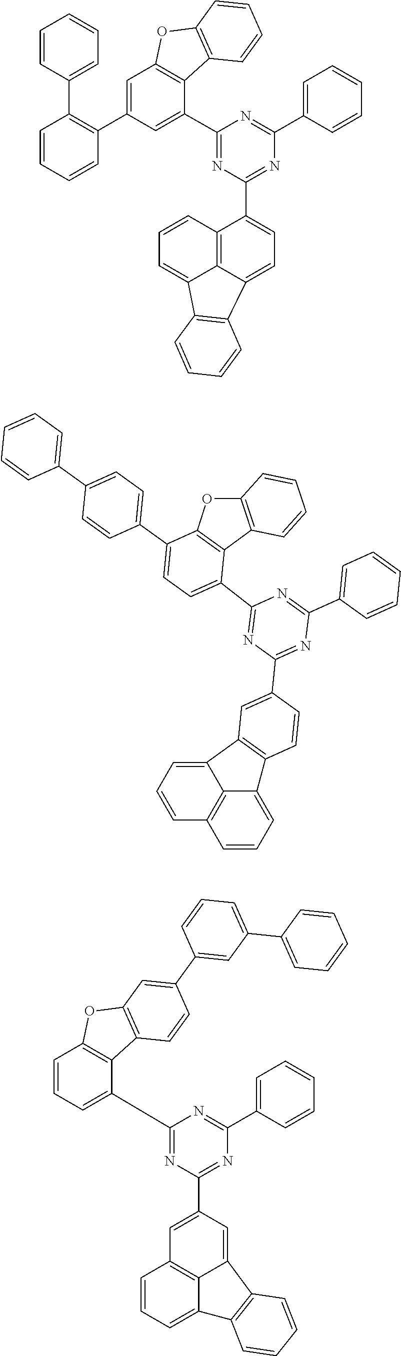 [SMILES: c1ccc(-c2nc(-c3ccc4c5c(cccc35)-c3ccccc3-4)nc(-c3cc(-c4ccccc4-c4ccccc4)cc4oc5ccccc5c34)n2)cc1 ; c1ccc(-c2ccc(-c3ccc(-c4nc(-c5ccccc5)nc(-c5ccc6c(c5)-c5cccc7cccc-6c57)n4)c4c3oc3ccccc34)cc2)cc1 ; c1ccc(-c2cccc(-c3ccc4c(c3)oc3cccc(-c5nc(-c6ccccc6)nc(-c6cc7c8c(cccc8c6)-c6ccccc6-7)n5)c34)c2)cc1]

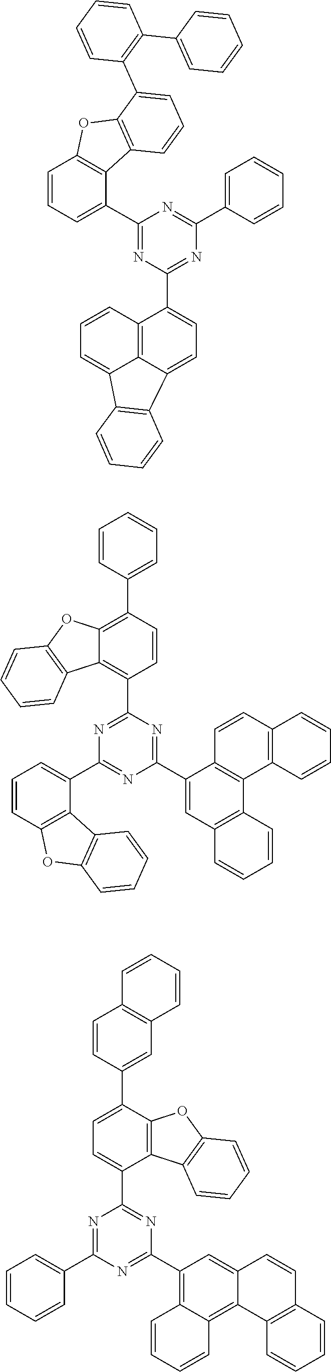 [SMILES: c1ccc(-c2nc(-c3ccc4c5c(cccc35)-c3ccccc3-4)nc(-c3cccc4oc5c(-c6ccccc6-c6ccccc6)cccc5c34)n2)cc1 ; c1ccc(-c2ccc(-c3nc(-c4cc5ccccc5c5c4ccc4ccccc45)nc(-c4cccc5oc6ccccc6c45)n3)c3c2oc2ccccc23)cc1 ; c1ccc(-c2nc(-c3cc4ccc5ccccc5c4c4ccccc34)nc(-c3ccc(-c4ccc5ccccc5c4)c4oc5ccccc5c34)n2)cc1]

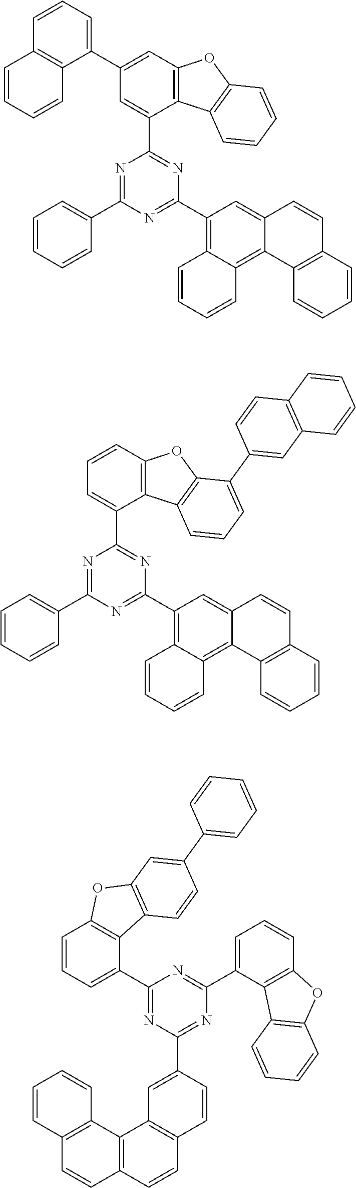 [SMILES: c1ccc(-c2nc(-c3cc4ccc5ccccc5c4c4ccccc34)nc(-c3cc(-c4cccc5ccccc45)cc4oc5ccccc5c34)n2)cc1 ; c1ccc(-c2nc(-c3cc4ccc5ccccc5c4c4ccccc34)nc(-c3cccc4oc5c(-c6ccc7ccccc7c6)cccc5c34)n2)cc1 ; c1ccc(-c2ccc3c(c2)oc2cccc(-c4nc(-c5ccc6ccc7ccc8ccccc8c7c6c5)nc(-c5cccc6oc7ccccc7c56)n4)c23)cc1]

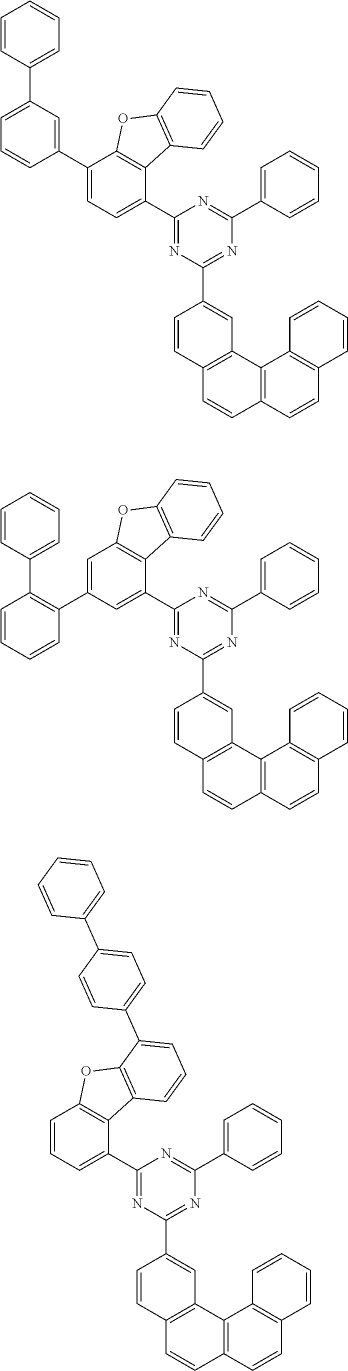 [SMILES: c1ccc(-c2cccc(-c3ccc(-c4nc(-c5ccccc5)nc(-c5ccc6ccc7ccc8ccccc8c7c6c5)n4)c4c3oc3ccccc34)c2)cc1 ; c1ccc(-c2nc(-c3ccc4ccc5ccc6ccccc6c5c4c3)nc(-c3cc(-c4ccccc4-c4ccccc4)cc4oc5ccccc5c34)n2)cc1 ; c1ccc(-c2ccc(-c3cccc4c3oc3cccc(-c5nc(-c6ccccc6)nc(-c6ccc7ccc8ccc9ccccc9c8c7c6)n5)c34)cc2)cc1]

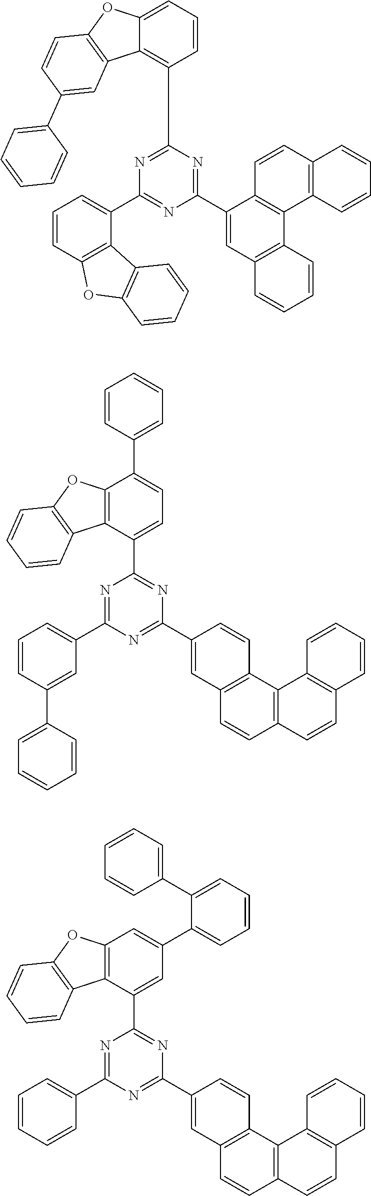 [SMILES: c1ccc(-c2ccc3oc4cccc(-c5nc(-c6cc7ccccc7c7c6ccc6ccccc67)nc(-c6cccc7oc8ccccc8c67)n5)c4c3c2)cc1 ; c1ccc(-c2cccc(-c3nc(-c4ccc5c(ccc6ccc7ccccc7c65)c4)nc(-c4ccc(-c5ccccc5)c5oc6ccccc6c45)n3)c2)cc1 ; c1ccc(-c2nc(-c3ccc4c(ccc5ccc6ccccc6c54)c3)nc(-c3cc(-c4ccccc4-c4ccccc4)cc4oc5ccccc5c34)n2)cc1]

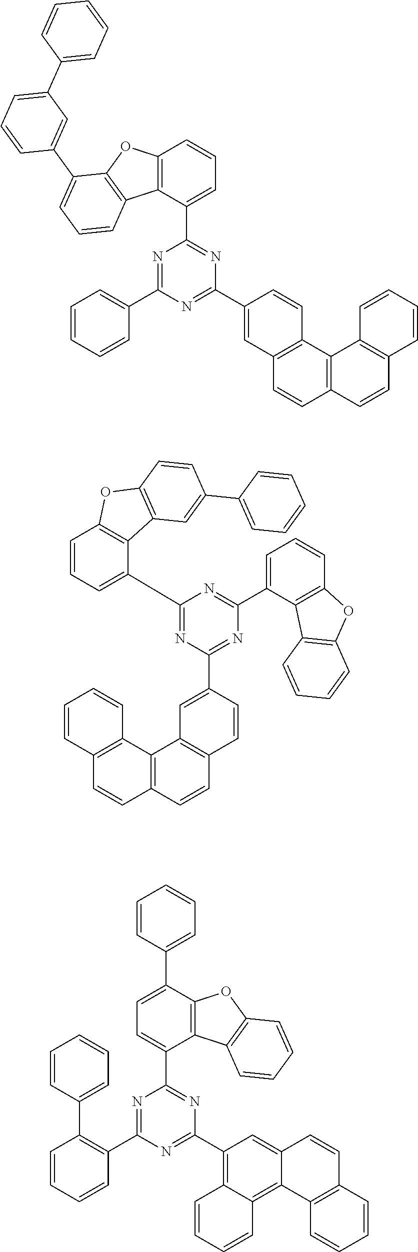 [SMILES: c1ccc(-c2cccc(-c3cccc4c3oc3cccc(-c5nc(-c6ccccc6)nc(-c6ccc7c(ccc8ccc9ccccc9c87)c6)n5)c34)c2)cc1 ; c1ccc(-c2ccc3oc4cccc(-c5nc(-c6ccc7ccc8ccc9ccccc9c8c7c6)nc(-c6cccc7oc8ccccc8c67)n5)c4c3c2)cc1 ; c1ccc(-c2ccccc2-c2nc(-c3cc4ccc5ccccc5c4c4ccccc34)nc(-c3ccc(-c4ccccc4)c4oc5ccccc5c34)n2)cc1]

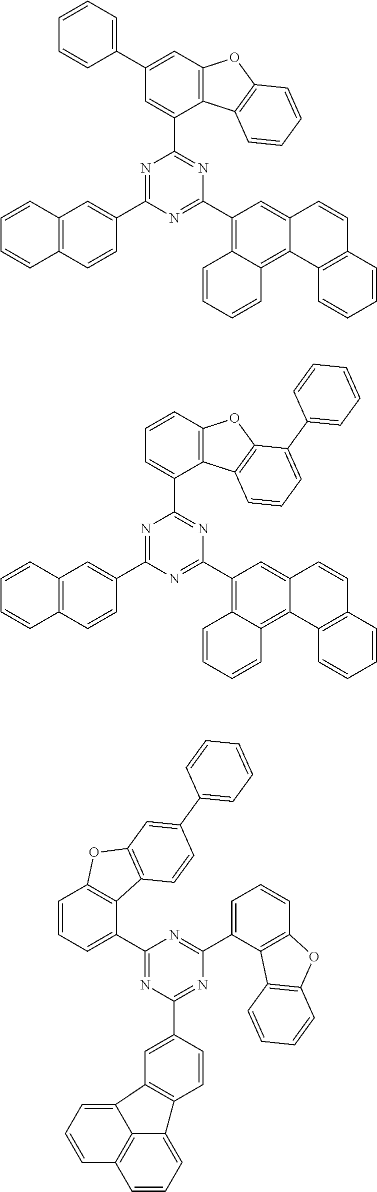 [SMILES: c1ccc(-c2cc(-c3nc(-c4ccc5ccccc5c4)nc(-c4cc5ccc6ccccc6c5c5ccccc45)n3)c3c(c2)oc2ccccc23)cc1 ; c1ccc(-c2cccc3c2oc2cccc(-c4nc(-c5ccc6ccccc6c5)nc(-c5cc6ccc7ccccc7c6c6ccccc56)n4)c23)cc1 ; c1ccc(-c2ccc3c(c2)oc2cccc(-c4nc(-c5ccc6c(c5)-c5cccc7cccc-6c57)nc(-c5cccc6oc7ccccc7c56)n4)c23)cc1]

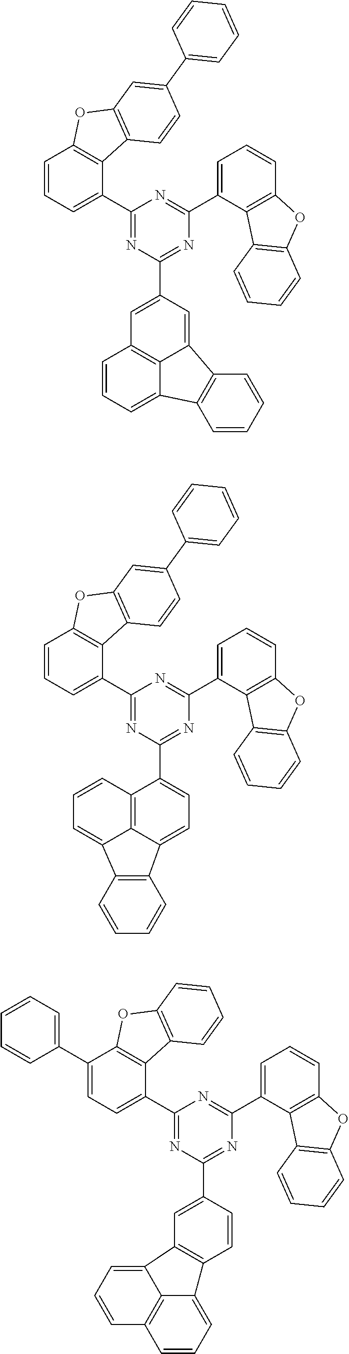 [SMILES: c1ccc(-c2ccc3c(c2)oc2cccc(-c4nc(-c5cc6c7c(cccc7c5)-c5ccccc5-6)nc(-c5cccc6oc7ccccc7c56)n4)c23)cc1 ; c1ccc(-c2ccc3c(c2)oc2cccc(-c4nc(-c5ccc6c7c(cccc57)-c5ccccc5-6)nc(-c5cccc6oc7ccccc7c56)n4)c23)cc1 ; c1ccc(-c2ccc(-c3nc(-c4ccc5c(c4)-c4cccc6cccc-5c46)nc(-c4cccc5oc6ccccc6c45)n3)c3c2oc2ccccc23)cc1]

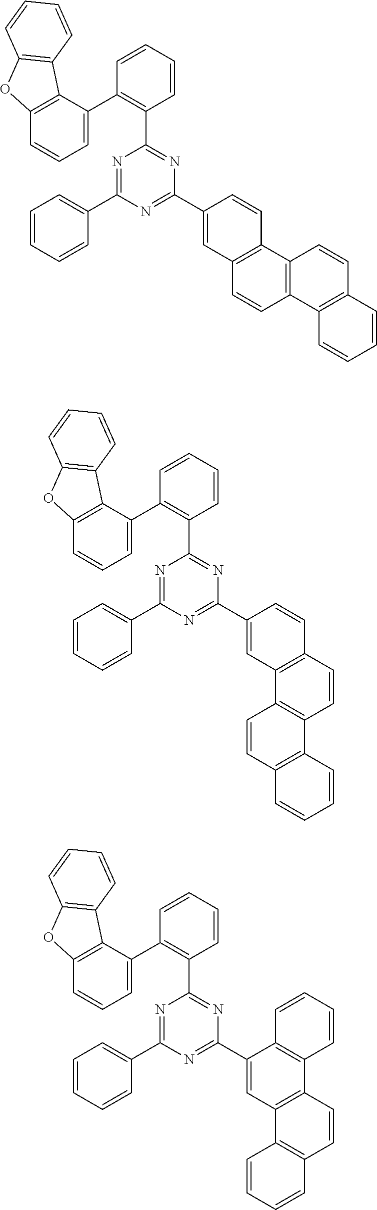 [SMILES: c1ccc(-c2nc(-c3ccc4c(ccc5c6ccccc6ccc45)c3)nc(-c3ccccc3-c3cccc4oc5ccccc5c34)n2)cc1 ; c1ccc(-c2nc(-c3ccc4ccc5c6ccccc6ccc5c4c3)nc(-c3ccccc3-c3cccc4oc5ccccc5c34)n2)cc1 ; c1ccc(-c2nc(-c3ccccc3-c3cccc4oc5ccccc5c34)nc(-c3cc4c5ccccc5ccc4c4ccccc34)n2)cc1]

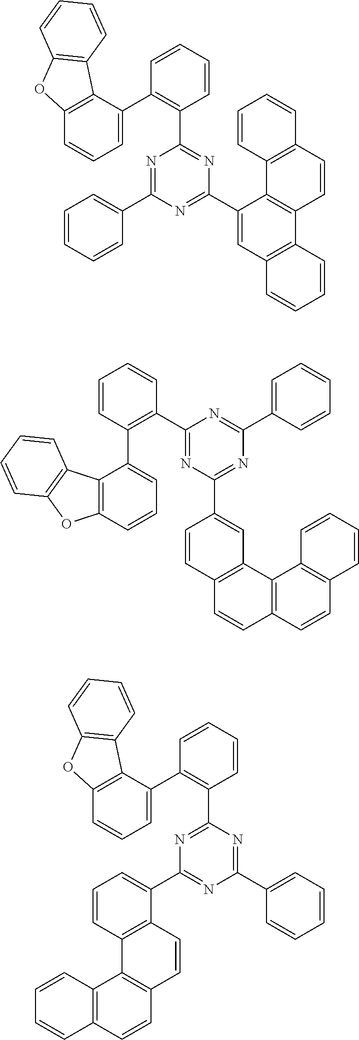 [SMILES: c1ccc(-c2nc(-c3ccccc3-c3cccc4oc5ccccc5c34)nc(-c3cc4ccccc4c4ccc5ccccc5c34)n2)cc1 ; c1ccc(-c2nc(-c3ccc4ccc5ccc6ccccc6c5c4c3)nc(-c3ccccc3-c3cccc4oc5ccccc5c34)n2)cc1 ; c1ccc(-c2nc(-c3ccccc3-c3cccc4oc5ccccc5c34)nc(-c3cccc4c3ccc3ccc5ccccc5c34)n2)cc1]

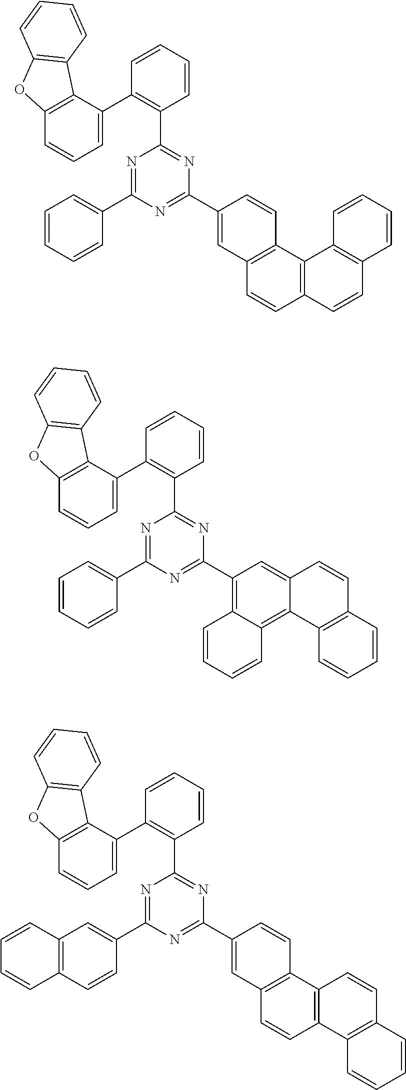 [SMILES: c1ccc(-c2nc(-c3ccc4c(ccc5ccc6ccccc6c54)c3)nc(-c3ccccc3-c3cccc4oc5ccccc5c34)n2)cc1 ; c1ccc(-c2nc(-c3ccccc3-c3cccc4oc5ccccc5c34)nc(-c3cc4ccc5ccccc5c4c4ccccc34)n2)cc1 ; c1ccc(-c2cccc3oc4ccccc4c23)c(-c2nc(-c3ccc4ccccc4c3)nc(-c3ccc4c(ccc5c6ccccc6ccc45)c3)n2)c1]

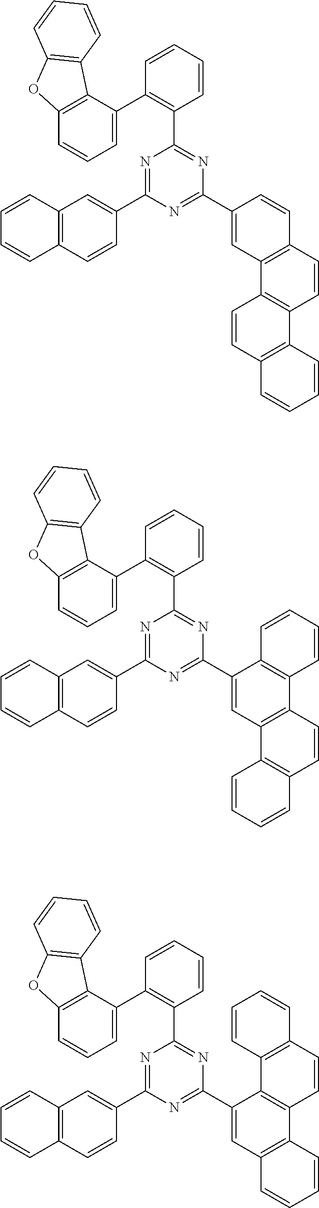 [SMILES: c1ccc(-c2cccc3oc4ccccc4c23)c(-c2nc(-c3ccc4ccccc4c3)nc(-c3ccc4ccc5c6ccccc6ccc5c4c3)n2)c1 ; c1ccc(-c2cccc3oc4ccccc4c23)c(-c2nc(-c3ccc4ccccc4c3)nc(-c3cc4c5ccccc5ccc4c4ccccc34)n2)c1 ; c1ccc(-c2cccc3oc4ccccc4c23)c(-c2nc(-c3ccc4ccccc4c3)nc(-c3cc4ccccc4c4ccc5ccccc5c34)n2)c1]

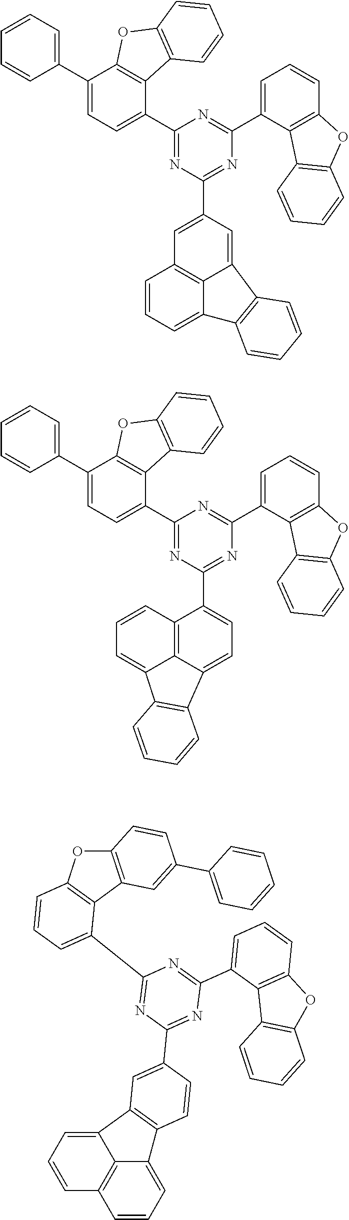 [SMILES: c1ccc(-c2ccc(-c3nc(-c4cc5c6c(cccc6c4)-c4ccccc4-5)nc(-c4cccc5oc6ccccc6c45)n3)c3c2oc2ccccc23)cc1 ; c1ccc(-c2ccc(-c3nc(-c4ccc5c6c(cccc46)-c4ccccc4-5)nc(-c4cccc5oc6ccccc6c45)n3)c3c2oc2ccccc23)cc1 ; c1ccc(-c2ccc3oc4cccc(-c5nc(-c6ccc7c(c6)-c6cccc8cccc-7c68)nc(-c6cccc7oc8ccccc8c67)n5)c4c3c2)cc1]

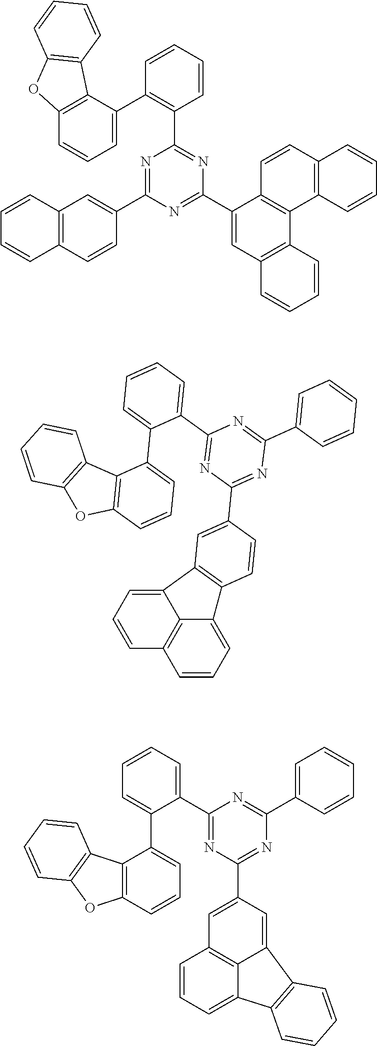 [SMILES: c1ccc(-c2cccc3oc4ccccc4c23)c(-c2nc(-c3ccc4ccccc4c3)nc(-c3cc4ccccc4c4c3ccc3ccccc34)n2)c1 ; c1ccc(-c2nc(-c3ccc4c(c3)-c3cccc5cccc-4c35)nc(-c3ccccc3-c3cccc4oc5ccccc5c34)n2)cc1 ; c1ccc(-c2nc(-c3cc4c5c(cccc5c3)-c3ccccc3-4)nc(-c3ccccc3-c3cccc4oc5ccccc5c34)n2)cc1]

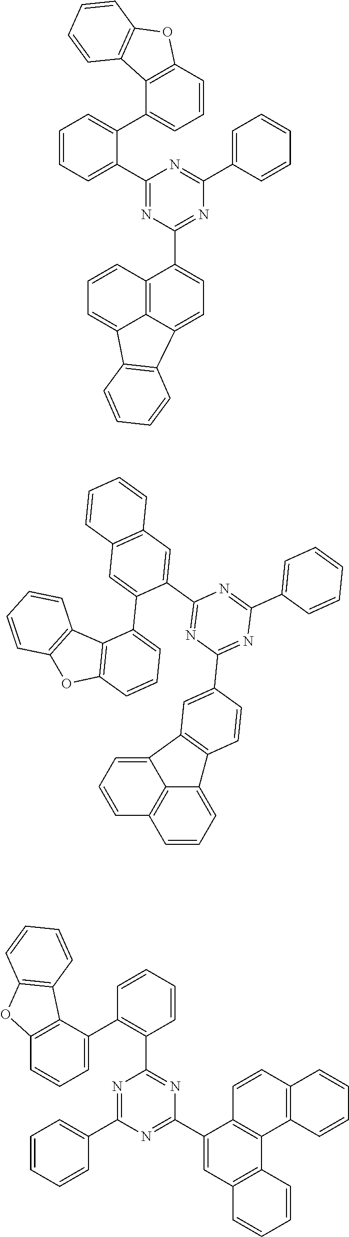 [SMILES: c1ccc(-c2nc(-c3ccccc3-c3cccc4oc5ccccc5c34)nc(-c3ccc4c5c(cccc35)-c3ccccc3-4)n2)cc1 ; c1ccc(-c2nc(-c3ccc4c(c3)-c3cccc5cccc-4c35)nc(-c3cc4ccccc4cc3-c3cccc4oc5ccccc5c34)n2)cc1 ; c1ccc(-c2nc(-c3ccccc3-c3cccc4oc5ccccc5c34)nc(-c3cc4ccccc4c4c3ccc3ccccc34)n2)cc1]

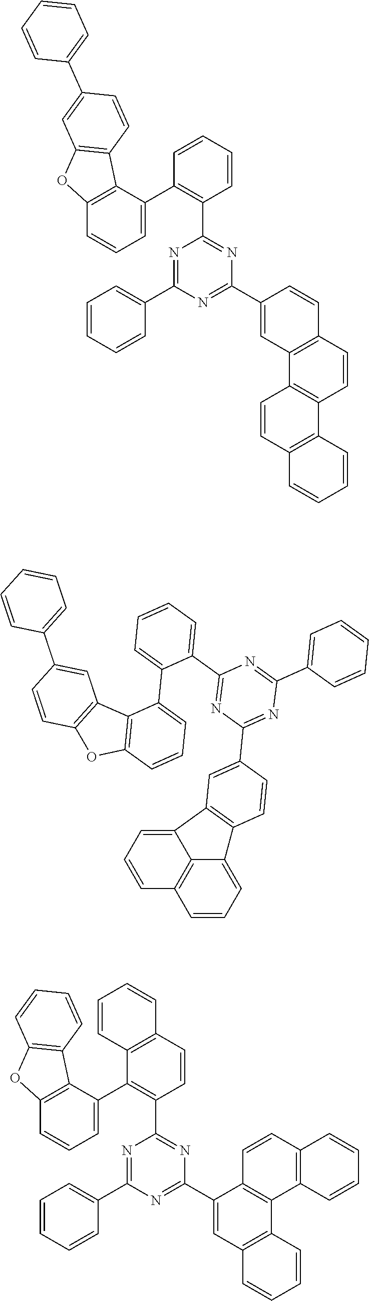 [SMILES: c1ccc(-c2ccc3c(c2)oc2cccc(-c4ccccc4-c4nc(-c5ccccc5)nc(-c5ccc6ccc7c8ccccc8ccc7c6c5)n4)c23)cc1 ; c1ccc(-c2ccc3oc4cccc(-c5ccccc5-c5nc(-c6ccccc6)nc(-c6ccc7c(c6)-c6cccc8cccc-7c68)n5)c4c3c2)cc1 ; c1ccc(-c2nc(-c3ccc4ccccc4c3-c3cccc4oc5ccccc5c34)nc(-c3cc4ccccc4c4c3ccc3ccccc34)n2)cc1]

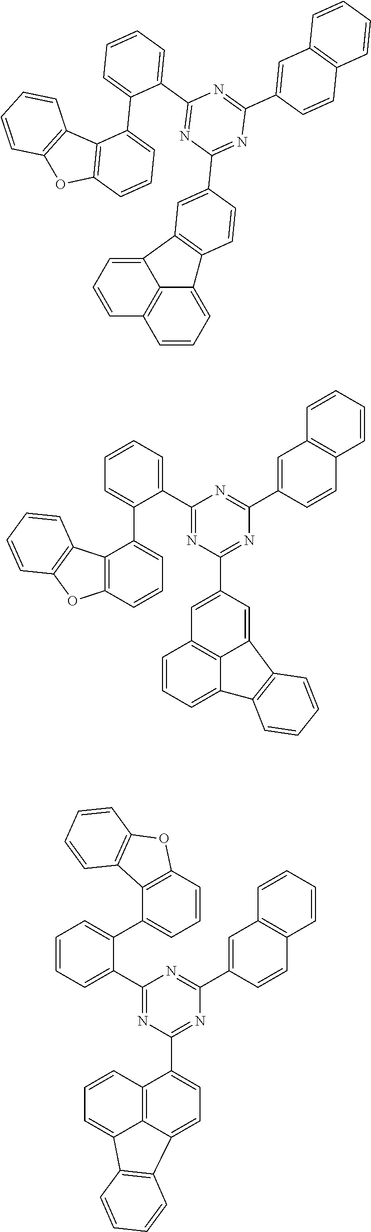 [SMILES: c1ccc(-c2cccc3oc4ccccc4c23)c(-c2nc(-c3ccc4c(c3)-c3cccc5cccc-4c35)nc(-c3ccc4ccccc4c3)n2)c1 ; c1ccc(-c2cccc3oc4ccccc4c23)c(-c2nc(-c3ccc4ccccc4c3)nc(-c3cc4c5c(cccc5c3)-c3ccccc3-4)n2)c1 ; c1ccc(-c2cccc3oc4ccccc4c23)c(-c2nc(-c3ccc4ccccc4c3)nc(-c3ccc4c5c(cccc35)-c3ccccc3-4)n2)c1]

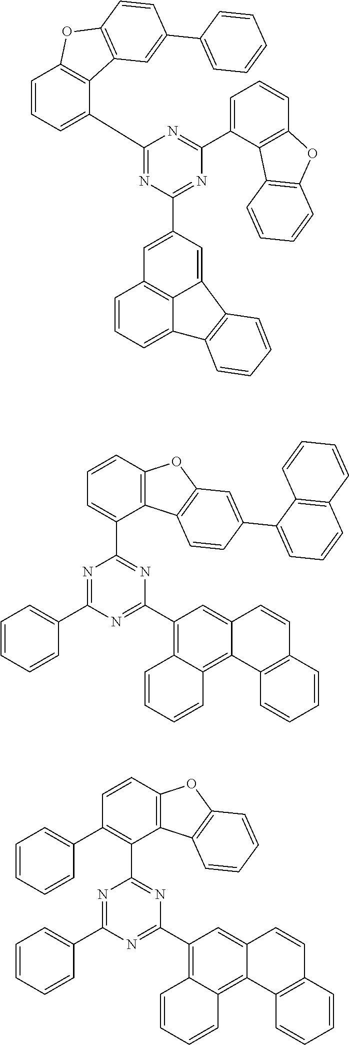 [SMILES: c1ccc(-c2ccc3oc4cccc(-c5nc(-c6cc7c8c(cccc8c6)-c6ccccc6-7)nc(-c6cccc7oc8ccccc8c67)n5)c4c3c2)cc1 ; c1ccc(-c2nc(-c3cc4ccc5ccccc5c4c4ccccc34)nc(-c3cccc4oc5cc(-c6cccc7ccccc67)ccc5c34)n2)cc1 ; c1ccc(-c2nc(-c3cc4ccc5ccccc5c4c4ccccc34)nc(-c3c(-c4ccccc4)ccc4oc5ccccc5c34)n2)cc1]

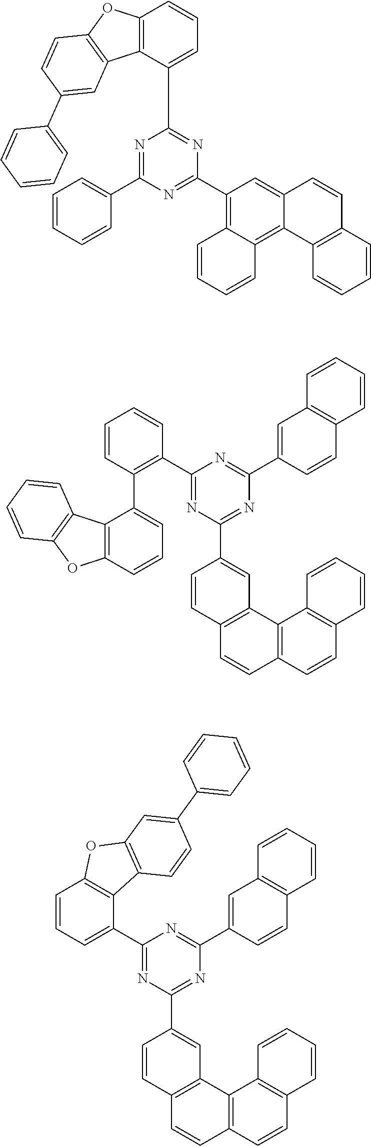 [SMILES: c1ccc(-c2ccc3oc4cccc(-c5nc(-c6ccccc6)nc(-c6cc7ccc8ccccc8c7c7ccccc67)n5)c4c3c2)cc1 ; c1ccc(-c2cccc3oc4ccccc4c23)c(-c2nc(-c3ccc4ccccc4c3)nc(-c3ccc4ccc5ccc6ccccc6c5c4c3)n2)c1 ; c1ccc(-c2ccc3c(c2)oc2cccc(-c4nc(-c5ccc6ccccc6c5)nc(-c5ccc6ccc7ccc8ccccc8c7c6c5)n4)c23)cc1]

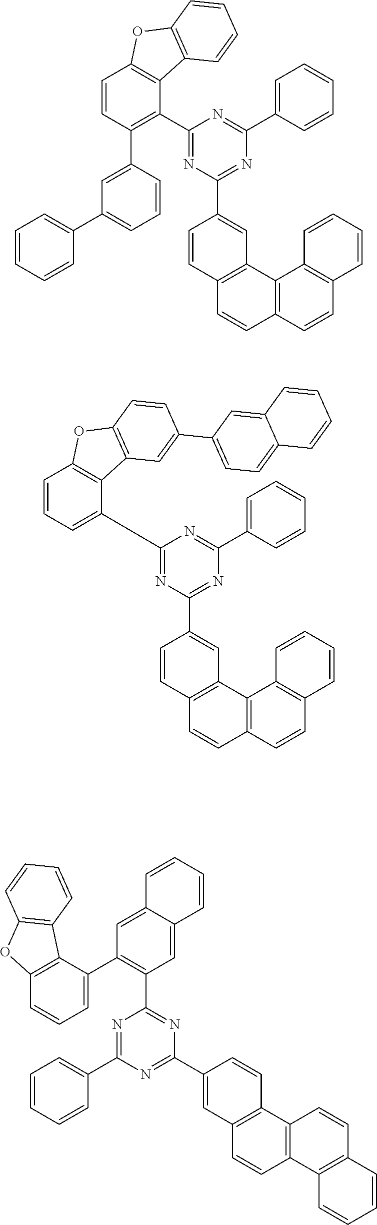 [SMILES: c1ccc(-c2cccc(-c3ccc4oc5ccccc5c4c3-c3nc(-c4ccccc4)nc(-c4ccc5ccc6ccc7ccccc7c6c5c4)n3)c2)cc1 ; c1ccc(-c2nc(-c3ccc4ccc5ccc6ccccc6c5c4c3)nc(-c3cccc4oc5ccc(-c6ccc7ccccc7c6)cc5c34)n2)cc1 ; c1ccc(-c2nc(-c3ccc4c(ccc5c6ccccc6ccc45)c3)nc(-c3cc4ccccc4cc3-c3cccc4oc5ccccc5c34)n2)cc1]

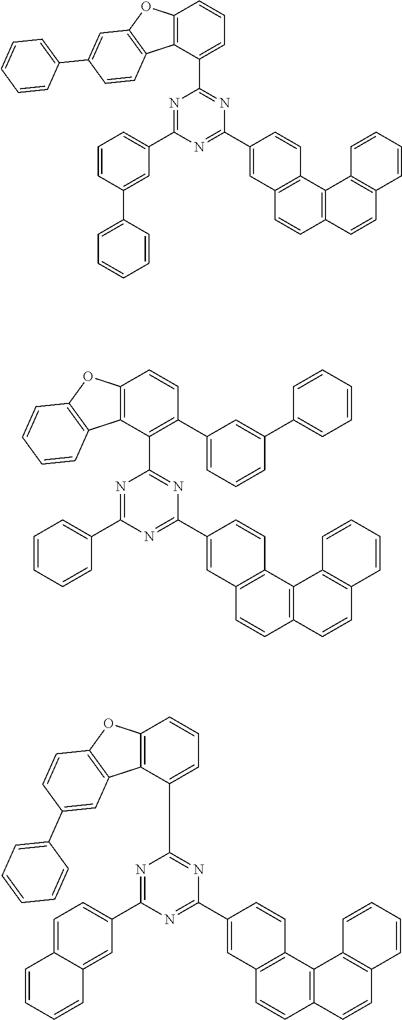 [SMILES: c1ccc(-c2cccc(-c3nc(-c4ccc5c(ccc6ccc7ccccc7c65)c4)nc(-c4cccc5oc6cc(-c7ccccc7)ccc6c45)n3)c2)cc1 ; c1ccc(-c2cccc(-c3ccc4oc5ccccc5c4c3-c3nc(-c4ccccc4)nc(-c4ccc5c(ccc6ccc7ccccc7c65)c4)n3)c2)cc1 ; c1ccc(-c2ccc3oc4cccc(-c5nc(-c6ccc7ccccc7c6)nc(-c6ccc7c(ccc8ccc9ccccc9c87)c6)n5)c4c3c2)cc1]

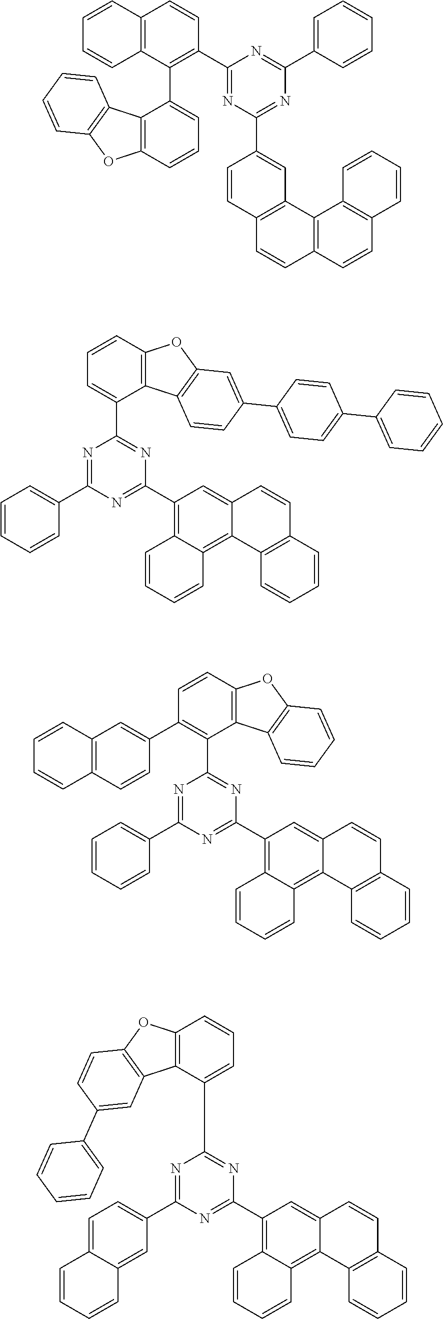 [SMILES: c1ccc(-c2nc(-c3ccc4ccc5ccc6ccccc6c5c4c3)nc(-c3ccc4ccccc4c3-c3cccc4oc5ccccc5c34)n2)cc1 ; c1ccc(-c2ccc(-c3ccc4c(c3)oc3cccc(-c5nc(-c6ccccc6)nc(-c6cc7ccc8ccccc8c7c7ccccc67)n5)c34)cc2)cc1 ; c1ccc(-c2nc(-c3cc4ccc5ccccc5c4c4ccccc34)nc(-c3c(-c4ccc5ccccc5c4)ccc4oc5ccccc5c34)n2)cc1 ; c1ccc(-c2ccc3oc4cccc(-c5nc(-c6ccc7ccccc7c6)nc(-c6cc7ccc8ccccc8c7c7ccccc67)n5)c4c3c2)cc1]

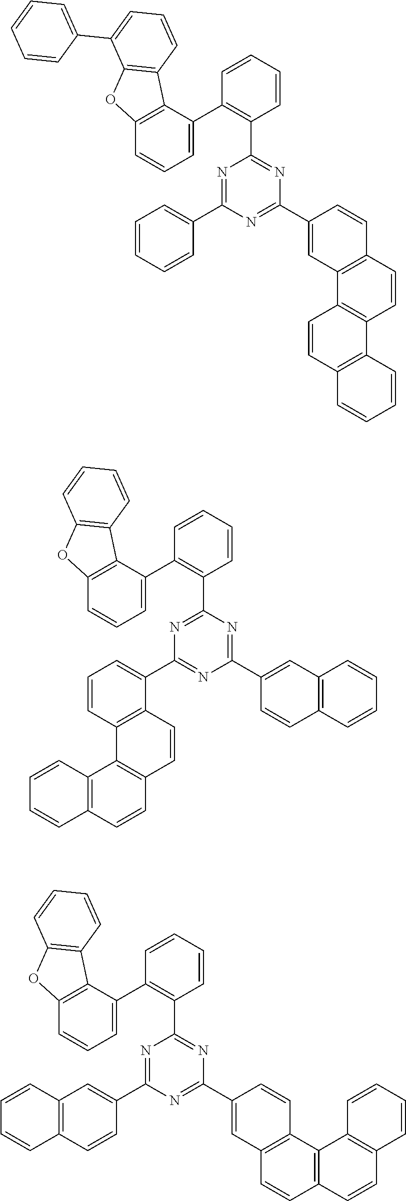 [SMILES: c1ccc(-c2nc(-c3ccc4ccc5c6ccccc6ccc5c4c3)nc(-c3ccccc3-c3cccc4oc5c(-c6ccccc6)cccc5c34)n2)cc1 ; c1ccc(-c2cccc3oc4ccccc4c23)c(-c2nc(-c3ccc4ccccc4c3)nc(-c3cccc4c3ccc3ccc5ccccc5c34)n2)c1 ; c1ccc(-c2cccc3oc4ccccc4c23)c(-c2nc(-c3ccc4ccccc4c3)nc(-c3ccc4c(ccc5ccc6ccccc6c54)c3)n2)c1]

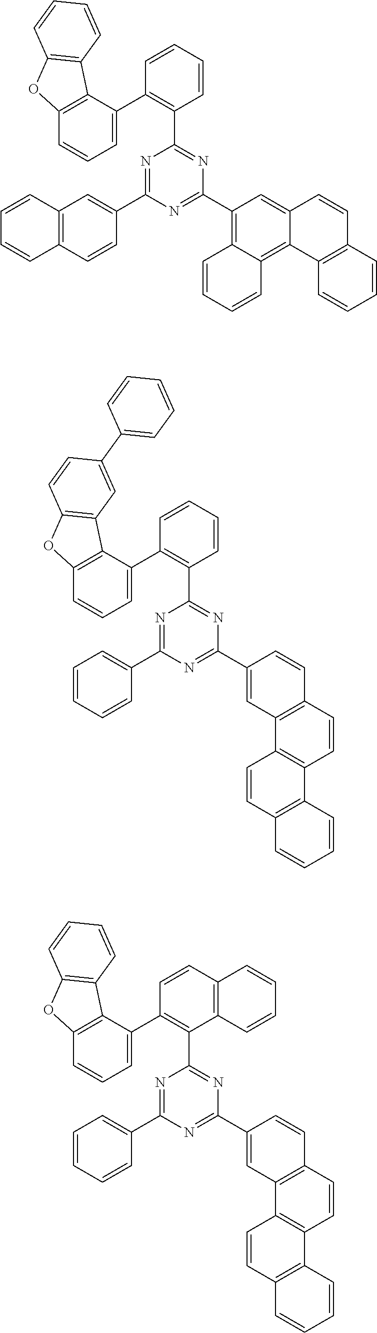 [SMILES: c1ccc(-c2cccc3oc4ccccc4c23)c(-c2nc(-c3ccc4ccccc4c3)nc(-c3cc4ccc5ccccc5c4c4ccccc34)n2)c1 ; c1ccc(-c2ccc3oc4cccc(-c5ccccc5-c5nc(-c6ccccc6)nc(-c6ccc7ccc8c9ccccc9ccc8c7c6)n5)c4c3c2)cc1 ; c1ccc(-c2nc(-c3ccc4ccc5c6ccccc6ccc5c4c3)nc(-c3c(-c4cccc5oc6ccccc6c45)ccc4ccccc34)n2)cc1]

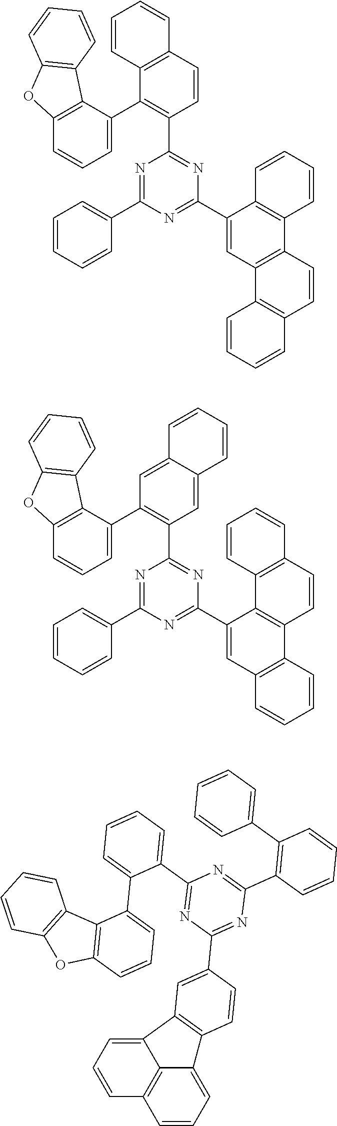 [SMILES: c1ccc(-c2nc(-c3ccc4ccccc4c3-c3cccc4oc5ccccc5c34)nc(-c3cc4c5ccccc5ccc4c4ccccc34)n2)cc1 ; c1ccc(-c2nc(-c3cc4ccccc4cc3-c3cccc4oc5ccccc5c34)nc(-c3cc4ccccc4c4ccc5ccccc5c34)n2)cc1 ; c1ccc(-c2ccccc2-c2nc(-c3ccc4c(c3)-c3cccc5cccc-4c35)nc(-c3ccccc3-c3cccc4oc5ccccc5c34)n2)cc1]

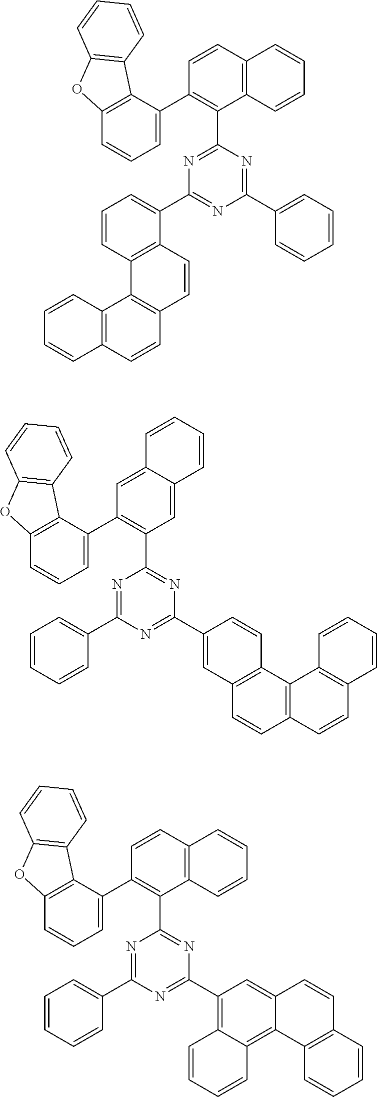 [SMILES: c1ccc(-c2nc(-c3c(-c4cccc5oc6ccccc6c45)ccc4ccccc34)nc(-c3cccc4c3ccc3ccc5ccccc5c34)n2)cc1 ; c1ccc(-c2nc(-c3ccc4c(ccc5ccc6ccccc6c54)c3)nc(-c3cc4ccccc4cc3-c3cccc4oc5ccccc5c34)n2)cc1 ; c1ccc(-c2nc(-c3c(-c4cccc5oc6ccccc6c45)ccc4ccccc34)nc(-c3cc4ccc5ccccc5c4c4ccccc34)n2)cc1]

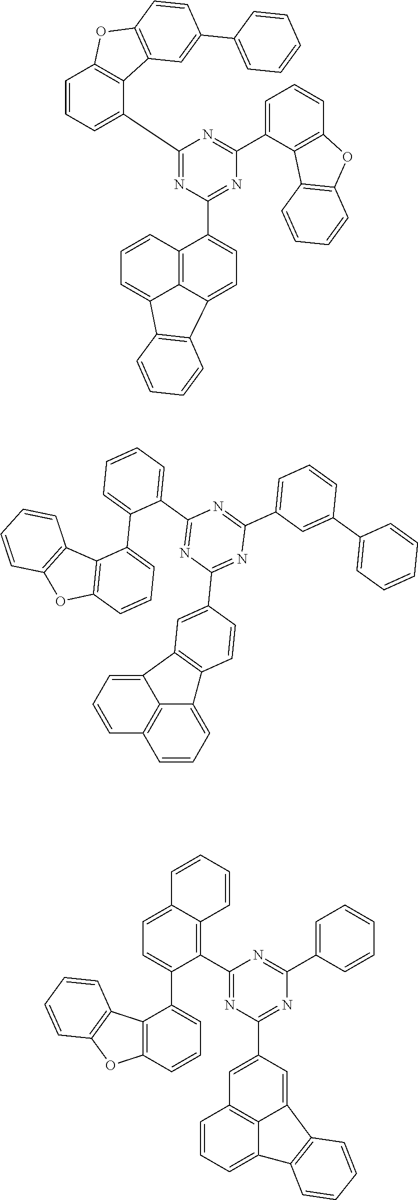 [SMILES: c1ccc(-c2ccc3oc4cccc(-c5nc(-c6ccc7c8c(cccc68)-c6ccccc6-7)nc(-c6cccc7oc8ccccc8c67)n5)c4c3c2)cc1 ; c1ccc(-c2cccc(-c3nc(-c4ccc5c(c4)-c4cccc6cccc-5c46)nc(-c4ccccc4-c4cccc5oc6ccccc6c45)n3)c2)cc1 ; c1ccc(-c2nc(-c3cc4c5c(cccc5c3)-c3ccccc3-4)nc(-c3c(-c4cccc5oc6ccccc6c45)ccc4ccccc34)n2)cc1]

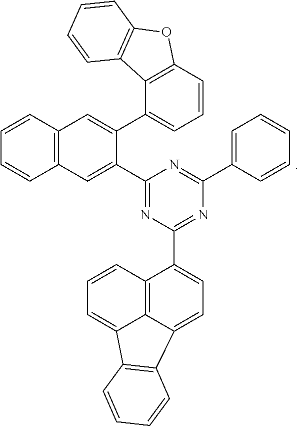 [SMILES: c1ccc(-c2nc(-c3cc4ccccc4cc3-c3cccc4oc5ccccc5c34)nc(-c3ccc4c5c(cccc35)-c3ccccc3-4)n2)cc1]

8. An organic light emitting device comprising:
a first electrode;
a second electrode that is opposite to the first electrode; and
an organic material layer that is between the first electrode and the second electrode, wherein the organic material layer comprises the compound of claim 1.

9. The organic light emitting device of claim 8, wherein:
the organic material layer comprising the compound is an electronic light emitting layer.
